(19) United States
(12) Patent Application Publication
(10) Pub. No.: US 2022/0388990 A1
(43) Pub. Date: Dec. 8, 2022

(54) INHIBITORS OF CYCLIN-DEPENDENT KINASES

(71) Applicant: Kinnate Biopharma Inc., San Diego, CA (US)

(72) Inventors: Toufike KANOUNI, Rancho Santa Fe, CA (US); Lee D. ARNOLD, Rancho Santa Fe, CA (US); Stephen W. KALDOR, San Diego, CA (US); Eric A. MURPHY, San Marcos, CA (US); John TYHONAS, San Diego, CA (US)

(21) Appl. No.: 17/605,111

(22) PCT Filed: Jul. 16, 2020
(86) PCT No.: PCT/US2020/042371

§ 371 Date: Oct. 20, 2021

Related U.S. Application Data
(60) Provisional application No. 62/875,168, filed on Jul. 17, 2019.

(51) Int. Cl. C07D 403/14; C07D 401/14; C07D 471/04; C07D 413/14

(52) U.S. Cl. CPC C07D 403/14; C07D 401/14; C07D 471/04; C07D 413/14

(57) ABSTRACT

Provided herein are compounds which are inhibitors of cyclin-dependent kinases (CDKs), pharmaceutical compositions comprising said compounds, and methods for using said compounds for the treatment of diseases.

### CROSS REFERENCE TO RELATED APPLICATIONS

This application claims the benefit of U.S. Provisional Application No. 62/875,168, filed Jul. 17, 2019, which is incorporated by reference in the disclosure of this application.

### BACKGROUND

Cyclin-dependent kinases (CDKs) are a family of multifunctional enzymes that modify various protein substrates involved in cell cycle progression. Specifically, CDKs phosphorylate their substrates by transferring phosphate groups from ATP to specific stretches of amino acids in the substrates. The deregulation of CDKs is involved in the etiology of many human diseases, including cancers.

### BRIEF SUMMARY OF THE INVENTION

Provided herein are inhibitors of cyclin-dependent kinases (CDKs), pharmaceutical compositions comprising said compounds, and methods for using said compounds for the treatment of diseases.

One embodiment provides a compound, or pharmaceutically acceptable salt or solvate thereof, having the structure of Formula (I):

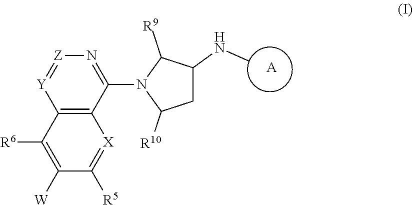

wherein,

Ring A is an optionally substituted hereroaryl selected from pyridine, pyrazine, pyrimidine, quinoline, isoquinoline, quinazoline, pyrazolopyridine, pyrazolopyrimidine, thienopyrimidine, thienopyridine, pyridopyridine, pyridopyrimidine, or triazene;

W is selected from a group having the structure:

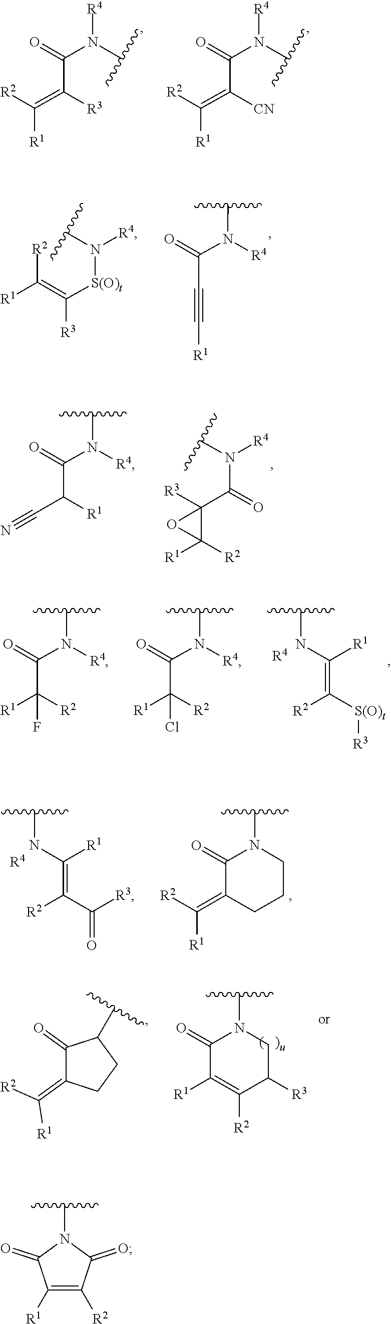

t is 1, or 2; u is 0, 1, or 2;

R¹ , R², and R³ are each independently selected from hydrogen, optionally substituted C1-C4 alkyl, or optional substituted heterocyclyl(alkyl);

R⁴ is hydrogen, or optionally substituted C1-C4 alkyl, or optionally, if R³ is optionally substituted C1-C4 alkyl and R⁴ is optionally substituted C1-C4 alkyl, then R³ and R⁴ together join to form a ring;

R⁵ is selected from hydrogen, —CN, —NH₂, halogen, optionally substituted C1-C4 alkyl, optionally substituted C1-C4 alkoxy, or optionally substituted C1-C4 aminoalkyl;

R⁶ is selected from hydrogen, —CN, —NH₂, halogen, optionally substituted C1-C4 alkyl, optionally substituted C1-C4 alkoxy, or optionally substituted C1-C4 aminoalkyl;

X is N or C—H,

Y is N, or C-L1-R¹¹;

Z is N, or C-L2-R⁷;

L1 and L2 are each independently a bond, —O—, or —N(R⁸)—,

R⁷ is selected from hydrogen, —CN, halogen, optionally substituted C1-C4 alkyl, optionally substituted C3-C7 carbocyclyl, optionally substituted carbocyclyl(alkyl), optionally substituted heterocyclyl, optionally substituted heterocyclyl(alkyl);

R⁸ is hydrogen, or optionally substituted C1-C4 alkyl;

R⁹ is selected from hydrogen, or optionally substituted C1-C4 alkyl;

R¹⁰ is selected from hydrogen, or optionally substituted C1-C4 alkyl; and

R¹¹ is selected from hydrogen, —CN, halogen, —NH_{2,} optionally substituted C1-C4 alkyl, optionally substituted C3-C7 carbocyclyl, optionally substituted carbocyclyl(alkyl), optionally substituted heterocyclyl, or optionally substituted heterocyclyl(alkyl).

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

One embodiment provides a method of treating cancer in a patient in need thereof comprising administering to the patient a pharmaceutical composition comprising a compound of Formula (I), or pharmaceutically acceptable salt or solvate thereof, and at least one pharmaceutically acceptable excipient.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist” of or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO2 radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tent-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)-N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR', —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), −S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)-R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(W)₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)-R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)tOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)-R^{a}, —N(R^{a})₂, —C(O)R^{a}, —C(O)OR^{a}, —C(O)N(R^{a})₂, —N(R^{a})C(O)OR^{a}, —OC(O)-N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aminoalkyl” refers to a —N(alkyl)2 radical, wherein each “alkyl” is independently as defined above, for example, dimethylamino ((CH₃)₂N-), ethyl(methyl)amino (C₂H₅N(CH₃)—, (2-aminoethyl)(methyl)amino (H₂N—CH₂CH₂N(CH₃)—), (2-(dimethylamino)ethyl)(methyl)amino ((CH₃)₂N—CH₂CH₂N(CH₃)—), and the like. In some embodiments, the alkyl part of the aminoalkyl radical is optionally substituted as defined above for an alkyl group.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π—electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R_{c}—C(O)N(R^{a})₂, —R^{b}—N(R^{a})C(O)OR^{a}, N(R^{a})C(O)R^{a}, —R^{b}—N(R^{a})S(O)ₜ R^{a} (where t is 1 or 2), —R^{b}—S(O)ₜR^{a} (where t is 1 or 2), —R^{b}—S(O)ₜOR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂,—R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O-R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

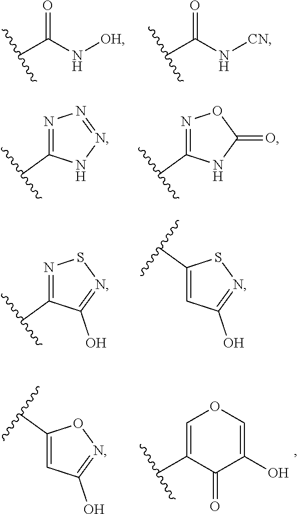

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and RC is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where RC is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π—electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7, 8-tetrahydrobenzo[4, 5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroaryl alkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})_{2,} —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and RC is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

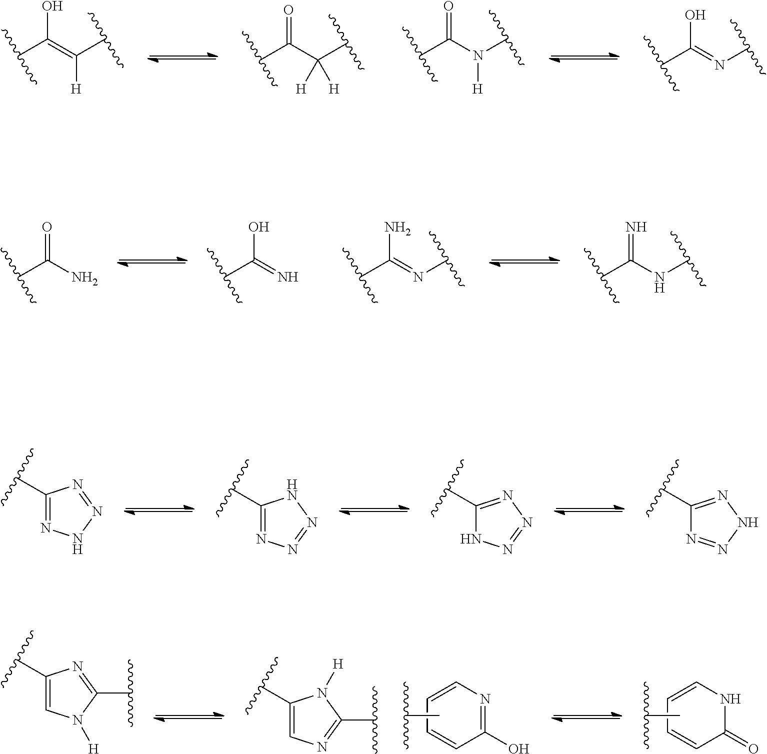

The compounds disclosed herein, in some embodiments, are used in different enriched isotopic forms, e.g., enriched in the content of ²H, ³H, ¹¹C ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14C) Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S. ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

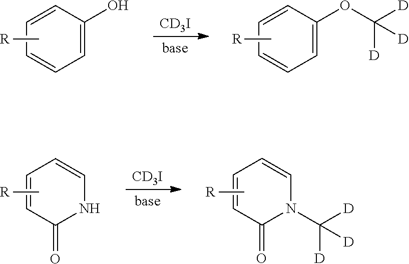

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

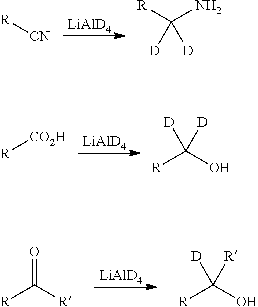

Deuterium gas and palladium catalyst are employed to reduce unsaturated carbon-carbon linkages and to perform a reductive substitution of aryl carbon-halogen bonds as illustrated, by way of example only, in the reaction schemes below.

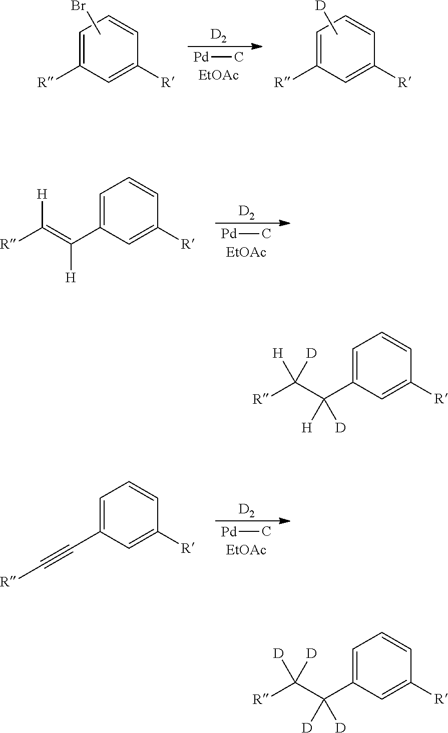

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of cyclin-dependent kinases (CDKs) compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S.M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Cyclin-Dependent Kinases

Cyclin-dependent kinases (CDKs) are a family of serine/threonine protein kinases that are known to function in the processes of cell cycle regulation, metabolism, gene transcription, RNA processing, and DNA repair, with each CDK playing a distinct role (Malumbres, M., 2014, Genome Biol. 15(6), 122-132; Lim et al., 2013, Development 140, 3079-3093). Inhibition of CDKs has long been of therapeutic interest in the treatment of conditions characterized by cellular hyperproliferation, such as cancer, psoriasis, and fungal infections (Coleman, K. G. et al., 1997, Annual Reports in Medicinal Chemistry 32, 171-179).

CDKs are characterized by as being dependent on one or more separate catalytic cyclin subunits in order to carry out specific functions (Malumbres, 2014). Structurally, CDKs comprise a conserved catalytic core containing an ATP-binding pocket, a cyclin binding domain, and an activating T-loop motif (Coleman, 1997; Enke et al., 1999, J. Biol. Chem. 274(4), 1949-1956).

Human cells are known to have at least 20 CDKs and 29 cyclins, which can be grouped into 8 subfamilies (Lim, 2013; Cao et al., 2014, BMC Evol. Biol. 14, 10-26). Therapies known in the art include selective inhibition of specific CDKs.

CDK7 and CDK9 are part of the subfamily of transcriptional CDKs which regulate gene transcription via the phosphorylation of the carboxy-terminal domain of RNA polymerase II (Lucking, U., 2017, ChemMedChem. 12(21), 1776-1793). Inhibitors of CDK7 and CDK9 are recognized in the art as being therapeutically beneficial against various types of cancers.

CDK7 is known to be required for activity-dependent neuronal gene expression, synaptic plasticity, and long-term memory (He et al., 2017, Front. Mol. Neurosci. 10, 365-377). CDK7 inhibition is known to suppress rheumatoid arthritis inflammation via blocking NF-kB activation and IL-1β/IL-6 secretion (Hong et al., 2017, J. Cell. Mol. Med. 22, 1292-1301), and has been shown to disrupt the cell cycle of high-grade glioma (Greenall et al., 2017, Oncogenesis 6(5), e336). The CDK7 inhibitor THZ1 has been shown to significantly affect transcription in T cell leukemia, neuroblastoma, small-cell lung cancer and triple-negative breast cancer cells in vitro (Gao et al., 2017, Cell Chem. Biol. 25, 1-8; Kwiatkowski et al., 2014, Nature 511(7511), 616-620). When screened against a panel of 1,151 cancer cell lines, a THZ1 concentration less than 200 nM exhibited an IC50 in 52% of those lines (Kwiatkowski, 2014, see Table 3a).

CDK9 is known to regulate the expression of antiapoptotic proteins for the survival of cancer cells

(Pang et al., 2017, Cancer Med. 6(10), 2398-2409) and is known to regulate the DNA damage response in complex with cyclin-K (Lim, 2013). Inhibitors of CDK9 have been shown to repress transcription of genes associated with B-cell lymphoma, the most common form of non-Hodgkin lymphoma (Dey et al., 2017, Sci. Rep. 7(1), 18007), hepatocellular carcinoma (Pang, 2017), NUT midline carcinoma (Brägelmann et al., 2017, Cell Rep. 20(12), 2833-2845), ovarian cancer, epithelial carcinoma, colorectal carcinoma, cervical carcinoma, prostate adenocarcinoma, breast adenocarcinoma, and pancreatic carcinoma (Lam et al., 2014, Oncotarget 5, 7691-7704).

CDK12 and CDK13 are transcription-associated CDKs, and are known to regulate RNA polymerase II transcription in complex with cyclin K (Lim, 2013), as well as axonal and transcriptional elongation (Chen et al., 2014, Exp. Neurol. 261, 10-21; Paculová et al., 2017, Cell Div. 12, 7-17).

It has been suggested that CDK12 has oncogenic properties, and is mutated or overexpressed in various types of cancer, leading to dysregulation of cell proliferation (Paculová, 2017). CDK12 inhibitors have been found to reduce gene expression in BRCA cells (Johnson et al., 2016, Cell Rep. 17(9), 2367-2381). Mutations of CDK12 have been shown to disrupt DNA repair, contributing to hyperproliferation and the pathogenesis of breast tumor cells (Tien et al., 2017, Nucleic Acids Res. 45(11), 6698-6716). It is estimated that CDK12 mutations are present in 13% of breast cancers and 5% of ovarian cancers (Tien, 2017; Cerami et al., 2012, Cancer Discov. 2, 401-404; Cancer Genome Atlas Research Network, 2011, Nature, 474, 609-615; Kandoth et al., 2013, Nature 502, 333-339; Cancer Genome Atlas Network, 2012, Nature 490, 61-70).

CDK13 is known to regulate processes associated with growth signaling (Greifenberg et al., 2016,

Cell Rep. 14, 320-331). CDK13 mutations affecting the protein kinase domain have been linked to congenital heart disease, developmental delay and intellectual disability (Hamilton et al., 2017, J. Med. Genet. 55(1), 28-38). CDK13 is known to interact with the splicing factor SRSF1 and regulate alternative splicing of HIV mRNA (Berro et al., 2008, J. Virol. 82, 7155-7166).

CDK inhibitory compounds have been described in the literature. See, for example: Gao et al., 2018, Cell Chem. Biol. 25(2), 135-142; WO 2017/044858; WO 2016/210296; WO 2016/201370; Ficarro et al., 2016, Anal. Chem. 88(24), 12248-12254; WO 2016/160617; Zhang et al., 2016, Nature Chem. Biol. 12(10), 876-884; WO 2016/105528; WO 2015/058126; and WO 2015/058163. Other examples include: WO 2015/124941; Ali et al., 2009, Cancer Res. 69(15), 6208-6215; WO 2016/193939; Bajrami et al., 2014, Cancer Res. 74(1), 287-297; Li et al., 2017, Cancer Res. 77(14), 3834-3845; Cayrol et al., 2017, Nature Commun. 8:14290, 1-11; Johnson et al., 2016, Cell Reports 17(9), 2367-2381; Kalan et al., 2017, Cell Reports 21(2), 467-481; Christensen et al., 2014, Cancer Cell 26(6), 909-922; Iniguez et al., 2018, Cancer Cell 33(2), 202-216; Mertins et al., 2016, Nature 534(7605), 55-62; Nagaraja et al., 2017, Cancer Cell 31(5), 635-652; Naidoo et al., 2017, Mol. Cancer Ther. 17(1), 306-315; Paculova et al., 2017, Cell Div. 12:7, 1-10; and Evan et al., 2017, Clin. Cancer Res. 23(7), 1647-1655.

Based on the role of CDKs in the processes of cell cycle regulation, metabolism, gene transcription, RNA processing, and DNA repair, compounds which alter CDKs activity are considered to be useful in treating or preventing various disorders, including cancer. In some embodiments, described herein is a small molecule inhibitor of cyclin-dependent kinases (CDKs). In some embodiments, described herein is a pharmaceutical composition comprising a small molecule inhibitor of cyclin-dependent kinases (CDKs). In other embodiments, a small molecule inhibitor of cyclin-dependent kinases (CDKs) is used to treat or prevent a disease or condition in a subject in need thereof.

In some embodiments, a heteroaromatic CDK inhibitory compound as described herein is used to treat or prevent cancer in a subject in need thereof. In some embodiments, a pharmaceutical composition comprising a heteroaromatic CDK inhibitory compound as described herein is used to treat or prevent cancer in a subject in need thereof. In some embodiments, disclosed herein is a method of treating cancer comprising administering to a subject in need thereof a therapeutically effective amount of a heteroaromatic CDK inhibitory compound as described herein. In some embodiments, disclosed herein is a method of treating cancer comprising administering to a subject in need thereof a therapeutically effective amount of a pharmaceutical composition comprising a heteroaromatic CDK inhibitory compound as described herein. In some embodiments, disclosed herein is a method of treating cancer comprising administering to a subject having been previously diagnosed with cancer a therapeutically effective amount of a heteroaromatic CDK inhibitory compound as described herein.

In some embodiments, a heteroaromatic CDK inhibitory compound is a heteroaromatic CDK7, CDK9, CDK12, and CDK13 inhibitory compound. In some embodiments, a heteroaromatic CDK inhibitory compound is a heteroaromatic CDK7 inhibitory compound. In some embodiments, a heteroaromatic CDK inhibitory compound is a heteroaromatic CDK9 inhibitory compound. In some embodiments, a heteroaromatic CDK inhibitory compound is a heteroaromatic CDK12 inhibitory compound. In some embodiments, a heteroaromatic CDK inhibitory compound is a heteroaromatic CDK13 inhibitory compound. In some embodiments, a heteroaromatic CDK inhibitory compound is a heteroaromatic CDK7 and CDK9 inhibitory compound. In some embodiments, a heteroaromatic CDK inhibitory compound is a heteroaromatic CDK7 and CDK12 inhibitory compound. In some embodiments, a heteroaromatic CDK inhibitory compound is a heteroaromatic CDK7 and CDK13 inhibitory compound. In some embodiments, a heteroaromatic CDK inhibitory compound is a heteroaromatic CDK9 and CDK12 inhibitory compound. In some embodiments, a heteroaromatic CDK inhibitory compound is a heteroaromatic CDK9 and CDK13 inhibitory compound. In some embodiments, a heteroaromatic CDK inhibitory compound is a heteroaromatic CDK12 and CDK13 inhibitory compound. In some embodiments, a heteroaromatic CDK inhibitory compound is a heteroaromatic CDK7, CDK9, and CDK12 inhibitory compound.

In some embodiments, a heteroaromatic CDK inhibitory compound is a heteroaromatic CDK7,

CDK9, and CDK13 inhibitory compound. In some embodiments, a heteroaromatic CDK inhibitory compound is a heteroaromatic CDK7, CDK12, and CDK13 inhibitory compound. In some embodiments, a heteroaromatic CDK inhibitory compound is a heteroaromatic CDK9, CDK12, and CDK13 inhibitory compound.

### Heteroaromatic CDK Inhibitory Compounds

In one aspect, provided herein is a heteroaromatic CDK inhibitory compound.

One embodiment provides a compound, or pharmaceutically acceptable salt or solvate thereof, having the structure of Formula (I):

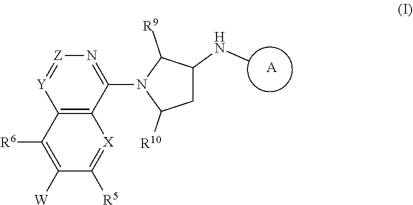

wherein,

Ring A is an optionally substituted hereroaryl selected from pyridine, pyrazine, pyrimidine, quinoline, isoquinoline, quinazoline, pyrazolopyridine, pyrazolopyrimidine, thienopyrimidine, thienopyridine, pyridopyridine, pyridopyrimidine, or triazene;

W is selected from a group having the structure:

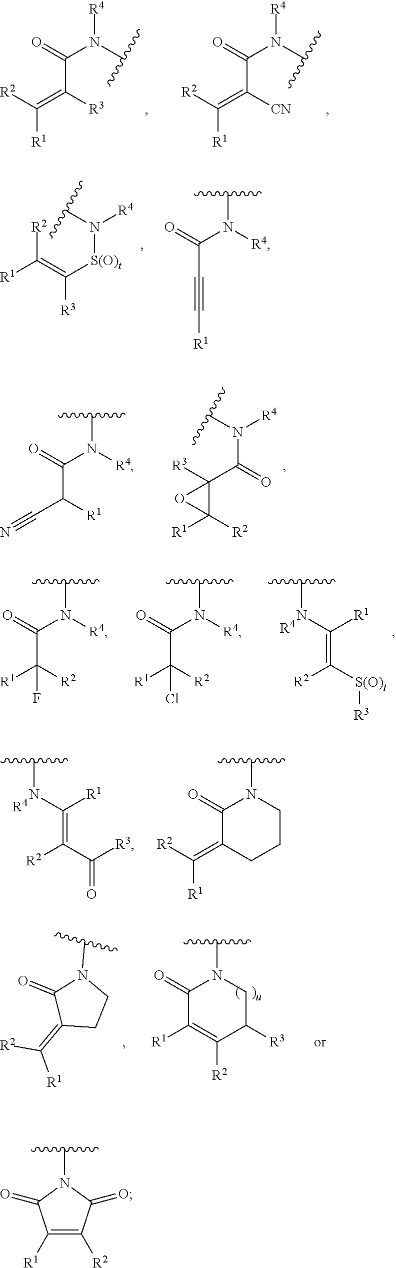

t is 1, or 2; u is 0, 1, or 2;

R′, R², and R³ are each independently selected from hydrogen, optionally substituted C1-C4 alkyl, or optional substituted heterocyclyl(alkyl);

R⁴ is hydrogen, or optionally substituted C1-C4 alkyl, or optionally, if R³ is optionally substituted C1-C4 alkyl and R⁴ is optionally substituted C1-C4 alkyl, then R³ and R⁴ together join to form a ring;

R⁵ is selected from hydrogen, —CN, —NH_{2,} halogen, optionally substituted C1-C4 alkyl, optionally substituted C1-C4 alkoxy, or optionally substituted C1-C4 aminoalkyl;

R⁶ is selected from hydrogen, —CN, —NH_{2,} halogen, optionally substituted C1-C4 alkyl, optionally substituted C1-C4 alkoxy, or optionally substituted C1-C4 aminoalkyl;

X is N or C—H, Y is N, or C-L1-R¹¹; Z is N, or C-L2-R⁷; L1 and L2 are each independently a bond, —O—, or —N(R⁸)—, R⁷ is selected from hydrogen, —CN, halogen, optionally substituted C1-C4 alkyl, optionally substituted C3-C7 carbocyclyl, optionally substituted carbocyclyl(alkyl), optionally substituted heterocyclyl, optionally substituted heterocyclyl(alkyl);

R⁸ is hydrogen, or optionally substituted C1-C4 alkyl;

R⁹ is selected from hydrogen, or optionally substituted C1-C4 alkyl;

R¹⁰ is selected from hydrogen, or optionally substituted C1-C4 alkyl; and

R¹¹ is selected from hydrogen, —CN, halogen, —NH_{2,} optionally substituted C1-C4 alkyl, optionally substituted C3-C7 carbocyclyl, optionally substituted carbocyclyl(alkyl), optionally substituted heterocyclyl, or optionally substituted heterocyclyl(alkyl).

One embodiment provides a compound of Formula (I), or pharmaceutically acceptable salt or solvate thereof, having the structure of Formula (Ia):

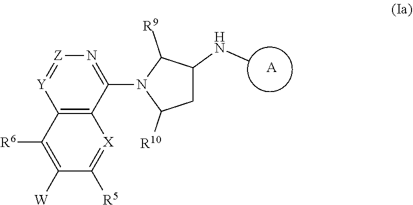

wherein,

Ring A is an optionally substituted hereroaryl selected from pyridine, pyrazine, pyrimidine, quinoline, isoquinoline, quinazoline, pyrazolopyridine, pyrazolopyrimidine, thienopyrimidine, thienopyridine, pyridopyridine, or pyridopyrimidine;

W is selected from a group having the structure:

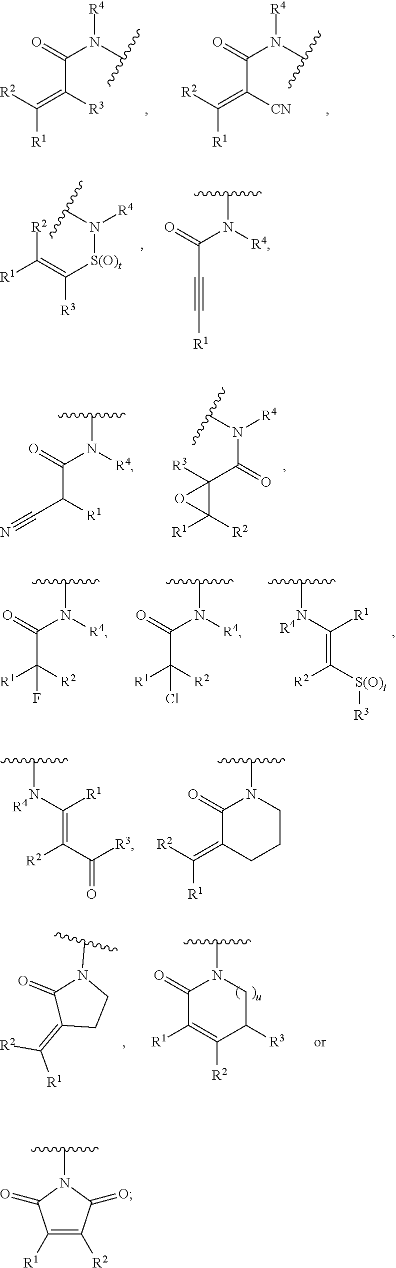

t is 1, or 2; u is 0, 1, or 2;

R′, R², and R³ are each independently selected from hydrogen, optionally substituted C1-C4 alkyl, or optional substituted heterocyclyl(alkyl);

R⁴ is hydrogen, or optionally substituted C1-C4 alkyl, or optionally, if R³ is optionally substituted C1-C4 alkyl and R⁴ is optionally substituted C1-C4 alkyl, then R³ and R⁴ together join to form a ring;

R⁵ is selected from hydrogen, —CN, —NH₂, halogen, optionally substituted C1-C4 alkyl, optionally substituted C1-C4 alkoxy, or optionally substituted Cl-C4 aminoalkyl;

R⁶ is selected from hydrogen, —CN, —NH₂, halogen, optionally substituted C1-C4 alkyl, optionally substituted C1-C4 alkoxy, or optionally substituted C1-C4 aminoalkyl;

X is N or C—H;

Y is N, or C-L1-R¹¹;

Z is N, or C-L2-R⁷;

L1 and L2 are each independently a bond, —O—, or —N(R⁸)—,

R⁷ is selected from hydrogen, —CN, halogen, optionally substituted C1-C4 alkyl, optionally substituted C3-C7 carbocyclyl, optionally substituted carbocyclyl(alkyl), optionally substituted heterocyclyl, optionally substituted heterocyclyl(alkyl);

R⁸ is hydrogen, or optionally substituted C1-C4 alkyl;

R⁹ is selected from hydrogen, or optionally substituted C1-C4 alkyl;

R¹⁰ is selected from hydrogen, or optionally substituted C1-C4 alkyl; and

R¹¹ is selected from hydrogen, —CN, halogen, —NH_{2,} optionally substituted C1-C4 alkyl, optionally substituted C3-C7 carbocyclyl, optionally substituted carbocyclyl(alkyl), optionally substituted heterocyclyl, or optionally substituted heterocyclyl(alkyl).

Another embodiment provides the compound, or pharmaceutically acceptable salt or solvate thereof, of Formula (I) or (Ia), wherein W is selected from the group consisting of:

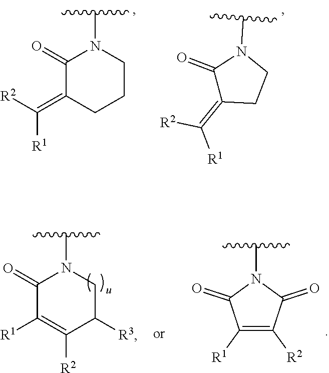

Another embodiment provides the compound, or pharmaceutically acceptable salt or solvate thereof, of Formula (I) or (Ia), wherein W is selected from the group consisting of:

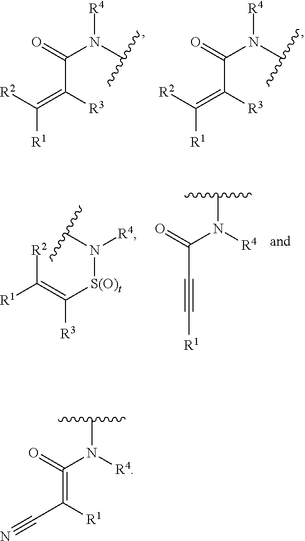

Another embodiment provides the compound, or pharmaceutically acceptable salt or solvate thereof, of Formula (I) or (Ia), wherein W is selected from the group consisting of:

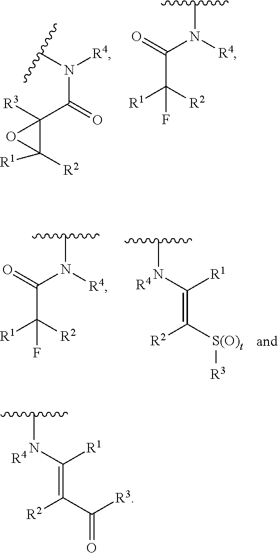

Another embodiment provides the compound, or pharmaceutically acceptable salt or solvate thereof, of Formula (I) or (ia), wherein W is selected from the group consisting of:

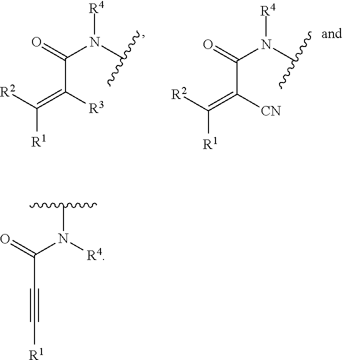

Another embodiment provides the compound, or pharmaceutically acceptable salt or solvate

thereof, of Formula (I) or (Ia), wherein W is:

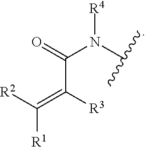

Another embodiment provides the compound, or pharmaceutically acceptable salt or solvate

thereof, of Formula (I) or (Ia), wherein W is:

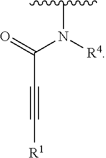

Another embodiment provides the compound, or pharmaceutically acceptable salt or solvate thereof, of Formula (I) or (Ia), wherein R² is hydrogen. Another embodiment provides the compound, or pharmaceutically acceptable salt or solvate thereof, of Formula (I) or (Ia), wherein R³ is hydrogen. Another embodiment provides the compound, or pharmaceutically acceptable salt or solvate thereof, of Formula (I) or (Ia), wherein R² and R³ is hydrogen. Another embodiment provides the compound, or pharmaceutically acceptable salt or solvate thereof, of Formula (I) or (Ia), wherein R¹ is hydrogen. Another embodiment provides the compound, or pharmaceutically acceptable salt or solvate thereof, of Formula (I) or (Ia), wherein R¹ is optionally substituted C1-C4 alkyl. Another embodiment provides the compound, or pharmaceutically acceptable salt or solvate thereof, of Formula (I) or (Ia), wherein R¹ is optionally substituted C1-C2 alkyl. Another embodiment provides the compound, or pharmaceutically acceptable salt or solvate thereof, of Formula (I) or (Ia), wherein le is optionally substituted C1 alkyl. Another embodiment provides the compound, or pharmaceutically acceptable salt or solvate thereof, wherein the Cl alkyl is substituted with an optionally substituted amino group. Another embodiment provides the compound, or pharmaceutically acceptable salt or solvate thereof, wherein the optionally substituted amino group is a dimethylamino. Another embodiment provides the compound, or pharmaceutically acceptable salt or solvate thereof, wherein R¹ is —CH₂—N(Me)₂. Another embodiment provides the compound, or pharmaceutically acceptable salt or solvate thereof, of Formula (I) or (Ia), wherein le is optionally substituted heterocyclylalkyl. Another embodiment provides the compound, or pharmaceutically acceptable salt or solvate thereof, wherein the optionally substituted heterocyclylalkyl comprises an optionally substituted Cl alkyl. Another embodiment provides the compound, or pharmaceutically acceptable salt or solvate thereof, wherein the optionally substituted heterocyclylalkyl comprises an optionally substituted N-linked heterocyclyl. Another embodiment provides the compound, or pharmaceutically acceptable salt or solvate thereof, wherein the optionally substituted N-linked heterocyclyl is an N-linked pyrrolidine or piperidine.

Another embodiment provides the compound, or pharmaceutically acceptable salt or solvate thereof, of Formula (I) or (Ia), wherein R⁴ is hydrogen. Another embodiment provides the compound, or pharmaceutically acceptable salt or solvate thereof, of Formula (I) or (Ia), wherein R⁴ is optionally substituted C1-C4 alkyl.

Another embodiment provides the compound, or pharmaceutically acceptable salt or solvate thereof, of Formula (I) or (Ia), wherein R⁵ is hydrogen. Another embodiment provides the compound, or pharmaceutically acceptable salt or solvate thereof, of Formula (I) or (Ia), wherein R⁶ is hydrogen.

Another embodiment provides the compound, or pharmaceutically acceptable salt or solvate thereof, of Formula (I) or (Ia), wherein X is N. Another embodiment provides the compound, or pharmaceutically acceptable salt or solvate thereof, of Formula (I) or (Ia), wherein X is C—H.

Another embodiment provides the compound, or pharmaceutically acceptable salt or solvate thereof, of Formula (I) or (Ia), wherein Y is N.

Another embodiment provides the compound, or pharmaceutically acceptable salt or solvate thereof, of Formula (I) or (Ia), wherein Y is C-L1-R¹¹. Another embodiment provides the compound, or pharmaceutically acceptable salt or solvate thereof, wherein L1is a bond. Another embodiment provides the compound, or pharmaceutically acceptable salt or solvate thereof, wherein L1is —O—. Another embodiment provides the compound, or pharmaceutically acceptable salt or solvate thereof, wherein L1is —NH—. Another embodiment provides the compound, or pharmaceutically acceptable salt or solvate thereof, wherein L1is —N(R⁸)—, and R⁸ is optionally substituted C1-C4 alkyl.

Another embodiment provides the compound, or pharmaceutically acceptable salt or solvate thereof, of Formula (I) or (Ia), wherein R¹¹ is hydrogen. Another embodiment provides the compound, or pharmaceutically acceptable salt or solvate thereof, of Formula (I), wherein R¹¹ is optionally substituted C1-C4 alkyl. Another embodiment provides the compound, or pharmaceutically acceptable salt or solvate thereof, of Formula (I), wherein R¹¹ is optionally substituted heterocyclyl.

Another embodiment provides the compound, or pharmaceutically acceptable salt or solvate thereof, of Formula (I) or (Ia), wherein Z is N.

Another embodiment provides the compound, or pharmaceutically acceptable salt or solvate thereof, of Formula (I) or (Ia), wherein Z is C-L2-R⁷. Another embodiment provides the compound, or pharmaceutically acceptable salt or solvate thereof, wherein L2 is a bond. Another embodiment provides the compound, or pharmaceutically acceptable salt or solvate thereof, wherein L2 is Another embodiment provides the compound, or pharmaceutically acceptable salt or solvate thereof, wherein L2 is —NH—. Another embodiment provides the compound, or pharmaceutically acceptable salt or solvate thereof, wherein L2 is —N(R⁸)—, and R⁸ is optionally substituted C1-C4 alkyl.

Another embodiment provides the compound, or pharmaceutically acceptable salt or solvate thereof, of Formula (I) or (Ia), wherein R⁷ is hydrogen. Another embodiment provides the compound, or pharmaceutically acceptable salt or solvate thereof, of Formula (I) or (Ia), wherein R⁷ is optionally substituted C1-C4 alkyl. Another embodiment provides the compound, or pharmaceutically acceptable salt or solvate thereof, of Formula (I) or (Ia), wherein R⁷ is optionally substituted heterocyclyl.

Another embodiment provides the compound, or pharmaceutically acceptable salt or solvate thereof, of Formula (I) or (Ia), wherein Ring A is an optionally substituted pyridine.

Another embodiment provides the compound, or pharmaceutically acceptable salt or solvate thereof, of Formula (I) or (Ia), wherein Ring A is an optionally substituted pyrazine.

Another embodiment provides the compound, or pharmaceutically acceptable salt or solvate thereof, of Formula (I) or (Ia), wherein Ring A is an optionally substituted pyrimidine.

Another embodiment provides the compound, or pharmaceutically acceptable salt or solvate thereof, of Formula (I) or (Ia), wherein Ring A is an optionally substituted pyrimidin-2-yl.

Another embodiment provides the compound, or pharmaceutically acceptable salt or solvate thereof, of Formula (I) or (Ia), wherein Ring A is an optionally substituted quinoline.

Another embodiment provides the compound, or pharmaceutically acceptable salt or solvate thereof, of Formula (I or (Ia)), wherein Ring A is an optionally substituted isoquinoline.

Another embodiment provides the compound, or pharmaceutically acceptable salt or solvate thereof, of Formula (I) or (Ia), wherein Ring A is an optionally substituted quinazoline.

Another embodiment provides the compound, or pharmaceutically acceptable salt or solvate thereof, of Formula (I) or (Ia), wherein Ring A is an optionally substituted quinazolin-2-yl.

Another embodiment provides the compound, or pharmaceutically acceptable salt or solvate thereof, of Formula (I) or (Ia), wherein Ring A is an optionally substituted pyrazolopyridine.

Another embodiment provides the compound, or pharmaceutically acceptable salt or solvate thereof, of Formula (I) or (Ia), wherein Ring A is an optionally substituted pyrazolopyrimidine.

Another embodiment provides the compound, or pharmaceutically acceptable salt or solvate thereof, of Formula (I) or (Ia), wherein Ring A is an optionally substituted thienopyrimidine.

Another embodiment provides the compound, or pharmaceutically acceptable salt or solvate thereof, of Formula (I) or (Ia), wherein Ring A is an optionally substituted thieno[3,2-d]pyrimidin-2-yl.

Another embodiment provides the compound, or pharmaceutically acceptable salt or solvate thereof, of Formula (I) or (Ia), wherein Ring A is an optionally substituted thienopyridine.

Another embodiment provides the compound, or pharmaceutically acceptable salt or solvate thereof, of Formula (I) or (Ia), wherein Ring A is an optionally substituted thieno[3,2-d]pyridine.

Another embodiment provides the compound, or pharmaceutically acceptable salt or solvate thereof, of Formula (I) or (Ia), wherein Ring A is an optionally substituted pyridopyridine.

Another embodiment provides the compound, or pharmaceutically acceptable salt or solvate thereof, of Formula (I) or (Ia), wherein Ring A is an optionally substituted pyridopyrimidine.

Another embodiment provides the compound, or pharmaceutically acceptable salt or solvate thereof, of Formula (I) or (Ia), wherein Ring A is an optionally substituted pyrido[3,4-d]pyrimidin-2-yl.

Another embodiment provides the compound, or pharmaceutically acceptable salt or solvate thereof, of Formula (I), wherein Ring A is an optionally substituted triazene.

Another embodiment provides the compound, or pharmaceutically acceptable salt or solvate thereof, of Formula (I) or (Ia), wherein Ring A is optionally substituted with a substituent selected from hydrogen, —CN, halogen, optionally substituted C1-C4 alkyl, optionally substituted C1-C6 alkenyl, optionally substituted C1-C6 alkynyl, optionally substituted C3-C7 carbocyclyl, optionally substituted carbocyclyl(alkyl), optionally substituted heterocyclyl, optionally substituted heterocyclyl(alkyl), optionally substituted C1-C4 alkoxy, optionally substituted C6 aryloxy, —NH₂, —OH, or optionally substituted C1-C4 aminoalkyl.

Another embodiment provides the compound, or pharmaceutically acceptable salt or solvate thereof, of Formula (I) or (Ia), wherein Ring A is selected from:

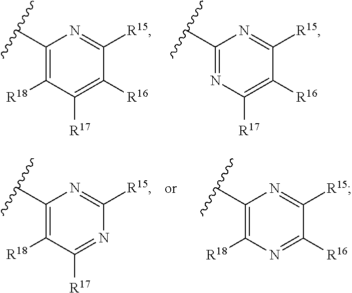

wherein,
R¹⁵ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂,—N(R²²)SO₂N(R²²)₂, —CON(R²²)₂, —N(R²²)CO₂R²¹, —N(R²²)CON(R²²)₂, N(R²²)CON(R²²)₂, —N(R²²)COR²¹, —OC(O)N(R²²)₂, —OSO₂N(R²²)₂, or —N(R²²)SO₃R²¹;
R¹⁶ is selected from hydrogen, halogen, —CN, optionally substituted alkyl, optionally substituted alkenyl, optionally substituted alkynyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²², —N(R22)₂, —SO₂R²¹, —N(R²²)SO₂R²¹, —SO2N(R²²)₂, —N(R²²)SO₂N(R²²)², —CON(R²²)², —N(R²²)CO₂R²¹, —N(R22)CON(R²²)₂, —N(R²²)COR²¹, —OC(O)N(R²²)₂, —OSO₂N(R²²)₂, OSO₂N(R²²)₂, or —N(R²²)SO₂R²¹;
R¹⁷ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R₂₂)₂, —N(R₂₂)CO₂R²¹, —N(R₂₂)CON(R₂₂)₂, —N(R²²)CO₂R²¹, —OC(O)N(R²²)₂, —^{OSO}₂N(R²²)₂, or —N(R²²)SO₃R²¹;
R¹⁸ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R²²)₂, —N(R²²)CO₂R²¹, —N(R²²)CON(R²²)₂, —N(R²²)COR²¹, —OC(O)N(R²²)₂, —OSO₂N(R²²)₂, or —N(R²²)SO₃R²¹;
each R²is independently selected from alkyl, cycloalkyl, cycloalkylalkyl, aryl, aralkyl, heterocyclyl, heterocyclylalkyl, heteroaryl, or heteroarylalkyl; and
each R²² is independently selected from hydrogen, alkyl, cycloalkyl, cycloalkylalkyl, aryl, aralkyl, heterocyclyl, heterocyclylalkyl, heteroaryl, or heteroarylalkyl.

Another embodiment provides the compound, or pharmaceutically acceptable salt or solvate thereof, of Formula (I) or (Ia), wherein Ring A is:

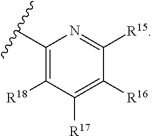

Another embodiment provides the compound, or pharmaceutically acceptable salt or solvate thereof, wherein R¹⁵ is selected from hydrogen, halogen, —CN, and optionally substituted alkyl. Another embodiment provides the compound, or pharmaceutically acceptable salt or solvate thereof, wherein, R¹⁵ is hydrogen. Another embodiment provides the compound, or pharmaceutically acceptable salt or solvate thereof, wherein R¹⁶ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²²)₂, —N(R²²)₂, SO₂R²¹, —N(R²²)SO₂R²¹, —SO₂N(R²²)₂, —N(R²²)SO₂N(R²²)₂, —CON(R²²)₂, —N(R²²)CO₂R²¹, —N(R²²)CON(R²²)₂, —N(R²²)COR²¹, OC(O)N(R²²)₂, —OSO₂N(R²²)₂, or —N(R²²)SO₃R²¹. Another embodiment provides the compound, or pharmaceutically acceptable salt or solvate thereof, wherein R¹⁶ is selected from hydrogen, halogen, —CN, and optionally substituted alkyl. Another embodiment provides the compound, or pharmaceutically acceptable salt or solvate thereof, wherein R¹⁶ is hydrogen. Another embodiment provides the compound, or pharmaceutically acceptable salt or solvate thereof, wherein R¹⁶ is selected from optionally substituted alkyl, optionally substituted alkenyl, or optionally substituted alkynyl. Another embodiment provides the compound, or pharmaceutically acceptable salt or solvate thereof, wherein R¹⁶ is selected from optionally substituted alkynyl.

Another embodiment provides the compound, or pharmaceutically acceptable salt or solvate thereof, of Formula (I) or (Ia), wherein Ring A is:

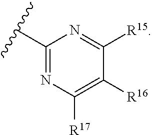

Another embodiment provides the compound, or pharmaceutically acceptable salt or solvate thereof, wherein R¹⁵ is selected from hydrogen, halogen, —CN, and optionally substituted alkyl. Another embodiment provides the compound, or pharmaceutically acceptable salt or solvate thereof, wherein, R¹⁵ is hydrogen. Another embodiment provides the compound, or pharmaceutically acceptable salt or solvate thereof, wherein R¹⁶ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R²²) ₂, —N(R²²) CO₂R²¹, —N(R²²)CON(R²²)₂, —N(R²²)COR²¹. —OC(O)N)R²²)₂, —OSO₂N(R²²)₂, or —N(R²²)SO₃R²¹. Another embodiment provides the compound, or pharmaceutically acceptable salt or solvate thereof, wherein R¹⁶ is selected from hydrogen, halogen, —CN, and optionally substituted alkyl. Another embodiment provides the compound, or pharmaceutically acceptable salt or solvate thereof, wherein R¹⁶ is hydrogen. Another embodiment provides the compound, or pharmaceutically acceptable salt or solvate thereof, wherein R¹⁶ is selected from optionally substituted alkyl, optionally substituted alkenyl, or optionally substituted alkynyl. Another embodiment provides the compound, or pharmaceutically acceptable salt or solvate thereof, wherein R¹⁶ is selected from optionally substituted alkynyl.

Another embodiment provides the compound, or pharmaceutically acceptable salt or solvate

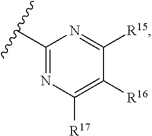

thereof, of Formula (I) or (Ia), wherein Ring A is: and R¹⁶ is not hydrogen.

Another embodiment provides the compound, or pharmaceutically acceptable salt or solvate

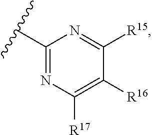

thereof, of Formula (I) or (Ia), wherein Ring A is: and R¹⁶ is halogen.

Another embodiment provides the compound, or pharmaceutically acceptable salt or solvate thereof, of Formula (I) or (Ia), wherein Ring A is:

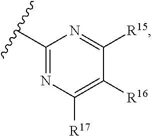

and R¹⁶ is selected from optionally substituted alkyl, optionally substituted alkenyl, or optionally substituted alkynyl.

Another embodiment provides the compound, or pharmaceutically acceptable salt or solvate thereof, of Formula (I) or (Ia), wherein Ring A is:

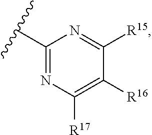

R¹⁵ is hydrogen, R¹⁶ is selected from optionally substituted alkynyl, and R¹⁷ is hydrogen or optionally substituted alkoxy.

Another embodiment provides the compound, or pharmaceutically acceptable salt or solvate thereof, of Formula (I) or (Ia), wherein Ring A is:

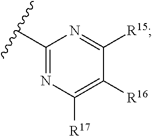

R¹⁵ is hydrogen, R¹⁶ is selected from halogen, —CN, optionally substituted alkyl, optionally substituted fluoroalkyl, optionally substituted alkenyl, optionally substituted alkynyl, and R¹⁷ is hydrogen or optionally substituted alkoxy. Another embodiment provides the compound, or pharmaceutically acceptable salt or solvate thereof, wherein R¹⁷ is hydrogen. Another embodiment provides the compound, or pharmaceutically acceptable salt or solvate thereof, wherein R¹⁵ is selected from hydrogen, halogen, —CN, and optionally substituted alkyl. Another embodiment provides the compound, or pharmaceutically acceptable salt or solvate thereof, wherein R¹⁸ is hydrogen. Another embodiment provides the compound, or pharmaceutically acceptable salt or solvate thereof, wherein R¹⁵ and R¹⁶ are hydrogen. Another embodiment provides the compound, or pharmaceutically acceptable salt or solvate thereof, wherein R¹⁷ and R¹⁸ are hydrogen. Another embodiment provides the compound, or pharmaceutically acceptable salt or solvate thereof, wherein R¹⁵ and R¹⁷ are hydrogen.

In some embodiments, the heteroaromatic CDK inhibitory compound of Formula (I) or (Ia) described herein has a structure provided in Table 1.

TABLE 1

| Synthetic | | |
|---|---|---|
| Chemistry | | |
| Example | Compound Structure | Compound Name |
| 1 | 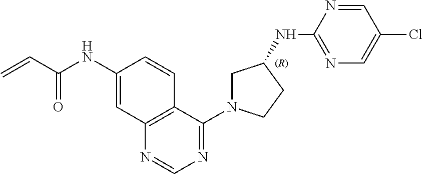 | (R)-N-(4-(3-((5-chloropyrimidin-2- yl)amino)pyrrolidin-1- yl)quinazolin-7- yl)acrylamide |
| 2 | 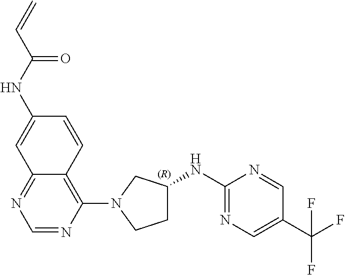 | (R)-N-(4-(3-((5- (trifluoromethyl)pyrimidin-2- yl)amino)pyrrolidin-1- yl)quinazolin-7- yl)acrylamide |
| 3 | 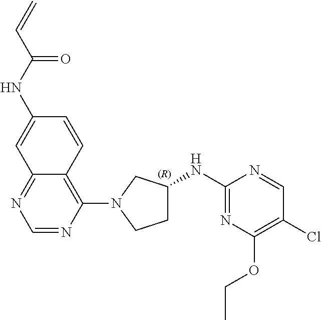 | (R)-N-(4-(3-((5-chloro-4- ethoxypyrimidin-2-yl) amino)pyrrolidin-1- yl)quinazolin-7-yl)acrylamide |
| 4 | 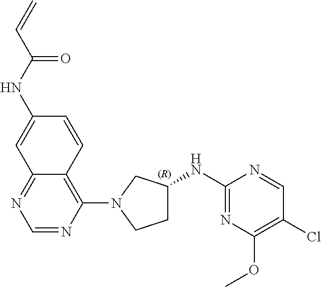 | (R)-N-(4-(3-((5-chloro-4- methoxypyrimidin-2-yl) amino)pyrrolidin- 1-yl)quinazolin-7-yl)acrylamide |
| 5 | 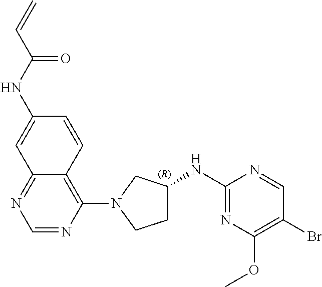 | (R)-N-(4-(3-((5-bromo-4- methoxypyrimidin-2-yl) amino)pyrrolidin- 1-yl)quinazolin-7-yl)acrylamide |
| 6 | 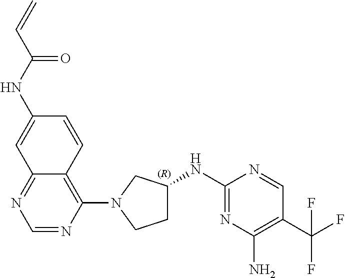 | (R)-N-(4-(3-((4-amino-5- (trifluoromethyl)pyrimidin-2- yl)amino)pyrrolidin-1- yl)quinazolin-7- yl)acrylamide |
| 7 | 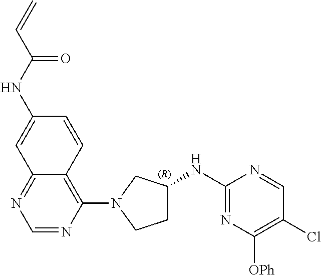 | (R)-N-(4-(3-((5-chloro-4- phenoxypyrimidin-2- yl)amino)pyrrolidin- 1-yl)quinazolin-7-yl)acrylamide |
| 8 | 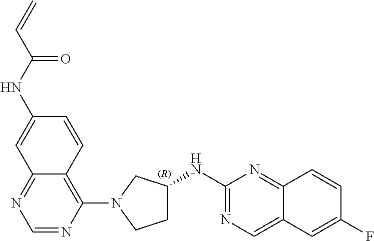 | (R)-N-(4-(3-((6-fluoroquinazolin-2- yl)amino)pyrrolidin-1- yl)quinazolin-7- yl)acrylamide |
| 9 | 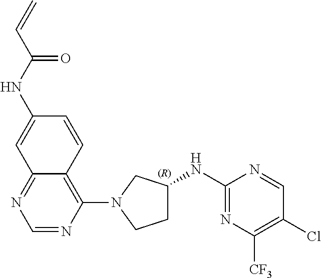 | (R)-N-(4-(3-((5-chloro-4- (trifluoromethyl)pyrimidin-2- yl)amino)pyrrolidin-1- yl)quinazolin-7- yl)acrylamide |
| 10 | 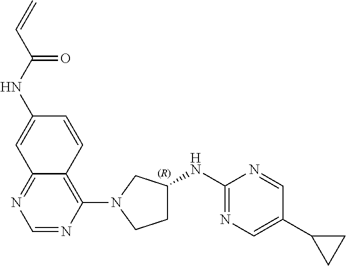 | (R)-N-(4-(3-((5- cyclopropylpyrimidin-2- yl)amino)pyrrolidin-1- yl)quinazolin-7- yl)acrylamide |
| 11 | 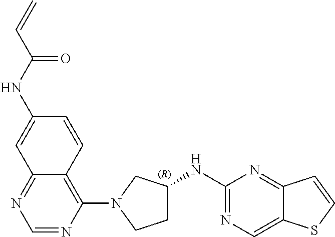 | (R)-N-(4-(3-(thieno [3,2-d]pyrimidin-2- ylamino)pyrrolidin-1- yl)quinazolin-7- yl)acrylamide |
| 12 | 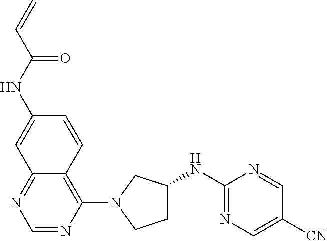 [SMILES: C=CC(=O)Nc1ccc2c(N3CC[C@@H](Nc4ncc(C#N)cn4)C3)ncnc2c1] | (R)-N-(4-(3-((5- cyanopyrimidin-2- yl)amino)pyrrolidin-1- yl)quinazolin-7- yl)acrylamide |
| 13 | 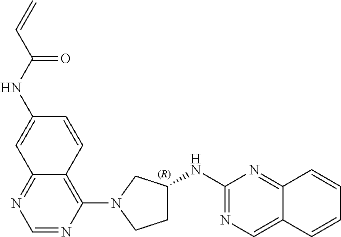 | (R)-N-(4-(3-(quinazolin-2- ylamino)pyrrolidin-1- yl)quinazolin-7- yl)acrylamide |
| 14 | 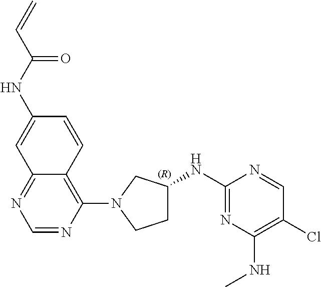 | (R)-N-(4-(3-((5-chloro-4- (methylamino)pyrimidin-2- yl)amino)pyrrolidin-1- yl)quinazolin-7- yl)acrylamide |
| 15 | 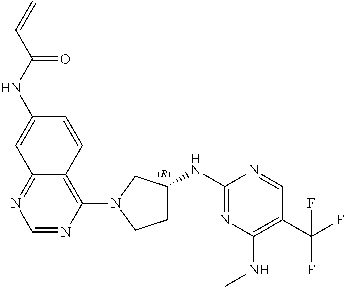 | (R)-N-(4-(3-((4-(methylamino)-5- (trifluoromethyl)pyrimidin-2- yl)amino)pyrrolidin-1- yl)quinazolin-7- yl)acrylamide |
| 16 | 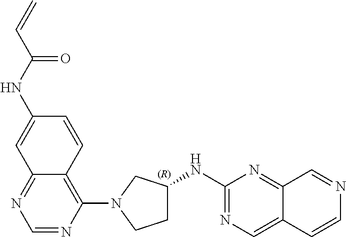 | (R)-N-(4-(3-(pyrido [3,4-d]pyrimidin-2- ylamino)pyrrolidin-1- yl)quinazolin-7- yl)acrylamide |
| 17 | 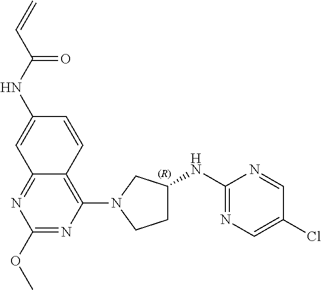 | (R)-N-(4-(3-((5- chloropyrimidin-2- yl)amino)pyrrolidin-1-yl)-2- methoxyquinazolin- 7-yl)acrylamide |
| 18 | 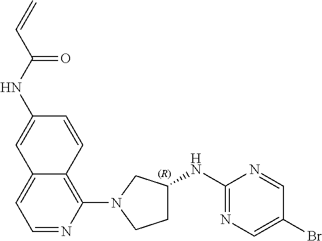 | (R)-N-(1-(3-((5- bromopyrimidin-2- yl)amino)pyrrolidin-1- yl)isoquinolin-6- yl)acrylamide |
| 19 | 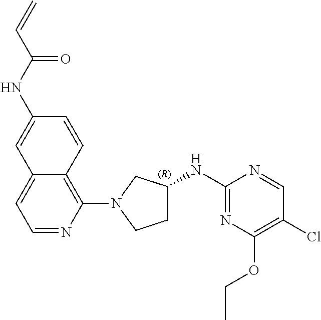 | (R)-N-(1-(3-((5-chloro-4- ethoxypyrimidin-2-yl) amino)pyrrolidin-1- yl)isoquinolin-6-yl)acrylamide |
| 20 | 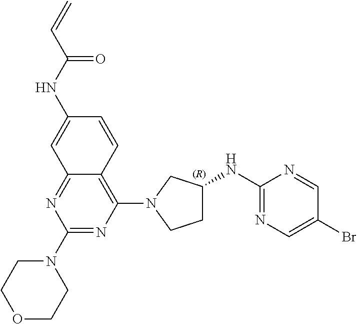 | (R)-N-(4-(3-((5- bromopyrimidin-2- yl)amino)pyrrolidin-1-yl)-2- morpholinoquinazolin- 7-yl)acrylamide |
| 21 | 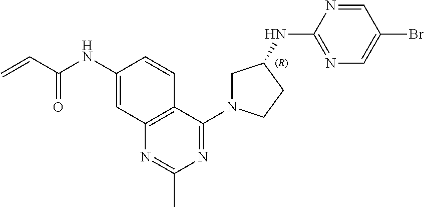 | (R)-N-(4-(3-((5- bromopyrimidin-2- yl)amino)pyrrolidin-1-yl)-2- methylquinazolin- 7-yl)acrylamide |
| 22 | 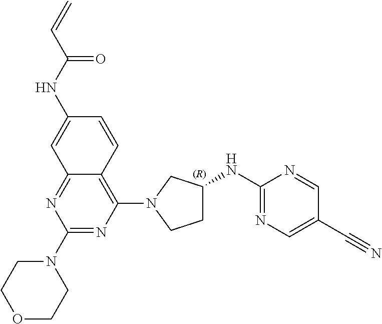 | (R)-N-(4-(3-((5- cyanopyrimidin-2- yl)amino)pyrrolidin-1-yl)-2- morpholinoquinazolin- 7-yl)acrylamide |
| 23 | 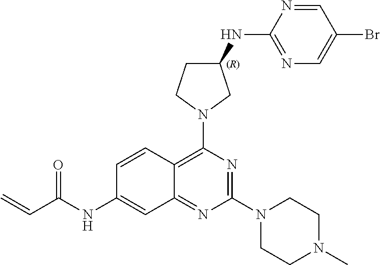 | (R)-N-(4-(3-((5- bromopyrimidin-2- yl)amino)pyrrolidin-1-yl)-2-(4- methylpiperazin-1- yl)quinazolin-7- yl)acrylamide |
| 24 | 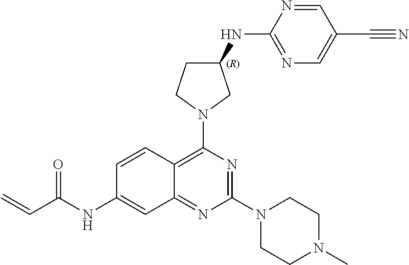 | (R)-N-(4-(3-((5- cyanopyrimidin-2- yl)amino)pyrrolidin-1-yl)-2-(4- methylpiperazin-1- yl)quinazolin-7- yl)acrylamide |
| 25 | 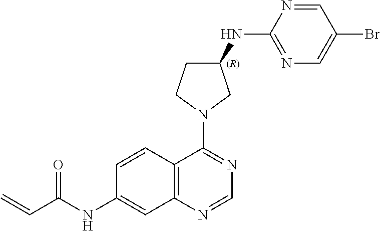 | (R)-N-(4-(3-((5-bromopyrimidin-2- yl)amino)pyrrolidin-1- yl)quinazolin-7- yl)acrylamide |
| 26 | 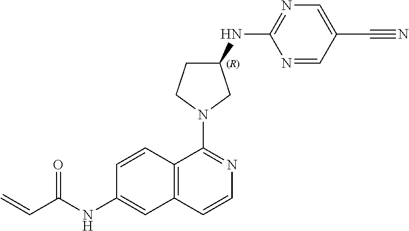 | (R)-N-(1-(3-((5-cyanopyrimidin-2- yl)amino)pyrrolidin-1- yl)isoquinolin-6- yl)acrylamide |
| 27 | 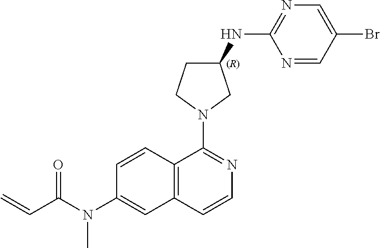 | (R)-N-(1-(3-((5-bromopyrimidin-2- yl)amino)pyrrolidin-1- yl)isoquinolin-6- yl)-N-methylacrylamide |
| 28 | 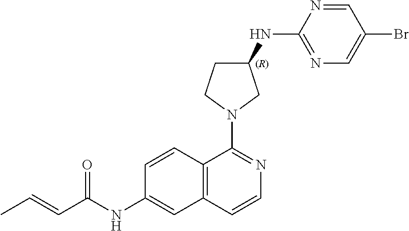 | (R,E)-N-(1-(3-((5- bromopyrimidin-2- yl)amino)pyrrolidin-1- yl)isoquinolin-6- yl)but-2-enamide |
| 29 | 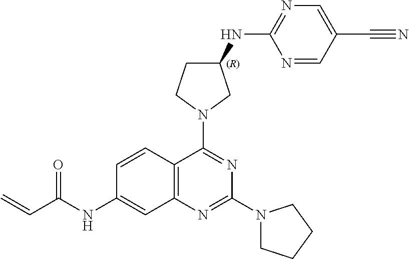 | (R)-N-(4-(3-((5-cyanopyrimidin-2- yl)amino)pyrrolidin-1- yl)-2-(pyrrolidin-1- yl)quinazolin-7-yl)acrylamide |
| 30 | 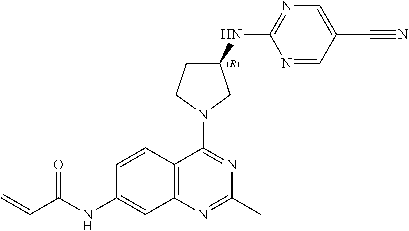 | (R)-N-(4-(3-((5-cyanopyrimidin-2- yl)amino)pyrrolidin-1-yl)-2- methylquinazolin-7- yl)acrylamide |
| 31 | 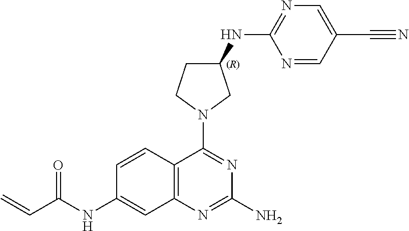 | (R)-N-(2-amino-4-(3- ((5-cyanopyrimidin- 2-yl)amino)pyrrolidin-1- yl)quinazolin-7- yl)acrylamide |
| 32 | 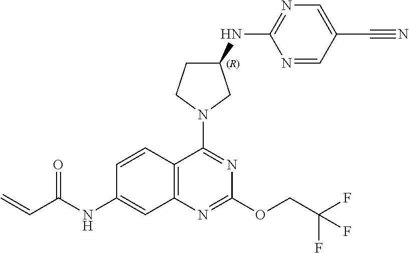 | (R)-N-(4-(3-((5-cyanopyrimidin-2- yl)amino)pyrrolidin-1-yl)-2-(2,2,2- trifluoroethoxy)quinazolin-7- yl)acrylamide |
| 33 | 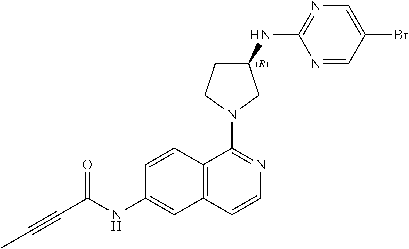 | (R)-N-(1-(3-((5-bromopyrimidin-2- yl)amino)pyrrolidin-1- yl)isoquinolin-6- yl)but-2-ynamide |
| 34 | 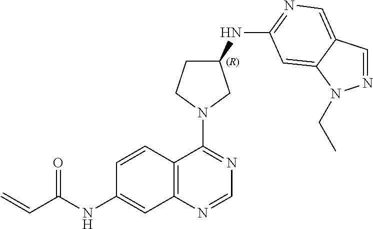 | (R)-N-(4-(3-((1-ethyl- 1H-pyrazolo[4,3- c]pyridin-6-yl)amino) pyrrolidin-1- yl)quinazolin-7-yl)acrylamide |
| 35 | 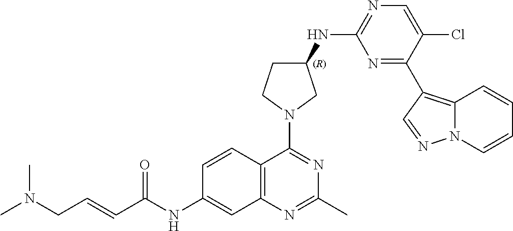 | (R,E)-N-(4-(3-((5-chloro- 4-(pyrazolo[1,5- a]pyridin-3-yl)pyrimidin-2- yl)amino)pyrrolidin-1-yl)-2- methylquinazolin-7-yl)-4- (dimethylamino)but-2-enamide |
| 36 | 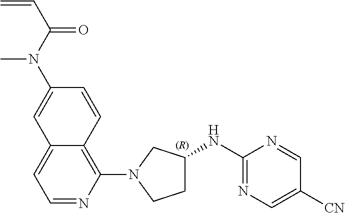 | (R)-N-(1-(3-((5-cyanopyrimidin-2- yl)amino)pyrrolidin-1- yl)isoquinolin-6- yl)-N-methylacrylamide |
| 37 | 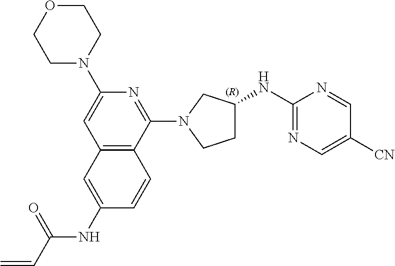 | (R)-N-(1-(3-((5-cyanopyrimidin-2- yl)amino)pyrrolidin-1-yl)-3- morpholinoisoquinolin- 6-yl)acrylamide |
| 38 | 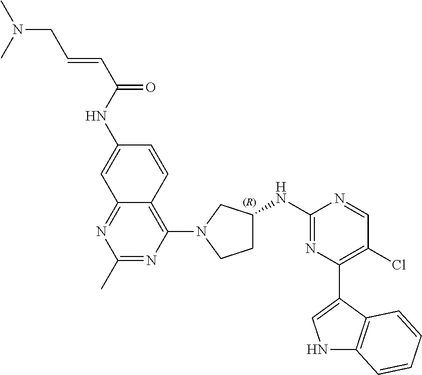 | (R,E)-N-(4-(3-((5-chloro- 4-(1H-indol-3- yl)pyrimidin-2-yl)amino) pyrrolidin-1-yl)- 2-methylquinazolin-7-yl)-4- (dimethylamino)but-2-enamide |
| 39 | 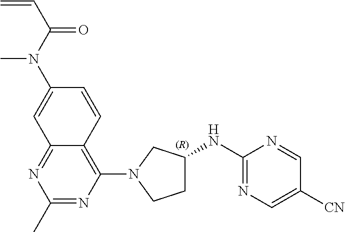 | (R)-N-(4-(3-((5-cyanopyrimidin-2- yl)amino)pyrrolidin-1-yl)-2- methylquinazolin-7-yl)-N- methylacrylamide |
| 40 | 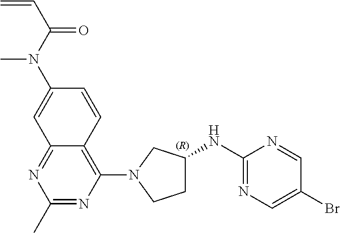 | (R)-N-(4-(3-((5-bromopyrimidin-2- yl)amino)pyrrolidin-1-yl)-2- methylquinazolin-7-yl)-N- methylacrylamide |
| 41 | 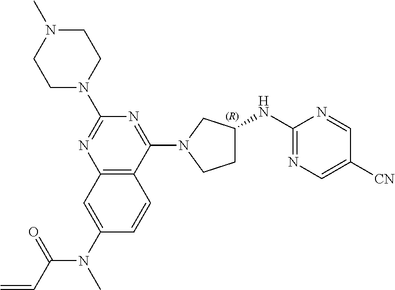 | (R)-N-(4-(3-((5-cyanopyrimidin-2- yl)amino)pyrrolidin-1-yl)-2-(4- methylpiperazin-1-yl) quinazolin-7-yl)-N- methylacrylamide |
| 42 | 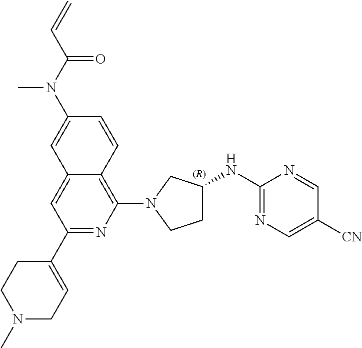 | (R)-N-(1-(3-((5-cyanopyrimidin-2- yl)amino)pyrrolidin-1- yl)-3-(1-methyl- 1,2,3,6-tetrahydropyridin-4- yl)isoquinolin-6-yl)-N- methylacrylamide |
| 43 | 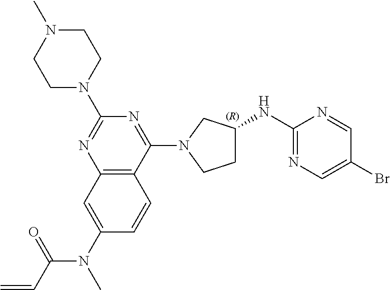 | (R)-N-(4-(3-((5-bromopyrimidin-2- yl)amino)pyrrolidin-1-yl)-2-(4- methylpiperazin-1-yl) quinazolin-7-yl)-N- methylacrylamide |
| 44 | 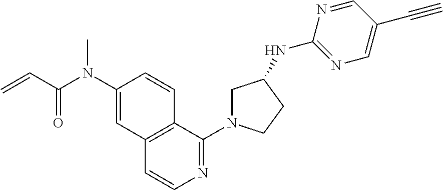 [SMILES: C#Cc1cnc(N[C@@H]2CCN(c3nccc4cc(N(C)C(=O)C=C)ccc34)C2)nc1] | (R)-N-(1-(3-((5- ethynylpyrimidin-2- yl)amino)pyrrolidin-1- yl)isoquinolin-6- yl)-N-methylacrylamide |
| 45 | 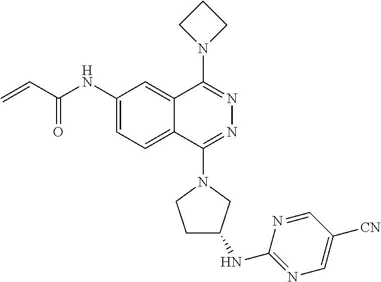 [SMILES: C=CC(=O)Nc1ccc2c(N3CC[C@@H](Nc4ncc(C#N)cn4)C3)nnc(N3CCC3)c2c1] | (R)-N-(4-(azetidin-1-yl)-1-(3-((5- cyanopyrimidin-2-yl) amino)pyrrolidin-1- yl)phthalazin-6-yl)acrylamide |
| 46 | 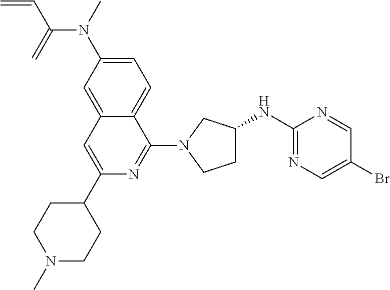 [SMILES: C=CC(=C)N(C)c1ccc2c(N3CC[C@@H](Nc4ncc(Br)cn4)C3)nc(C3CCN(C)CC3)cc2c1] | (R)-N-(1-(3-((5-bromopyrimidin-2- yl)amino)pyrrolidin-1-yl)-3-(1- methylpiperidin-4-yl) isoquinolin-6-yl)-N- methylacrylamide |
| 47 | 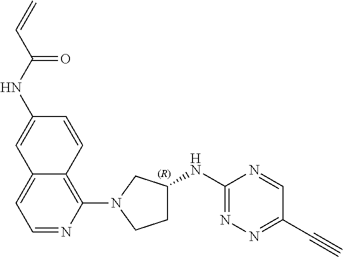 | (R)-N-(1-(3-((6-ethynyl- 1,2,4-triazin-3- yl)amino)pyrrolidin-1- yl)isoquinolin-6- yl)acrylamide |
| 48 | 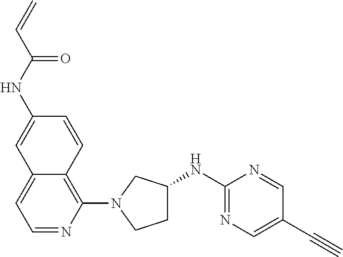 [SMILES: C#Cc1cnc(N[C@@H]2CCN(c3nccc4cc(NC(=O)C=C)ccc34)C2)nc1] | (R)-N-(1-(3-((5- ethynylpyrimidin-2- yl)amino)pyrrolidin-1- yl)isoquinolin-6- yl)acrylamide |
| 49 | 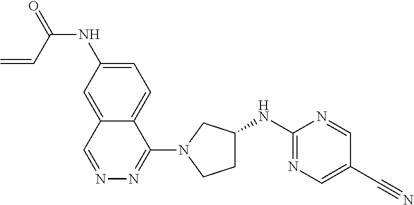 [SMILES: C=CC(=O)Nc1ccc2c(N3CC[C@@H](Nc4ncc(C#N)cn4)C3)nncc2c1] | (R)-N-(1-(3-((5-cyanopyrimidin-2- yl)amino)pyrrolidin-1- yl)phthalazin-6- yl)acrylamide |
| 50 | 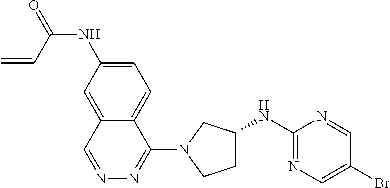 [SMILES: C=CC(=O)Nc1ccc2c(N3CC[C@@H](Nc4ncc(Br)cn4)C3)nncc2c1] | (R)-N-(1-(3-((5-bromopyrimidin-2- yl)amino)pyrrolidin-1- yl)phthalazin-6- yl)acrylamide |
| 51 | 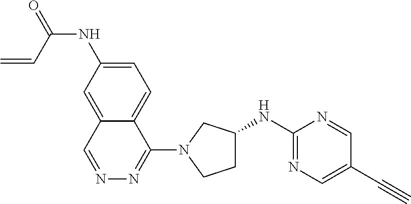 [SMILES: C#Cc1cnc(N[C@@H]2CCN(c3nncc4cc(NC(=O)C=C)ccc34)C2)nc1] | (R)-N-(1-(3-((5-ethynylpyrimidin-2- yl)amino)pyrrolidin-1- yl)phthalazin-6- yl)acrylamide |
| 52 | 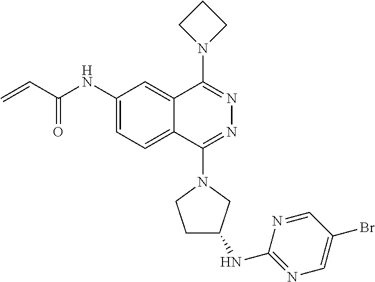 [SMILES: C=CC(=O)Nc1ccc2c(N3CC[C@@H](Nc4ncc(Br)cn4)C3)nnc(N3CCC3)c2c1] | (R)-N-(4-(azetidin-1-yl)-1-(3-((5- bromopyrimidin-2-yl) amino)pyrrolidin-1- yl)phthalazin-6-yl)acrylamide |
| 53 | 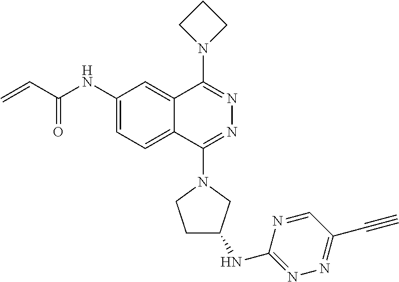 [SMILES: C#Cc1cnc(N[C@@H]2CCN(c3nnc(N4CCC4)c4cc(NC(=O)C=C)ccc34)C2)nn1] | (R)-N-(4-(azetidin-1- yl)-1-(3-((6-ethynyl- 1,2,4-triazin-3-yl) amino)pyrrolidin-1- yl)phthalazin-6-yl)acrylamide |
| 54 | 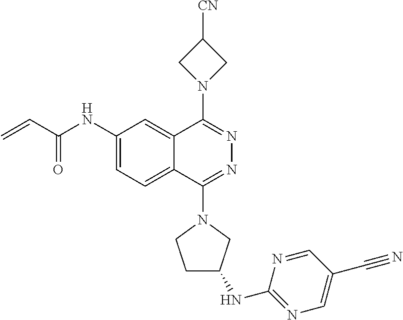 [SMILES: C=CC(=O)Nc1ccc2c(N3CC[C@@H](Nc4ncc(C#N)cn4)C3)nnc(N3CC(C#N)C3)c2c1] | (R)-N-(4-(3-cyanoazetidin- 1-yl)-1-(3-((5- cyanopyrimidin-2-yl) amino)pyrrolidin-1- yl)phthalazin-6-yl)acrylamide |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa. ), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, CN), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. 0. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; OteR^{a}, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the heteroaromatic CDK inhibitory compounds described herein is P. H. Stahl & C. G. Wermuth “Handbook of Pharmaceutical Salts”, Verlag Helvetica Chimica Acta, Zurich, 2002.

### Modification of Cyclin-Dependent Kinase

One embodiment provides a method of inhibiting a CDK enzyme comprising contacting the enzyme with a compound of Formula (I) or (Ia), or a compound disclosed in Table 1. Another embodiment provides the method wherein the CDK enzyme is CDK12.

One embodiment provides a modified CDK12 polypeptide wherein the active site cysteine of an unmodified CDK12 has been modified with a substituent having the structure of Formula (X):

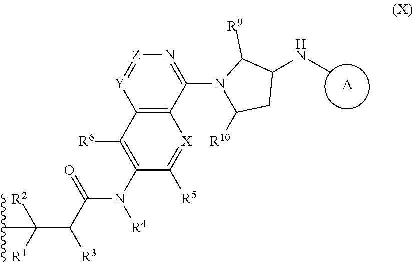

wherein,

Ring A is an optionally substituted 6-membered nitrogen-containing monocyclic heteroaryl, or a 9- or 10-membered nitrogen-containing bicyclic heteroaryl;

R¹ R², and R³ are each independently selected from hydrogen, optionally substituted C1-C4 alkyl, or optional substituted heterocyclyl(alkyl);

R⁴ is hydrogen, or optionally substituted C1-C4 alkyl, or optionally, if R³ is optionally substituted C1-C4 alkyl and R⁴ is optionally substituted C1-C4 alkyl, then R³ and R⁴ together join to form a ring;

R⁵ is selected from hydrogen, —CN, —NH₂, halogen, optionally substituted C1-C4 alkyl, optionally substituted C1-C4 alkoxy, or optionally substituted C1-C4 aminoalkyl;

R⁶ is selected from hydrogen, —CN, —NH₂, halogen, optionally substituted C1-C4 alkyl, optionally substituted C1-C4 alkoxy, or optionally substituted C1-C4 aminoalkyl;

X is N or C—H;

Y is N, or C-L1-R¹¹;

Z is N, or C-L2-R⁷;

L1 and L2 are each independently a bond, —O—, or —N(R⁸)—,

R⁷ is selected from hydrogen, —CN, halogen, optionally substituted C1-C4 alkyl, optionally substituted C3-C7 carbocyclyl, optionally substituted carbocyclyl(alkyl), optionally substituted heterocyclyl, optionally substituted heterocyclyl(alkyl);

R⁸ is hydrogen, or optionally substituted C1-C4 alkyl;

R⁹ is selected from hydrogen, or optionally substituted C1-C4 alkyl;

R¹⁰ is selected from hydrogen, or optionally substituted C1-C4 alkyl; and

R¹¹ is selected from hydrogen, —CN, halogen, —NH₂, optionally substituted C1-C4 alkyl, optionally substituted C3-C7 carbocyclyl, optionally substituted carbocyclyl(alkyl), optionally substituted heterocyclyl, or optionally substituted heterocyclyl(alkyl).

In some embodiments, Formula (X) Ring A is selected from pyridine, pyrazine, pyrimidine, or pyridazine.

In some embodiments, Formula (X) Ring A is selected from quinoline, isoquinoline, quinazoline, pyrazolopyridine, pyrazolopyrimidine, thienopyrimidine, thienopyridine, pyridopyridine, or pyridopyrimidine.

In some embodiments, Formula (X) R² is hydrogen.

In some embodiments, Formula (X) R³ is hydrogen.

In some embodiments, Formula (X) R² and R³ is hydrogen.

In some embodiments, Formula (X) R¹ is hydrogen.

In some embodiments, Formula (X) R¹ is optionally substituted C1-C4 alkyl.

In some embodiments, Formula (X) R¹ is optionally substituted C1-C2 alkyl.

In some embodiments, Formula (X) R¹ is optionally substituted C1 alkyl. In some embodiments the C1 alkyl is substituted with an optionally substituted amino group. In some embodiments the optionally substituted amino group is a dimethylamino.

In some embodiments, Formula (X) R¹is —CH₂—N(Me)₂.

In some embodiments, Formula (X) R¹is optionally substituted heterocyclylalkyl. In some embodiments the optionally substituted heterocyclylalkyl comprises an optionally substituted C1 alkyl. In some embodiments the optionally substituted heterocyclylalkyl comprises an optionally substituted N-linked heterocyclyl. In some embodiments the optionally substituted N-linked heterocyclyl is an N-linked pyrrolidine or piperidine.

In some embodiments, Formula (X) R⁴ is hydrogen.

In some embodiments, Formula (X) R⁴ is optionally substituted C1-C4 alkyl.

In some embodiments, Formula (X) R⁵ is hydrogen.

In some embodiments, Formula (X) R⁶ is hydrogen.

In some embodiments, Formula (X) X is N.

In some embodiments, Formula (X) X is C—H.

In some embodiments, Formula (X) Y is N.

In some embodiments, Formula (X) Y is C-L1-R¹¹.

In some embodiments, Formula (X) L1 is a bond.

In some embodiments, Formula (X) L1 is —O—.

In some embodiments, Formula (X) L1 is —NH—.

In some embodiments, Formula (X) L1 is —N(R⁸)—, and R⁸ is optionally substituted C1-C4 alkyl.

In some embodiments, Formula (X) R¹¹ is hydrogen.

In some embodiments, Formula (X) R¹¹ is optionally substituted C1-C4 alkyl.

In some embodiments, Formula (X) R¹¹ is optionally substituted heterocyclyl.

In some embodiments, Formula (X) Z is N.

In some embodiments, Formula (X) Z is C-L2-R⁷.

In some embodiments, Formula (X) L2 is a bond.

In some embodiments, Formula (X) L2 is —O—.

In some embodiments, Formula (X) L2 is —NH—.

In some embodiments, Formula (X) L2 is —N(R⁸)—, and R⁸ is optionally substituted C1-C4 alkyl.

In some embodiments, Formula (X) R⁷ is hydrogen.

In some embodiments, Formula (X) R⁷ is optionally substituted C1-C4 alkyl.

In some embodiments, Formula (X) R⁷ is optionally substituted heterocyclyl.

Another embodiment provides the modified CDK12 polypeptide wherein the unmodified CDK12 polypeptide is isoform 1 (homo sapiens).

Another embodiment provides the modified CDK12 polypeptide wherein the unmodified CDK12 polypeptide is isoform 2 (homo sapiens).

Another embodiment provides the modified CDK12 polypeptide wherein the unmodified CDK12 polypeptide is an isoform 1 (homo sapiens) variant. Another embodiment provides the modified CDK12 polypeptide wherein the unmodified CDK12 polypeptide is isoform 1 (homo sapiens) variant I1131V. Another embodiment provides the modified CDK12 polypeptide wherein the unmodified CDK12 polypeptide is isoform 1 (homo sapiens) variant L1189Q. Another embodiment provides the modified CDK12 polypeptide wherein the unmodified CDK12 polypeptide is isoform 1 (homo sapiens) variant T1195M.

Another embodiment provides the modified CDK12 polypeptide wherein the unmodified CDK12 polypeptide is a SEQID selected from a SEQID provided in Table 2 or 3.

TABLE 2
CDK12 Sequences

| | Ensembi | |
|---|---|---|
| | Protein ID | |
| SEQ | (Accession | |
| ID NO | Number) | Amino Acid Sequence |
| 1 | ENSP00000398880 | MPNSERHGGKKDGSGGASGTLQPSSGGGSSNSRERHRLVSKHKRHKSKHSKDMG |
| | (NP_057591.2) | LVTPEAASLGTVIKPLVEYDDISSDSDTFSDDMAFKLDRRENDERRGSDRSDRL |
| | | HKHRHHQHRRSRDLLKAKQTEKEKSQEVSSKSGSMKDRISGSSKRSNEETDDYG |
| | | KAQVAKSSSKESRSSKLHKEKTRKERELKSGHKDRSKSHRKRETPKSYKTVDSP |
| | | KRRSRSPHRKWSDSSKQDDSPSGASYGQDYDLSPSRSHTSSNYDSYKKSPGSTS |
| | | RRQSVSPPYKEPSAYQSSTRSPSPYSRRQRSVSPYSRRRSSSYERSGSYSGRSP |
| | | SPYGRRRSSSPFLSKRSLSRSPLPSRKSMKSRSRSPAYSRHSSSHSKKKRSSSR |
| | | SRHSSISPVRLPLNSSLGAELSRKKKERAAAAAAAKMDGKESKGSPVFLPRKEN |
| | | SSVEAKDSGLESKKLPRSVKLEKSAPDTELVNVTHLNTEVKNSSDTGKVKLDEN |
| | | SEKHLVKDLKAQGTRDSKPIALKEEIVTPKETETSEKETPPPLPTIASPPPPLP |
| | | TTTPPPQTPPLPPLPPIPALPQQPPLPPSQPAFSQVPASSTSTLPPSTHSKTSA |
| | | VSSQANSQPPVQVSVKTQVSVTAAIPHLKTSTLPPLPLPPLLPGDDDMDSPKET |
| | | LPSKPVKKEKEQRTRHLLTDLPLPPELPGGDLSPPDSPEPKAITPPQQPYKKRP |
| | | KICCPRYGERRQTESDWGKRCVDKFDIIGIIGEGTYGQVYKAKDKDTGELVALK |
| | | KVRLDNEKEGFPITAIREIKILRQLIHRSVVNMKEIVTDKQDALDFKKDKGAFY |
| | | LVFEYMDHDLMGLLESGLVHFSEDHIKSFMKQLMEGLEYCHKKNFLHRDIKCSN |
| | | ILLNNSGQIKLADFGLARLYNSEESRPYTNKVITLWYRPPELLLGEERYTPAID |
| | | VWSCGCILGELFTKKPIFQANLELAQLELISRLCGSPCPAVWPDVIKLPYFNTM |
| | | KPKKQYRRRLREEFSFIPSAALDLLDHMLTLDPSKRCTAEQTLQSDFLKDVELS |
| | | KMAPPDLPHWQDCHELWSKKRRRQRQSGWVEEPPPSKTSRKETTSGTSTEPVKN |
| | | SSPAPPQPAPGKVESGAGDAIGLADITQQLNQSELAVLLNLLQSQTDLSIPQMA |
| | | QLLNIHSNPEMQQQLEALNQSISALTEATSQQQDSETMAPEESLKEAPSAPVIL |
| | | PSAEQTTLEASSTPADMQNILAVLLSQLMKTQEPAGSLEENNSDKNSGPQGPRR |
| | | TPTMPQEEAAACPPHILPPEKRPPEPPGPPPPPPPPPLVEGDLSSAPQELNPAV |
| | | TAALLQLLSQPEAEPPGHLPHEHQALRPMEYSTRPRPNRTYGNTDGPETGFSAI |
| | | DTDERNSGPALTESLVQTLVKNRTFSGSLSHLGESSSYQGTGSVQFPGDQDLRF |
| | | ARVPLALHPVVGQPFLKAEGSSNSVVHAETKLQNYGELGPGTTGASSSGAGLHW |
| | | GGPTQSSAYGKLYRGPTRVPPRGGRGRGVPY |
| 2 | ENSP00000407720 | MPNSERHGGKKDGSGGASGTLQPSSGGGSSNSRERHRLVSKHKRHKSKHSKDMG |
| | (NP_055898.1) | LVTPEAASLGTVIKPLVEYDDISSDSDTFSDDMAFKLDRRENDERRGSDRSDRL |
| | | HKHRHHQHRRSRDLLKAKQTEKEKSQEVSSKSGSMKDRISGSSKRSNEETDDYG |
| | | KAQVAKSSSKESRSSKLHKEKTRKERELKSGHKDRSKSHRKRETPKSYKTVDSP |
| | | KRRSRSPHRKWSDSSKQDDSPSGASYGQDYDLSPSRSHTSSNYDSYKKSPGSTS |
| | | RRQSVSPPYKEPSAYQSSTRSPSPYSRRQRSVSPYSRRRSSSYERSGSYSGRSP |
| | | SPYGRRRSSSPFLSKRSLSRSPLPSRKSMKSRSRSPAYSRHSSSHSKKKRSSSR |
| | | SRHSSISPVRLPLNSSLGAELSRKKKERAAAAAAAKMDGKESKGSPVFLPRKEN |
| | | SSVEAKDSGLESKKLPRSVKLEKSAPDTELVNVTHLNTEVKNSSDTGKVKLDEN |
| | | SEKHLVKDLKAQGTRDSKPIALKEEIVTPKETETSEKETPPPLPTIASPPPPLP |
| | | TTTPPPQTPPLPPLPPIPALPQQPPLPPSQPAFSQVPASSTSTLPPSTHSKTSA |
| | | VSSQANSQPPVQVSVKTQVSVTAAIPHLKTSTLPPLPLPPLLPGDDDMDSPKET |
| | | LPSKPVKKEKEQRTRHLLTDLPLPPELPGGDLSPPDSPEPKAITPPQQPYKKRP |
| | | KICCPRYGERRQTESDWGKRCVDKFDIIGIIGEGTYGQVYKAKDKDTGELVALK |
| | | KVRLDNEKEGFPITAIREIKILRQLIHRSVVNMKEIVTDKQDALDFKKDKGAFY |
| | | LVFEYMDHDLMGLLESGLVHFSEDHIKSFMKQLMEGLEYCHKKNFLHRDIKCSN |
| | | ILLNNSGQIKLADFGLARLYNSEESRPYTNKVITLWYRPPELLLGEERYTPAID |
| | | VWSCGCILGELFTKKPIFQANLELAQLELISRLCGSPCPAVWPDVIKLPYFNTM |
| | | KPKKQYRRRLREEFSFIPSAALDLLDHMLTLDPSKRCTAEQTLQSDFLKDVELS |
| | | KMAPPDLPHWQDCHELWSKKRRRQRQSGWVEEPPPSKTSRKETTSGTSTEPVKN |
| | | SSPAPPQPAPGKVESGAGDAIGLADITQQLNQSELAVLLNLLQSQTDLSIPQMA |
| | | QLLNIHSNPEMQQQLEALNQSISALTEATSQQQDSETMAPEESLKEAPSAPVIL |
| | | PSAEQTTLEASSTPADMQNILAVLLSQLMKTQEPAGSLEENNSDKNSGPQGPRR |
| | | TPTMPQEEAAEKRPPEPPGPPPPPPPPPLVEGDLSSAPQELNPAVTAALLQLLS |
| | | QPEAEPPGHLPHEHQALRPMEYSTRPRPNRTYGNTDGPETGFSAIDTDERNSGP |
| | | ALTESLVQTLVKNRTFSGSLSHLGESSSYQGTGSVQFPGDQDLRFARVPLALHP |
| | | VVGQPFLKAEGSSNSVVHAETKLQNYGELGPGTTGASSSGAGLHWGGPTQSSAY |
| | | GKLYRGPTRVPPRGGRGRGVPY |
| 3 | ENSP00000464641 | MPNSERHGGKKDGSGGASGTLQPSSGGGSSNSRERHRLVSKHKRHKSKHSKDMG |
| | | LVTPEAASLGTVIKPLVEYDDISSDSDTFSDDMAFKLDRRENDERRGSDRSDRL |
| | | HKHRHHQHRRSRDLLKAKQTEKEKSQEVSSKSGSMKDRISGSSKRSNEETDDYG |
| | | KAQVAKSSSKESRSSKLHKEKTRKERELKSGHKDRSKSHRKRETPKSYKTVDSP |
| | | KRRSRSPHRKWSDSSKQDDSPSGASYGQDYDLSPSRSHTSSNYDSYKKSPGSTS |
| | | RRQSVSPPYKEPSAYQSSTRSPSPYSRRQRSVSPYSRRRSSSYERSGSYSGRSP |
| | | SPYGRRRSSSPFLSKRSLSRSPLPRKSMKSRSRSPAYSRHSSSHSKKKRSSSRS |
| | | RHSSISPVRLPLNSSLGAELSRKKKERAAAAAAAKMDGKESKGSPVFLPRKENS |
| | | SVEAKDSGLESKKLPRSVKLEKSAPDTELVNVTHLNTEVKNSSDTGKVKLDENS |
| | | EKHLVKDLKAQGTRDSKPIALKEEIVTPKETETSEKETPPPLPTIASPPPPLPT |
| | | TTPPPQTPPLPPLPPIPALPQQPPLPPSQPAFSQVPASSTSTLPPSTHSKTSAV |
| | | SSQANSQPPVQVSVKTQVSVTAAIPHLKTSTLPPLPLPPLLPGDDDMDSPKETL |
| | | PSKPVKKEKEQRTRHLLTDLPLPPELPGGDLSPPDSPEPKAITPPQQPYKKRPK |
| | | ICCPRYGERRQTESDWGKRCVDKFDIIGIIGEGTYGQVYKAKDKDTGELVALKK |
| | | VRLDNEKEGFPITAIREIKILRQLIHRSVVNMKEIVTDKQDALDFKKDKGAFYL |
| | | VFEYMDHDLMGLLESGLVHFSEDHIKSFMKQLMEGLEYCHKKNFLHRDIKCSNI |
| | | LLNNSGQIKLADFGLARLYNSEESRPYTNKVITLWYRPPELLLGEERYTPAIDV |
| | | WSCGCILGELFTKKPIFQANLELAQLELISRLCGSPCPAVWPDVIKLPYFNTMK |
| | | PKKQYRRRLREEFSFIPSAALDLLDHMLTLDPSKRCTAEQTLQSDFLKDVELSK |
| | | MAPPDLPHWQDCHELWSKKRRRQRQSGWVEEPPPSKTSRKETTSGTSTEPVKNS |
| | | SPAPPQPAPGKVESGAGDAIGLADITQQLNQSELAVLLNLLQSQTDLSIPQMAQ |
| | | LLNIHSNPEMQQQLEALNQSISALTEATSQQQDSETMAPEESLKEAPSAPVILP |
| | | SAEQTTLEASST |
| 4 | ENSP00000453329 | XADITQQLNQSELAVLLNLLQSQTDLSIPQMAQLLNIHSNPEMQQQLEALNQSI |
| | | SALTEATSQQQDSETMAPEESLKEAPSAPVILPSAEQTTLEASSTPADMQNILA |
| | | VLLSQLMKTQEPAGSLEENNSDKNSGPQGPRRTPTMPQEEAAGRSNGGNAL |

TABLE 3
Variant CDK12 Sequences

| SEQ ID NO | Amino Acid Sequence |
|---|---|
| 5 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPSRKSMKSRSRSPAYSRHSSSHS |
| | KKKRSSSRSRHSSISPVRLPLNSSLGAELSRKKKERAAAAAAAKMDGKESKGSPVFLPRKENSSVEAKDSGLES |
| | KKLPRSVKLEKSAPDTELVNVTHLNTEVKNSSDTGKVKLDENSEKHLVKDLKAQGTRDSKPIALKEEIVTPKET |
| | ETSEKETPPPLPTIASPPPPLPTTTPPPQTPPLPPLPPIPALPQQPPLPPSQPAFSQVPASSTSTLPPSTHSKT |
| | SAVSSQANSQPPVQVSVKTQVSVTAAIPHLKTSTLPPLPLPPLLPGDDDMDSPKETLPSKPVKKEKEQRTRHLL |
| | TDLPLPPELPGGDLSPPDSPEPKAITPPQQPYKKRPKICCPRYGERRQTESDWGKRCVDKFDIIGIIGEGTYGQ |
| | VYKAKDKDTGELVALKKVRLDNEKEGFPITAIREIKILRQLIHRSVVNMKEIVTDKQDALDFKKDKGAFYLVFE |
| | YMDHDLMGLLESGLVHFSEDHIKSFMKQLMEGLEYCHKKNFLHRDIKCSNILLNNSGQIKLADFGLARLYNSEE |
| | SRPYTNKVITLWYRPPELLLGEERYTPAIDVWSCGCILGELFTKKPIFQANLELAQLELISRLCGSPCPAVWPD |
| | VIKLPYFNTMKPKKQYRRRLREEFSFIPSAALDLLDHMLTLDPSKRCTAEQTLQSDFLKDVELSKMAPPDLPHW |
| | QDCHELWSKKRRRQRQSGVVVEEPPPSKTSRKETTSGTSTEPVKNSSPAPPQPAPGKVESGAGDAIGLADITQQ |
| | LNQSELAVLLNLLQSQTDLSVPQMAQLLNIHSNPEMQQQLEALNQSISALTEATSQQQDSETMAPEESLKEAPS |
| | APVILPSAEQTTLEASSTPADMQNILAVLLSQLMKTQEPAGSLEENNSDKNSGPQGPRRTPTMPQEEAAACPPH |
| | ILPPEKRPPEPPGPPPPPPPPPLVEGDLSSAPQELNPAVTAALLQLLSQPEAEPPGHLPHEHQALRPMEYSTRP |
| | RPNRTYGNTDGPETGFSAIDTDERNSGPALTESLVQTLVKNRTFSGSLSHLGESSSYQGTGSVQFPGDQDLRFA |
| | RVPLALHPVVGQPFLKAEGSSNSVVHAETKLQNYGELGPGTTGASSSGAGLHWGGPTQSSAYGKLYRGPTRVPP |
| | RGGRGRGVPY |
| 6 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPSRKSMKSRSRSPAYSRHSSSHS |
| | KKKRSSSRSRHSSISPVRLPLNSSLGAELSRKKKERAAAAAAAKMDGKESKGSPVFLPRKENSSVEAKDSGLES |
| | KKLPRSVKLEKSAPDTELVNVTHLNTEVKNSSDTGKVKLDENSEKHLVKDLKAQGTRDSKPIALKEEIVTPKET |
| | ETSEKETPPPLPTIASPPPPLPTTTPPPQTPPLPPLPPIPALPQQPPLPPSQPAFSQVPASSTSTLPPSTHSKT |
| | SAVSSQANSQPPVQVSVKTQVSVTAAIPHLKTSTLPPLPLPPLLPGDDDMDSPKETLPSKPVKKEKEQRTRHLL |
| | TDLPLPPELPGGDLSPPDSPEPKAITPPQQPYKKRPKICCPRYGERRQTESDWGKRCVDKFDIIGIIGEGTYGQ |
| | VYKAKDKDTGELVALKKVRLDNEKEGFPITAIREIKILRQLIHRSVVNMKEIVTDKQDALDFKKDKGAFYLVFE |
| | YMDHDLMGLLESGLVHFSEDHIKSFMKQLMEGLEYCHKKNFLHRDIKCSNILLNNSGQIKLADFGLARLYNSEE |
| | SRPYTNKVITLWYRPPELLLGEERYTPAIDVWSCGCILGELFTKKPIFQANLELAQLELISRLCGSPCPAVWPD |
| | VIKLPYFNTMKPKKQYRRRLREEFSFIPSAALDLLDHMLTLDPSKRCTAEQTLQSDFLKDVELSKMAPPDLPHW |
| | QDCHELWSKKRRRQRQSGVVVEEPPPSKTSRKETTSGTSTEPVKNSSPAPPQPAPGKVESGAGDAIGLADITQQ |
| | LNQSELAVLLNLLQSQTDLSIPQMAQLLNIHSNPEMQQQLEALNQSISALTEATSQQQDSETMAPEESLKEAPS |
| | APVIQPSAEQTTLEASSTPADMQNILAVLLSQLMKTQEPAGSLEENNSDKNSGPQGPRRTPTMPQEEAAACPPH |
| | ILPPEKRPPEPPGPPPPPPPPPLVEGDLSSAPQELNPAVTAALLQLLSQPEAEPPGHLPHEHQALRPMEYSTRP |
| | RPNRTYGNTDGPETGFSAIDTDERNSGPALTESLVQTLVKNRTFSGSLSHLGESSSYQGTGSVQFPGDQDLRFA |
| | RVPLALHPVVGQPFLKAEGSSNSVVHAETKLQNYGELGPGTTGASSSGAGLHWGGPTQSSAYGKLYRGPTRVPP |
| | RGGRGRGVPY |
| 7 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPSRKSMKSRSRSPAYSRHSSSHS |
| | KKKRSSSRSRHSSISPVRLPLNSSLGAELSRKKKERAAAAAAAKMDGKESKGSPVFLPRKENSSVEAKDSGLES |
| | KKLPRSVKLEKSAPDTELVNVTHLNTEVKNSSDTGKVKLDENSEKHLVKDLKAQGTRDSKPIALKEEIVTPKET |
| | ETSEKETPPPLPTIASPPPPLPTTTPPPQTPPLPPLPPIPALPQQPPLPPSQPAFSQVPASSTSTLPPSTHSKT |
| | SAVSSQANSQPPVQVSVKTQVSVTAAIPHLKTSTLPPLPLPPLLPGDDDMDSPKETLPSKPVKKEKEQRTRHLL |
| | TDLPLPPELPGGDLSPPDSPEPKAITPPQQPYKKRPKICCPRYGERRQIESDWGKRCVDKFDIIGIIGEGTYGQ |
| | VYKAKDKDTGELVALKKVRLDNEKEGFPITAIREIKILRQLIHRSVVNMKEIVTDKQDALDFKKDKGAFYLVFE |
| | YMDHDLMGLLESGLVHFSEDHIKSFMKQLMEGLEYCHKKNFLHRDIKCSNILLNNSGQIKLADFGLARLYNSEE |
| | SRPYTNKVITLWYRPPELLLGEERYTPAIDVWSCGCILGELFTKKPIFQANLELAQLELISRLCGSPCPAVWPD |
| | VIKLPYFNTMKPKKQYRRRLREEFSFIPSAALDLLDHMLTLDPSKRCTAEQTLQSDFLKDVELSKMAPPDLPHW |
| | QDCHELWSKKRRRQRQSGVVVEEPPPSKTSRKETTSGTSTEPVKNSSPAPPQPAPGKVESGAGDAIGLADITQQ |
| | LNQSELAVLLNLLQSQTDLSIPQMAQLLNIHSNPEMQQQLEALNQSISALTEATSQQQDSETMAPEESLKEAPS |
| | APVILPSAEQMTLEASSTPADMQNILAVLLSQLMKTQEPAGSLEENNSDKNSGPQGPRRTPTMPQEEAAACPPH |
| | ILPPEKRPPEPPGPPPPPPPPPLVEGDLSSAPQELNPAVTAALLQLLSQPEAEPPGHLPHEHQALRPMEYSTRP |
| | RPNRTYGNTDGPETGFSAIDTDERNSGPALTESLVQTLVKNRTFSGSLSHLGESSSYQGTGSVQFPGDQDLRFA |
| | RVPLALHPVVGQPFLKAEGSSNSVVHAETKLQNYGELGPGTTGASSSGAGLHWGGPTQSSAYGKLYRGPTRVPP |
| | RGGRGRGVPY |
| 8 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPSRKSMKSRSRSPAYSRHSSSHS |
| | KKKRSSSRSRHSSISPVRLPLNSSLGAELSRKKKERAAAAAAAKMDGKESKGSPVFLPRKENSSVEAKDSGLES |
| | KKLPRSVKLEKSAPDTELVNVTHLNTEVKNSSDTGKVKLDENSEKHLVKDLKAQGTRDSKPIALKEEIVTPKET |
| | ETSEKETPPPLPTIASPPPPLPTTTPPPQTPPLPPLPPIPALPQQPPLPPSQPAFSQVPASSTSTLPPSTHSKT |
| | SAVSSQANSQPPVQVSVKTQVSVTAAIPHLKTSTLPPLPLPPLLPGDDDMDSPKETLPSKPVKKEKEQRTRHLL |
| | TDLPLPPELPGGDLSPPDSPEPKAITPPQQPYKKRPKICCPRYGERRQIESDWGKRCVDKFDIIGIIGEGTYGQ |
| | VYKAKDKDTGELVALKKVRLDNEKEGFPITAIREIKILRQLIHRSVVNMKEIVTDKQDALDFKKDKGAFYLVFE |
| | YMDHDLMGLLESGLVHFSEDHIKSFMKQLMEGLEYCHKKNFLHRDIKCSNILLNNSGQIKLADFGLARLYNSEE |
| | SRPYTNKVITLWYRPPELLLGEERYTPAIDVWSCGCILGELFTKKPIFQANLELAQLELISRLCGSPCPAVWPD |
| | VIKLPYFNTMKPKKQYRRRLREEFSFIPSAALDLLDHMLTLDPSKRCTAEQTLQSDFLKDVELSKMAPPDLPHW |
| | QDCHELWSKKRRRQRQSGVVVEEPPPSKTSRKETTSGTSTEPVKNSSPAPPQPAPGKVESGAGDAIGLADITQQ |
| | LNQSELAVLLNLLQSQTDLSVPQMAQLLNIHSNPEMQQQLEALNQSISALTEATSQQQDSETMAPEESLKEAPS |
| | APVILPSAEQTTLEASSTPADMQNILAVLLSQLMKTQEPAGSLEENNSDKNSGPQGPRRTPTMPQEEAAEKRPP |
| | EPPGPPPPPPPPPLVEGDLSSAPQELNPAVTAALLQLLSQPEAEPPGHLPHEHQALRPMEYSTRPRPNRTYGNT |
| | DGPETGFSAIDTDERNSGPALTESLVQTLVKNRTFSGSLSHLGESSSYQGTGSVQFPGDQDLRFARVPLALHPV |
| | VGQPFLKAEGSSNSVVHAETKLQNYGELGPGTTGASSSGAGLHWGGPTQSSAYGKLYRGPTRVPPRGGRGRGVP |
| | Y |
| 9 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPSRKSMKSRSRSPAYSRHSSSHS |
| | KKKRSSSRSRHSSISPVRLPLNSSLGAELSRKKKERAAAAAAAKMDGKESKGSPVFLPRKENSSVEAKDSGLES |
| | KKLPRSVKLEKSAPDTELVNVTHLNTEVKNSSDTGKVKLDENSEKHLVKDLKAQGTRDSKPIALKEEIVTPKET |
| | ETSEKETPPPLPTIASPPPPLPTTTPPPQTPPLPPLPPIPALPQQPPLPPSQPAFSQVPASSTSTLPPSTHSKT |
| | SAVSSQANSQPPVQVSVKTQVSVTAAIPHLKTSTLPPLPLPPLLPGDDDMDSPKETLPSKPVKKEKEQRTRHLL |
| | TDLPLPPELPGGDLSPPDSPEPKAITPPQQPYKKRPKICCPRYGERRQTESDWGKRCVDKFDIIGIIGEGTYGQ |
| | VYKAKDKDTGELVALKKVRLDNEKEGFPITAIREIKILRQLIHRSVVNMKEIVTDKQDALDFKKDKGAFYLVFE |
| | YMDHDLMGLLESGLVHFSEDHIKSFMKQLMEGLEYCHKKNFLHRDIKCSNILLNNSGQIKLADFGLARLYNSEE |
| | SRPYTNKVITLWYRPPELLLGEERYTPAIDVWSCGCILGELFTKKPIFQANLELAQLELISRLCGSPCPAVWPD |
| | VIKLPYFNTMKPKKQYRRRLREEFSFIPSAALDLLDHMLTLDPSKRCTAEQTLQSDFLKDVELSKMAPPDLPHW |
| | QDCHELWSKKRRRQRQSGVVVEEPPPSKTSRKETTSGTSTEPVKNSSPAPPQPAPGKVESGAGDAIGLADITQQ |
| | LNQSELAVLLNLLQSQTDLSIPQMAQLLNIHSNPEMQQQLEALNQSISALTEATSQQQDSETMAPEESLKEAPS |
| | APVIQPSAEQTTLEASSTPADMQNILAVLLSQLMKTQEPAGSLEENNSDKNSGPQGPRRTPTMPQEEAAEKRPP |
| | EPPGPPPPPPPPPLVEGDLSSAPQELNPAVTAALLQLLSQPEAEPPGHLPHEHQALRPMEYSTRPRPNRTYGNT |
| | DGPETGFSAIDTDERNSGPALTESLVQTLVKNRTFSGSLSHLGESSSYQGTGSVQFPGDQDLRFARVPLALHPV |
| | VGQPFLKAEGSSNSVVHAETKLQNYGELGPGTTGASSSGAGLHWGGPTQSSAYGKLYRGPTRVPPRGGRGRGVP |
| | Y |
| 10 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPSRKSMKSRSRSPAYSRHSSSHS |
| | KKKRSSSRSRHSSISPVRLPLNSSLGAELSRKKKERAAAAAAAKMDGKESKGSPVFLPRKENSSVEAKDSGLES |
| | KKLPRSVKLEKSAPDTELVNVTHLNTEVKNSSDTGKVKLDENSEKHLVKDLKAQGTRDSKPIALKEEIVTPKET |
| | ETSEKETPPPLPTIASPPPPLPTTTPPPQTPPLPPLPPIPALPQQPPLPPSQPAFSQVPASSTSTLPPSTHSKT |
| | SAVSSQANSQPPVQVSVKTQVSVTAAIPHLKTSTLPPLPLPPLLPGDDDMDSPKETLPSKPVKKEKEQRTRHLL |
| | TDLPLPPELPGGDLSPPDSPEPKAITPPQQPYKKRPKICCPRYGERRQTESDWGKRCVDKFDIIGIIGEGTYGQ |
| | VYKAKDKDTGELVALKKVRLDNEKEGFPITAIREIKILRQLIHRSVVNMKEIVTDKQDALDFKKDKGAFYLVFE |
| | YMDHDLMGLLESGLVHFSEDHIKSFMKQLMEGLEYCHKKNFLHRDIKCSNILLNNSGQIKLADFGLARLYNSEE |
| | SRPYTNKVITLWYRPPELLLGEERYTPAIDVWSCGCILGELFTKKPIFQANLELAQLELISRLCGSPCPAVWPD |
| | VIKLPYFNTMKPKKQYRRRLREEFSFIPSAALDLLDHMLTLDPSKRCTAEQTLQSDFLKDVELSKMAPPDLPHW |
| | QDCHELWSKKRRRQRQSGVVVEEPPPSKTSRKETTSGTSTEPVKNSSPAPPQPAPGKVESGAGDAIGLADITQQ |
| | LNQSELAVLLNLLQSQTDLSIPQMAQLLNIHSNPEMQQQLEALNQSISALTEATSQQQDSETMAPEESLKEAPS |
| | APVILPSAEQMTLEASSTPADMQNILAVLLSQLMKTQEPAGSLEENNSDKNSGPQGPRRTPTMPQEEAAEKRPP |
| | EPPGPPPPPPPPPLVEGDLSSAPQELNPAVTAALLQLLSQPEAEPPGHLPHEHQALRPMEYSTRPRPNRTYGNT |
| | DGPETGFSAIDTDERNSGPALTESLVQTLVKNRTFSGSLSHLGESSSYQGTGSVQFPGDQDLRFARVPLALHPV |
| | VGQPFLKAEGSSNSVVHAETKLQNYGELGPGTTGASSSGAGLHWGGPTQSSAYGKLYRGPTRVPPRGGRGRGVP |
| | Y |
| 11 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPRKSMKSRSRSPAYSRHSSSHSK |
| | KKRSSSRSRHSSISPVRLPLNSSLGAELSRKKKERAAAAAAAKMDGKESKGSPVFLPRKENSSVEAKDSGLESK |
| | KLPRSVKLEKSAPDTELVNVTHLNTEVKNSSDTGKVKLDENSEKHLVKDLKAQGTRDSKPIALKEEIVTPKETE |
| | TSEKETPPPLPTIASPPPPLPTTTPPPQTPPLPPLPPIPALPQQPPLPPSQPAFSQVPASSTSTLPPSTHSKTS |
| | AVSSQANSQPPVQVSVKTQVSVTAAIPHLKTSTLPPLPLPPLLPGDDDMDSPKETLPSKPVKKEKEQRTRHLLT |
| | DLPLPPELPGGDLSPPDSPEPKAITPPQQPYKKRPKICCPRYGERRQTESDWGKRCVDKFDIIGIIGEGTYGQV |
| | YKAKDKDTGELVALKKVRLDNEKEGFPITAIREIKILRQLIHRSVVNMKEIVTDKQDALDFKKDKGAFYLVFEY |
| | MDHDLMGLLESGLVHFSEDHIKSFMKQLMEGLEYCHKKNFLHRDIKCSNILLNNSGQIKLADFGLARLYNSEES |
| | RPYTNKVITLWYRPPELLLGEERYTPAIDVWSCGCILGELFTKKPIFQANLELAQLELISRLCGSPCPAVWPDV |
| | IKLPYFNTMKPKKQYRRRLREEFSFIPSAALDLLDHMLTLDPSKRCTAEQTLQSDFLKDVELSKMAPPDLPHWQ |
| | DCHELWSKKRRRQRQSGVVVEEPPPSKTSRKETTSGTSTEPVKNSSPAPPQPAPGKVESGAGDAIGLADITQQL |
| | NQSELAVLLNLLQSQTDLSVPQMAQLLNIHSNPEMQQQLEALNQSISALTEATSQQQDSETMAPEESLKEAPSA |
| | PVILPSAEQTTLEASST |
| 12 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPRKSMKSRSRSPAYSRHSSSHSK |
| | KKRSSSRSRHSSISPVRLPLNSSLGAELSRKKKERAAAAAAAKMDGKESKGSPVFLPRKENSSVEAKDSGLESK |
| | KLPRSVKLEKSAPDTELVNVTHLNTEVKNSSDTGKVKLDENSEKHLVKDLKAQGTRDSKPIALKEEIVTPKETE |
| | TSEKETPPPLPTIASPPPPLPTTTPPPQTPPLPPLPPIPALPQQPPLPPSQPAFSQVPASSTSTLPPSTHSKTS |
| | AVSSQANSQPPVQVSVKTQVSVTAAIPHLKTSTLPPLPLPPLLPGDDDMDSPKETLPSKPVKKEKEQRTRHLLT |
| | DLPLPPELPGGDLSPPDSPEPKAITPPQQPYKKRPKICCPRYGERRQTESDWGKRCVDKFDIIGIIGEGTYGQV |
| | YKAKDKDTGELVALKKVRLDNEKEGFPITAIREIKILRQLIHRSVVNMKEIVTDKQDALDFKKDKGAFYLVFEY |
| | MDHDLMGLLESGLVHFSEDHIKSFMKQLMEGLEYCHKKNFLHRDIKCSNILLNNSGQIKLADFGLARLYNSEES |
| | RPYTNKVITLWYRPPELLLGEERYTPAIDVWSCGCILGELFTKKPIFQANLELAQLELISRLCGSPCPAVWPDV |
| | IKLPYFNTMKPKKQYRRRLREEFSFIPSAALDLLDHMLTLDPSKRCTAEQTLQSDFLKDVELSKMAPPDLPHWQ |
| | DCHELWSKKRRRQRQSGVVVEEPPPSKTSRKETTSGTSTEPVKNSSPAPPQPAPGKVESGAGDAIGLADITQQL |
| | NQSELAVLLNLLQSQTDLSIPQMAQLLNIHSNPEMQQQLEALNQSISALTEATSQQQDSETMAPEESLKEAPSA |
| | PVIQPSAEQTTLEASST |
| 13 | MPNSERHGGKKDGSGGASGTLQPSSGGGSSNSRERHRLVSKHKRHKSKHSKDMGLVTPEAASLGTVIKPLVEYD |
| | DISSDSDTFSDDMAFKLDRRENDERRGSDRSDRLHKHRHHQHRRSRDLLKAKQTEKEKSQEVSSKSGSMKDRIS |
| | GSSKRSNEETDDYGKAQVAKSSSKESRSSKLHKEKTRKERELKSGHKDRSKSHRKRETPKSYKTVDSPKRRSRS |
| | PHRKWSDSSKQDDSPSGASYGQDYDLSPSRSHTSSNYDSYKKSPGSTSRRQSVSPPYKEPSAYQSSTRSPSPYS |
| | RRQRSVSPYSRRRSSSYERSGSYSGRSPSPYGRRRSSSPFLSKRSLSRSPLPRKSMKSRSRSPAYSRHSSSHSK |
| | KKRSSSRSRHSSISPVRLPLNSSLGAELSRKKKERAAAAAAAKMDGKESKGSPVFLPRKENSSVEAKDSGLESK |
| | KLPRSVKLEKSAPDTELVNVTHLNTEVKNSSDTGKVKLDENSEKHLVKDLKAQGTRDSKPIALKEEIVTPKETE |
| | TSEKETPPPLPTIASPPPPLPTTTPPPQTPPLPPLPPIPALPQQPPLPPSQPAFSQVPASSTSTLPPSTHSKTS |
| | AVSSQANSQPPVQVSVKTQVSVTAAIPHLKTSTLPPLPLPPLLPGDDDMDSPKETLPSKPVKKEKEQRTRHLLT |
| | DLPLPPELPGGDLSPPDSPEPKAITPPQQPYKKRPKICCPRYGERRQTESDWGKRCVDKFDIIGIIGEGTYGQV |
| | YKAKDKDTGELVALKKVRLDNEKEGFPITAIREIKILRQLIHRSVVNMKEIVTDKQDALDFKKDKGAFYLVFEY |
| | MDHDLMGLLESGLVHFSEDHIKSFMKQLMEGLEYCHKKNFLHRDIKCSNILLNNSGQIKLADFGLARLYNSEES |
| | RPYTNKVITLWYRPPELLLGEERYTPAIDVWSCGCILGELFTKKPIFQANLELAQLELISRLCGSPCPAVWPDV |
| | IKLPYFNTMKPKKQYRRRLREEFSFIPSAALDLLDHMLTLDPSKRCTAEQTLQSDFLKDVELSKMAPPDLPHWQ |
| | DCHELWSKKRRRQRQSGVVVEEPPPSKTSRKETTSGTSTEPVKNSSPAPPQPAPGKVESGAGDAIGLADITQQL |
| | NQSELAVLLNLLQSQTDLSIPQMAQLLNIHSNPEMQQQLEALNQSISALTEATSQQQDSETMAPEESLKEAPSA |
| | PVILPSAEQMTLEASST |
| 14 | XADITQQLNQSELAVLLNLLQSQTDLSVPQMAQLLNIHSNPEMQQQLEALNQSISALTEATSQQQDSETMAPEE |
| | SLKEAPSAPVILPSAEQTTLEASSTPADMQNILAVLLSQLMKTQEPAGSLEENNSDKNSGPQGPRRTPTMPQEE |
| | AAGRSNGGNAL |
| 15 | XADITQQLNQSELAVLLNLLQSQTDLSIPQMAQLLNIHSNPEMQQQLEALNQSISALTEATSQQQDSETMAPEE |
| | SLKEAPSAPVIQPSAEQTTLEASSTPADMQNILAVLLSQLMKTQEPAGSLEENNSDKNSGPQGPRRTPTMPQEE |
| | AAGRSNGGNAL |
| 16 | XADITQQLNQSELAVLLNLLQSQTDLSIPQMAQLLNIHSNPEMQQQLEALNQSISALTEATSQQQDSETMAPEE |
| | SLKEAPSAPVILPSAEQMTLEASSTPADMQNILAVLLSQLMKTQEPAGSLEENNSDKNSGPQGPRRTPTMPQEE |
| | AAGRSNGGNAL |

### Pharmaceutical Compositions

In certain embodiments, the heteroaromatic CDK inhibitory compound described herein is administered as a pure chemical. In other embodiments, the heteroaromatic CDK inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one heteroaromatic CDK inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I) or (Ia), or a compound disclosed in Table 1, or a pharmaceutically acceptable salt or solvate thereof.

One embodiment provides a method of preparing a pharmaceutical composition comprising mixing a compound of Formula (I) or (Ia), or a pharmaceutically acceptable salt or solvate thereof, and a pharmaceutically acceptable carrier.

In certain embodiments, the heteroaromatic CDK inhibitory compound as described by Formula (I) or (Ia), or a compound disclosed in Table 1, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

In some embodiments, the heteroaromatic CDK inhibitory compound as described by Formula (I) or (Ia), or a compound disclosed in Table 1, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heteroaromatic CDK inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I) or (Ia), or a compound disclosed in Table 1, or a pharmaceutically acceptable salt or solvate thereof, for use in a method of treatment of the human or animal body.

One embodiment provides a compound of Formula (I) or (Ia), or a compound disclosed in Table 1, or a pharmaceutically acceptable salt or solvate thereof, for use in a method of treatment of cancer, neoplastic disease, or hyperproliferative disorder.

One embodiment provides a use of a compound of Formula (I) or (Ia), or a compound disclosed in Table 1, or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I) or (Ia), or a pharmaceutically acceptable salt or solvate thereof. In some embodiments, described herein is a method of treating cancer in a patient in need thereof comprising administering to the patient a compound disclosed in Table 1, or a pharmaceutically acceptable salt or solvate thereof. In some embodiments, also described herein is a method of treating cancer in a patient in need thereof comprising administering to the patient a pharmaceutical composition comprising a compound of Formula (I) or (Ia), or a pharmaceutically acceptable salt or solvate thereof, and a pharmaceutically acceptable excipient. In some embodiments, also described herein is a method of treating cancer in a patient in need thereof comprising administering to the patient a pharmaceutical composition comprising a compound disclosed in Table 1, or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### EXAMPLES

### 1. Chemical Synthesis

In some embodiments, the heteroaromatic CDK inhibitory compounds disclosed herein are synthesized according to the following examples. As used below, and throughout the description of the invention, the following abbreviations, unless otherwise indicated, shall be understood to have the following meanings:

° C. degrees Celsius
δ_{H} chemical shift in parts per million downfield from tetramethylsilane
DCM dichloromethane (CH₂C₁₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
T temperature
TFA trifluoroacetic acid
THF tetrahydrofuran

### Example 1: Synthesis of ((R)—N-(4-(3-((5-chloropyrimidin-2-yl)amino)pyrrolidin-1-yl)quinazolin-7-yl)acrylamide

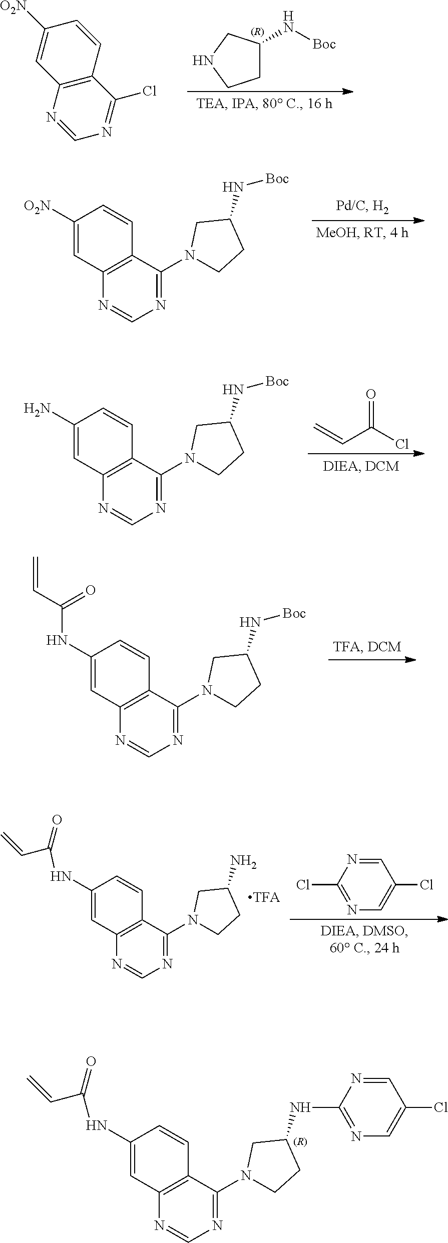

Step 1: (R)-tert-butyl (1-(7-nitroquinazolin-4-yl)pyrrolidin-3-yl)carbamate

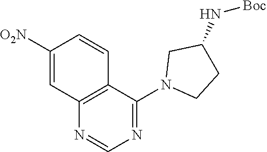 [SMILES: CC(C)(C)OC(=O)N[C@@H]1CCN(c2ncnc3cc([N+](=O)[O-])ccc23)C1]

A mixture of 4-chloro-7-nitroquinazoline (4.0 g, 19.0 mmol), (R)-tert-butyl pyrrolidin-3-ylcarbamate (5.3 g, 28.5 mml) and TEA (7.4 mL, 57.0 mmol) in i-PrOH (60 mL) was stirred at 80° C. for 16 hrs. The mixture was cooled and concentrated. The resultant solid was diluted with ACN (50 mL) and stirred for 20 min at rt. The reaction was filtered and washed with ACN (20 mL) to afford [1-(7-Nitro-quinazolin-4-yl)-pyrrolidin-3-yl]-carbamic acid tert-butyl ester (5.0 g, 73%) as a yellow solid. [M+H] Calc'd for C₁₇H₂₁N₅O₄, 360.2; Found, 360.2

Step 2: (R)-tert-butyl (1-(7-aminoquinazolin-4-yl)pyrrolidin-3-yl)carbamate

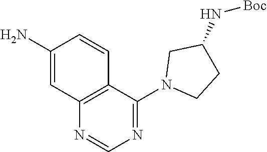 [SMILES: CC(C)(C)OC(=O)N[C@@H]1CCN(c2ncnc3cc(N)ccc23)C1]

A mixture of (R)-tert-butyl (1-(7-nitroquinazolin-4-yl)pyrrolidin-3-yl)carbamate (5.0 g, 13.9 mmol) and Pd/C (800 mg) in MeOH (80 mL) was stirred at rt under H2 balloon atmosphere for 4h. The reaction mixture was filtered and concentrated. The resultant solid was purified by silica gel chromatography (DCM/MeOH=20/1) to afford (R)-tert-butyl (1-(7-aminoquinazolin-4-yl)pyrrolidin-3-yl)carbamate (24.2 g, 91%) as a white solid. [M+H] Calc'd for C₁₇H₂₃N₅O₂, 330.2; Found, 330.1

Step 3: (R)-tert-butyl (1-(7-acrylamidoquinazolin-4-yl)pyrrolidin-3-yl)carbamate

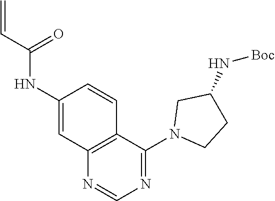 [SMILES: C=CC(=O)Nc1ccc2c(N3CC[C@@H](NC(=O)OC(C)(C)C)C3)ncnc2c1]

A mixture of (R)-tert-butyl (1-(7-aminoquinazolin-4-yl)pyrrolidin-3-yl)carbamate (1.00 g, 3.03 mmol) and DIEA (2.49 mL, 15.15 mmol) in DCM (40 mL) was stirred at 0° C. under nitrogen atmosphere. A solution of acryloyl chloride (0.22 mL, 2.72 mmol) in DCM (5 mL) was added dropwise and the mixture was warmed to r.t. and stirred for 3 hrs. The mixture was concentrated and submitted to prep-hplc to afford (R)-tert-butyl (1-(7-acrylamidoquinazolin-4-yl)pyrrolidin-3-yl)carbamate (220 mg, 19%) as a white solid. [M+H] Calc'd for C₂₀H₂₅N₅O₃, 384.2; Found, 384.1

Step 4: (R)—N-(4-(3-aminopyrrolidin-1-yl)quinazolin-7-yl)acrylamide

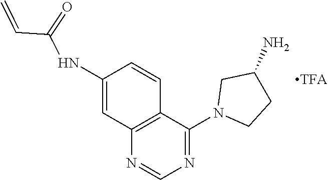 [SMILES: C=CC(=O)Nc1ccc2c(N3CC[C@@H](N)C3)ncnc2c1]

To a solution of (R)-tert-butyl (1-(7-acrylamidoquinazolin-4-yl)pyrrolidin-3-yl)carbamate (220 mg, 0.57 mmol) in DCM (20 mL) was added TFA (1 mL) and stirred at r.t. for 2 hrs. The mixture was concentrated to afford the TFA salt of (R)—N-(4-(3-aminopyrrolidin-1-yl)quinazolin-7-yl)acrylamide (162 mg, crude) as green liquid. [M+H] Calc'd for C₁₅H₁₇N₅O, 284.1; Found, 284.1

Step 5: (R)—N-(4-(3-((5-chloropyrimidin-2-yl)amino)pyrrolidin-1-yl)quinazolin-7-yl)acrylamide

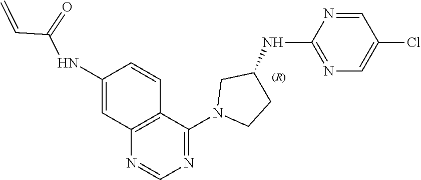

A mixture of the TFA salt of (R)—N-(4-(3-aminopyrrolidin-1-yl)quinazolin-7-yl)acrylamide (150 mg, 0.53 mmol), 2,5-Dichloro-pyrimidine (118 mg, 0.79 mmol) and DIEA (205 mg, 1.59 mmol) in DMSO (10 mL) was stirred at 60° C. under nitrogen atmosphere for 24 hrs. The reaction mixture was concentrated. The residue was purified by prep-HPLC to afford (R)—N-(4-(3-((5-chloropyrimidin-2-yl)amino)pyrrolidin-1-yl)quinazolin-7-yl)acrylamide (15.9 mg, 7.6%) as a white solid. ¹H NMR (400 MHz, DMSO-d6): 2.10-2.14 (m, 1H), 2.25-2.29 (m, 1H), 3.83-4.22 (m, 4H), 4.45-4.49 (m, 1H), 5.83-5.87 (m, 1H), 6.32-6.58 (m, 1H), 6.47-6.56 (m, 1H), 7.63-7.67 (m, 1H), 7.90-7.92 (m, 1H), 8.17- 8.25 (m, 2H), 8.40 (s, 3H), 10.54 (s, 1H). [M+H] Calc'd for C₁₉H₁₈CIN—O, 396.1; Found, 396.1.

### Example 2: Synthesis of (R)—N-(4-(3-((5-(trifluoromethyl)pyrimidin-2-yl)amino)pyrrolidin-1-yl) quinazolin-7-yl)acrylamide

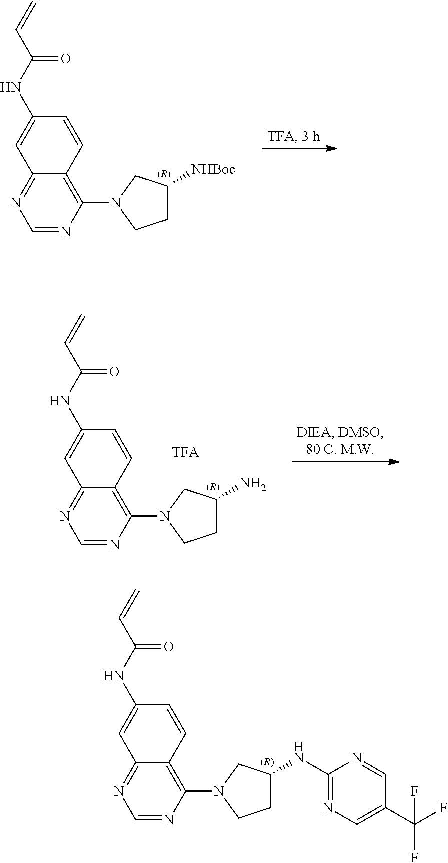

Step 1: (R)—N-(4-(3-aminopyrrolidin-1-yl)quinazolin-7-yl)acrylamide

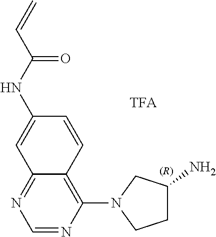

To a solution of (R)-tert-butyl (1-(7-acrylamidoquinazolin-4-yl) pyrrolidin-3-yl)carbamate (600 mg, 0.78 mmol) in dichloromethane (10 mL) was added TFA (2 mL) at rt. The reaction mixture was stirred at rt for 3h. The reaction mixture was concentrated to afford (R)—N-(4-(3-aminopyrrolidin-1-yl) quinazolin-7-yl)acryl amide (TFA salt) (600 mg, crude) as yellow oil. [M+H] Calc'd for C₁₅H₁₇N₅O, 284.1; Found, 284.1

Step 2: Synthesis of (R)—N-(4-(3-((5-(trifluoromethy)pyrimidin-2-yl)amino)pyrrolidin-1-yl)quinazolin-7-yl)acrylamide

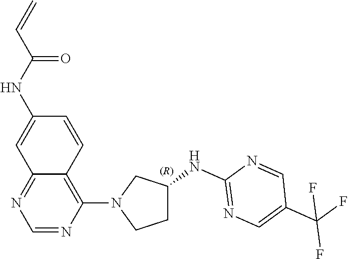

A mixture of (R)—N-(4-(3-aminopyrrolidin-1-yl)quinazolin-7-yl)acryl amide (TFA salt) (100 mg, 0.26 mmol), 2-chloro-5-(trifluoromethyl)pyrimidine (72 mg, 0.38 mmol) and DIEA (166 mg, 1.30 mmol) in DMSO (6 mL) was heated to 80° C. under nitrogen atmosphere for 30 min in microwave. The reaction mixture was cooled and extracted with DCM (10 mL×3). The combined organic layer was washed with brine (20 mL), dried over Na₂SO_{4,} filtered and concentrated in vacuum. The residue was purified by prep-HPLC to afford (R)—N-(4-(3((5-(trifluoromethyl)pyrimidin-2-yl)amino)pyrrolidin-2-yl)quinazolin-7-yl)pacrylamide (TFA salt) (64.1 mg, 59%) as a white solid. ¹H NMR (400 MHz, DMSO-d₆): δ 2.11-2.40 (m, 2H), 4.06-4.59 (m, 4H), 4.66 (s, 1H), 5.91 (dd, J=2.0, 10.0 Hz, 1H), 6.36-6.54 (m, 2H), 7.70 (d, J=9.2 Hz, 1H), 8.40-8.51 (m, 3H), 8.68-8.78 (m, 3H), 10.97 (s, 1H). [M+H] Calc'd for C₂₀H₁₈F₃N₇O, 430.2; Found, 430.1

### Example 3: Synthesis of (R)—N-(4-(3-((5-chloro-4-ethoxypyrimidin-2-yl)amino)pyrrolidin-1-yl)quinazolin-7-yl)acrylamide

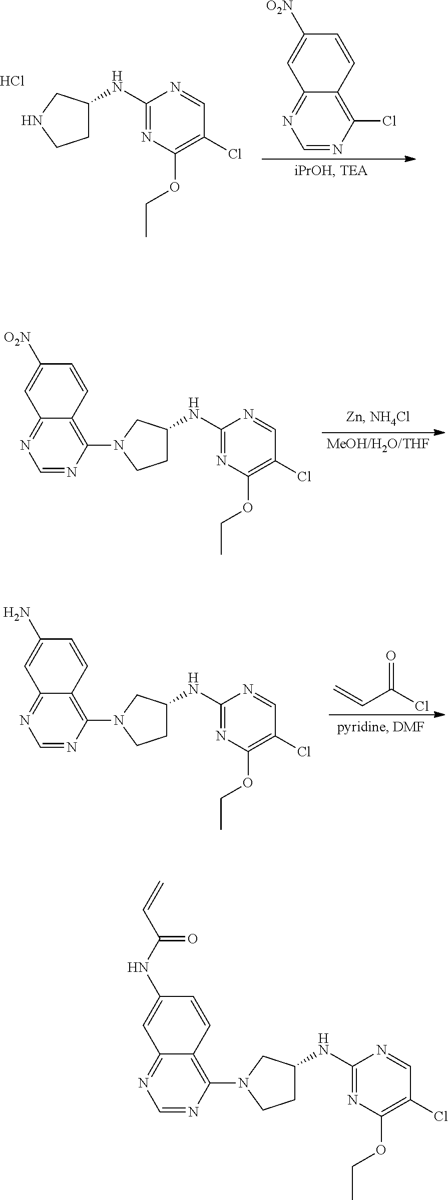

Step 1: (R)-5-chloro-4-ethoxy-N-(1-(7-nitroquinazolin-4-yl)pyrrolidin-3-yl)pyrimidin-2-amine

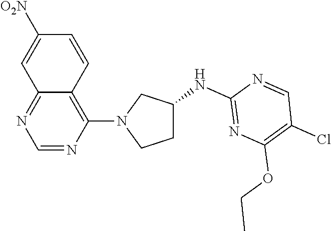 [SMILES: CCOc1nc(N[C@@H]2CCN(c3ncnc4cc([N+](=O)[O-])ccc34)C2)ncc1Cl]

A mixture of 4-chloro-7-nitroquinazoline (2.0 g, 9.5 mmol), (R)-5-chloro-4-ethoxy-N-(pyrrolidin-3-yl)pyrimidin-2-amine hydrochloride (2.9 g, 10.5 mml) and TEA (5.8 g, 57.2 mmol) in iPrOH (50 mL) was stirred at 80° C. for 6 hrs. The reaction mixture was cooled and concentrated. The residue was purified by silica gel chromatography (PE/EA=1/1) to afford (R)-5-chloro-4-ethoxy-N-(1-(7-nitroquinazolin-4-yl)pyrrolidin-3-yl)pyrimidin-2-amine (2.6 g, 66%) as a yellow solid. [M+H] Calc'd for C₁₅H₁₈CIN₇O₃, 416.1; Found, 416.1

Step 2: (R)-4-(3-((5-chloro-4-ethoxypyrimidin-2-yl)amino)pyrrolidin-1-yl)quinazolin-7-amine

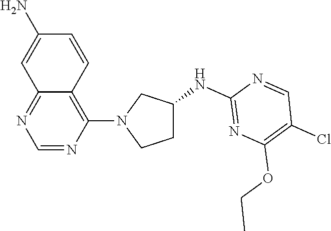 [SMILES: CCOc1nc(N[C@@H]2CCN(c3ncnc4cc(N)ccc34)C2)ncc1Cl]

A mixture of (R)-5-chloro-4-ethoxy-N-(1-(7-nitroquinazolin-4-yl)pyrrolidin-3-yl)pyrimidin-2-amine (2.6 g, 6.3 mmol), Zn (4.1 g, 62.7 mmol) and NH₄Cl (6.7 g, 125.3 mmol) in MeOH/THF/H₂O (100 mL/50 mL/50 mL) was stirred at 40° C. overnight. The reaction mixture was filtered and concentrated. The residue was purified by silica gel chromatography (DCM/MeOH=10/1) to afford (R)-4-(3-((5-chloro-4-ethoxypyrimidin-2-yl)amino)pyrrolidin-1-yl)quinazolin-7-amine (2.3 g, 97%) as red oil. [M+H] Calc'd for C₁₈H₂₀CIN₇O, 386.1; Found, 386.1

Step 3: (R)—N-(4-(3-((5-chloro-4-ethoxypyrimidin-2-yl)amino)pyrrolidin-1-yl)quinazolin-7-yl)acrylamide

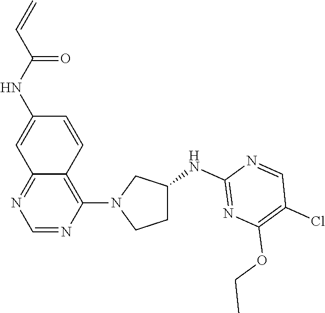 [SMILES: C=CC(=O)Nc1ccc2c(N3CC[C@@H](Nc4ncc(Cl)c(OCC)n4)C3)ncnc2c1]

A solution of (R)-4-(3-((5-chloro-4-ethoxypyrimidin-2-yl)amino)pyrrolidin-1-yl)quinazolin-7-amine (300 mg, 0.77 mmol) in dimethylformamide (10 mL) was stirred at 0° C. under nitrogen atmosphere. Then pyridine (369 mg, 4.67 mmol) was added, followed by acryloyl chloride (140 mg, 1.55 mmol) dropwise. Then the reaction mixture was stirred at 35° C. for 3 hrs. The reaction mixture was concentrated. The residue was purified by prep-HPLC to afford (R)—N-(4-(3-((5-chloro-4-ethoxypyrimidin-2-yl)amino)pyrrolidin-1-yl)quinazolin-7-yl)acrylamide (10.5 mg, 3%) as a white solid. ¹H NMR (400 MHz, DMSO-d₆): 1.30-1.34 (m, 3H), 2.07-2.12 (m, 1H), 2.22-2.26 (m, 1H), 3.82-3.93 (m, 2H), 4.01-4.06 (m, 1H), 4.17-4.19 (m, 1H), 4.39-4.46 (m, 3H), 5.81-5.84 (m, 1H), 6.30- 6.35 (m, 1H), 6.45-6.52 (m, 1H), 7.60-7.63 (m, 1H), 7.68-7.72 (m, 1H), 8.13-8.15 (m, 2H), 8.21-8.23 (m, 1H), 8.38 (s, 1H), 10.49 (s, 1H). [M+H] Calc'd for C₂₁H₂₂ClN₇O₂, 440.2; Found, 440.2

### Example 4: (R)—N-(4-(3-(5-chloro-4-methoxypyrimidin-2-yl)amino)pyrrolidin-1-yl)quinazolin-7-yl)acrylamide

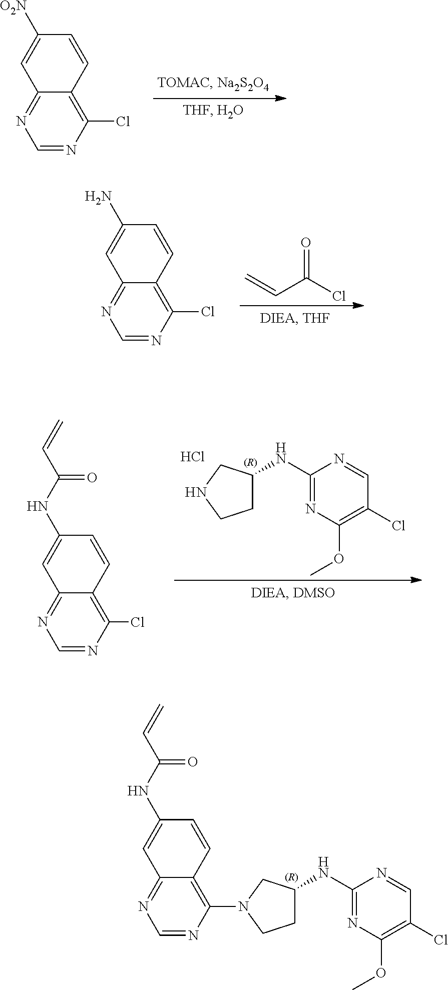

Step 1:4-chloroquinazolin-7-amine

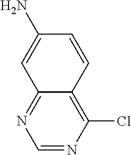 [SMILES: Nc1ccc2c(Cl)ncnc2c1]

A mixture of 4-chloro-7-nitroquinazoline (1.2 g, 5.7 mmol), Na₂S₂O₄ (4.0 g, 22.9 mmol) and Methyl trioctyl ammonium chloride (TOMAC) (1.1 g, 2.7 mmol) in THF/H₂O (36 mL/12 mL) was stirred at r.t. for 40 min. The organic layer was separated, dried over Na₂SO₄, filtered and concentrated to afford 4-chloroquinazolin-7-amine (1.7 g, crude) as yellow oil. [M+H] Calc'd for C₈H₆ClN₃, 180.0; Found, 180.0

Step 2: N-(4-chloroquinazolin-7-yl)acrylamide

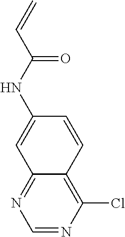 [SMILES: C=CC(=O)Nc1ccc2c(Cl)ncnc2c1]

A mixture of 4-chloroquinazolin-7-amine (1.70 g, 2.86 mmol) and DIEA (1.10 g, 8.5 mmol) in

THF (50 mL) was stirred at 0° C. under nitrogen atmosphere. A solution of acryloyl chloride in THF (2.86 mL, 1.0 mmol/L, 2.86 mmol) was added dropwise. The reaction mixture was allowed to warm to r.t. and stirred for 3 hrs. The reaction mixture was concentrated to afford N-(4-chloroquinazolin-7-yl)acrylamide (1.82 g, crude) as a yellow solid. [M+H] Calc'd for C₁₁H₈ClN₃O, 234.0; Found, 234.0

Step 3: (R)—N-(4-(3-((5-chloro-4-methoxypyrimidin-2-yl)amino)pyrrolidin-1-yl)quinazolin-7-yl)acrylamide

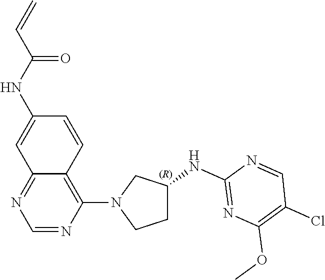

A mixture of N-(4-chloroquinazolin-7-yl)acrylamide (180 mg, 0.76 mmol), (R)-5-chloro-4-methoxy-N-(pyrrolidin-3-yl)pyrimidin-2-amine hydrochloride (244 mg, 0.92 mml) and DIEA (995 mg, 7.7 mmol) in DMSO (10 mL) was stirred at 40° C. under nitrogen atmosphere for 1.5 hrs. The reaction mixture was concentrated. The reaction mixture was purified by prep-HPLC to afford (R)—N-(4-(3-((5-chloro-4-methoxypyrimidin-2-yl)amino)pyrrolidin-1-yl)quinazolin-7-yl)acrylamide (10.7 mg, 3%) as a white solid. ¹H NMR (400 MHz, DMSO-d₆): 2.08-2.12 (m, 1H), 2.23-2.26 (m, 1H), 3.82-3.91 (m, 5H), 4.02-4.07 (m, 1H), 4.18 (m, 1H), 4.45-4.49 (m, 1H), 5.81-5.84 (m, 1H), 6.30-6.35 (m, 1H), 6.45-6.52 (m, 1H), 7.61-7.64 (m, 1H), 7.74-7.76 (m, 1H), 8.14-8.15 (m, 2H), 8.21-8.23 (m, 1H), 8.38 (s, 1H), 10.49 (s, 1H). [M+H] Calc'd for C₂₀H₂₀ClN₇O₂, 426.1; Found, 426.1

### Example 5: (R)—N-(4-(3((5-bromo-4-methoxypyrimidin-2-yl)amino)pyrrolidin-1-yl)quinazolin-7-yl)acrylamide

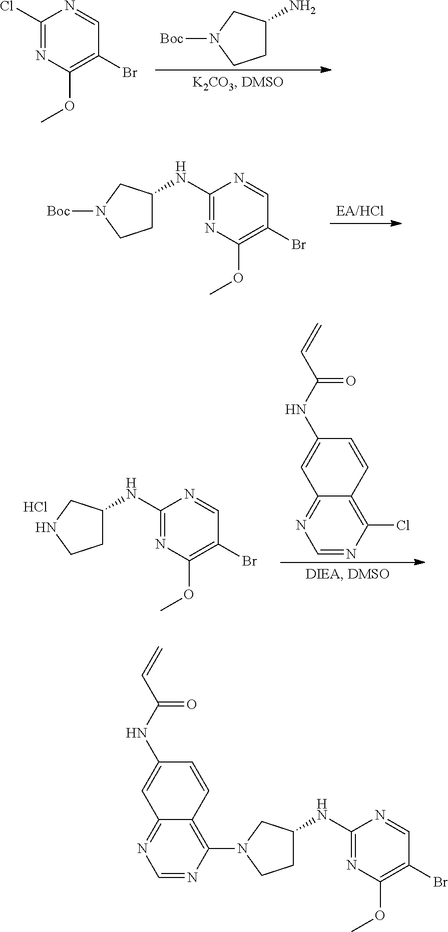

Step 1: (R)-tert-butyl 3-((5-bromo-4-methoxypyrimidin-2-yl)amino)pyrrolidine-1-carboxylate

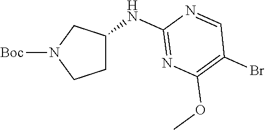 [SMILES: COc1nc(N[C@@H]2CCN(C(=O)OC(C)(C)C)C2)ncc1Br]

A mixture of 5-bromo-2-chloro-4-methoxypyrimidine (2.0 g, 8.9 mmol), (R)-tert-butyl 3-aminopyrrolidine-1-carboxylate (2.5 g, 13.4 mml) and K₂CO₃ (2.5 g, 11.9 mmol) in DMSO (50 mL) was stirred at 140° C. under nitrogen atmosphere for 4 hrs. The mixture was cooled to rt, poured into 200 mL H₂O and extracted with DCM (50 mL*3). The combined organic layer was dried over Na₂SO₄, filtered and concentrated. The residue was purified by silica gel chromatography (PE/EA=5/1) to afford (R)-tert-butyl 3-((5-bromo-4-methoxypyrimidin-2-yl)amino)pyrrolidine-1-carboxylate (804 mg, 24%) as a white solid. [M+H] Calc'd for C₁₄H₂₁BrN₄O₃, 373.1; Found, 373.1

Step 2: (R)-5-bromo-4-methoxy-N-(pyrrolidin-3-yl)pyrimidin-2-amine hydrochloride

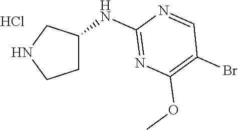

A solution of (R)-tert-butyl 3-((5-bromo-4-methoxypyrimidin-2-yl)amino)pyrrolidine-1-carboxylate (804 mg, 2.16 mmol) in EA/HC1 (10 mL, 1.0 M) was stirred at r.t. for 1 hr. The mixture was concentrated to afford (R)-5-bromo-4-methoxy-N-(pyrrolidin-3-yl)pyrimidin-2-amine hydrochloride (809 mg, crude) as a white solid. [M+H] Calc'd for C₉H₁₃BrN₄O, 273.0; Found, 273.0

Step 3: (R)—N-(4-(3-((5-bromo-4-methoxypyrimidin-2-yl)amino)pyrrolidin-1-yl)quinazolin-7-yl)acrylamide

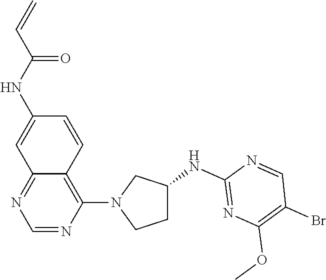 [SMILES: C=CC(=O)Nc1ccc2c(N3CC[C@@H](Nc4ncc(Br)c(OC)n4)C3)ncnc2c1]

A mixture of N-(4-chloroquinazolin-7-yl)acrylamide (400 mg, 1.71 mmol), (R)-5-bromo-4-methoxy-N-(pyrrolidin-3-yl)pyrimidin-2-amine hydrochloride (530 mg, 1.71 mml) and DIEA (2.21 g, 17.16 mmol) in DMSO (8 mL) was stirred at 40° C. under nitrogen atmosphere for 1.5 hrs. The reaction mixture was concentrated. The residue was purified by prep-HPLC to afford (R)—N-(4-(3-((5-bromo-4-methoxypyrimidin-2-yl)amino)pyrrolidin-1-yl)quinazolin-7-yl)acrylamide (11.2 mg, 1%) as a white solid. ⁱH NMR (400 MHz, DMSO-d₆): 2.08-2.13 (m, 1H), 2.23-2.26 (m, 1H), 3.83-3.90 (m, 5H), 4.02-4.07 (m, 1H), 4.16-4.20 (m, 1H), 4.45-4.49 (m, 1H), 5.81-5.84 (m, 1H), 6.30-6.35 (m, 1H), 6.45-6.52 (m, 1H), 7.61-7.64 (m, 1H), 7.76 (s, 1H), 8.14-8.15 (m, 1H), 8.21-8.23 (m, 2H), 8.39 (s, 1H), 10.49 (s, 1H). [M+H] Calc'd for C₂₀H₂₀BrN₇O₂, 470.1; Found, 470.1

### Example 6: Synthesis of (R)—N-(4-(3(4-amino-5-(trifluoromethyl)pyrimidin-2-yl)amino)pyrrolidin-1-yl)quinazolin-7-yflacrylamide

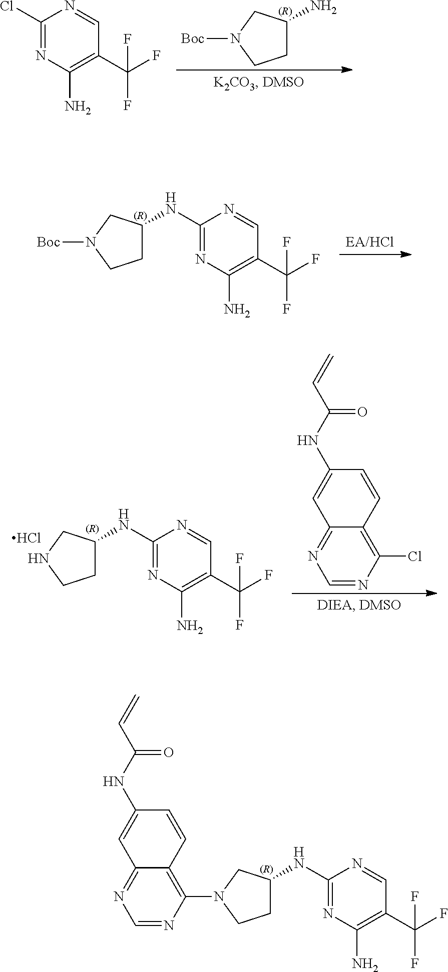

Step 1: (R)-tert-butyl 3-((4-amino-5-(trifluoromethyl)pyrimidin-2-yl)amino)pyrrolidine-1-carboxylate

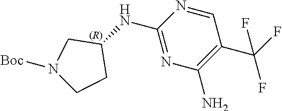

A mixture of 2-chloro-5-(trifluoromethyl)pyrimidin-4-amine (400 mg, 2.0 mmol), (R)-tert-butyl 3-aminopyrrolidine-1-carboxylate (755 mg, 4.0 mmol) and K₂CO₃ (552 mg, 4.0 mmol) in DMSO (10 mL) was heated to 170° C. for 2 h under microwave. The mixture was was cooled to rt, poured into 50 mL H₂O and extracted with EA (50 mL*3). The combined organic phase was dried over Na₂SO_{4,} filtered and concentrated. The residue was purified by silica gel chromatography (PE/EA=1/10 to 1/5) to afford (R)-tert-butyl 3-((4-amino-5-(trifluoromethyl)pyrimidin-2-yl)amino)pyrrolidine-1-carboxylate (500 mg, 72%) as a white solid. [M+H] Calc'd for C₁₄H₂₀F₃N₅O₂, 348.2; Found, 348.2

Step 2: (R)—N2-(pyrrolidin-3-yl)-5-(trifluoromethyl)pyrimidine-2,4-diamine hydrochloride

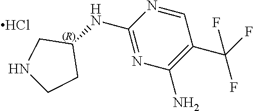

A mixture of (R)-tert-butyl 3-(4-amino-5-(trifluoromethyppyrimidin-2-yl)amino)pyrrolidine-1-carboxylate (500 mg, 1.4 mmol) in HCl/EA (10 mL, 2.0 M) was stirred at RT for 2 hrs. The mixture was concentrated to afford (R)—N2-(pyrrolidin-3-yl)-5-(trifluoromethyl)pyrimidine-2,4-diamine hydrochloride (395 mg, 100%) as a white solid. [M+H] Calc'd for C₉H₁₂F₃N₅, 248.1; Found, 248.1

Step 3: (R)—N-(4-(3-((4-amino-5-(trifluoromethyppyrimidin-2-yl)amino)pyrrolidin-1-yl)quinazolin-7-yl)acrylamide

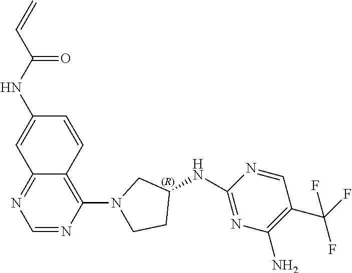

A mixture of N-(4-chloroquinazolin-7-yl)acrylamide (311 mg, 1.3 mmol), (R)—N2-(pyrrolidin-3-yl)-5-(trifluoromethyl)pyrimidine-2,4-diamine hydrochloride (395 mg, 1.6 mmol) and DIEA (168 mg, 1.3 mmol) in DMSO (10 mL) was stirred at RT for 1 h. The mixture was diluted with H₂O (10 mL) and extracted with EA (10 mL*3). The combined organic layer was dried over Na₂SO_{4,} filtered and concentrated. The residue was purified by prep-HPLC to afford (R)—N-(4-(3-((4-amino-5-(trifluoromethyl)pyrimidin-2-yl)amino)pyrrolidin-1-yl)quinazolin-7-yl)acrylamide (13.9 mg, 2.4%) as a white solid. ¹H NMR (400 MHz, DMSO-d₆): δ 2.05-2.25 (m, 2H), 3.78-3.94 (m, 2H), 4.01-4.15(m, 2H), 4.47-4.49 (m, 1H), 5.82 (dd, J=8.0, 12.0 Hz, 1H), 6.30-6.35 (m, 1H), 6.45-6.52 (m, 1H), 6.70-6.83 (m, 2H), 7.43-7.64 (m, 2H), 8.06 (br s, 1H), 8.14-8.22 (m, 2H), 8.38 (s, 1H), 10.47 (s, 1H). [M+H] Calc'd for C₂₀H₁₉F₃N₈O, 445.2; Found, 445.2

### Example 7: Synthesis of N-{4-[3-(5-Chloro-4-phenoxy-pyrimidin-2-ylamino)-pyrrolidin-1-yl]-quinazolin-7-yl}-acrylamide

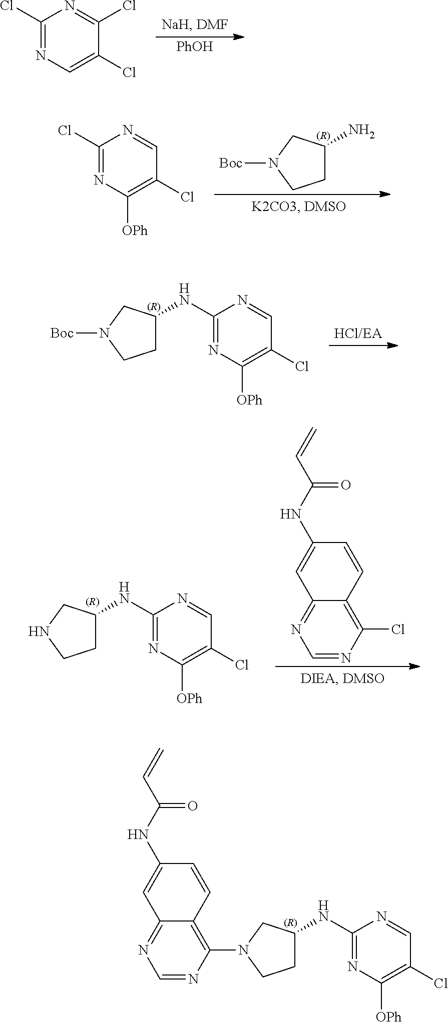

Step 1: 2,5-dichloro-4-phenoxypyrimidine

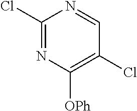 [SMILES: Clc1ncc(Cl)c(Oc2ccccc2)n1]

To a mixture of 2,4,5-trichloropyrimidine (1.0 g, 5.5 mmol) in dimethylformamide (20 mL) was added NaH (220 mg, 60%, 5.49 mmol) at 0° C. under N_{2.} The mixture was stirred at 0° C. for 15 min and then added PhOH (516 mg, 5.49 mmol). The reaction was stirred at RT for 3 hrs. The mixture was quenched with H₂O (5 mL) and extracted with EA (30 mL). The organic phase was dried over Na₂SO₄, filtered and concentrated. The residue was purified by silica gel chromatography (PE/EA=1/10) to afford 2,5-dichloro-4-phenoxypyrimidine (1.2 g, 92%) as a white solid. [M+H] Calc'd for C₁₀H₆Cl₂N₂O, 241.0; Found, 241.0

Step 2: (R)-tert-butyl 3-((5-chloro-4-phenoxypyrimidin-2-yl)amino)pyrrolidine-1-carboxylate

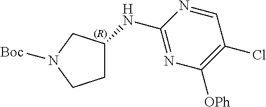

A mixture of 2,5-dichloro-4-phenoxypyrimidine (1.2 g, 5.0 mmol), 3-Amino-pyrrolidine-1-carboxylic acid tert-butyl ester (1.4 g, 7.5 mmol) and K₂CO₃ (1.4 g, 10.0 mmol) in DMSO (25 mL) was heated to 140° C. for 4 hrs. The mixture was cooled and concentrated. The residue was purified by silica gel chromatography (PE/EA=1/10 to 1/3) to afford (R)-tert-butyl 3-((5-chloro-4-phenoxypyrimidin-2-yl)amino)pyrrolidine-1-carboxylate (1.2 g, 62%) as colorless oil. [M+H] Calc'd for C₁₉H₂₃ClN₄O₃, 391.0; Found, 391.0

Step 3: (R)-5-chloro-4-phenoxy-N-(pyrrolidin-3-yl)pyrimidin-2-amine

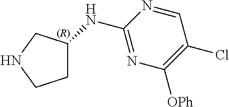

A mixture of (R)-tert-butyl 3-((5-chloro-4-phenoxypyrimidin-2-yl)amino)pyrrolidine-1-carboxylate (300 mg, 0.77 mmol) in HCl/EA (10 mL, 1.0 M) was stirred at RT for 2 hrs. The mixture was concentrated to afford (R)-5-chloro-4-phenoxy-N-(pyrrolidin-3-yl)pyrimidin-2-amine (250 mg, 100%) as a yellow solid. [M+H] Calc'd for C₁₄H₁₅ClN₄O, 291.0; Found, 291.0

Step 4: (R)—N-(4-(3-((5-chloro-4-phenoxypyrimidin-2-yl)amino)pyrrolidin-1-yl)quinazolin-7-yl)acrylamide

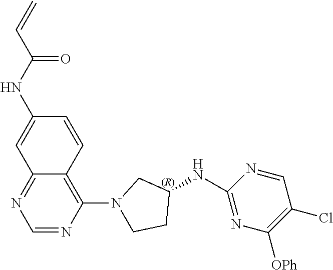

A mixture of N-(4-chloroquinazolin-7-yl)acrylamide (179 mg, 0.76 mmol), (R)-5-chloro-4-phenoxy-N-(pyrrolidin-3-yl)pyrimidin-2-amine (250 mg, 0.76 mmol) and DIEA (198 mg, 1.51 mmol) in DMSO (10 mL) was heated to 40° C. for 1 hr. The reaction mixture was added H₂O (5 mL). Then the mixture was filtered. The filtered cake was purified by prep-HPLC to afford (R)—N-(4-(3-((5-chloro-4-phenoxypyrimidin-2-yl)amino)pyrrolidin-1-yl)quinazolin-7-yl)acrylamide (4.4 mg, 12%) as a white solid. ¹HNMR (400 MHz, CDC1₃): δ 1.97-2.07 (m, 2H), 3.78-4.01 (m, 5H), 5.81-5.84 (m, 1H), 6.30-6.35 (m, 1H), 6.46-6.52 (m, 1H), 7.21-7.53 (m, 7H), 8.15 (s, 2H), 8.32-8.36 (m, 2H), 10.50 (s, 1H). [M+H] Calc'd for C₂₅H₂₂ClN₇O₂, 488.0; Found, 488.0

### Example 8: (R)—N-(4-(3-((6-fluoroquinazolin-2-yl)amino)pyrrolidin-1-yl)quinazolin-7-yl)acrylamide

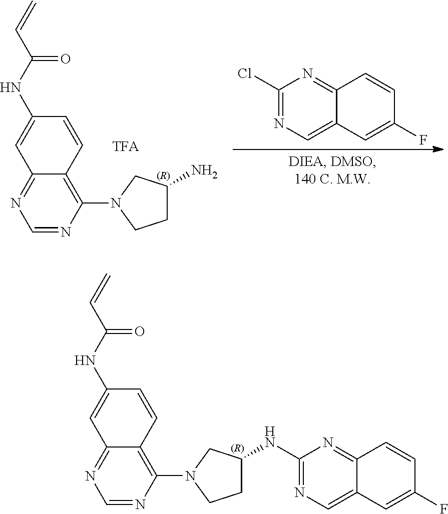

A mixture of (R)—N-(4-(3-aminopyrrolidin-1-yl)quinazolin-7-yl)acrylamide (TFA salt) (100 mg, 0.26 mmol), 2-chloro-6-fluoroquinazoline (69 mg, 0.38 mmol) and DIEA (166 mg, 1.30 mmol) in DMSO (6 mL) was heated to 140° C. under nitrogen atmosphere for 30 min under microwave. The mixture was cooled to rt, diluted with water (20 mL) and extracted with DCM (10 mL* 3). The combined organic layer was washed with brine (20 mL), dried over Na₂SO₄, filtered and concentrated in vacuo. The residue was purified by prep-HPLC to afford (R)—N-(4-(3-((6-fluoroquinazolin-2-yl)amino)pyrrolidin-1-yl)quinazolin-7-yl)acrylamide (TFA salt) (34.1 mg, 32%) as a yellow solid. ¹HNMR (400 MHz, DMSO-d₆): δ 2.11-2.40 (m, 2H), 4.06-4.95 (m, 4H), 5.90 (d, J=11.2 Hz, 1H), 6.36-6.54 (m, 2H), 7.58-7.68 (m, 4H), 7.95 (s, 1H), 8.37-8.51 (m, 2H), 8.78 (s, 1H), 9.17 (s, 1H), 10.96 (s, 1H), 14.25 (br s, 1H). [M+H] Calc'd for C₂₃H₂₀FN₇O, 430.1; Found, 430.1.

### Example 9: (R)—N-(4-(3-((5-chloro-4-(trifluoromethyl)pyrimidin-2-yl)amino)pyrrolidin-1-yl)quinazolin-7-yl)acrylamide

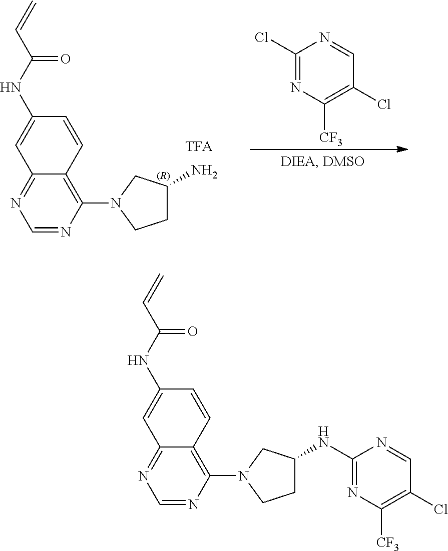

A mixture of (R)—N-(4-(3-aminopyrrolidin-1-yl)quinazolin-7-yl)acrylamide trifluoroacetate (75 mg, 0.18 mmol), 2,5-dichloro-4-(trifluoromethyl)pyrimidine (61 mg, 0.28 mml) and DIEA (123 mg, 0.9 mmol) in DMSO (3 mL) was heated at 140° C. in microwave for 40 min. Concentrated and the mixture was purified by prep-HPLC to afford (R)—N-(4-(3-((5-chloro-4-(trifluoromethyl)pyrimidin-2-yl)amino)pyrrolidin-1-yl)quinazolin-7-yl)acrylamide (14.5 mg, 16%) as a white solid. ¹H NMR (400 MHz, DMSO-d₆): 2.09-2.14 (m, 1H), 2.25-2.30 (m, 1H), 3.85-3.88 (m, 2H), 3.94-3.95 (m, 1H), 4.06-4.08 (m, 1H), 4.16-4.20 (m, 1H), 4.50 (s, 1H), 5.81-5.84 (m, 1H), 6.30-6.35 (m, 1H), 6.45-6.52 (m, 1H), 7.61-7.64 (m, 1H), 8.14-8.22 (m, 1H), 8.39 (s, 1H), 8.51 (m, 1H), 8.71 (m, 1H), 10.48 (s, 1H). [M+H] Calc'd for C₂₀H₁₇ClF₃N₇O, 464.1; Found, 464.1

### Example 10: (R)—N-(4-(3-((5-cyclopropylpyrimidin-2-yl)amino)pyrrolidin-1-yl)quinazolin-7-yl)acrylamide

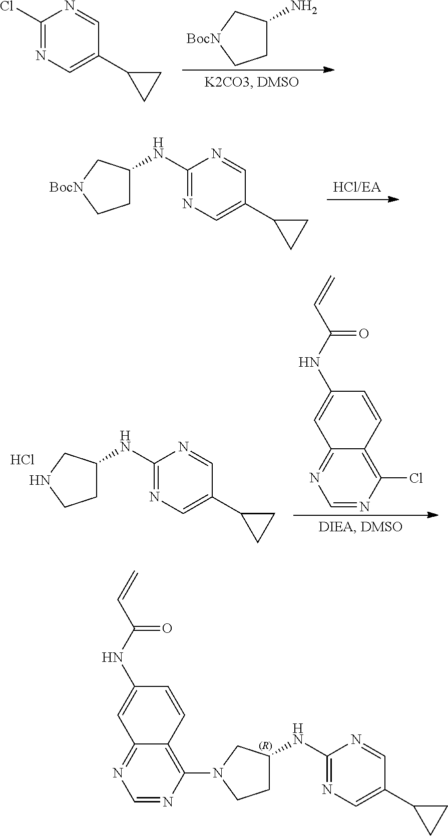

Step 1: (R)-tert-butyl 3-((5-cyclopropylpyrimidin-2-yl)amino)pyrrolidine-1-carboxylate

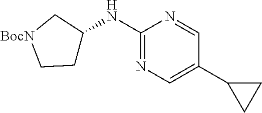 [SMILES: CC(C)(C)OC(=O)N1CC[C@@H](Nc2ncc(C3CC3)cn2)C1]

To a solution of 2-chloro-5-cyclopropylpyrimidine (500 mg, 3.25 mmol) and (R)-tert-butyl 3-aminopyrrolidine-1-carboxylate (906 mg, 4.87 mmol) in DMSO (10 mL) was added K₂CO₃ (894 mg, 6.48 mmol) at rt. The mixture was stirred at 170° C. for 2.5 h under microwave. The mixture was cooled, diluted with water (20 mL) and extracted with DCM (10 mL*3). The combined organic layer was washed with brine (20 mL), dried over Na₂SO_{4,} filtered and concentrated in vacuo. The residue was purified by column (PE/EA=3/1) to give (R)-tert-butyl 3-((5-cyclopropylpyrimidin-2-yl)amino)pyrrolidine-1-carboxylate (170 mg, 17%) as yellow oil. [M+H] Calc'd for C₁₆H₂₄N₄O₂, 305.2; Found, 305.2

Step 2: (R)-5-cyclopropyl-N-(pyrrolidin-3-yl)pyrimidin-2-amine (HC1 salt)

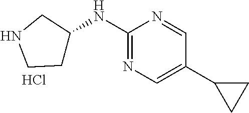 [SMILES: c1nc(N[C@@H]2CCNC2)ncc1C1CC1 ; Cl]

A mixture of (R)-tert-butyl 3-((5-cyclopropylpyrimidin-2-yl)amino)pyrrolidine-1-carboxylate (170 mg, 0.56 mmol) in HC1/EA (5 mL, 1.0 M) was stirred at RT for 2 h. The reaction mixture was concentrated to afford (R)-5-cyclopropyl-N-(pyrrolidin-3-yl)pyrimidin-2-amine (HCl salt) (120 mg, 89%) as a yellow solid. [M+H] Calc'd for C₁₁H₁₆N₄, 205.1; Found, 205.1

Step 3: (R)—N-(4-(3-((5-cyclopropylpyrimidin-2-yl)amino)pyrrolidin-1-yl)quinazolin-7-yl)acrylamide

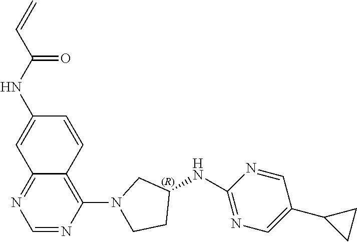

A mixture of N-(4-chloroquinazolin-7-yl)acrylamide (270 mg, 1.16 mmol), (R)-5-cyclopropyl-N-(pyrrolidin-3-yl)pyrimidin-2-amine (HCl salt) (120 mg, 0.58 mml) and DIEA (347 mg, 2.89 mmol) in DMSO (4 mL) was stirred at 40° C. for 1 h. The mixture was diluted with water (20 mL) and extracted with DCM and THF (DCM:THF=1:1, 10 mL* 3). The combined organic layer was washed with brine (20 mL), dried over Na₂SO₄, filtered and concentrated. The residue was purified by prep-HPLC to afford (R)—N-(4-(3-((5-cyclopropylpyrimidin-2-yl)amino)pyrrolidin-1-yl)quinazolin-7-yl)acrylamide (17.5 mg, 7.5%) as a white solid. ¹H NMR (400 MHz, DMSO-d₆): 0.64-0.68 (m, 2H), 0.82-0.86 (m, 2H), 1.71-1.76 (m, 1H), 2.07-2.10 (m, 1H), 2.22-2.25 (m, 1H), 3.81-4.18 (m, 4H), 4.45- 4.46 (m, 1H), 5.83 (dd, J=10.0, 1.6 Hz, 1H), 6.30-6.35 (m, 1H), 6.47-6.54 (m, 1H), 7.35 (d, J=6.4 Hz, 1H), 7.63 (dd, J=2.0, 9.6 Hz, 1H), 8.11-8.24 (m, 4H), 8.41 (s, 1H), 10.48 (s, 1H). [M+H] Calc'd for C₂₂H₂₃N₇O, 402.2; Found, 402.2

### Example 11: (R)—N-(4-(3-(thieno[3,2-d]pyrimidin-2-ylamino)pyrrolidin-1-yl)quinazolin-7-yl)acrylamide

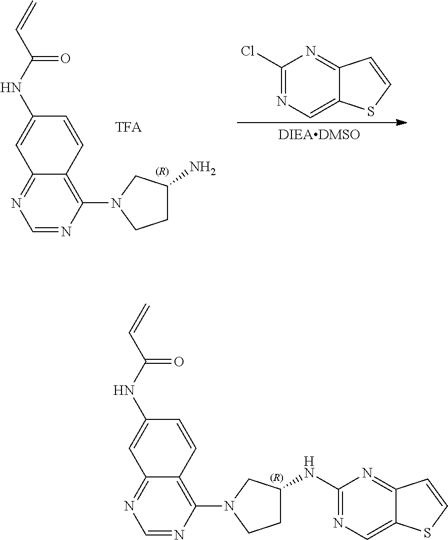

A mixture of (R)—N-(4-(3-aminopyrrolidin-1-yl)quinazolin-7-yl)acrylamide (TFA salt) (100 mg, 0.26 mmol), 2-chlorothieno[3,2-d]pyrimidine (65 mg, 0.38 mmol) and DIEA (166 mg, 1.30 mmol) in DMSO (3 mL) was heated to 170° C. under nitrogen atmosphere for 1.5 h under microwave. The mixture was cooled, diluted with water (20 mL) and extracted with DCM (10 mL* 3). The combined organic layer was washed with brine (20 mL), dried over Na₂SO₄, filtered and concentrated in vacuo. The residue was purified by prep-HPLC to afford (R)—N-(4-(3-(thieno[3,2-d]pyrimidin-2-ylamino)pyrrolidin-1-yl)quinazolin-7-yl)acrylamide (3.9 mg, 3.8%) as a white solid. ¹H NMR (400 MHz, DMSO-d₆): δ 2.03-2.20 (m, 1H), 2.28-2.32 (m, 1H), 3.90-4.11 (m, 4H), 4.58-4.59 (m, 1H), 5.83 (dd, J=2.0, 10.4 Hz, 1H), 6.31-6.35 (m, 1H), 6.48-6.54 (m, 1H), 7.23 (d, J=5.2 Hz, 1H), 7.54 (d, J=6.0 Hz, 1H), 7.66 (dd, J=2.0, 9.2 Hz, 1H), 8.20-8.28 (m, 3H), 8.45 (s, 1H), 9.01 (s, 1H), 10.62 (s, 1H). [M+H] Calc'd for C₂₁H₁₉N₇OS, 418.1; Found, 418.1

### Example 12: (R)—N-(4-(3-((5-cyanopyrimidin-2-ynamino)pyrrolidin-1-yl)quinazolin-7-yl)nacrylamide

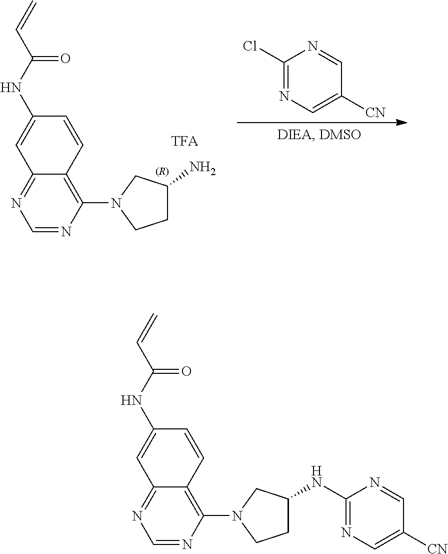

A mixture of (R)—N-(4-(3-aminopyrrolidin-1-yl)quinazolin-7-yl)acrylamide trifluoroacetate (75 mg, 0.18 mmol), 2-chloropyrimidine-5-carbonitrile (39 mg, 0.28 mml) and DIEA (123 mg, 0.9 mmol) in DMSO (3 mL) was reacted at 140° C. for 1 h under microwave. The reaction mixture was cooled and concentrated. The residue was purified by prep-HPLC to afford (R)—N-(4-(3-((5-cyanopyrimidin-2-yl)amino)pyrrolidin-1-yl)quinazolin-7-yl)acrylamide (18.9 mg, 26%) as a white solid. ¹H NMR (400 MHz, DMSO-d₆): 2.10-2.13 (m, 1H), 2.26-2.29 (m, 1H), 3.85-3.87 (m, 1H), 3.95-3.96 (m, 1H), 4.05-4.08 (m, 1H), 4.16-4.20 (m, 1H), 4.57-4.59 (m, 1H), 5.81-5.84 (m, 1H), 6.30-6.35 (m, 1H), 6.46-6.52 (m, 1H), 7.62-7.65 (m, 1H), 8.15-8.16 (m, 1H), 8.20-8.23 (m, 1H), 8.39-8.41 (m, 1H), 8.69-8.71 (m, 2H), 8.78-8.79 (m, 1H), 10.51 (s, 1H). [M+H] Calc'd for C₂₀H₁₈N8O, 387.2; Found, 387.2

### Example 13: (R)—N-(4-(3-(quinazolin-2-ylamino)pyrrolidin-1-yl)quinazolin-7-yflacrylamide

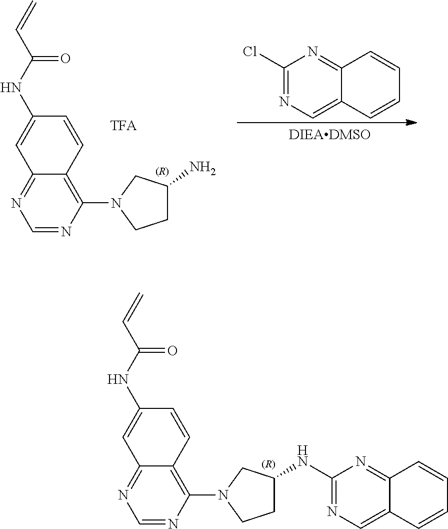

A mixture of (R)—N-(4-(3-aminopyrrolidin-1-yl)quinazolin-7-yl)acrylamide (TFA salt) (44 mg, 0.16 mmol), 2-chloroquinazoline (38 mg, 0.24 mmol) and DIEA (60 mg, 0.47 mmol) in DMSO (3 mL) was heated to 140° C. for 1 h under microwave. The mixture was diluted with water (20 mL) and extracted with DCM (10 mL*3). The combined organic layer was washed with brine (20 mL), dried over Na₂SO₄, filtered and concentrated in vacuo. The residue was purified by prep-HPLC to afford (R)—N-(4-(3-(quinazolin-2-ylamino)pyrrolidin-1-yl)quinazolin-7-yl)acrylamide (6.4 mg, 10%) as a white solid. ¹H NMR (400 MHz, DMSO-d₆): δ 2.16-2.19 (m, 1H), 2.30-2.33 (m, 1H), 3.89-4.25 (m, 4H), 4.64-4.66 (m, 1H), 5.82 (dd, J=2.0, 10.0 Hz, 1H), 6.30-6.35 (m, 1H), 6.45-6.52 (m, 1H), 7.26 (t, J=7.2 Hz, 1H), 7.49-7.52 (m, 1H), 7.62 (dd, J=2.0, 9.2 Hz, 1H), 7.69-7.73 (m, 1H), 7.74-7.84 (m, 2H), 8.16 (d, J=2.0 Hz, 1H), 8.25 (d, J=9.2 Hz, 1H), 8.40 (s, 1H), 9.15 (s, 1H), 10.49 (s, 1H). [M+H] Calc'd for C₂₃H₂₁N₇O, 412.1; Found, 412.1

### Example 14: (R)—N-(4-(3-((5-chloro-4-(methylamino)pyrimidin-2-yl)amino)pyrrolidin-1-yl)quinazolin-7-yl)acrylamide

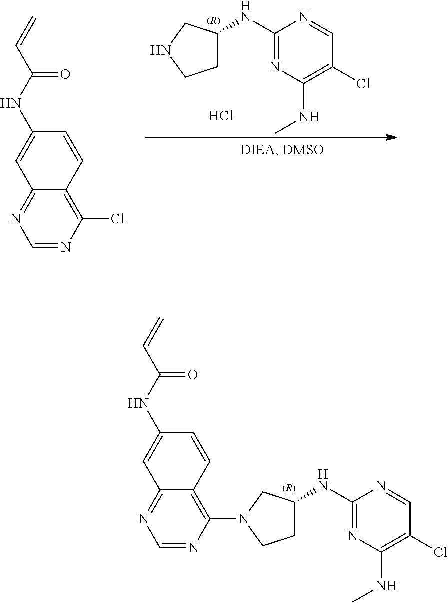

A mixture of N-(4-chloroquinazolin-7-yl)acrylamide (200 mg, 0.85 mmol), (R)-5-chloro-N4-methyl-N2-(pyrrolidin-3-yl)pyrimidine-2,4-diamine (HC1 salt) (120 mg, 0.45 mmol) and DIEA (580 mg, 4.5 mmol) in DMSO (6 mL) was heated to 40° C. for 1 h. The mixture was diluted with water (20 mL) and extracted with DCM (10 mL*3). The combined organic was washed with brine (20 mL), dried over Na₂SO_{4,} filtered and concentrated in vacuo. The residue was purified by prep-HPLC to afford (R)—N-(4-(3 -((5-chloro-4-(methylamino)pyrimidin-2-yl)amino)pyrrolidin-1-yl)quinazolin-7-yl)acrylamide (15.6 mg, 8.2%) as a white solid. ¹H NMR (400 MHz, DMSO-d₆): δ 2.16-2.19 (m, 1H), 2.30-2.33 (m, 1H), 2.82 (d, J=4.0 Hz, 3H), 3.89-4.15 (m, 4H), 4.44-4.46 (m, 1H), 5.84 (dd, J =1.2, 10.0 Hz, 1H), 6.31-6.36 (m, 1H), 6.46-6.53 (m, 1H), 6.96 (s, 1H), 7.12 (m, 1H), 7.64 (dd, J=1.6, 8.8 Hz, 1H), 7.77 (s, 1H), 8.20-8.26 (m, 2H), 8.45 (s, 1H), 10.59 (s, 1H). [M+H] Calc'd for C₂₀H₂₁ClN₈O, 425.1; Found, 425.1

### Example 15: (R)—N-(4-(3-((4-(methylamino)-5-(trifluoromethyl)pyrimidin-2-ynamino)pyrrolidin-1-yl)quinazolin-7-yl)acrylamide

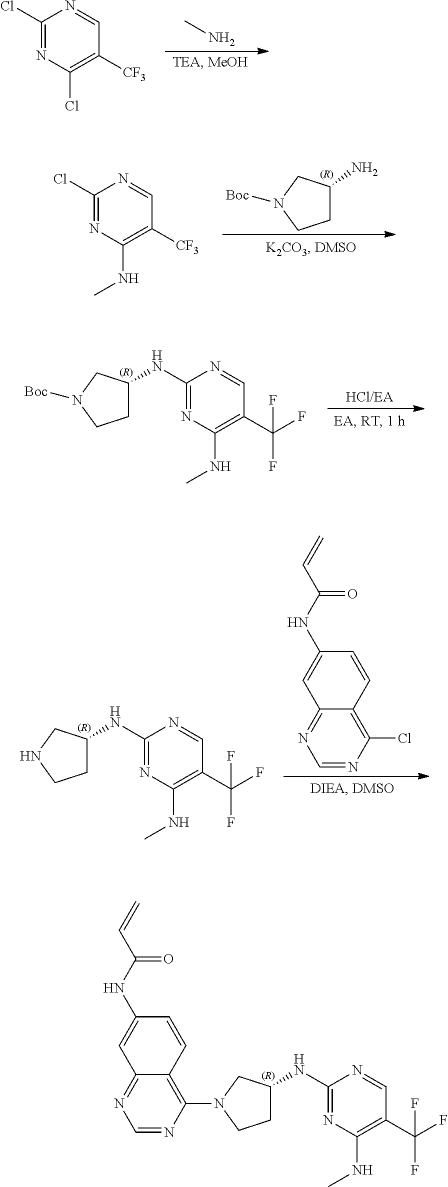

Step 1: 2-chloro-N-methyl-5-(trifluoromethyl)pyrimidin-4-amine

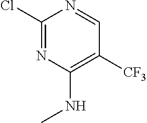

To a cooled solution of 2,4-dichloro-5-(trifluoromethyl)pyrimidine (6.7 g, 30.8 mmol) in MeOH (150 mL) was added TEA (4.3 mL, 30.8 mmol) and methanamine (15.6 mL, 2.0 M in THF, 31.1 mmol) at rt. The mixture was stirred at room temperature for 16 hrs. The mixture was concentrated and the residue was purified by silica gel chromatography (PE/EA=10/1 to 5/1) to afford 2-chloro-N-methyl-5-(trifluoromethyl)pyrimidin-4-amine (1.2 g, 18.5%) as a white solid. [M+H] Calc' d for C₆H₅ClF₃N₃, 212; Found, 212.

Step 2: (R)-tert-butyl 3-((4-(methylamino)-5-(trifluoromethyl)pyrimidin-2-yl)amino)pyrrolidine-1-carboxylate

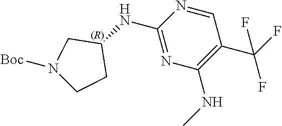

To a solution of 2-chloro-N-methyl-5-(trifluoromethyl)pyrimidin-4-amine (760 mg, 3.59 mmol) and (R)-tert-butyl 3-aminopyrrolidine-1-carboxylate (1.34 g, 7.18 mmol) in DMSO (20 mL) was added K₂CO₃ (992 mg, 7.18 mmol) ar rt. The mixture was stirred at 170° C. for 1.5 hrs under microwave. The mixture was concentrated in-vacuo. The residue was purified by silica gel chromatography (PE/EA=10/1 to 5/1) to afford (R)-tert-butyl 3-((4-(methylamino)-5-(trifluoromethyl)pyrimidin-2-yl)amino)pyrrolidine-1-carboxyl ate (958 mg, 73.7%) as a white solid. [M+H] Calc'd for C₁₅H₂₂F₃N₅O₂, 362; Found, 362.

Step 3: (R)—N4-methyl-N2-(pyrrolidin-3-yl)-5-(trifluoromethyl)pyrimidine-2,4-diamine hydrochloride

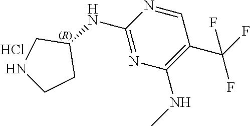

To a cooled solution of (R)-tert-butyl 3-aminopyrrolidine-1-carboxylate (958 mg, 2.65 mmol) in EtOAc (2 mL) was added HCl (15.0 mL, 2 M in EtOAc, 30.0 mmol) at rt. The mixture was stirred at room temperature for 1 hr. The mixture was concentrated to afford (R)—N4-methyl-N2-(pyrrolidin-3-yl)-5-(trifluoromethyl)pyrimidine-2,4-diamine hydrochloride (789 mg, crude) as a white solid. [M+H] Calc'd for C₁₀H₁₄F₃N₅, 262; Found, 262.

Step 4: (R)—N-(4-(3((-4-(methylamino)-5-(trifluoromethyppyrimidin-2-yl)amino)pyrrolidin-1-yl)quinazolin-7-yl)acrylamide

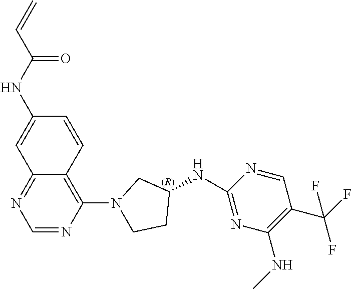

To a solution of (R)—N4-methyl-N2-(pyrrolidin-3-yl)-5-(trifluoromethyl)pyrimidine-2,4-diamine hydrochloride (789 mg, 2.65 mmol) in DMSO (20 mL) was added DIEA (3.43 g, 26.5 mmol) and N-(4-chloroquinazolin-7-yl)acrylamide (618 mg, 2.65 mmol). The mixture was stirred at 40° C. for 30 min. The mixture was diluted with water (50 mL) and extracted with THF (50 mL*3). The combined organic layer was dried over Na₂SO₄, filtered and removed. The residue was purified by prep-HPLC to afford (R)—N-(4-(3-((4-(methylamino)-5-(trifluoromethyl)pyrimidin-2-yl)amino)pyrrolidin-1-yl)quinazolin-7-yl)acrylamide (28 mg, 2.3%) as white solid. ¹HNMR (400 MHz, DMSO-d₆): δ 2.11 (s, 1H), 2.25 (s, 1H), 2.87-2.95 (m, 3H), 3.84-3.93 (m, 2H), 4.02-4.08 (m, 1H), 4.19 (s, 1H), 4.54 (s, 1H), 5.83 (dd, J=2.0, 12.0 Hz, 1H), 6.33 (dd, J=1.6, 18.8 Hz, 1H), 6.49 (dd, J=10.0, 27.0 Hz, 1H), 6.86-6.92 (m, 1H), 7.57-7.70 (m, 2H), 8.04-8.08 (m, 1H), 8.14 (d, J=2.0 Hz, 1H), 8.22 (d, J=9.2 Hz, 1H), 8.39 (s, 1H), 10.47 (s, 1H). [M+H] Calc'd for C₂₁H₂₁F₃N₈O, 459; Found, 459.

### Example 16: (R)—N-(4-(3-(pyrido[3,4-d]pyrimidin-2-ylamino)pyrrolidin-1-yl)quinazolin-7-yl)acrylamide

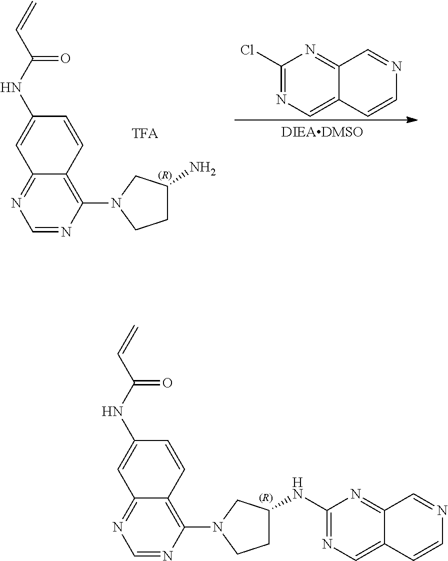

A mixture of (R)—N-(4-(3-aminopyrrolidin-1-yl)quinazolin-7-yl)acrylamide (TFA salt) (44 mg, 0.15 mmol), 2-chloropyrido[3,4-d]pyrimidine (31 mg, 0.18 mmol) and DIEA (97 mg, 0.75 mmol) in DMSO (3 mL) was heated to 140° C. for 30 min under microwave. The mixture was diluted with water (20 mL) and extracted with DCM (10 mL*3). The combined organic layer was washed with brine (20 mL), dried over Na2SO4, filtered and concentrated in vacuo. The residue was purified by prep-HPLC to afford (R)—N-(4-(3-(pyrido[3,4-d]pyrimidin-2-ylamino)pyrrolidin-1-yl)quinazolin-7-yl)acrylamide (3.7 mg, 6.0%) as a white solid.

¹H NMR (400 MHz, DMSO-d₆): δ 2.18-2.32 (m, 1H), 2.32-2.35 (m, 1H), 3.91-4.00 (m, 2H), 4.10-4.13 (m, 1H), 4.23-4.28 (m, 1H), 4.67-4.69 (m, 1H), 5.82 (dd, J=2.0, 10.0 Hz, 1H), 6.30-6.35 (m, 1H), 6.45-6.52 (m, 1H), 7.63 (dd, J=2.4, 9.2 Hz, 1H), 7.72 (dd, J=0.8, 5.2 Hz, 1H), 8.15 (d, J=2.4 Hz, 1H), 8.23-8.26 (m, 2H), 8.35 (d, J=5.2 Hz, 1H), 8.40 (s, 1H), 8.96 (s, 1H), 9.31 (s, 1H), 10.47 (s, 1H). [M+H] Calc'd for C₂₂H₂₀N₈O, 413.1; Found, 413.1

### Example 17: (R)—N-(4-(34(5-chloropyrimidin-2-yl)amino)pyrrolidin-1-yl)-2-methoxyquinazolin-7-yl)acrylamide

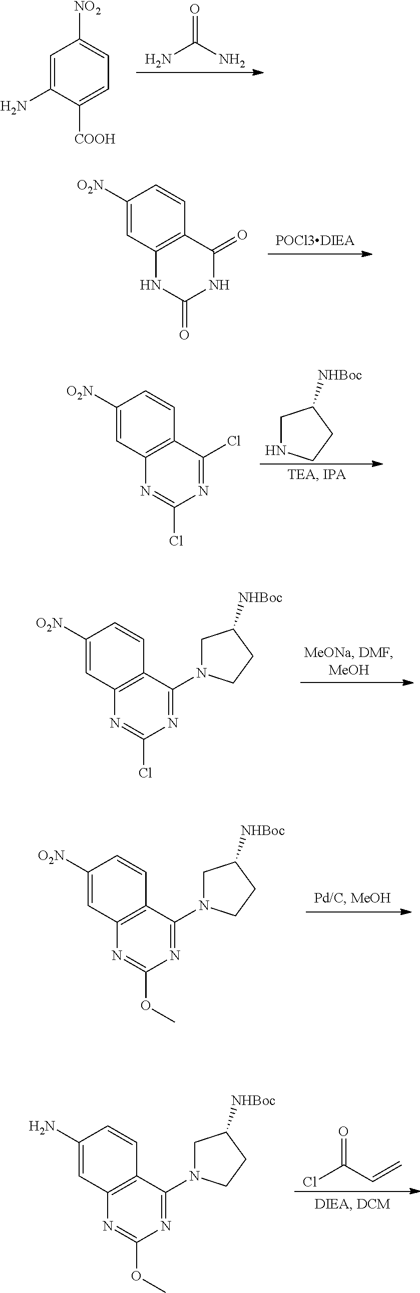

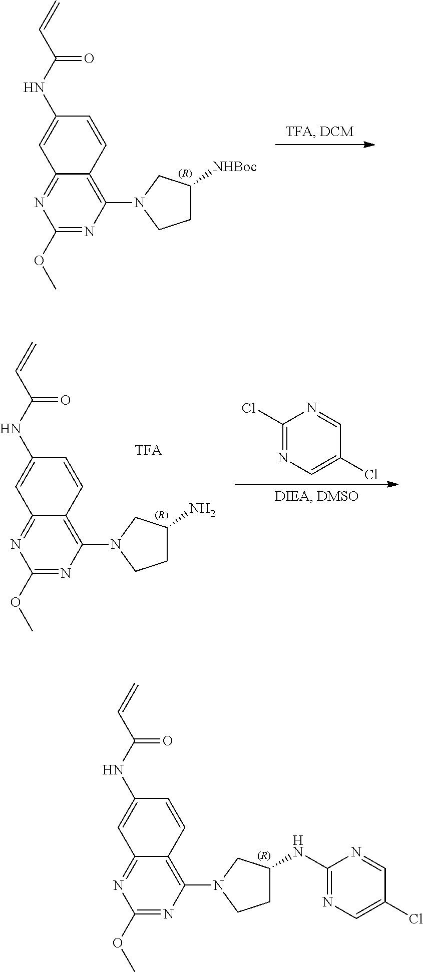

Step 1: 7-nitroquinazoline-2,4(1H,3H)-dione

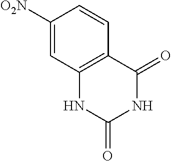 [SMILES: O=c1[nH]c(=O)c2ccc([N+](=O)[O-])cc2[nH]1]

A mixture of 2-amino-4-nitrobenzoic acid (10.0 g, 55.0 mmol) and urea (33.0 g, 55.0 mmol) was heated to 160° C. for 6 h. Then the reaction mixture was cooled to 100° C. and water (60 mL) was added. The solution was stirred for 5 min. The formed precipitate was filtered off, washed with cold water and further suspended in 0.5N NaOH (50 mL). The mixture was refluxed for 5 min. Then the reaction mixture was cooled to RT and filtered. The filtrate was adjusted to pH=2 with conc. HCl. The crude product was filtered off, washed with MeOH/H₂O=1:1 (100 mL) and dried in vacuo to give 7-nitroquinazoline-2,4(1H,3H)-dione (10.4 g, 91%) as a yellow solid. Calc'd for C₈H₅N₃O₄, 208.0; Found, 208.0

Step 2: 2,4-dichloro-7-nitroquinazoline

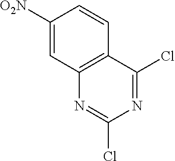 [SMILES: O=[N+]([O-])c1ccc2c(Cl)nc(Cl)nc2c1]

To a mixture of 7-nitroquinazoline-2,4(1H,3H)-dione (3.0 g, 14.4 mmol) in phosphoryl trichloride (20 mL) was added DIEA (3.7 g, 28.9 mmol) slowly at rt. The reaction mixture was heated to 140° C. for 3 h. Then the reaction mixture was cooled and concentrated in vacuo. The residue was purified by column chromatography (PE:EA=5:1) to afford 2,4-dichloro-7-nitroquinazoline (2.5 g, 71%) as a yellow solid.

¹HNMR (400 MHz, CDCl₃): δ 8.48-8.49 (m, 2H), 8.86 (s, 1H).

Step 3: (R)-tert-butyl (1-(2-chloro-7-nitroquinazolin-4-yl)pyrrolidin-3-yl)carbamate

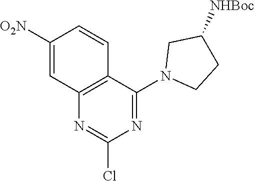 [SMILES: CC(C)(C)OC(=O)N[C@@H]1CCN(c2nc(Cl)nc3cc([N+](=O)[O-])ccc23)C1]

To a mixture of 2,4-dichloro-7-nitroquinazoline (2.5 g, 10.3 mmol) and (R)-tert-butyl pyrrolidin-3-ylcarbamate (1.9 g, 10.3 mmol) in IPA (40 mL) was added TEA (3.1 g, 30.9 mmol) at rt. The reaction mixture was heated to 80° C. overnight. The reaction mixture was cooled and concentrated in vacuo. The residue was purified by column chromatography (PE:EA=3:1) to afford (R)-tert-butyl (1-(2-chloro-7-nitroquinazolin-4-yl)pyrrolidin-3-yl)carbamate (2.0 g, 50%) as a yellow solid. [M+H] Calc'd for C₁₇H₂₀ClN₅O₄, 394.1; Found, 394.1

Step 4: (R)-tert-butyl (1-(2-methoxy-7-nitroquinazolin-4-yl)pyrrolidin-3-yl)carbamate

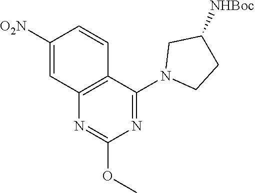 [SMILES: COc1nc(N2CC[C@@H](NC(=O)OC(C)(C)C)C2)c2ccc([N+](=O)[O-])cc2n1]

To a solution of (R)-tert-butyl (1-(2-chloro-7-nitroquinazolin-4-yl)pyrrolidin-3-yl)carbamate (2.0 g, 5.0 mmol) in DMF (15 mL) was added a solution of MeONa (270 mg, 5.0 mmol) in MeOH (5 mL). The reaction mixture was heated to 80° C. for 3 h. The reaction mixture was cooled and concentrated in-vacuo. The residue was purified by column (PE:EA=5:1) to afford (R)-tert-butyl (1-(2-methoxy-7-nitroquinazolin-4-yl)pyrrolidin-3-yl)carbamate (1.5 g, 77%) as a yellow solid. [M+H] Calc'd for C₁₈H₂₃N₅O₅, 390.1; Found, 390.1

Step 5: (R)-tert-butyl (1-(7-amino-2-methoxyquinazolin-4-yl)pyrrolidin-3-yl)carbamate

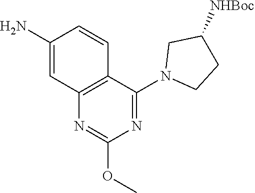 [SMILES: COc1nc(N2CC[C@@H](NC(=O)OC(C)(C)C)C2)c2ccc(N)cc2n1]

A solution of (R)-tert-butyl (1-(2-methoxy-7-nitroquinazolin-4-yl)pyrrolidin-3-yl)carbamate (1.5 g, 3.8 mmol) and Pd/C (300 mg, 20%) in MeOH (30 mL) was stirred at RT overnight under 1 atm H₂.

The reaction mixture was filtered and concentrated to afford (R)-tert-butyl (1-(7-amino-2-methoxyquinazolin-4-yl)pyrrolidin-3-yl)carbamate (1.0 g, 72%) as a yellow solid. [M+H] Calc'd for C₁₈H₂₅N₅O₃, 360.2; Found, 360.2

Step 6: (R)-tert-butyl (1-(7-acrylamido-2-methoxyquinazolin-4-yl)pyrrolidin-3-yl)carbamate

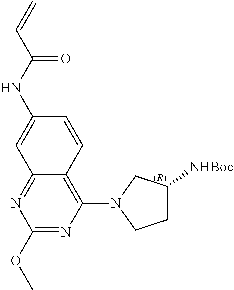

To a solution of (R)-tert-butyl (1-(7-amino-2-methoxyquinazolin-4-yl)pyrrolidin-3-yl)carbamate (1.0 g, 3.0 mmol) and DIEA (1.9 g, 15 mmol) in DCM (20 mL) was added a solution of acryloyl chloride (317 mg, 3.5 mmol) in DCM (0.5 mL) at 0° C. The mixture was then slowly warmed to RT and stirred overnight under N₂. The mixture was diluted with water (20 mL) and extracted with DCM (10 mL*3). The combined organic layer was washed with brine (20 mL), dried over Na₂SO₄, filtered and concentrated in-vacuo. The residue was purified by column chromatography (DCM:MeOH=20:1) to afford (R)-tert-butyl (1-(7-acrylamido-2-methoxyquinazolin-4-yl)pyrrolidin-3-yl)carbamate (450 mg, 37.5%) as a brown solid. [M+H] Calc'd for C₂₁H₂₇N₅O₄, 414.2; Found, 414.2

Step 7: (R)—N-(4-(3-aminopyrrolidin-1-yl)-2-methoxyquinazolin-7-yl)acrylamide (TFA salt)

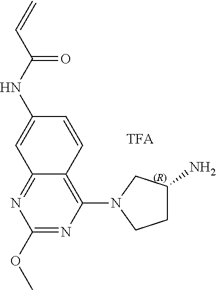

A solution of (R)-tert-butyl (1-(7-acrylamido-2-methoxyquinazolin-4-yl)pyrrolidin-3-yl)carbamate (200 mg, 0.48 mmol) and TFA (1 mL) in DCM (10 mL) was stirred at RT for 3 h. The reaction mixture was concentrated in vacuo to afford (R)—N-(4-(3-aminopyrrolidin-1-yl)-2-methoxyquinazolin-7-yl)acrylamide (TFA salt) (200 mg, crude) as a yellow solid. [M+H] Calc'd for C₁₆H₁₉N₅O₂, 314.1; Found,314.1

Step 8: (R)—N-(4-(3-((5-chloropyrimidin-2-yl)amino)pyrrolidin-1-yl)-2-methoxyquinazolin-7-yl)acrylamide (TFA salt)

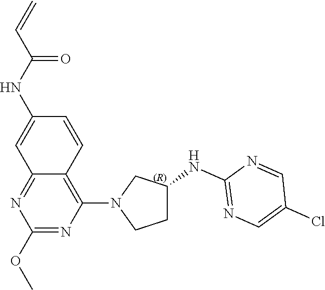

A mixture of (R)—N-(4-(3-aminopyrrolidin-1-yl)-2-methoxyquinazolin-7-yl)acrylamide (TFA salt) (200 mg, 0.48 mmol), 2,5-dichloropyrimidine (71 mg, 0.48 mmol) and DIEA (309 mg, 2.4 mmol) in DMSO (10 mL) was heated to 60° C. overnight. The reaction mixture was cooled, diluted with water (20 mL) and extracted with DCM (10 mL*3). The combined organic layer was washed with brine (20 mL), dried over Na₂SO₄, filtered and concentrated in vacuo. The residue was purified by prep-HPLC to afford (R)—N-(4-(3-((5-chloropyrimidin-2-yl)amino)pyrrolidin-1-yl)-2-methoxyquinazolin-7-yl)acrylamide (TFA salt) (15.0 mg, 7.0%) as a yellow solid.

¹HNMR (400 MHz, DMSO-d₆): δ 2.07-2.40 (m, 2H), 4.04-4.10 (m, 6H), 4.56 (m, 1H), 5.89 (dd, J=10.0, 1.6 Hz, 1H), 6.34-6.38 (m, 1H), 6.45-6.52 (m, 1H), 7.55 (d, J=8.4 Hz, 1H), 7.92 (d, J=2.0 Hz, 1H), 8.26-8.41 (m, 4H), 10.84 (s, 1H), 13.61 (br s, 1H). [M+H] Calc'd for C₂₀H₂₀ClN₇O₂, 426.1; Found, 426.1

### Example 18: (R)—N-(1-(3-((5-bromopyrimidin-2-yl)amino)pyrrolidin-1-yl)isoquinolin-6-yl)acrylamide

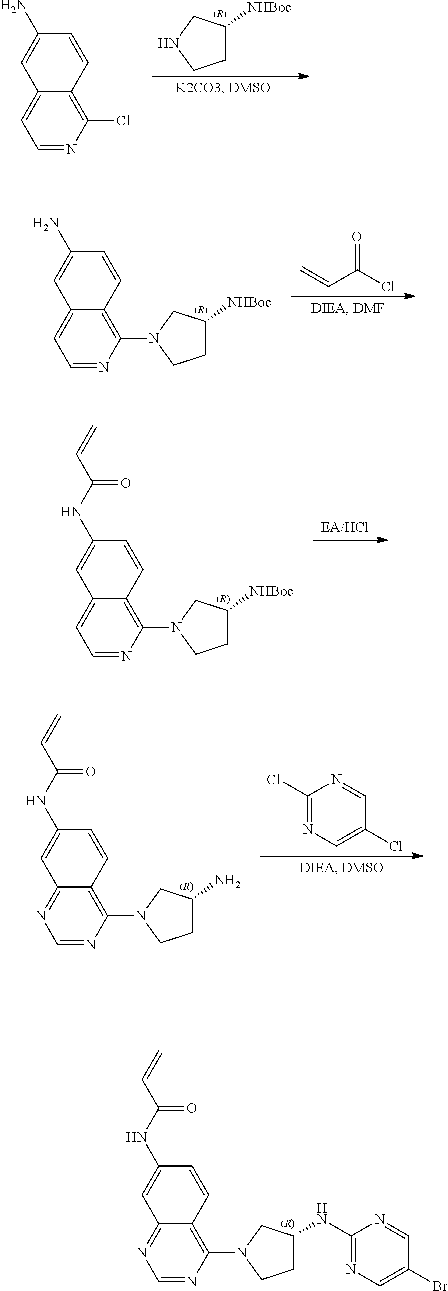

Step 1: (R)-tert-butyl (1-(6-aminoisoquinolin-1-yl)pyrrolidin-3-yl)carbamate

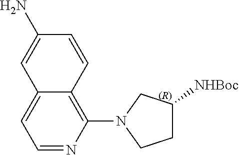

A mixture of 1-chloroisoquinolin-6-amine (355 mg, 1.99 mmol), (R)-tert-butyl pyrrolidin-3-ylcarbamate (1.11 g, 5.98 mml) and K₂CO₃ (413 mg, 2.99 mmol) in DMSO (5 mL) was stirred at 180° C. for 5 hrs under microwave. The mixture was cooled, poured into 50 mL of H₂O and extracted with EA (50 mL*3). The combined organic layer was dried over Na₂SO₄, filtered and concentrated. The residue was purified by silica gel chromatography (PE/EA=1/1) to afford (R)-tert-butyl (1-(6-aminoisoquinolin-1-yl)pyrrolidin-3-yl)carbamate (360 mg, 55%) as a white solid. [M+H] Calc'd for C₁₈H₂₄N₄O₂, 329.1; Found, 329.1

Step 2: (R)-tert-butyl (1-(6-aminoisoquinolin-1-yl)pyrrolidin-3-yl)carbamate

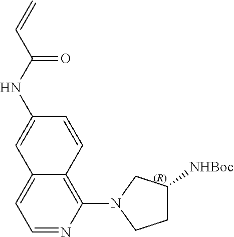

A mixture of (R)-tert-butyl (1-(6-aminoisoquinolin-1-yl)pyrrolidin-3-yl)carbamate (320 mg, 0.98 mmol) and DIEA (629 mg, 4.88 mmol) in DMF (10 mL) was stirred at 0° C. under nitrogen atmosphere. A solution of acryloyl chloride (88 mg, 0.98 mmol) in DMF (1 mL) was added dropwise and the mixture was warmed to r.t for 1 hrs. The mixture was poured into 50 mL of H₂O and extracted with EA (50 mL*3). The combined organic layer was dried over NaSO₄, filtered and concentrated. The residue was purified by silica gel chromatography (PE/EA=2/1) to (R)-tert-butyl (1-(6-acrylamidoisoquinolin-1-yl)pyrrolidin-3-yl)carbamate (220 mg, 59%) as yellow solid. [M+H] Calc'd for C₂₁H₂₆N₄O₃, 383.4; Found, 383.4

Step 3: (R)-tert-butyl (1-(6-aminoisoquinolin-1-yl)pyrrolidin-3-yl)carbamate

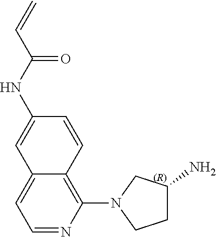

A solution (R)-tert-butyl (1-(6-acrylamidoisoquinolin-1-yl)pyrrolidin-3-yl)carbamate (220 mg, 0.58 mmol) and TFA (1 mL) in DCM (10 mL) was stirred at RT for 2 h. The reaction mixture was concentrated in-vacuo to afford (R)—N-(1-(3-aminopyrrolidin-1-yl)isoquinolin-6-yl)acrylamide (TFA salt) (190 mg, crude) as yellow oil. [M+H] Calc'd for C₁₆H₁₈N₄O, 283.1; Found,283.1

Step 4:(R)—N-(1-(3-((5-bromopyrimidin-2-yl)amino)pyrrolidin-1-yl)isoquinolin-6-yl)acrylamide

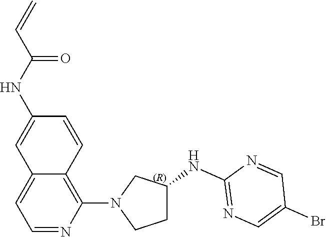

A mixture of (R)—N-(1-(3-aminopyrrolidin-1-yl)isoquinolin-6-yl)acrylamide (120 mg, 0.30 mmol), 2,5-dichloro-4-ethoxypyrimidine (58 mg, 0.30 mml) and DIEA (194 mg, 1.51 mmol) in DMSO (3 mL) was stirred at 60° C. for 16 hrs. The reaction mixture was cooled and concentrated. The residue was purified by prep-HPLC to afford (R)—N-(1-(3-((5-bromopyrimidin-2-yl)amino)pyrrolidin-1-yl)isoquinolin-6-yl)acrylamide (60.3 mg, 47%) as a gray solid. ¹H NMR (400 MHz, DMSO-d₆): 2.03-2.06 (m, 1H), 2.21-2.22 (m, 1H), 3.69-3.73 (m, 1H), 3.81-3.83 (m, 1H), 3.92-3.94 (m, 1H), 4.03-4.07 (m, 1H), 4.39-4.40 (m, 1H), 5.80-5.83 (m, 1H), 6.29-6.34 (m, 1H), 6.46-6.53 (m, 1H), 6.95-6.96 (m, 1H), 7.55-7.58 (m, 1H), 7.80-7.82 (m, 1H), 7.86 (d, J=5.6 Hz, 1H), 8.17-8.20 (m, 2H), 8.40 (s, 2H), 10.41 (s, 1H). [M+H] Calc'd for C₂₀H₁₉BrN₆O₂, 440.1; Found, 440.1

### Example 19: (R)—N-(1-(3-((5-chloro-4-ethoxypyrimidin-2-yl)amino)pyrrolidin-1-yl)isoquinolin-6-yflacrylamide

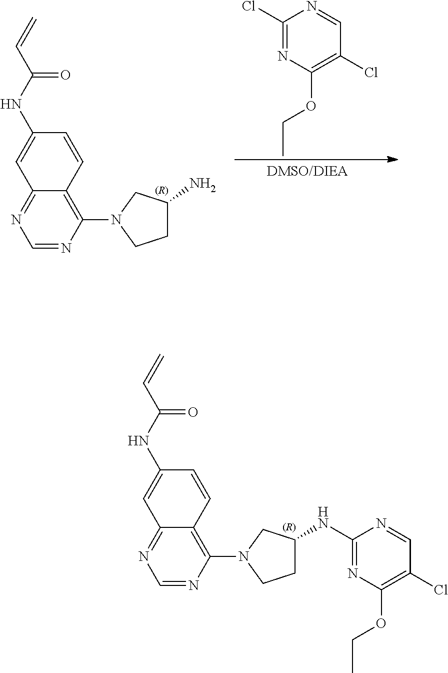

A mixture of (R)—N-(1-(3-aminopyrrolidin-1-yl)isoquinolin-6-yl)acrylamide (50 mg, 0.13 mmol), 2,5-dichloro-4-ethoxypyrimidine (24 mg, 0.13 mml) and DIEA (81 mg, 0.63 mmol) in DMSO (3 mL) was stirred at 150° C. for 30 min under microwave conditions. The reaction mixture was cooled and concentrated. The residue was purified by prep-HPLC to afford (R)—N-(1-(3-((5-chloro-4-ethoxypyrimidin-2-yl)amino) pyrrolidin-1-yl)isoquinolin-6-yl) acrylamide (10.6 mg, 19%) as a white solid. ¹H NMR (400 MHz, DMSO-d₆): 1.23-1.33 (m, 3H), 2.03-2.08 (m, 1H), 2.22-2.24 (m, 1H), 3.70-3.74 (m, 1H), 3.81-3.83 (m, 1H), 3.90-3.91 (m, 1H), 3.92-3.94 (m, 1H), 4.06-4.08 (m, 1H), 4.16-4.20 (m, 1H), 4.50 (s, 1H), 5.81-5.84 (dd, J=10.4, 2.0, 1H), 6.30-6.35 (m, 1H), 6.47-6.54 (m, 1H), 6.96-6.98 (m, 1H),7.56-7.59 (m, 1H), 7.68 (br s, 1H), 8.13 (m, 1H), 8.20-8.22 (m, 1H), 10.44 (s, 1H). [M+H] Calc'd for C₂₂H₂₃ClN₆O₂, 439.9; Found, 439.9

### Example 20: (R)—N-(4-(34(5-bromopyrimidin-2-yl)amino)pyrrolidin-1-yl)-2-morpholinoquinazolin-7-yl)acrylamide

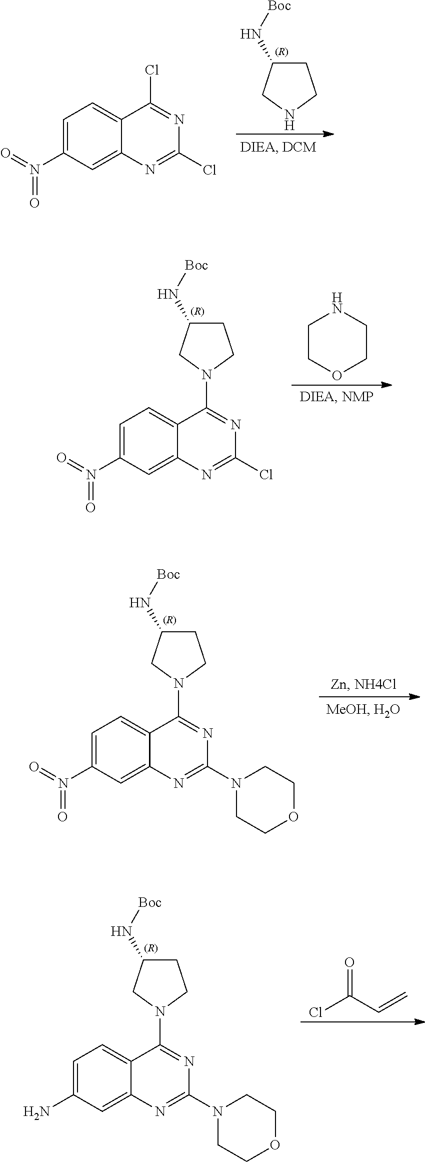

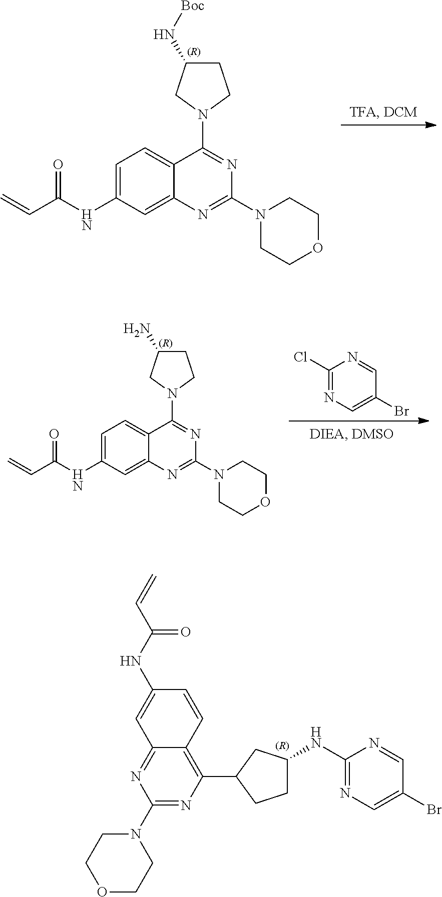

Step 1: (R)-tert-butyl (1-(2-chloro-7-nitroquinazolin-4-yl)pyrrolidin-3-yl)carbamate

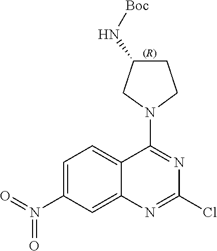

To a mixture of 2,4-dichloro-7-nitroquinazoline (3.0 g, 12.3 mmol) and (R)-tert-butyl pyrrolidin-3-ylcarbamate (2.3 g, 12.3 mmol) in DCM (50 mL) was added DIEA (4.76 g, 36.9 mmol) at rt and stirred for 2 hrs. The mixture was washed with water (100 mL) and concentrated. The residue was purified by silica gel chromatography (PE:EA=1.5:1) to afford (R)-tert-butyl (1-(2-chloro-7-nitroquinazolin-4-yl)pyrrolidin-3-yl)carbamate (4.8 g, 99%) as a yellow solid. [M+H] Calc'd for C₁₇H₂₀ClN₅O₄, 394.1; Found, 394.1

Step 2: (R)-tert-butyl (1-(2-morpholino-7-nitroquinazolin-4-yl)pyrrolidin-3-yl)carbamate

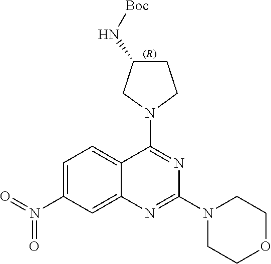

A solution of (R)-tert-butyl (1-(2-chloro-7-nitroquinazolin-4-yl)pyrrolidin-3-yl)carbamate (1.2 g, 3.0 mmol), Morpholine (313 mg, 3.6 mmol) and DIEA (1.16 g, 9.0 mmol) in NMP (10 mL) was stirred at 90° C. for 3 hrs. The mixture was cooled, diluted with EtOAc (200 mL), washed with water (100 mL*3) and brine (50 mL), dried over Na₂SO_{4,} filtered and concentrated in-vacuo to afford (R)-tert-butyl (1-(2-morpholino-7-nitroquinazolin-4-yl)pyrrolidin-3 -yl)carbamate (1.3 g, 98%) as a red solid. [M+H] Calc'd for C₂₁H₂₈N₆O₅, 445.2; Found, 445.2

Step 3: (R)-tert-butyl (1-(7-amino-2-morphohnoquinazolin-4-yl)pyrrolidin-3-yl)carbamate

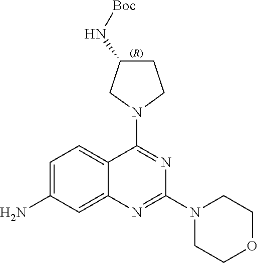

A mixture of (R)-tert-butyl (1-(2-morpholino-7-nitroquinazolin-4-yl)pyrrolidin-3-yl)carbamate (1.33 g, 3.0 mmol) and NH₄Cl (1.6 g, 30.0 mmol) in MeOH (30 mL) and water (10 mL) was stirred at 80° C. for 2 hrs. Then Zn (1.6 g, 30.0 mmol) was added. The mixture was stirred at 80° C. for 2 hrs. The mixture was cooled, filtered and concentrated. The residue was purified by column chromatography (DCM:MeOH=10:1) to afford (R)-tert-butyl (1-(7-amino-2-morpholinoquinazolin-4-yl)pyrrolidin-3-yl)carbamate (800 mg, 65%) as a red solid. [M+H] Calc'd for C₂₁H₃₀N₆O₃, 415.2; Found, 415.2

Step 4: (R)-tert-butyl (1-(7-acrylamido-2-morpholinoquinazolin-4-yl)pyrrolidin-3-yl)carbamate

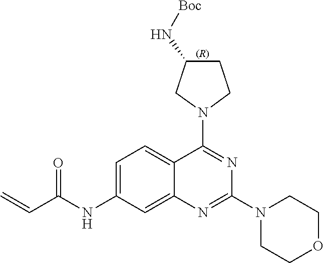

To a solution of (R)-tert-butyl (1-(7-amino-2-morpholinoquinazolin-4-yl)pyrrolidin-3-yl)carbamate (1.0 g, 2.41 mmol) in DCM (10 mL) was added DIEA (622 mg, 4.82 mmol) and acryloyl chloride (219 mg, 2.41 mmol). The mixture was stirred at rt for 2 hrs. The mixture was washed with water (10 mL) and extracted with DCM (40 mL). The organic layer was concentrated. The residue was purified by column chromatography (DCM:EA:THF=4:1:0.5) to afford (R)-tert-butyl (1-(7-acrylamido-2-morpholinoquinazolin-4-yl)pyrrolidin-3-yl)carbamate (800 mg, 71%) as a red solid. [M+H] Calc'd for C₂₄H₃₂N₆O₄, 469.2; Found, 469.2.

Step 5: (R)—N-(4-(3-aminopyrrolidin-1-yl)-2-morphohnoquinazolin-7-yl)acrylamide

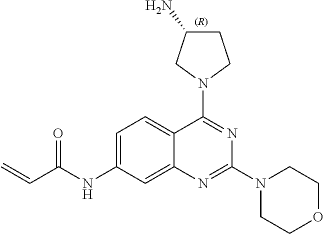

A solution of (R)-tert-butyl (1-(7-acrylamido-2-morpholinoquinazolin-4-yl)pyrrolidin-3-yl)carbamate (200 mg, 0.43 mmol) and TFA (1.5 mL) in DCM (6 mL) was stirred at RT for 1 h. The reaction mixture was concentrated in-vacuo to afford (R)—N-(4-(3-aminopyrrolidin-1-yl)-2-morpholinoquinazolin-7-yl)acrylamide (TFA salt) (157 mg, 100%) as red oil. [M+H] Calc'd for C₁₉H₂₄N₆O₂, 369.2; Found,369.2

Step 6: (R)—N-(4-(3-((5-bromopyrimidin-2-yl)amino)pyrrolidin-1-yl)-2-morpholinoquinazolin-7-yl)acrylamide

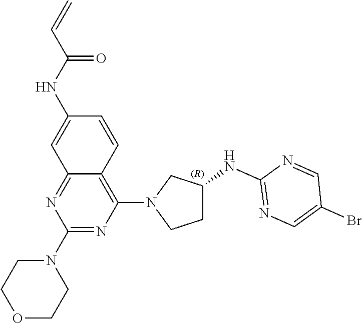

A mixture of (R)—N-(4-(3-aminopyrrolidin-1-yl)-2-morpholinoquinazolin-7-yl)acrylamide (TFA salt) (157 mg, 0.43 mmol), 5-Bromo-2-chloro-pyrimidine (82 mg, 0.43 mmol) and DIEA (278 mg, 2.15 mmol) in DMSO (3 mL) was heated to 60° C. overnight. The reaction mixture was cooled, diluted with water (20 mL) and extracted with DCM (40 mL). The organic layer was washed with water (50 mL*3) and brine (20 mL), dried over Na₂SO₄, filtered and concentrated in-vacuo. The residue was purified by prep-HPLC to afford (R)—N-(4-(3-((5-bromopyrimidin-2-yl)amino)pyrrolidin-1-yl)-2-morpholinoquinazolin-7-yl)acrylamide (64.2 mg, 28%) as a white solid.

¹H NMR (400 MHz, DMSO-d₆): δ 2.03-2.07 (m, 1H), 2.20-2.22 (m, 1H), 3.64-3.81 (m, 8H), 3.77-3.81 (m, 1H), 3.86-3.91 (m, 1H), 4.01-4.13 (m, 2H), 4.43-4.45 (m, 1H), 5.79 (dd, J=2.0, 10.0 Hz, 1H), 6.27-6.32 (m, 1H), 6.43-6.50 (m, 1H), 7.21 (dd, J=2.4, 9.2 Hz, 1H), 7.83-7.87 (m, 2H), 7.98 (d, J=9.2 Hz, 1H), 8.13 (s, 1H), 8.42 (s, 2H), 10.27 (s, 1H). [M+H] Calc'd for C₂₃H₂₅BrN₈O₂, 525.1; Found,525.1

### Example 21: Synthesis of (R)—N-(443-((5-bromopyrimidin-2-yl)amino)pyrrolidin-1-yl)-2-methylquinazolin-7-yl)acrylamide

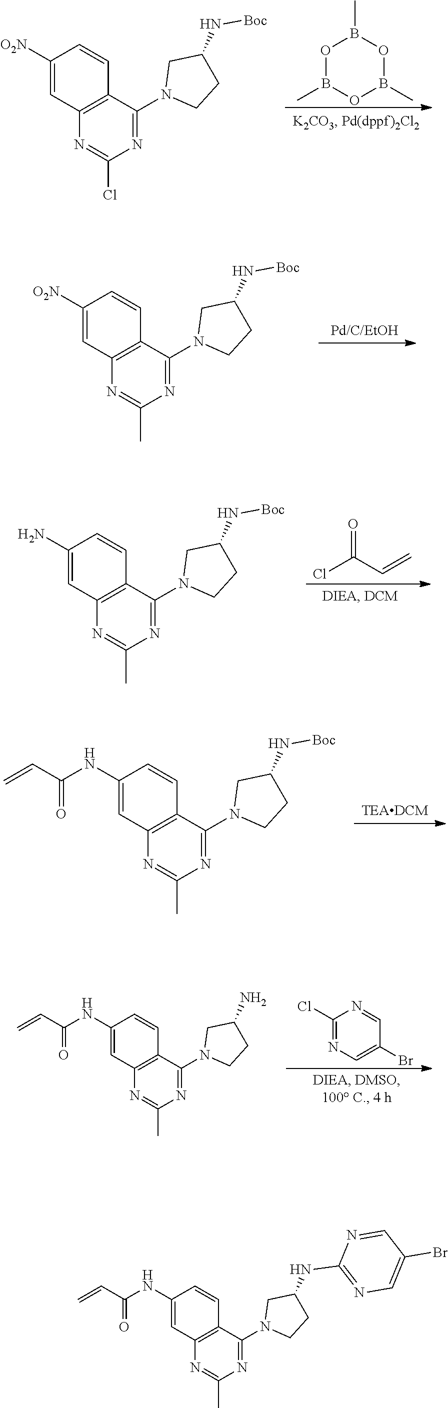

Step 1: (R)-tert-butyl (1-(2-methyl-7-nitroquinazolin-4-yl)pyrrolidin-3-yl)carbamate

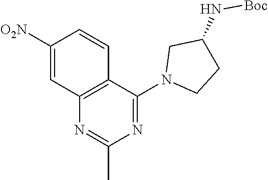 [SMILES: Cc1nc(N2CC[C@@H](NC(=O)OC(C)(C)C)C2)c2ccc([N+](=O)[O-])cc2n1]

A mixture of (R)-tert-butyl (1-(2-chloro-7-nitroquinazolin-4-yl)pyrrolidin-3-yl)carbamate (500 mg, 1.26 mmol), 2,4,6-trimethyl-1,3,5,2,4,6-trioxatriborinane (540 mg, 1.90 mmol), Pd(dppf)₂C₁₂ (103 mg, 0.12 mmol) and K₂CO₃ (520 mg, 3.80 mmol) in 1,4-Dioxane (6 mL) was stirred at 115° C. under microwave for 2 hrs. The solvent was removed under reduced pressure and the residue was diluted with DCM (30 mL), then washed with water (20 mL*2) and brine (20 mL), dried over Na₂SO_{4,} filtered and concentrated. The residue was purified by coloumn chromatography with (1:1, Pet ether:Ethyl acetate) to afford(R)-tert-butyl (1-(2-methyl-7-nitroquinazolin-4-yl)pyrrolidin-3-yl)carbamate (200 mg, 42.2%) as a yellow solid. [M+H] Calc'd for C₁₈H₂₃N₅O₄ 374.0; Found, 374.0.

Step 2: (R)-tert-butyl (1-(7-amino-2-methylquinazolin-4-yl)pyrrolidin-3-yl)carbamate

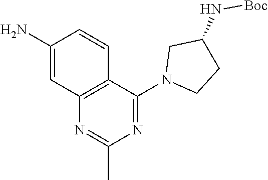 [SMILES: Cc1nc(N2CC[C@@H](NC(=O)OC(C)(C)C)C2)c2ccc(N)cc2n1]

A mixture of (R)-tert-butyl (1-(2-methyl-7-nitroquinazolin-4-yl)pyrrolidin-3-yl)carbamate (1.4 g, 3.8 mmol) in EtOH (50 mL) was added Pd/C (0.8 g). The reaction was stirred at R.T under hydrogen atmosphere for 3 hrs. The mixture was filtered and concentrated to afford (R)-tert-butyl (1-(7-amino-2-methylquinazolin-4-yl)pyrrolidin-3-yl)carbamate (1.2 g, crude) as an off white solid. [M+H] Calc'd for C₁₈H₂₅N₅O₂, 344.3; Found, 344.3.

Step 3: (R)-tert-butyl (1-(7-acrylamido-2-methylquinazolin-4-yl)pyrrolidin-3-yl)carbamate

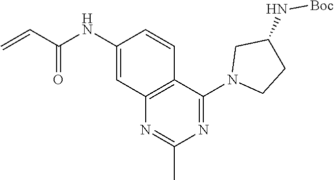 [SMILES: C=CC(=O)Nc1ccc2c(N3CC[C@@H](NC(=O)OC(C)(C)C)C3)nc(C)nc2c1]

Acryloyl chloride (0.27 mL, 3.41 mmol) was slowly added to a mixture of (R)-tert-butyl (1-(7-amino-2-methylquinazolin-4-yl)pyrrolidin-3-yl)carbamate (1.3 g, 3.78 mmol) and DIEA (1.67 mL, 11.3 mmol) in DCM (80 mL) at 0° C. The mixture was stirred at RT for 3 h. The reaction mixture was concentrated. The residue was purified by prep-HPLC to afford (R)-tert-butyl (1-(7-acrylamido-2-methylquinazolin-4-yl)pyrrolidin-3-yl)carbamate (12 mg, 9.2%) as a white solid. [M+H] Calc'd 398.4; Found, 398.4.

Step 4: (R)—N-(4-(3-aminopyrrolidin-1-yl)-2-methylquinazolin-7-yl)acrylamide

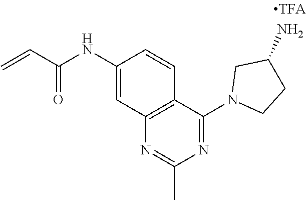 [SMILES: C=CC(=O)Nc1ccc2c(N3CC[C@@H](N)C3)nc(C)nc2c1]

TFA (0.2 mL) was added to a solution of (R)-tert-butyl (1-(7-acrylamido-2-methylquinazolin-4-yl)pyrrolidin-3-yl)carbamate (135 mg, 0.34 mmol) in dichloromethane (3 mL) at RT. The mixture was stirred at RT for 3 h. The reaction mixture was concentrated to afford TFA salt of (R)—N-(4-(3-aminopyrrolidin-1-yl)-2-methylquinazolin-7-yl)acrylamide (200 mg, crude) as brown liquid. [M+H] Calc'd 298.2; Found, 298.3.

Step 5: (R)—N-(4-(3-((5-bromopyrimidin-2-yl)amino)pyrrolidin-1-yl)-2-methylquinazolin-7-yl)acrylamide

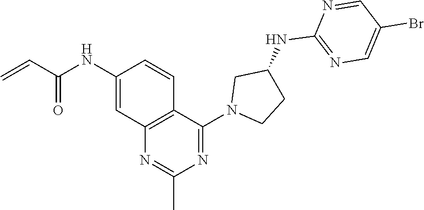 [SMILES: C=CC(=O)Nc1ccc2c(N3CC[C@@H](Nc4ncc(Br)cn4)C3)nc(C)nc2c1]

A mixture of (R)—N-(4-(3-aminopyrrolidin-1-yl)-2-methylquinazolin-7-yl)acrylamide (100 mg, 0.33 mmol), 5-Bromo-2-chloro-pyrimidine (104 mg, 0.53 mml) and DIEA (0.55 mL, 3.36 mmol) in DMSO (10 mL) was stirred at 100° C. under nitrogen atmosphere for 4 hrs. The reaction mixture was cooled and concentrated. The residue was purified by prep-HPLC to afford (R)—N-(4-(3-((5-bromopyrimidin-2-yl)amino)pyrrolidin-1-yl)-2-methylquinazolin-7-yl)acrylamide (10.3 mg, 8.3%) as a white solid. 1HNMR (400 MHz, DMSO-d₆): 2.07 (m, 1H), 2.02 (m, 1H), 2.41 (s, 3H), 3.82-4.03 (m, 4H), 4.421-4.44 (m, 1H), 5.81 (dd, J=2.0, 10.0 Hz, 1H), 6.29-6.34 (m, 1H), 6.45-6.52 (m, 1H), 7.55 (dd, J=2.4, 9.2 Hz,1H), 7.84 (d, J=6.0 Hz, 1H), 8.048 (d, J=2.4 Hz, 1H), 8.15 (d, J=9.6 Hz, 1H), 8.42 (s, 2H), 10.42 (s, 1H). [M+H] Calc'd 455.3; Found, 455.3.

### Example 22: Synthesis of (R)—N-(4-(3-((5-cyanopyrimidin-2-yl)amino)pyrrolidin-1-yl)-2-morpholinoquinazolin-7-yl)acrylamide

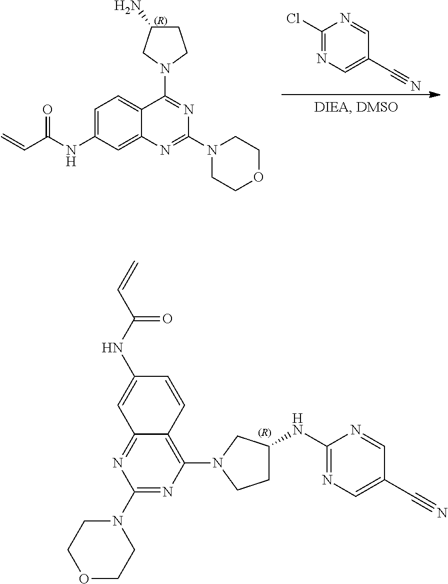

A mixture of (R)—N-(4-(3-aminopyrrolidin-1-yl)-2-morpholinoquinazolin-7-yl)acrylamide (TFA salt) (146 mg, 0.4 mmol), 2-chloropyrimidine-5-carbonitrile (55 mg, 0.4 mmol) and DIEA (258 mg, 2.0 mmol) in DMSO (2 mL) was heated to 70° C. for 2 hrs. The residue was cooled, diluted with water (20 mL) and extracted with DCM (30 mL*2). The combined organic layers were washed with water (30 mL*2) and brine (20 mL), dried over Na₂SO₄, filtered and concentrated in vacuo. The residue was purified by prep-HPLC to afford (R)—N-(4-(3-((5-cyanopyrimidin-2-yl)amino)pyrrolidin-1-yl)-2-morpholinoquinazolin-7-yl)acrylamide (41.4 mg, 22%) as a yellow solid. ¹H NMR (400 MHz, DMSO-d6): δ 2.07-2.11 (m, 1H), 2.23-2.28 (m, 1H), 3.64-3.71 (m, 8H), 3.79-3.83 (m, 1H), 3.89-3.93 (m, 1H), 4.02-4.05 (m, 1H), 4.11-4.15 (m, 1H), 4.55-4.59 (m, 1H), 5.79 (dd, J=10.0, 2.0 Hz, 1H), 6.27-6.32 (m, 1H), 6.44-6.50 (m, 1H), 7.22 (dd, J=2.0, 8.8 Hz, 1H), 7.86 (d, J=2.0 Hz, 1H), 7.97 (d, J=9.2 Hz, 1H), 8.14 (s, 1H), 8.65-8.69 (m, 2H), 8.77 (d, J=2.4 Hz, 1H), 10.26 (s, 1H). [M+H] Calc'd for C₂₄H₂₅N₉O₂, 472.2; Found, 472.2.

### Example 23: (R)—N-(4-(3-((5-bromopyrimidin-2-yl)amino)pyrrolidin-1-yl)-2-(4-methylpiperazin-1-yl)quinazolin-7-yl)acrylamide

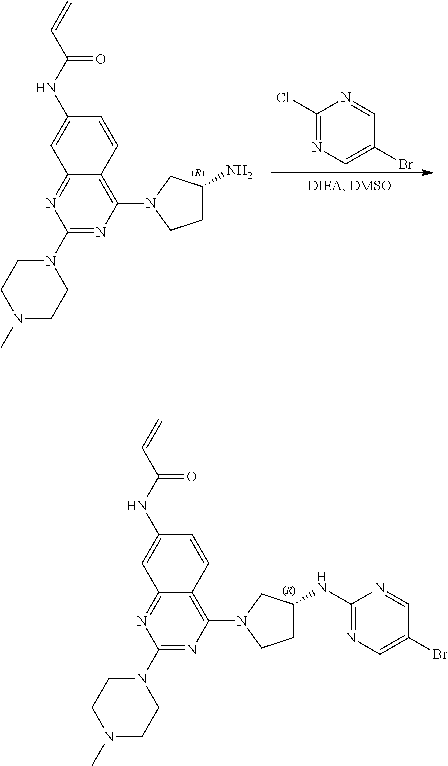

Step 1: (R)—N-(4-(3-((5-bromopyrimidin-2-ypamino)pyrrolidin-1-yl)-2-(4-methylpiperazin-1-yl)quinazolin-7-yl)acrylamide

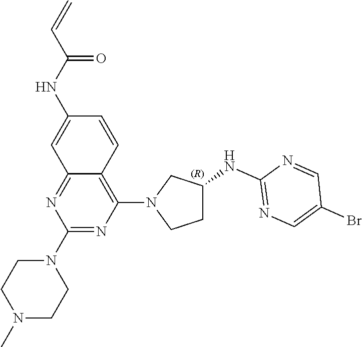

A mixture of (R)—N-(4-(3-aminopyrrolidin-1-yl)-2-(4-methylpiperazin-1-yl)quinazolin-7-yl)acrylamide (139 mg, 0.36 mmol), 5-bromo-2-chloropyrimidine (75 mg, 0.36 mmol) and DIEA (232 mg, 26.00 mmol) in DMSO (2 mL) was heated to 60° C. overnight. The reaction mixture was cooled, diluted with water (10 mL) and extracted with DCM (10 mL*2). The combined organic layer was washed with water (10 mL*2) and brine (10 mL), dried over Na₂SO_{4,} filtered and concentrated in vacuo. The residue was purified by prep-HPLC to afford (R)—N-(4-(3-((5-bromopyrimidin-2-yl)amino)pyrrolidin-1-yl)-2-(4-methylpiperazin-1-yl)quinazolin-7-yl)acrylamide (2.5 mg, 1%) as a white solid. ¹H NMR (400 MHz, DMSO-d6): δ 1.23-1.29 (m, 1H), 1.44-1.46 (m, 1H), 2.00-2.07 (m, 3H), 2.19-2.24 (m, 2H), 2.38-2.41 (m, 2H), 3.73-4.12 (m, 8H), 4.39-4.42 (m, 1H), 5.79 (dd, J=2.0, 10.0 Hz, 1H), 6.26-6.31 (m, 1H), 6.43-6.47 (m, 1H), 7.19 (dd, J=2.4, 9.2 Hz, 1H), 7.82-7.83 (m, 2H), 7.96 (d, J=9.2 Hz, 1H), 8.42 (s, 2H), 10.24 (s, 1H). [M+H] MS Calc'd for C₂₄H₂₈BrN₉O, 538.2; Found, 538.2.

### Example 24: (R)—N-(4434(5-cyanopyrimidin-2-yl)amino)pyrrolidin-1-yl)-244-methylpiperazin-1-yl)quinazolin-7-yl)acrylamide

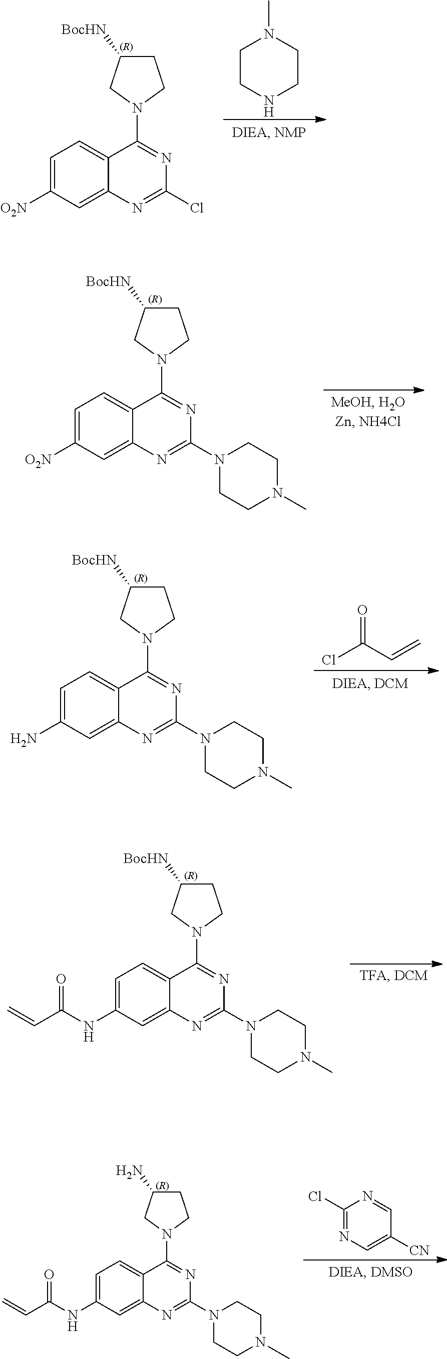

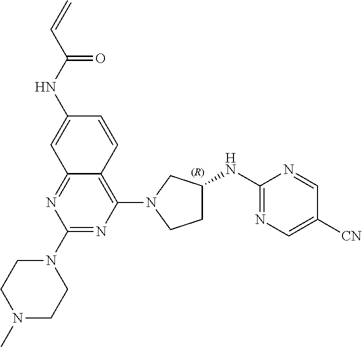

Step 1: (R)-tert-butyl (1-(2-(4-methylpiperazin-1-yl)-7-nitroquinazolin-4-yl)pyrrolidin-3-yl)carbamate

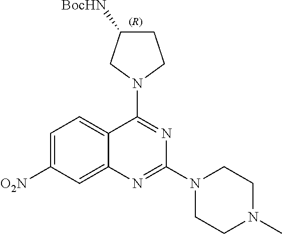

A solution of (R)-tert-butyl (1-(2-chloro-7-nitroquinazolin-4-yl)pyrrolidin-3-yl)carbamate (10.0 g, 25.4 mmol), 1-methylpiperazine (2.6 g, 26.0 mmol) and DIEA (6.6 g, 50.8 mmol) in NMP (100 mL) was stirred at 90° C. for 3 hrs. The reaction mixture was cooled and extracted with EtOAc (200 mL*2). The combined organic layer was washed with water (100 mL*3) and brine (100 mL), dried over anhydrous sodium sulfate, filtered and concentrated under reduced pressure. The residue was purified by column chromatography on silica gel (DCM:MeOH=10:1) to give (R)-tert-butyl (1-(2-(4-methylpiperazin-1-yl)-7-nitroquinazolin-4-yl)pyrrolidin-3-yl)carbamate (9.0 g, 77%) as a red solid. [M+H] MS Calc'd for C₂₂H₃₁N₇O₄, 458.2; Found, 458.2.

Step 2: (R)-tert-butyl (1-(7-amino-2-(4-methylpiperazin-1-yl)quinazolin-4-yl)pyrrolidin-3-yl)carbamate

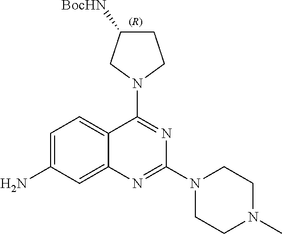

A mixture of (R)-tert-butyl (1-(2-(4-methylpiperazin-1-yl)-7-nitroquinazolin-4-yl)pyrrolidin-3-yl)carbamate (9.0 g, 19.7 mmol) and NH₄Cl (10.4 g, 197.0 mmol) in MeOH (90 mL) and water (30 mL) was stirred at 80° C. for 2 hrs. Then Zn (12.8 g, 197.0 mmol) was added. The mixture was stirred at 80° C. for 2 hrs. The mixture was cooled, filtered and concentrated. The residue was purified by column (DCM:MeOH=10:1) to afford (R)-tert-butyl (1-(7-amino-2-(4-methylpiperazin-1-yl)quinazolin-4-yl)pyrrolidin-3-yl)carbamate (6.7 g, 79%) as a red solid. [M+H] MS Calc'd for C₂₂H₃₃N₇O₂, 428.3; Found, 428.3.

Step 3: (R)-tert-butyl (1-(7-acrylamido-2-(4-methylpiperazin-1-yl)quinazolin-4-yl)pyrrolidin-3-yl)carbamate

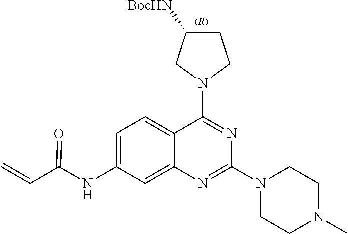

To a solution of (R)-tert-butyl (1-(7-amino-2-(4-methylpiperazin-1-yl)quinazolin-4-yl)pyrrolidin-3-yl)carbamate (6.7 g, 15.6 mmol) in DCM (50 mL) was added DIEA (8.0 mL, 46.8 mmol) and acryloyl chloride (1.4 g, 15.6 mmol) at 0° C. The reaction mixture was stirred at rt for 2 hrs. The mixture was diluted with water (50 mL) and extracted with DCM (50 mL*2). The combined organic layer was concentrated. The residue was purified by column (DCM:MeOH=10:1) to afford (R)-tert-butyl (1-(7-acrylamido-2-(4-methylpiperazin-1-yl)quinazolin-4-yl)pyrrolidin-3-yl)carbamate (2.2 g, 29%) as a yellow solid. [M+H] MS Calc'd for C₂₅H₃₅N₇O₃, 482.3; Found, 482.3.

Step 4: (R)—N-(4-(3-aminopyrrolidin-1-yl)-2-(4-methylpiperazin-1-yl)quinazolin-7-yl)acrylamide TFA salt

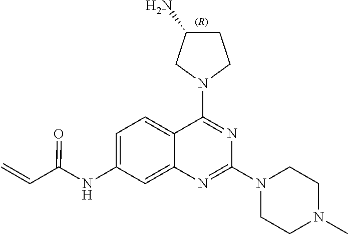

A solution of (R)-tert-butyl (1-(7-acrylamido-2-(4-methylpiperazin-1-yl)quinazolin-4-yl)pyrrolidin-3-yl)carbamate (2.2 g, 5.6 mmol) and TFA (6 mL) in DCM (20 mL) was stirred at RT for 1 h. The reaction mixture was concentrated in vacuo to afford TFA salt of (R)—N-(4-(3-aminopyrrolidin-1-yl)-2-(4-methylpiperazin-1-yl)quinazolin-7-yl)acrylamide (1.74 g) as yellow oil. [M+H] MS Calc'd for C₂₀H₂₇N₇O, 382.2; Found,382.2.

Step 5: (R)—N-(4-(3-((5-cyanopyrimidin-2-yl)amino)pyrrolidin-1-yl)-2-(4-methylpiperazin-1-yl)quinazolin-7-yl)acrylamide HCCOH salt

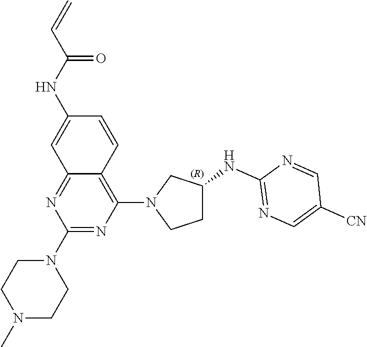

A mixture of TFA salt of (R)—N-(4-(3-aminopyrrolidin-1-yl)-2-(4-methylpiperazin-1-yl)quinazolin-7-yl)acrylamide (2.0 g, 5.2 mmol), 2-chloropyrimidine-5-carbonitrile (729 mg, 5.2 mmol) and DIEA (4.6 mL, 26.0 mmol) in DMSO (20 mL) was heated to 30° C. for 2 h. The reaction mixture was cooled, diluted with water (40 mL) and extracted with DCM (40 mL*2). The combined organic layer was washed with water (50 mL*2) and brine (50 mL), dried over Na₂SO_{4,} filtered and concentrated in vacuo. The residue was purified by prep-HPLC to afford HCOOH salt of (R)—N-(4-(3-((5-cyanopyrimidin-2-yl)amino)pyrrolidin-1-yl)-2-(4-methylpiperazin-1-yl)quinazolin-7-yl)acrylamide (295.0 mg, 12%) as a yellow solid. ¹H NMR (400 MHz, DMSO-d₆): δ 2.03-2.10 (m, 1H), 2.20-2.27 (m, 1H), 2.53-2.57 (m, 3H), 2.67-2.81 (m, 4H), 3.80-4.15 (m, 8H), 4.57-4.59 (m, 1H), 5.79 (dd, J=2.0, 10.0 Hz, 1H), 6.27-6.32 (m, 1H), 6.43-6.50 (m, 1H), 7.22 (dd, J=2.4, 9.2 Hz, 1H), 7.95-8.02 (m, 2H), 8.13 (s, 1H), 8.65-8.70 (m, 2H), 8.78 (s, 1H), 10.31 (s, 1H). [M+H] MS Calc'd for C₂₅H₂₈N₁₀O, 485.2; Found, 485.2.

### Example 25: (R)—N-(4-(3-((5-bromopyrimidin-2-yl)amino)pyrrolidin-1-yl)quinazolin-7-yl)acrylamide

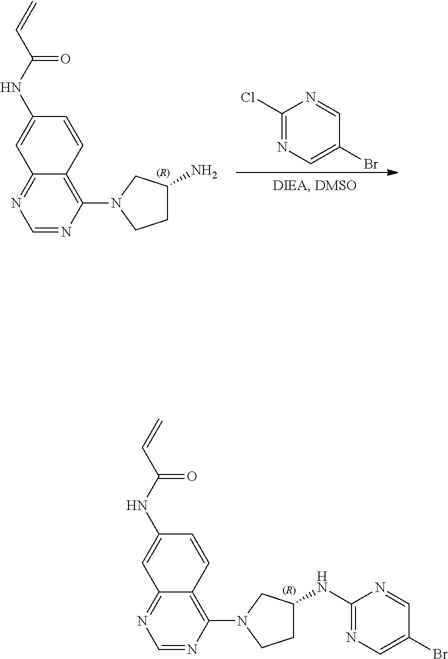

Step 1: (R)—N-(4-(3-((5-bromopyrimidin-2-yl)amino)pyrrolidin-1-yl)quinazolin-7-yl)acrylamide HCOOH salt

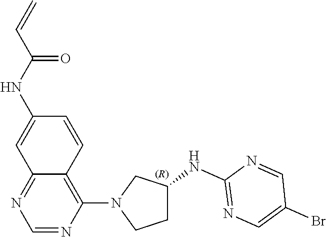

A mixture of (R)—N-(4-(3-aminopyrrolidin-1-yl)quinazolin-7-yl)acrylamide (TFA salt) (170 mg, 0.60 mmol), 5-bromo-2-chloropyrimidine (116 mg, 0.60 mmol) and DIEA (0.5 mL, 3.0 mmol) in DMSO (10 mL) was heated to 60° C. for 2 days. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford HCOOH salt of (R)—N-(4-(3-((5-bromopyrimidin-2-yl)amino)pyrrolidin-1-yl)quinazolin-7-yl)acrylamide (29.8 mg, 11%) as a white solid. ¹H NMR (400 MHz, DMSO-d₆): δ 2.03-2.10 (m, 1H), 2.20-2.27 (m, 1H), 3.82-3.85 (m, 1H), 3.91-3.97 (m, 1H), 4.03-4.10 (m, 1H), 4.15-4.19 (m, 1H), 4.43-4.47 (m, 1H), 5.83 (dd, J=2.4, 10.2 Hz, 1H), 6.30-6.35 (m, 1H), 6.45-6.52 (m, 1H), 7.63 (dd, J=2.4, 9.2 Hz, 1H), 7.87 (d, J=6.0 Hz, 1H), 8.13-8.16 (m, 2H), 8.22 (d, J=9.2 Hz, 1H), 8.38-8.42 (m, 3H), 10.47 (s, 1H). [M+H] MS Calc'd for C₁₉H₈BrN₇O, 440.1; Found, 440.0.

### Example 26: (R)—N-(1-(3-((5-cyanopyrimidin-2-yl)amino)pyrrolidin-1-yl)isoquinolin-6-yl)acrylamide

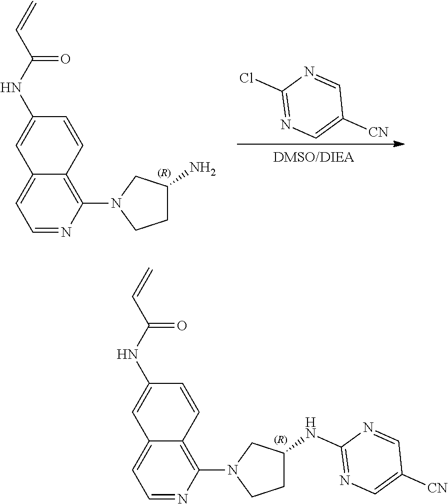

Step 1: (R)—N-(1-(3-((5-cyanopyrimidin-2-yl)amino)pyrrolidin-1-yl)isoquinolin-6-yl)acrylamide HCOOH salt

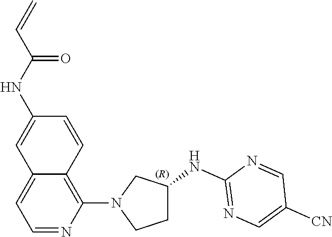

A mixture of (R)—N-(1-(3-aminopyrrolidin-1-yl)isoquinolin-6-yl)acrylamide (160 mg, 0.56 mmol), 2-chloropyrimidine-5-carbonitrile (79 mg, 0.56 mml) and DIEA (365 mg, 2.83 mmol) in DMSO (10 mL) was stirred at 40° C. for 1 h. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford HCOOH salt of (R)—N-(1-(3-((5-cyanopyrimidin-2-yDamino)pyrrolidin-1-yl)isoquinolin-6-yl)acrylamide (165.8 mg, 76.7%) as a yellow solid. ¹H NMR (400 MHz, DMSO-d₆): δ 2.06-2.09 (m, 1H), 2.23-2.26 (m, 1H), 3.72-3.75 (m, 1H), 3.82-3.85 (m, 1H), 3.94-3.96 (m, 1H), 4.05-4.09 (m, 1H), 4.51-4.54 (m, 1H), 5.81 (dd, J=2.0, 10.4 Hz, 1H), 6.29-6.33 (m, 1H), 6.46-6.52 (m, 1H), 6.98 (d, J=5.6 Hz, 1H), 7.58 (dd, J=2.0, 9.2 Hz, 1H), 7.87 (d, J=5.6 Hz, 1H), 8.13-8.20 (m, 3H), 8.67-8.76 (m, 3H), 10.42 (s, 1H), 12.61 (br s, 1H). [M+H] MS Calc'd for C₂₁H₁₉N₇O, 386.2; Found, 386.2

### Example 27: (R)—N4143-((5-bromopyrimidin-2-yl)amino)pyrrolidin-1-yl)isoquinolin-6-yl)-N-methylacrylamide

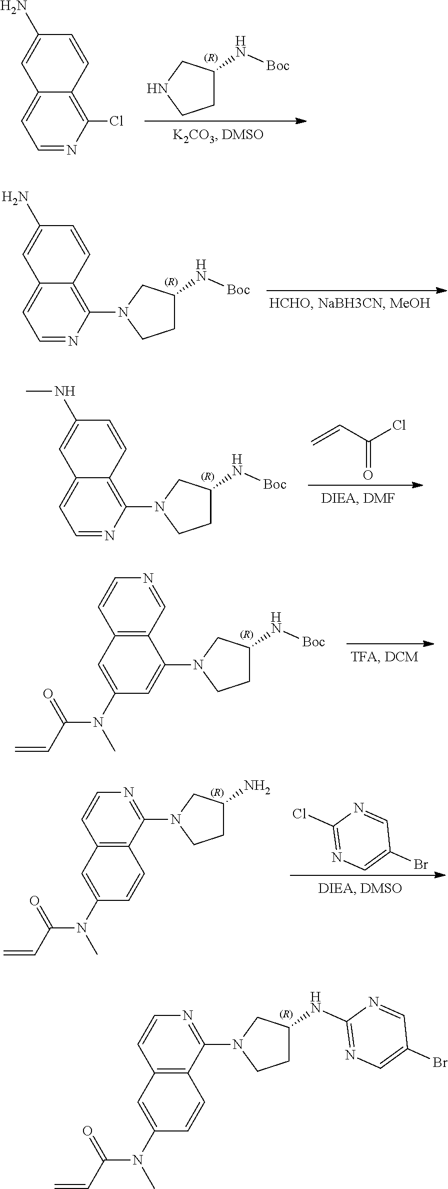

Step 1: (R)-tert-butyl (1-(6-aminoisoquinolin-1-yl)pyrrolidin-3-yl)carbamate

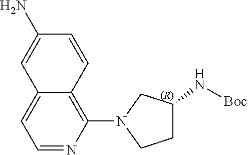

A mixture of 1-chloroisoquinolin-6-amine (500 mg, 2.8 mmol), (R)-tert-butyl pyrrolidin-3-ylcarbamate (1.0 g, 5.6 mml) and K₂CO₃ (580 mg, 4.2 mmol) in DMSO (10 mL) was stirred at 160° C. for 1 h in microwave. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by column chromatography on silica gel (PE:EA=0:1) to afford (R)-tert-butyl (1-(6-aminoisoquinolin-1-yl)pyrrolidin-3-yl)carbamate (1.4 g, 39%) as a yellow solid. [M+H] MS Calc'd for C₁₈H₂₄N₄O₂, 329.2; Found, 329.2.

Step 2: (R)-tert-butyl (1-(6-(methylamino)isoquinolin-1-yl)pyrrolidin-3-yl)carbamate

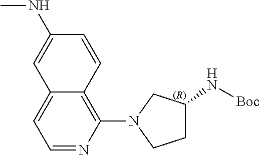

To a solution of (R)-tert-butyl (1-(6-aminoisoquinolin-1-yl)pyrrolidin-3-yl)carbamate (500 mg, 1.5 mmol) in MeOH (20 mL) was added HCHO (183 mg, 30% in water, 6.0 mmol). The reaction mixture was stirred at RT for 1 h. Then NaBH₃CN (144 mg, 12.0 mmol) was added. The mixture was stirred at 60° C. overnight. The mixture was cooled, diluted with 2N HCl solvent (5 mL) and extracted with EA (20 mL*2). The combined organic layer was concentrated. The residue was purified by column chromatography on silica gel (PE:EA=0:1) to afford (R)-tert-butyl (1-(6-(methylamino)isoquinolin-1-yl)pyrrolidin-3-yl)carbamate (300 mg, 53%) as a yellow solid. [M+H] MS Calc'd for C₁₉H₂₆N₄O₂, 343.2; Found, 343.2.

Step 3: (R)-tert-butyl (1-(6-(N-methylacrylamido)isoquinolin-1-yl)pyrrolidin-3-yl)carbamate

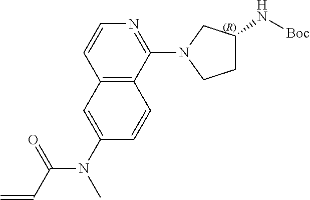

To a mixture of (R)-tert-butyl (1-(6-(methylamino)isoquinolin-1-yl)pyrrolidin-3-yl)carbamate (250 mg, 0.72 mmol) and DIEA (283 mg, 2.21 mmol) in DCM (20 mL) was added acryloyl chloride (73 mg, 0.80 mmol) at 0° C. under nitrogen atmosphere slowly. The mixture was stirred at RT overnight. The mixture was concentrated and purified by column chromatography on silica gel (PE:EA=0:1) to afford (R)-tert-butyl (1-(6-(N-methylacrylamido)isoquinolin-1-yl)pyrrolidin-3-yl)carbamate (200 mg, 58%) as yellow oil. [M+H] MS Calc'd for C₂₂H₂₈N₄O₃, 397.2; Found, 397.2.

Step 4: (R)—N-(1-(3-aminopyrrolidin-1-yl)isoquinolin-6-yl)-N-methylacrylamide TFA salt

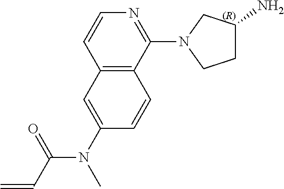

A solution of (R)-tert-butyl (1-(6-(N-methylacrylamido)isoquinolin-1-yl)pyrrolidin-3-yl)carbamate (200 mg, 0.50 mmol) and TFA (5 mL) in DCM (5 mL) was stirred at RT for 2 hrs. The mixture was concentrated to afford TFA salt of (R)—N-(1-(3-aminopyrrolidin-1-yl)isoquinolin-6-yl)-N-methylacrylamide (148 mg) as brown oil. [M+H] MS Calc'd for C₁₇H₂₀N₄O, 297.2; Found, 297.2.

Step 5: (R)—N-(1-(3-((5-bromopyrimidin-2-yl)amino)pyrrolidin-1-yl)isoquinolin-6-yl)-N-methylacrylamide

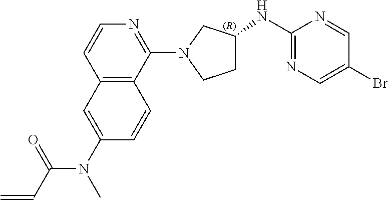

A mixture of (R)—N-(1-(3-aminopyrrolidin-1-yl)isoquinolin-6-yl)-N-methylacrylamide (100 mg, 0.34 mmol), 5-bromo-2-chloropyrimidine (98 mg, 0.51 mmol) and DIEA (132 mg, 0.90 mmol) in DMSO (10 mL) was stirred at 80° C. for 1 h in microwave. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford (R)—N-(1-(3-((5-bromopyrimidin-2-yl)amino)pyrrolidin-1-yl)isoquinolin-6-yl)-N-methylacrylamide (27.5 mg, 12%) as a white solid. ¹HNMR (400 MHz, DMSO-d₆): δ 2.04-2.07 (m, 1H), 2.21-2.25 (m, 1H), 3.33 (s, 3H), 3.71-3.75 (m, 1H), 3.83-3.85 (m, 1H), 3.95-3.98 (m, 1H), 4.06-4.11 (m, 1H), 4.40-4.42 (m, 1H), 5.59 (dd, J=3.2, 9.6 Hz, 1H), 6.15-6.18 (m, 2H), 7.04 (d, J=5.6 Hz, 1H), 7.36 (dd, J=2.0, 9.2 Hz, 1H), 7.64 (d, J=2.0 Hz, 1H), 7.82 (d, J=6.4 Hz, 1H), 7.95 (d, J=5.6 Hz, 1H), 8.28 (d, J=8.8 Hz, 1H), 8.40 (s, 2H). [M+H] MS Calc'd for C₂₁H₂₁BrN₆O, 453.1; Found, 453.2.

### Example 28: (R)—N-(1434(5-bromopyrimidin-2-yflamino)pyrrolidin-1-yl)isoquinolin-6-yl)-N-methylacrylamide

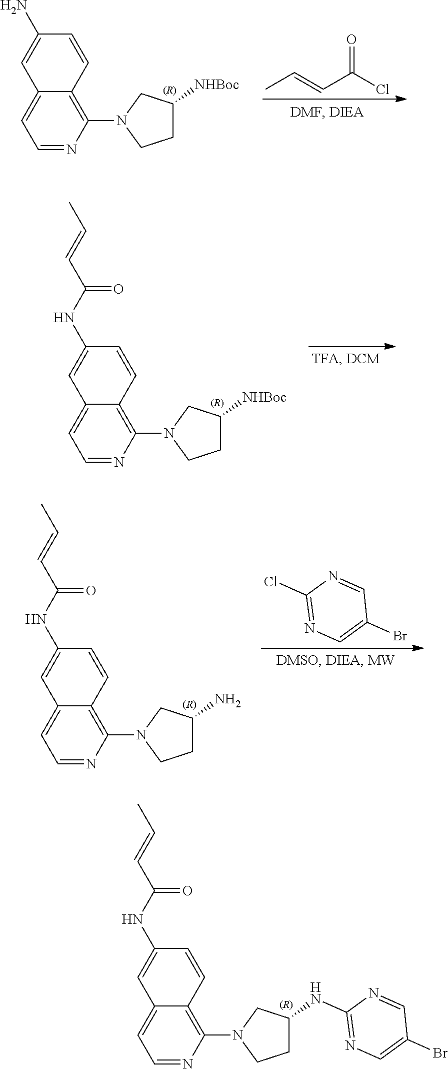

Step 1: (R,E)-tert-butyl (1-(6-(but-2-enamido)isoquinolin-1-yl)pyrrolidin-3-yl)carbamate

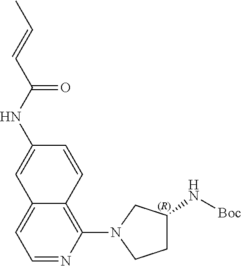

To a mixture of (R)-tert-butyl (1-(6-aminoisoquinolin-1-yl)pyrrolidin-3-yl)carbamate (600 mg, 1.83 mmol) and DIEA (708 mg, 5.48 mmol) in DCM (30 mL) was added (E)-but-2-enoyl chloride (190 mg, 1.83 mmol) at 0° C. under nitrogen atmosphere slowly. The mixture was stirred at RT overnight. The reaction mixture was concentrated and purified by column chromatography on silica gel (DCM:MeOH=20:1) to afford (R,E)-tert-butyl (1-(6-(but-2-enamido)isoquinolin-1-yl)pyrrolidin-3-yl)carbamate (280 mg, 38.6%) as a yellow solid. [M+H] MS Calc'd for C₂₂H₂₈N₄O₃, 397.2; Found, 397.2.

Step 2: (R,E)—N-(1-(3-aminopyrrolidin-1-yl)isoquinolin-6-yl)but-2-enamide TFA salt

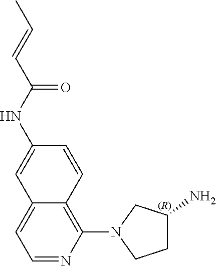

A solution of (R,E)-tert-butyl (1-(6-(but-2-enamido)isoquinolin-1-yl)pyrrolidin-3-yl)carbamate (280 mg, 0.71 mmol) and TFA (10 mL) in DCM (10 mL) was stirred at RT for 2 hrs. The mixture was concentrated to afford TFA salt of (R,E)—N-(1-(3-aminopyrrolidin-1-yl)isoquinolin-6-yl)but-2-enamide (210 mg) as brown oil. [M+H] MS Calc'd for C₁₇H₂₀N₄O, 297.2; Found, 297.2.

Step 3: (R,E)—N-(1-(3-((5-bromopyrimidin-2-yl)amino)pyrrolidin-1-yl)isoquinolin-6-yl)but-2-enamide HCOOH salt

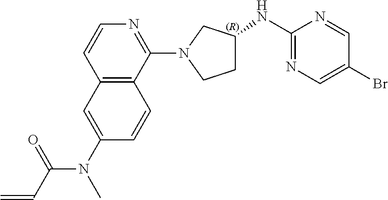

A mixture of (R,E)—N-(1-(3-aminopyrrolidin-1-yl)isoquinolin-6-yl)but-2-enamide (150 mg, 0.51 mmol), 5-bromo-2-chloropyrimidine (98 mg, 0.51 mmol) and DIEA (327 mg, 2.53 mmol) in DMSO (10 mL) was stirred at 80° C. for 1 h in microwave. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford HCOOH salt of (R,E)—N-(1-(3-((5-bromopyrimidin-2-yl)amino)pyrrolidin-1-yl)isoquinolin-6-yl)but-2-enamide (27.5 mg, 12%) as a white solid. ¹H NMR (400 MHz, DMSO-d₆): δ 1.89 (dd, J=1.2,7.2 Hz, 3H), 2.02-2.05 (m, 1H), 2.21-2.23 (m, 1H), 3.68-3.72 (m, 1H), 3.79-3.82 (m, 1H), 3.91-3.93 (m, 1H), 4.02-4.17 (m, 1H), 4.38-4.40 (m, 1H), 6.18 (dd, J=1.6, 15.2 Hz, 1H), 6.83-6.88 (m, 1H), 6.94 (d, J=6.0 Hz, 1H), 7.52-7.55 (m, 1H), 7.82-7.85 (m, 2H), 8.13-8.17 (m, 3H), 8.40 (s, 2H), 10.23 (s, 1H), 12.78 (br s, 1H). [M+H] MS Calc'd for C₂₁H₂₁BrN₆O, 453.1; Found, 453.2.

### Example 29: (R)—N-(1-(34(5-bromopyrimidin-2-yl)amino)pyrrolidin-1-yl)isoquinolin-6-yl)-N-methylacrylamide

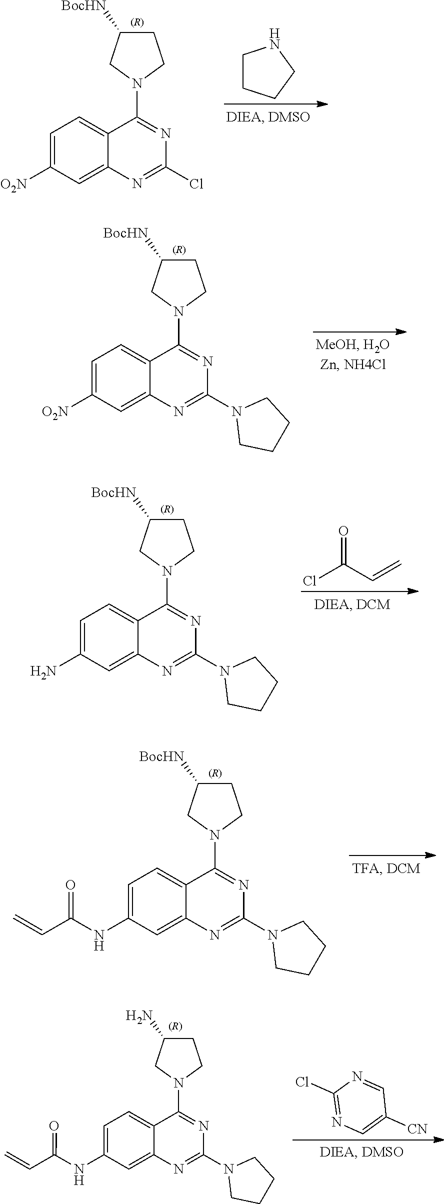

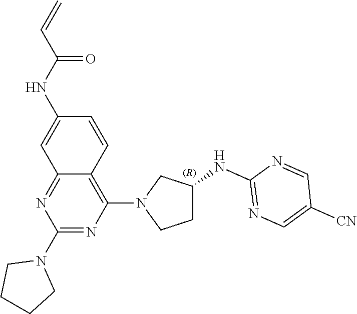

Step1: (R)-tert-butyl (1-(7-nitro-2-(pyrrolidin-1-yl)quinazolin-4-yl)pyrrolidin-3-yl)carbamate

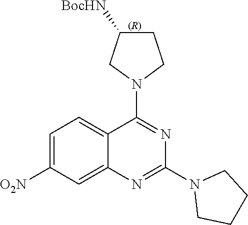

A solution of (R)-tert-butyl (1-(2-chloro-7-nitroquinazolin-4-yl)pyrrolidin-3-yl)carbamate (1.5 g, 3.8 mmol), pyrrolidine (409 mg, 5.7 mmol) and DIEA (1.5 g, 11.4 mmol) in DMSO (30 mL) was stirred at 90° C. for 2 hrs. The mixture was diluted with water (70 mL) and extracted with EtOAc (50 mL*2). The combined organic layer was washed with water (50 mL*3) and brine (50 mL), dried over anhydrous sodium sulfate, filtered and concentrated under reduced pressure. The residue was purified by column chromatography on silica gel (PE:EA=2:1) to give (R)-tert-butyl (1-(7-nitro-2-(pyrrolidin-1-yl)quinazolin-4-yl)pyrrolidin-3-yl)carbamate (1.6 g, 98%) as a yellow solid. [M+H] MS Calc'd for C₂₁ H₂₈N₆O₄, 429.2; Found, 429.2.

Step 2: (R)-tert-butyl (1-(7-amino-2-(pyrrolidin-1-yl)quinazolin-4-yl)pyrrolidin-3-yl)carbamate

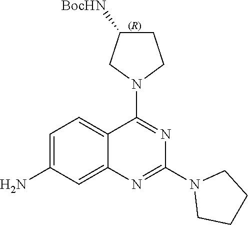

A mixture of (R)-tert-butyl (1-(7-nitro-2-(pyrrolidin-1-yl)quinazolin-4-yl)pyrrolidin-3-yl)carbamate (1.6 g, 3.7 mmol) and NH₄Cl (1.9 g, 37.0 mmol) in MeOH (40 mL) and water (8 mL) was stirred at 80° C. for 2 hrs. Then Zn (2.4 g, 37.0 mmol) was added. The mixture was stirred at 80° C. for 2 hrs. The mixture was cooled, filtered and concentrated. The residue was purified by column (DCM:MeOH=10:1) to afford (R)-tert-butyl (1-(7-amino-2-(pyrrolidin-1-yl)quinazolin-4-yl)pyrrolidin-3-yl)carbamate (1.5 g, 98%) as a yellow solid. [M+H] MS Calc'd for C₂₁H₃₀N₆O₂, 399.2; Found, 399.2.

Step 3: (R)-tert-butyl (1-(7-acrylamido-2-(pyrrolidin1l-yl)quinazolin-4-yl)pyrrolidin-3-yl)carbamate

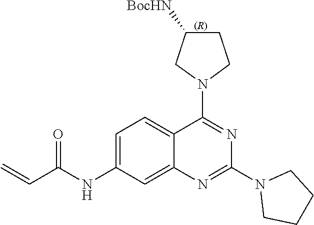

To a solution of (R)-tert-butyl (1-(7-amino-2-(pyrrolidin-1-yl)quinazolin-4-yl)pyrrolidin-3-yl)carbamate (1.4 g, 3.5 mmol) in DCM (40 mL) was added DIEA (1.4 g, 10.5 mmol) and acryloyl chloride (317 mg, 3.5 mmol) at 0° C. The mixture was stirred at rt for 2 hrs. The mixture was washed with water (50 mL) and extracted with DCM (50 mL*2). The combined organic layer was concentrated. The residue was purified by column (DCM:MeOH=10:1) to afford (R)-tert-butyl (1-(7-acrylamido-2-(pyrrolidin-1-yl)quinazolin-4-yl)pyrrolidin-3-yl)carbamate (600 mg, 40%) as a yellow solid. [M+H] MS Calc'd for C₂₄H₃₂N₆O₃, 453.3; Found, 453.3.

Step 4: (R)—N-(4-(3-aminopyrrolidin-1-yl)-2-(pyrrolidin-1-yl)quinazolin-7-yl)acrylamide TFA salt

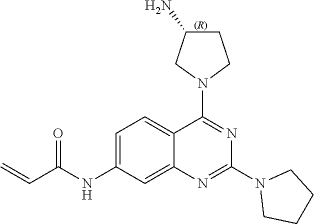

A solution of (R)-tert-butyl (1-(7-acrylamido-2-(pyrrolidin-1-yl)quinazolin-4-yl)pyrrolidin-3-yl)carbamate (600 mg, 1.3 mmol) and TFA (10 mL) in DCM (10 mL) was stirred at RT for 1 h. The reaction mixture was concentrated in vacuo to afford TFA salt of (R)—N-(4-(3-aminopyrrolidin-1-yl)-2-(pyrrolidin-1-yl)quinazolin-7-yl)acrylamide (467 mg) as a yellow solid. [M+H] MS Calc'd for C₁₉H₂₄N₆O, 353.2; Found,353.2.

Step 5: (R)—N-(4-(3-((5-cyanopyrimidin-2-yl)amino)pyrrolidin-1-yl)-2-(pyrrolidin-1-yl)quinazolin-7-yl)acrylamide HCOOH salt

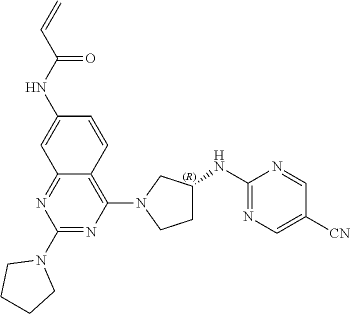

A mixture of (R)—N-(4-(3-aminopyrrolidin-1-yl)-2-(pyrrolidin-1-yl)quinazolin-7-yl)acrylamide (TFA salt) (367 mg, 1.04 mmol), 2-chloropyrimidine-5-carbonitrile (217 mg, 1.60 mmol) and DIEA (402 mg, 3.12 mmol) in DMSO (15 mL) was heated to 30° C. for 2 h. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford HCOOH salt of (R)—N-(4-(3-(5-cyanopyrimidin-2-yl)amino)pyrrolidin-1-yl)-2-(pyrrolidin-1-yl)quinazolin-7-yl)acrylamide (328.5 mg, 54%) as a white solid. ¹H NMR (400 MHz, DMSO-d₆): δ 1.88-1.91 (m, 4H), 2.05-2.09 (m, 1H), 2.23-2.27 (m, 1H), 3.50-3.53 (m, 4H), 3.80-3.84 (m, 1H), 3.89-3.92 (m, 1H), 4.02-4.05 (m, 1H), 4.11-4.15 (m, 1H), 4.55-4.57 (m, 1H), 5.79 (dd, J=1.6, 10.0 Hz, 1H), 6.27-6.32 (m, 1H), 6.43-6.50 (m, 1H), 7.15 (dd, J=2.0, 9.2 Hz, 1H), 7.89-7.96 (m, 2H), 8.16 (s, 1H), 8.65-8.69 (m, 2H), 8.78 (d, J=2.4 Hz, 1H), 10.23 (s, 1H). [M+H] MS Calc'd for C₂₄H₂₅N₉O, 456.2; Found, 456.2.

### Example 30: (R)—N-(4-(3-((5-cyanopyrimidin-2-ynamino)pyrrolidin-1-yl)-2-methylquinazolin-7-yl)acrylamide

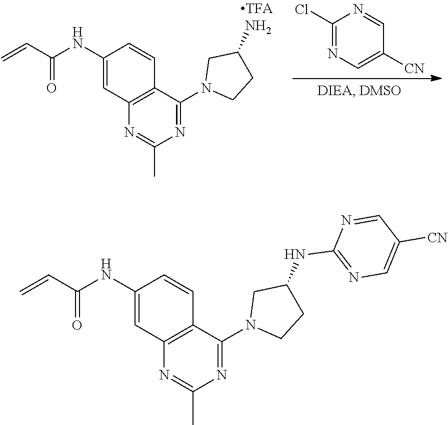 [SMILES: C=CC(=O)Nc1ccc2c(N3CC[C@@H](N)C3)nc(C)nc2c1 ; N#Cc1cnc(Cl)nc1 ; C=CC(=O)Nc1ccc2c(N3CC[C@@H](Nc4ncc(C#N)cn4)C3)nc(C)nc2c1]

A mixture of (R)—N-(4-(3-aminopyrrolidin-1-yl)-2-methylquinazolin-7-yl)acrylamide (165 mg, 0.55 mmol), 2-chloropyrimidine-5-carbonitrile (62 mg, 0.44 mml) and DIEA (0.46 mL, 2.77 mmol) in DMSO (10 mL) was stirred at 40° C. under nitrogen atmosphere for 2 hrs. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford (R)—N-(4-(3-((5-cyanopyrimidin-2-yl)amino)pyrrolidin-1-yl)-2-methylquinazolin-7-yl)acrylamide (58.5 mg, 26.3%) as a white solid. 1HNMR (400 MHz, DMSO-d₆): 2.07-2.10 (m, 1H), 2.26-2.28 (m, 1H), 2.41 (s, 3H), 3.82-3.85 (m, 1H), 3.87-3.94 (m, 1H), 4.04-4.07 (m, 1H), 4.14-4.19 (m, 1H), 4.55-4.57 (m, 1H), 5.82 (dd, J=1.6, 10.0 Hz, 1H), 6.29-6.34 (m, 1H), 6.45-6.52 (m, 1H), 7.56 (dd, J=2.4, 8.2 Hz, 1H), 8.05 (d, J=2.0 Hz, 1H), 8.16 (d, J=9.2 Hz, 1H), 8.67-8.69 (m, 2H), 8.79 (d, J=2.8 Hz, 1H), 10.44 (s, 1H). [M+H] MS Calc'd for C₂₁H₂₀N₈O, 401.2; Found, 401.2.

### Example 31: (R)—N-(2-amino-4-(34(5-cyanopyrimidin-2-yl)amino)pyrrolidin-1-yl)quinazolin-7-yl)acrylamide

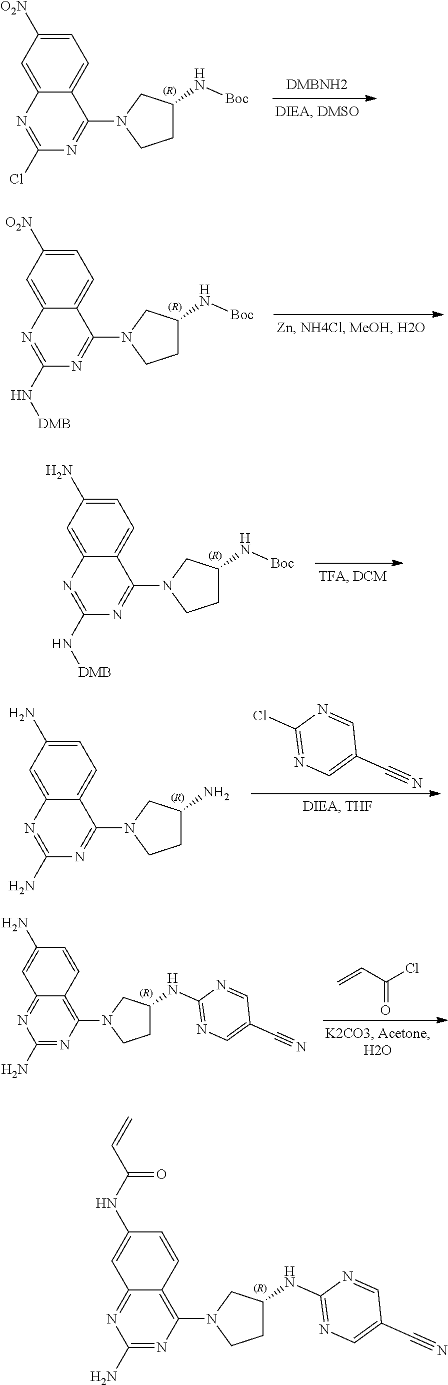

Step 1: (R)-tert-butyl (1-(2-((2,4-dimethoxybenzyl)amino)-7-nitroquinazolin-4-yl)pyrrolidin-3-yl)carbamate

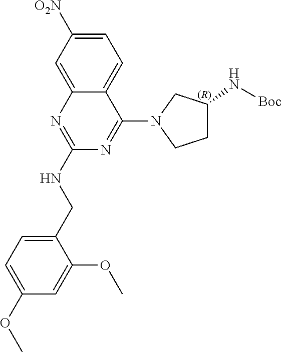

A mixture of (R)-tert-butyl (1-(2-chloro-7-nitroquinazolin-4-yl)pyrrolidin-3-yl)carbamate (2.1 g, 5.6 mmol), DMBNH2 (1.4 g, 8.4 mml) and DIEA (3.6 g, 27.9 mmol) in DMSO (20 mL) was stirred at 90° C. under nitrogen atmosphere overnight. The reaction mixture was cooled, diluted with water (50 mL) and extracted with DCM (50 mL*2). The combined organic layer was washed with water (50 mL*2) and brine (50 mL), dried over Na₂SO₄, filtered and concentrated in vacuo. The residue was purified by column chromatography on silica gel (PE:EA=2:1) to afford (R)-tert-butyl (1-(2-((2,4-dimethoxybenzyl)amino)-7-nitroquinazolin-4-yl)pyrrolidin-3-yl)carbamate (2.3 g, 78%) as a red solid. [M+H] MS Calc'd for C₂₆H₃₂N₆O₆, 525.2; Found, 525.2.

Step 2: (R)-tert-butyl (1-(7-amino-2-((2,4-dimethoxybenzyl)amino)quinazolin-4-yl)pyrrolidin-3-yl)carbamate

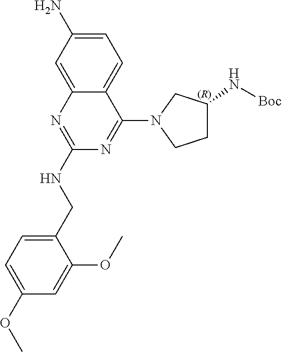

A mixture of (R)-tert-butyl (1-(2-((2,4-dimethoxybenzyl)amino)-7-nitroquinazolin-4-yl)pyrrolidin-3-yl)carbamate (2.3 g, 4.4 mmol) and NH₄Cl (2.4 g, 44.0 mmol) in MeOH (40 mL) and water (8 mL) was stirred at 80° C. for 2 hrs. Then Zn (2.4 g, 44.0 mmol) was added. The mixture was stirred at 80° C. for 2 hrs. The mixture was cooled, filtered and concentrated. The residue was purified by column chromatography on silica gel (DCM:MeOH=10:1) to afford (R)-tert-butyl (1-(7-amino-2-((2,4-dimethoxybenzyl)amino)quinazolin-4-yl)pyrrolidin-3-yl)carbamate (2.2 g, 100%) as a yellow solid. [M+H] MS Calc'd for C₂₆H₃₄N₆O₄, 495.3; Found, 495.3.

Step 3: (R)-4-(3-aminopyrrolidin-1-yl)quinazoline-2,7-diamine

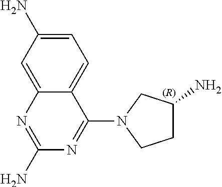

A solution of (R)-tert-butyl (1-(7-amino-2-((2,4-dimethoxybenzyl)amino)quinazolin-4-yl)pyrrolidin-3-yl)carbamate (2.0 g, 4.0 mmol) and TFA (8 mL) in DCM (20 mL) was stirred at RT for 1 h. The reaction mixture was concentrated in vacuo to afford TFA salt of (R)-4-(3-aminopyrrolidin-1-yl)quinazoline-2,7-diamine (987 mg) as a black oil. [M+H] MS Calc'd for C₁₂H₁₆N₆, 245.1; Found, 245.1.

Step 4: (R)-2-((1-(2,7-diaminoquinazolin-4-yl)pyrrolidin-3-yl)amino)pyrimidine-5-carbonitrile

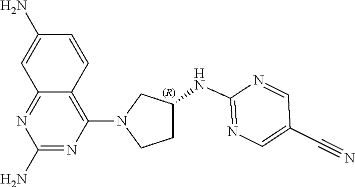

A mixture of (R)-4-(3-aminopyrrolidin-1-yl)quinazoline-2,7-diamine (1.0 g, 4.1 mmol), 2-chloropyrimidine-5-carbonitrile (510 mg, 4.1 mml) and DIEA (1.6 g, 12.3 mmol) in THF (20 mL) was stirred at 30° C. under nitrogen atmosphere for 2 hrs. The residue was concentrated and purified by column chromatography on silica gel (DCM:MeOH=10:1) to afford (R)-2-((1-(2,7-diaminoquinazolin-4-yl)pyrrolidin-3-yl)amino)pyrimidine-5-carbonitrile (1.2 g, 85%) as yellow oil. [M+H] MS Calc'd for C₁₇H₁₇N₉, 348.2; Found, 348.2.

Step 5: (R)—N-(2-amino-4-(3-((5-cyanopyrimidin-2-yl)amino)pyrrolidin-1-yl)quinazolin-7-yl)acrylamide

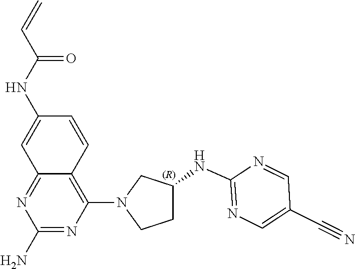

To a solution of (R)-2-(1-(2,7-diaminoquinazolin-4-yl)pyrrolidin-3-yl)amino)pyrimidine-5-carbonitrile (600 mg, 1.7 mmol) in acetone (8 mL) and H₂O(4 mL) was added K₂CO₃ (716 mg, 5.2 mmol) and acryloyl chloride (155 mg, 1.7 mmol) at 0° C. The mixture was stirred at 0° C. for 1 h. The mixture was concentrated and purified by prep-HPLC to afford HCOOH salt of (R)—N-(2-amino-4-(3-((5-cyanopyrimidin-2-yl)amino)pyrrolidin-1-yl)quinazolin-7-yl)acrylamide (24.3 mg, 4%) as a white solid. ¹H NMR (400 MHz, DMSO-d₆): 2.07-2.11 (m, 1H), 2.23-2.28 (m, 1H), 3.71-3.81 (m, 1H), 3.87-3.93 (m, 1H), 4.00-4.05 (m, 1H), 4.10-4.15 (m, 1H), 4.52-4.56 (m, 1H), 5.78 (dd, J=2.0, 10.0 Hz, 1H), 6.27-6.31 (m, 1H), 6.43-6.50 (m, 3H), 7.29 (d, J=8.8 Hz, 1H), 7.73 (s, 1H), 7.97 (d, J=9.2 Hz, 1H), 8.26 (s, 1H), 8.67-8.69 (m, 2H), 8.78 (s, 1H), 10.33 (s, 1H). [M+H] MS Calc'd for C₂₀H₁₉N₉O, 402.2; Found, 402.1.

### Example 32: (R)—N-(4-(34(5-cyanopyrimidin-2-yl)amino)pyrrolidin-1-yl)-2-(2,2,2-trifluoroethoxy)quinazolin-7-yl)acrylamide

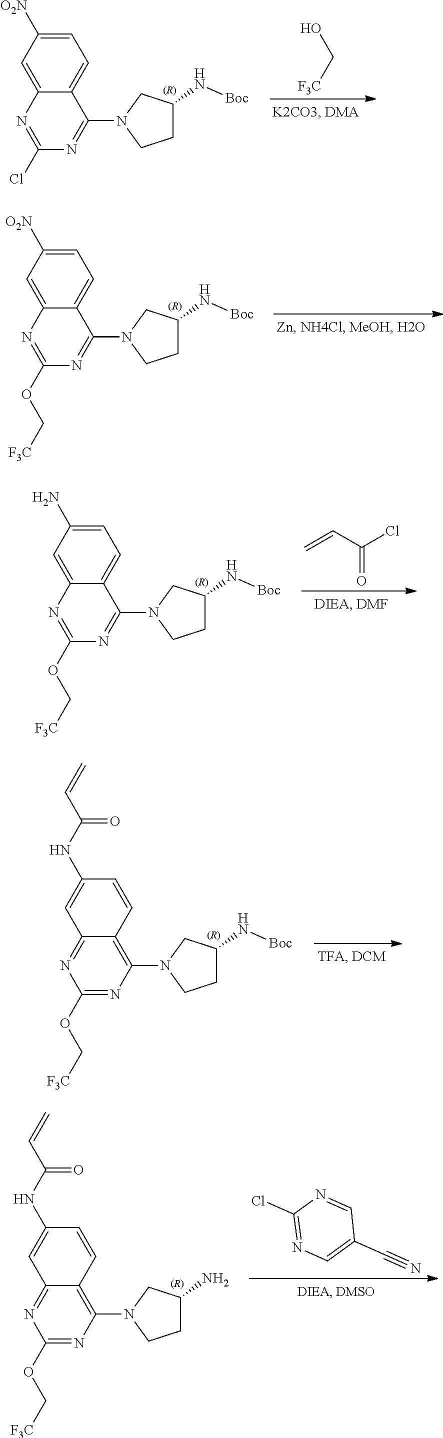

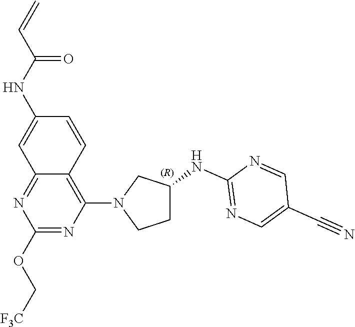

Step 1: (R)-tert-butyl (1-(7-nitro-2-(2,2,2-trifluoroethoxy)quinazolin-4-yl)pyrrolidin-3-yl)carbamate

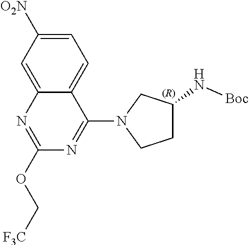

A mixture of (R)-tert-butyl (1-(2-chloro-7-nitroquinazolin-4-yl)pyrrolidin-3-yl)carbamate (5.0 g, 12.7 mmol), 2,2,2-trifluoroethanol (1.5 g, 15.3 mml) and K₂CO₃ (2.6 g, 19.0 mmol) in DMA (40 mL) was stirred at 110° C. under nitrogen atmosphere overnight. The reaction mixture was cooled, diluted with water (100 mL) and extracted with DCM (100 mL*2). The combined organic layer was washed with water (100 mL*2) and brine (100 mL), dried over Na₂SO₄, filtered and concentrated in vacuo. The residue was purified by column chromatography on silica gel (PE:EA=2:1) to afford (R)-tert-butyl (1-(7-nitro-2-(2,2,2-trifluoroethoxy)quinazolin-4-yl)pyrrolidin-3-yl)carbamate (4.0 g, 69%) as a red solid. [M+H] MS Calc'd for C₁₉H₂₂F₃N₅O₅, 458.2; Found, 458.2.

Step 2: (R)-tert-butyl (1-(7-amino-2-(2,2,2-trifluoroethoxy)quinazolin-4-yl)pyrrolidin-3-yl)carbamate

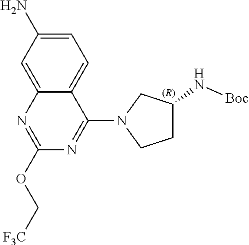

A mixture of (R)-tert-butyl (1-(7-nitro-2-(2,2,2-trifluoroethoxy)quinazolin-4-yl)pyrrolidin-3-yl)carbamate (3.5 g, 7.7 mmol) and NH₄Cl (4.0 g, 77.0 mmol) in MeOH (40 mL) and water (8 mL) was stirred at 80° C. for 2 hrs. Then Zn (5.0 g, 77.0 mmol) was added. The mixture was stirred at 80° C. for 2 hrs. The mixture was cooled, filtered and concentrated. The residue was purified by column chromatography on silica gel (DCM:MeOH=10:1) to afford (R)-tert-butyl (1-(7-amino-2-(2,2,2-trifluoroethoxy)quinazolin-4-yl)pyrrolidin-3-yl)carbamate (800 mg, 25%) as a yellow solid. [M+H] MS Calc'd for C₁₉H₂₄F₃N₅O₃, 428.2; Found, 428.2.

Step 3: (R)-tert-butyl (1-(7-acrylamido-2-(2,2,2-trifluoroethoxy)quinazolin-4-yl)pyrrolidin-3-yl)carbamate

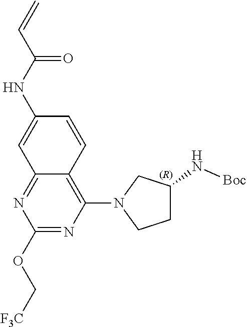

To a solution of (R)-tert-butyl (1-(7-amino-2-(2,2,2-trifluoroethoxy)quinazolin-4-yl)pyrrolidin-3-yl)carbamate (427 mg, 1.0 mmol) in DMF (20 mL) was added DIEA (387 mg, 3.0 mmol) and acryloyl chloride (90 mg, 1.0 mmol) at 0° C. The mixture was stirred at RT for 5 h. The mixture was concentrated and purified by column chromatography on silica gel to afford (R)-tert-butyl (1-(7-acrylamido-2-(2,2,2-trifluoroethoxy)quinazolin-4-yl)pyrrolidin-3-yl)carbamate (280 mg, 58%) as a yellow solid. [M+H] MS Calc'd for C₂₂H₂₆F₃N₅O₄, 482.2; Found, 482.2.

Step 4: (R)—N-(4-(3-aminopyrrolidin-1-yl)-2-(2,2,2-trifluoroethoxy)quinazolin-7-yl)acrylamide TFA salt

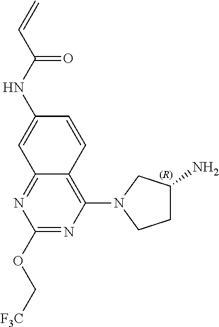

A solution of (R)-tert-butyl (1-(7-acrylamido-2-(2,2,2-trifluoroethoxy)quinazolin-4-yl)pyrrolidin-3-yl)carbamate (280 mg, 0.58 mmol) and TFA (10 mL) in DCM (10 mL) was stirred at RT for 1 h.

The reaction mixture was concentrated in vacuo to afford TFA salt of (R)—N-(4-(3-aminopyrrolidin-1-yl)-2-(2,2,2-trifluoroethoxy)quinazolin-7-yl)acrylamide (222 mg) as a yellow solid. [M+H] MS Calc'd for C₁₇H₁₈F₃N₅O₂, 382.1; Found, 382.1.

Step 5: (R)—N-(4-(3-((5-cyanopyrimidin-2-yl)amino)pyrrolidin-1-yl)-2-(2,2,2-trifluoroethoxy)quinazolin-7-yl)acrylamide

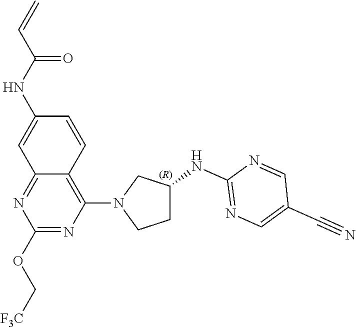

A solution of (R)—N-(4-(3-aminopyrrolidin-1-yl)-2-(2,2,2-trifluoroethoxy)quinazolin-7-yl)acrylamide (222 mg, 0.58 mmol), 2-chloropyrimidine-5-carbonitrile (121 mg, 1.87 mmol) and DIEA (224 mg, 1.74 mmol) in DMSO (10 mL) was stirred at 30° C. for 2 h. The reaction mixture was diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford (R)—N-(4-(3-((5-cyanopyrimidin-2-yl)amino)pyrrolidin-1-yl)-2-(2,2,2-trifluoroethoxy)quinazolin-7-yl)acrylamide (97.7 mg, 35%) as a yellow solid. 1HNMR (400 MHz, DMSO-d₆): 2.09-2.13 (m, 1H), 2.26-2.31 (m, 1H), 3.85-3.88 (m, 1H), 3.89-3.96 (m, 1H), 4.00-4.06 (m, 1H), 4.10-4.19 (m, 1H), 4.57-4.61 (m, 1H), 4.98-5.01 (m, 2H), 5.82 (dd, J=1.6, 10.0 Hz, 1H), 6.30-6.34 (m, 1H), 6.44-6.51 (m, 1H), 7.47 (dd, J=2.4, 9.2 Hz, 1H), 8.06 (d, J=2.0 Hz, 1H), 8.18 (d, J=9.2 Hz, 1H), 8.67-8.69 (m, 2H), 8.78 (d, J=2.4 Hz, 1H), 10.44 (s, 1H). [M+H]MS Calc'd for C₂₂H₁₉F₃N₈O₂, 485.2; Found, 485.1.

### Example 33: (R)—N-(1-(3-((5-bromopyrimidin-2-yl)amino)pyrrolidin-1-yl)isoquinolin-6-yl)but-2-ynamide

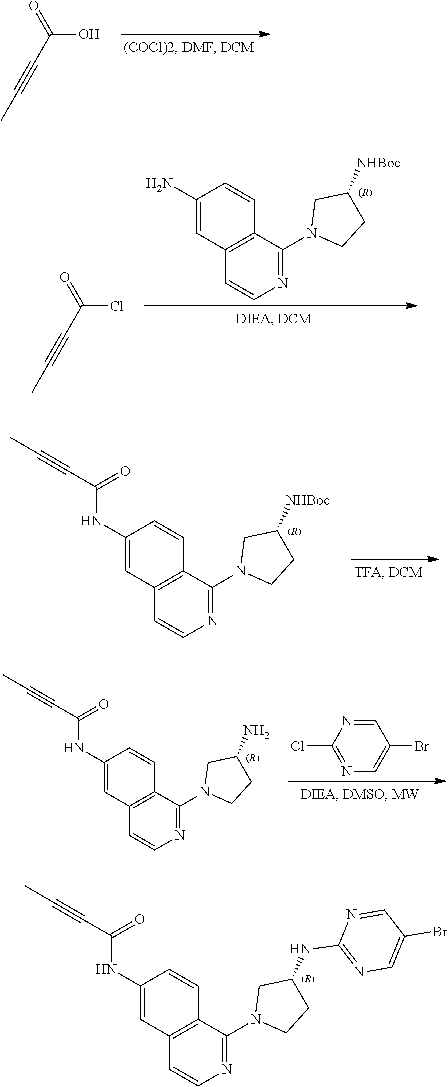

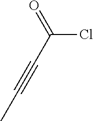

Step 1: but-2-ynoyl chloride

To a solution of but-2-ynoic acid (697 mg, 8.3 mmol) and DMF(1 drop) in DCM (5 mL) was added (COC1)₂ (1.05 g, 8.3 mmol) at 0° C. The reaction mixture was stirred at 0° C. for 1 h. Then the reaction mixture was concentrated to give crude but-2-ynoyl chloride (700 mg, 100%).

Step 2: (R)-tert-butyl (1-(6-(but-2-ynamido)isoquinolin-1-yl)pyrrolidin-3-yl)carbamate

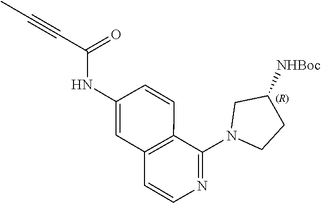

To a solution of (R)-tert-butyl (1-(6-aminoisoquinolin-1-yl)pyrrolidin-3-yl)carbamate (680 mg, 2.1 mmol) and DIEA (1.06 g, 8.29 mmol) in DCM (10 mL) was added but-2-ynoyl chloride (700 mg) at 0° C. The mixture was stirred at 0° C. for 30 min. The reaction mixture was diluted with water (20 mL) and extracted with DCM (20 mL*2). The combined organic layer was washed with water (20 mL*2) and brine (20 mL), dried over Na₂SO₄, filtered, concentrated in vacuo and purified by column chromatography on silica gel (DCM:MeOH=10:1) to afford (R)-tert-butyl (1-(6-(but-2-ynamido)isoquinolin-1-yl)pyrrolidin-3-yl)carbamate (2.3 g, 100%) as brown oil. [M+H] MS Calc'd for C₂₂H₂₆N₄O₃, 395.2; Found, 395.2.

Step 3: (R)—N-(1-(3-aminopyrrolidin-1-yl)isoquinolin-6-yl)but-2-ynamide

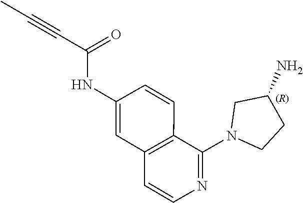

A solution of (R)-tert-butyl (1-(6-(but-2-ynamido)isoquinolin-1-yl)pyrrolidin-3-yl)carbamate (2.0 g, 2.1 mmol) and TFA (9 mL) in DCM (9 mL) was stirred at RT for 1 h. The reaction mixture was concentrated in vacuo and purified by column chromatography on silica gel (DCM:MeOH=4:1) to afford (R)—N-(1-(3-aminopyrrolidin-1-ypisoquinolin-6-yl)but-2-ynamide (477 mg, 61%) as a brown solid. [M+H] MS Calc'd for C₁₇H₁₈N₄O, 295.1; Found, 295.1.

Step 4: (R)—N-(1-(3-((5-bromopyrimidin-2-yl)amino)pyrrolidin-1-yl)isoquinolin-6-yl)but-2-ynamide

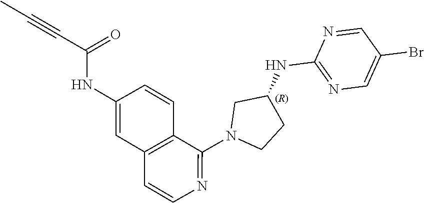

A solution of (R)—N-(1-(3-aminopyrrolidin-1-yl)isoquinolin-6-yl)but-2-ynamide (400 mg, 1.36 mmol), 5-bromo-2-chloropyrimidine (523 mg, 2.71 mmol) and DIEA (877 mg, 6.80 mmol) in DMSO (5 mL) was stirred at 80° C. for 2 h under microwave. The reaction mixture was diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford (R)—N-(1-(3-((5-bromopyrimidin-2-yl)amino)pyrrolidin-1-yl)isoquinolin-6-yl)but-2-ynamide (98.9 mg, 16.2%) as a white solid. 1HNMR (400 MHz, DMSO-d₆): 2.03-2.07 (m, 4H), 2.21-2.32 (m, 1H), 3.67-3.71 (m, 1H), 3.77-3.83 (m, 1H), 3.88-3.92 (m, 1H), 4.03-4.06 (m, 1H), 4.36-4.40 (m, 1H), 6.94 (d, J=5.6 Hz, 1H), 7.50 (d, J=8.8 Hz, 1H), 7.81-7.68 (m, 2H), 8.08 (s, 1H), 8.13-8.16 (m, 1H), 8.40 (s, 2H), 10.89 (s, 1H). [M+H] MS Calc'd for C₂₁H₁₉BrN₆O, 452.2; Found, 452.1.

### Example 34: (R)—N-(4-(3-((1-ethyl-1H-pyrazolo14,3-cl pyridin-6-yl)amino)pyrrolidin-1-yl)quinazolin-7-yl)acrylamide

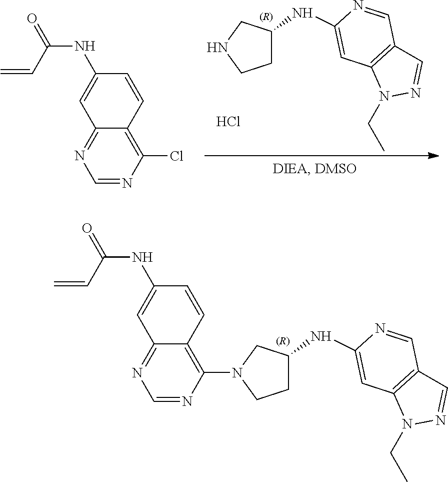

A solution of N-(4-chloroquinazolin-7-yl)acrylamide (180 mg, 0.77 mmol), HCl salt of (R)-1-ethyl-N-(pyrrolidin-3-yl)-1H-pyrazolo[4,3-c]pyridin-6-amine (247 mg, 0.97 mmol) and DIEA (1.3 mL, 7.72 mmol) in DMSO (10 mL) was stirred at 40° C. for 2 h under N₂. The reaction mixture was cooled, diluted with water (20 mL) and extracted with EA (20 mL*2). The combined organic layers were washed with brine (20 mL), dried over anhydrous sodium sulfate, filtered and concentrated under reduced pressure. The residue was purified by prep-HPLC to give (R)—N-(4-(3-((1-ethyl-1H-pyrazolo[4,3-c]pyridin-6-yl)amino)pyrrolidin-1-yl)quinazolin-7-yl)acrylamide (38.4 mg, 11.5%). ¹H NMR (400 MHz, DMSO-d6): 1.33 (t, J=7.2 Hz, 3H), 2.07-2.12 (m, 1H), 2.29-2.33 (m, 1H), 3.80-3.84 (m, 1H), 3.95-3.97 (m, 1H), 4.06-4.09 (m, 1H), 4.21-4.28 (m, 3H), 4.44-4.45 (m, 1H), 5.83 (dd, J=10.0, 1.6 Hz, 1H), 6.30-6.35 (m, 1H), 6.43-6.52 (m, 2H), 6.75 (d, J=6.0 Hz, 1H), 7.62 (dd, J=9.2, 2.0 Hz, 1H), 7.96 (s, 1H), 8.15 (d, J=2.4 Hz, 1H), 8.24 (d, J=8.8 Hz, 1H), 8.40 (s, 1H), 8.59 (s, 1H), 10.48 (s, 1H). [M+H] MS Calc'd C23H24N80, 429.2; Found: 429.1.

### Example 35: (R,E)—N-(4-(3-((5-chloro-4-(pyrazolo[1,5-a]pyridin-3-yl)pyrimidin-2-ynamino)pyrrolidin-1-yl)-2-methylquinazolin-7-yl)-4-(dimethylamino)but-2-enamide

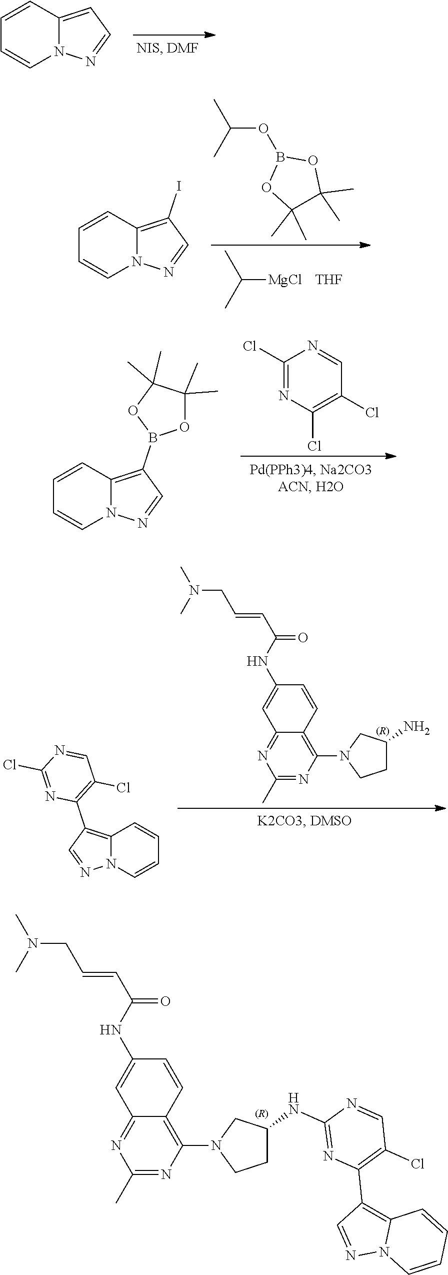

Step 1: 3-iodopyrazolo [1,5-a] pyridine

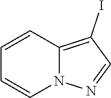

A solution of pyrazolo[1,5-a]pyridine (900 mg, 7.63 mmol) and NIS (2.1 g, 9.15 mmol) in DMF (20 mL) was stirred at RT for 16 h. The reaction mixture was diluted with water (50 mL) and extracted with EA (20 mL*2). The combined organic layer was washed with water (20 mL*2) and brine (20 mL), dried over Na₂SO₄, filtered and concentrated in vacuo. The residue was purified by column chromatography on silica gel to afford 3-iodopyrazolo[1,5-a]pyridine (1.75 g, 87.5%) as a white solid. [M+H] MS Calc'd for C₇H₅IN₂, 244.9; Found, 244.9.

Step 2: 3-(4,4,5,5-tetramethyl-1,3,2-dioxaborolan-2-yl)pyrazolo[1,5-a]pyridine

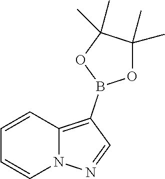 [SMILES: CC1(C)OB(c2cnn3ccccc23)OC1(C)C]

To a solution of 3-iodopyrazolo[1,5-a]pyridine (1.7 g, 6.9 mmol) and 2-isopropoxy-4,4,5,5-tetramethyl-1,3,2-dioxaborolane (2.6 mL, 13.8 mmol) in THF (40 mL) was added isopropylmagnesium chloride (5.87 mL, 2N in THF) slowly under an ice-bath. The mixture was stirred at 0° C. for 2 h. The reaction mixture was diluted with water (50 mL) and extracted with EA (20 mL*2). The combined organic layer was washed with water (20 mL*2) and brine (20 mL), dried over Na₂SO₄, filtered and concentrated in vacuo. The residue was purified by column chromatography on silica gel (PE:EA=10:1) to afford 3-(4,4,5,5-tetramethyl-1,3,2-dioxaborolan-2-yl)pyrazolo[1,5-a]pyridine (1.0 g, 58.8%) as a white solid. [M+H] MS Calc'd for C₁₃H₁₇BN₂O₂, 245.1; Found, 245.1.

Step 3: 3-(2,5-dichloropyrimidin-4-yl)pyrazolo[1,5-a]pyridine

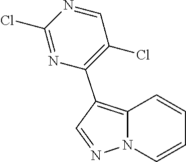 [SMILES: Clc1ncc(Cl)c(-c2cnn3ccccc23)n1]

To a solution of 3-(4,4,5,5-tetramethyl-1,3,2-dioxaborolan-2-yl)pyrazolo[1,5-a]pyridine (938 mg, 3.85 mmol) and 2,4,5-trichloropyrimidine (700 mg, 3.85 mmol) in ACN (30 mL) and H₂O(5 mL) was added Pd(PPh3)4 (443 mg, 0.38 mmol) and Na₂CO₃ (815 mg, 7.69 mmol) at RT. The mixture was stirred at 90° C. for 4 h. The reaction mixture was diluted with water (50 mL) and extracted with DCM (30 mL*2). The combined organic layer was washed with water (30 mL*2) and brine (20 mL), dried over Na₂SO₄, filtered and concentrated in vacuo. The residue was purified by column chromatography on silica gel (PE:EA=10:1) to afford 3-(2,5-dichloropyrimidin-4-yl)pyrazolo[1,5-a]pyridine (500 mg, 49%) as a white solid. [M+H]MS Calc'd for C₁₁H₆C₁₂N₄, 265.0; Found, 265.0.

Step 4: (R,E)—N-(4-(3-((5-chloro-4-(pyrazolo[1,5-a]pyridin-3-yl)pyrimidin-2-yl)amino)pyrrolidin-1-yl)-2-methylquinazolin-7-yl)-4-(dimethylamino)but-2-enamide

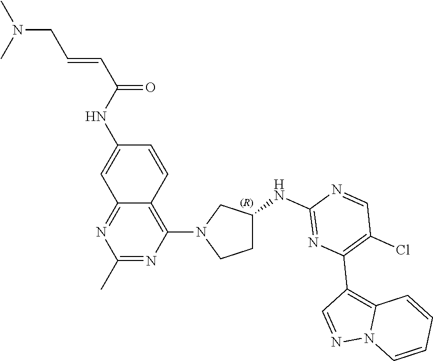

A solution of 3-(2,5-dichloropyrimidin-4-yl)pyrazolo[1,5-a]pyridine (371 mg, 1.69 mmol), (R,E)—N-(4-(3-aminopyrrolidin-1-yl)-2-methylquinazolin-7-yl)-4-(dimethylamino)but-2-enamide (500 mg, 1.41 mmol) and K₂CO₃ (777 mg, 5.63 mmol) in DMSO (10 mL) was stirred at 100° C. for 4 h. The reaction mixture was cooled, diluted with water (20 mL) and extracted with DCM (20 mL*2). The combined organic layer was washed with water (20 mL*2) and brine (20 mL), dried over Na₂SO₄, filtered and concentrated in vacuo. The residue was purified by prep-HPLC to afford (R,E)—N-(4-(3-((5-chloro-4-(pyrazolo[1,5-a]pyridin-3 -yl)pyrimidin-2-yl)amino)pyrrolidin-1-yl)-2-methylquinazolin-7-yl)-4-(dimethylamino)but-2-enamide (18.7 mg, 2.2%) as a white solid. ¹H NMR (400 MHz, DMSO-d₆): 1.95-2.00 (m, 1H), 2.02-2.19 (m, 7H), 2.41 (s, 3H), 3.08 (d, J=4.8 Hz, 2H), 3.80-3.91 (m, 2H), 4.03-4.22 (m, 2H), 4.50-4.57 (m, 1H), 6.30-6.34 (m, 1H), 6.77-6.83 (m, 1H), 7.13-7.15 (m, 1H), 7.52-7.54 (m, 2H), 7.80 (d, J=5.2 Hz, 1H), 8.04 (s, 1H), 8.18 (d, J=9.2 Hz, 1H), 8.37 (s, 1H), 8.87-8.95 (m, 3H), 10.36 (s, 1H). [M+H] MS Calc'd for C₃₀H₃₁ClN₁₀O, 583.2; Found, 583.2.

### Example 36: Synthesis of (R)—N-(1434(5-cyanopyrimidin-2-yl)amino)pyrrolidin-1-yl)isoquinolin-6-yl)-N-methylacrylamide

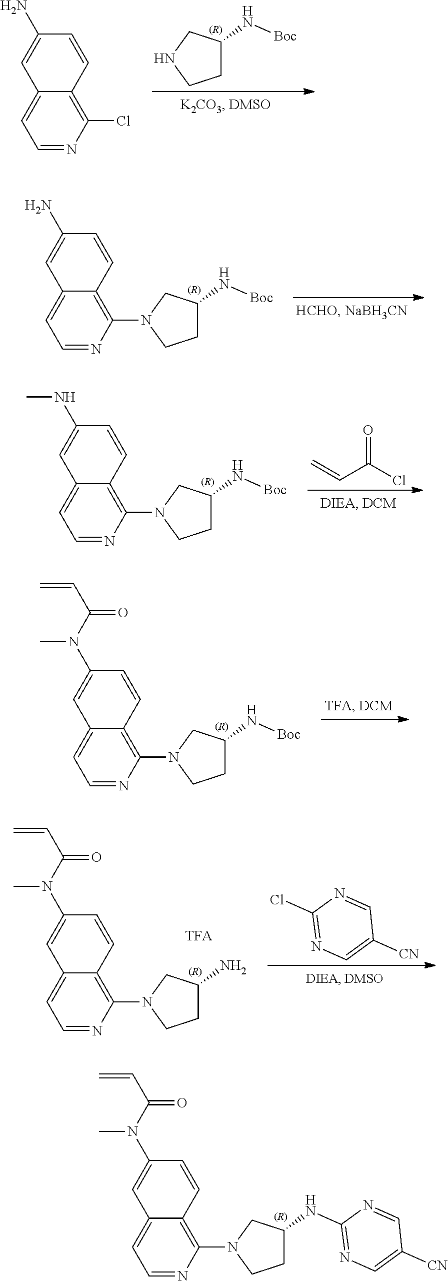

Step 1: (R)-tert-butyl (1-(6-aminoisoquinolin-1-yl)pyrrolidin-3-yl)carbamate

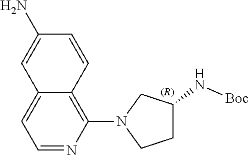

A solution of 1-chloroisoquinolin-6-amine (1.5 g, 8.4 mmol), (R)-tert-butyl pyrrolidin-3-ylcarbamate (3.0 g, 16.8 mmol) and K₂CO₃ (1.7 g, 12.6 mmol) in DMSO (15 mL) was stirred at 160° C. for 5 h under microwave conditions. The reaction mixture was cooled, diluted with water (100 mL) and extracted with EA (50 mL*2). The combined extracts were washed with water (100 mL*2) and brine (100 mL), dried over anhydrous sodium sulfate, filtered and purified by FCC (PE/EA=1: 1) to afford (R)-tert-butyl (1-(6-aminoisoquinolin-1-yl)pyrrolidin-3-yl)carbamate (1.4 g, 39%) as a yellow solid. [M+H] Calc'd for C₁₈H₂₄N₄O₂, 329.2 Found, 329.2.

Step 2: (R)-tert-butyl (1-(6-(methylamino)isoquinolin-1-yl)pyrrolidin-3-yl)carbamate

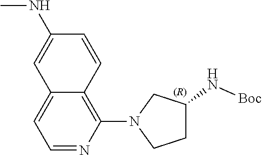

To a mixture of (R)-tert-butyl (1-(6-aminoisoquinolin-1-yl)pyrrolidin-3-yl)carbamate (656 mg, 2.00 mmol) in MeOH (40 mL) was added HCHO (649 mg, 37% in H₂O, 4.00 mmol) in an ice-bath. After stirring for 30 min, NaBH₃CN (992 mg, 15.74 mmol) was added to the mixture at 0° C. The reaction mixture was then stirred at 60° C. for 15 h. The reaction mixture was concentrated. To the residue was added DCM (20 mL) and washed with 0.5 N HCl (10 mL) then the organic extracts dried over Na₂SO₄, filtered and concentrated. The residue was purified by FCC (DCM/MeOH=10:1) to afford (R)-tert-butyl (1-(6-(methylamino)isoquinolin-1-yl)pyrrolidin-3 -yl)carbamate (439 mg, 59%) as a white solid. [M+H] Calc'd for C₁₉H₂₆N₄O₂, 343.2; Found, 343.2.

Step 3: (R)-tert-butyl (1-(6-(N-methylacrylamido)isoquinolin-1-yl)pyrrolidin-3-yl)carbamate

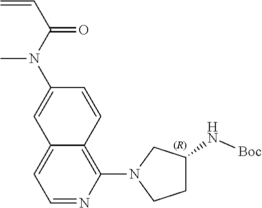

To a solution of (R)-tert-butyl (1-(6-(methylamino)isoquinolin-1-yl)pyrrolidin-3-yl)carbamate (430 mg, 1.25 mmol) and DIEA (484 mg, 3.75 mmol) in DCM (20 ml) was added acryloyl chloride (124 mg, 1.38 mmol) at 0° C. The reaction mixture was stirred at RT for 3 h. The mixture was concentrated and purified by FCC (PE/EA=1:1) to afford (R)-tert-butyl (1-(6-(N-methylacrylamido)isoquinolin-1-yl)pyrrolidin-3-yl)carbamate (300 mg, 61%) as a yellow solid. [M+H] Calc'd for C₂₂H₂₈N₄O₃, 397.2; Found, 397.2.

Step 4: (R)—N-(1-(3-aminopyrrolidin-1-yl)isoquinolin-6-yl)-N-methylacrylamide TFA salt

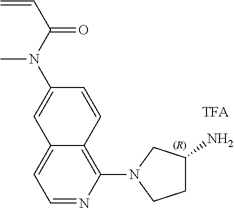

A solution of (R)-tert-butyl (1-(6-(N-methylacrylamido)isoquinolin-1-yl)pyrrolidin-3-yl)carbamate (300 mg, 0.76 mmol) in TFA/DCM (5 mL/ 10 mL) was stirred at RT for 2 h. The mixture was concentrated to afford TFA salt of (R)—N-(1-(3-aminopyrrolidin-1-yl)isoquinolin-6-yl)-N-methylacrylamide (224 mg, 100%) as a yellow solid. [M+H] Calc'd for C₁₇H₂₀N₄O, 297.2 Found, 297.2.

Step 5: (R)—N-(1-(3-((5-cyanopyrimidin-2-yl)amino)pyrrolidin-1-yl)isoquinolin-6-yl)-N-methylacrylamide

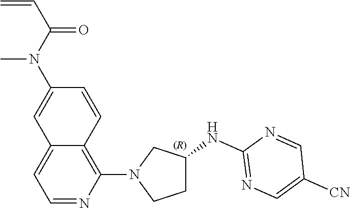

A solution of TFA salt of (R)—N-(1-(3-aminopyrrolidin-1-yl)isoquinolin-6-yl)-N-methylacrylamide (224 mg, 0.57 mmol), 2-chloropyrimidine-5-carbonitrile (158 mg, 1.13 mmol) and DIEA (294 mg, 2.28 mmol) in DMSO (10 ml) was stirred at 30° C. for 2 h. The mixture was diluted with water (50 mL) and extracted with EA (50 mL*2). The combined extracts were washed with water (100 mL*2) and brine (100 mL), dried over anhydrous sodium sulfate, filtered and concentrated. The residue was purified by prep-HPLC to give (R)—N-(1-(3-((5-cyanopyrimidin-2-yl)amino)pyrrolidin-1-yl)isoquinolin-6-yl)-N-methylacrylamide (220.4 mg, 73%) as a white solid. ¹H NMR (400 MHz, DMSO-d₆): δ 2.07-2.09 (m, 1H), 2.25-2.27 (m, 1H), 3.29 (s, 3H), 3.74-3.78 (m, 1H), 3.85-3.87 (m, 1H), 3.97-4.00 (m, 1H), 4.09-4.13 (m, 1H), 4.54-4.56 (m, 1H), 5.58-5.61 (m, 1H), 6.15-6.19 (m, 2H), 7.05 (d, J=6.0 Hz, 1H), 7.37 (dd, J=2.0, 8.8 Hz, 1H), 7.65 (d, J=2.0 Hz, 1H), 7.96 (d, J=5.6 Hz, 1H), 8.28 (d, J=9.2 Hz, 1H), 8.66-8.69 (m, 2H), 8.76-8.77 (s, 1H). [M+H] Calc'd for C₂₂H₂₁N₇O, 400.1; Found, 400.1.

### Example 37: Synthesis of (R)—N-(1-(3-((5-cyanopyrimidin-2-yl)amino)pyrrolidin-1-yl)-3-morpholinoisoquinolin-6-yl)acrylamide

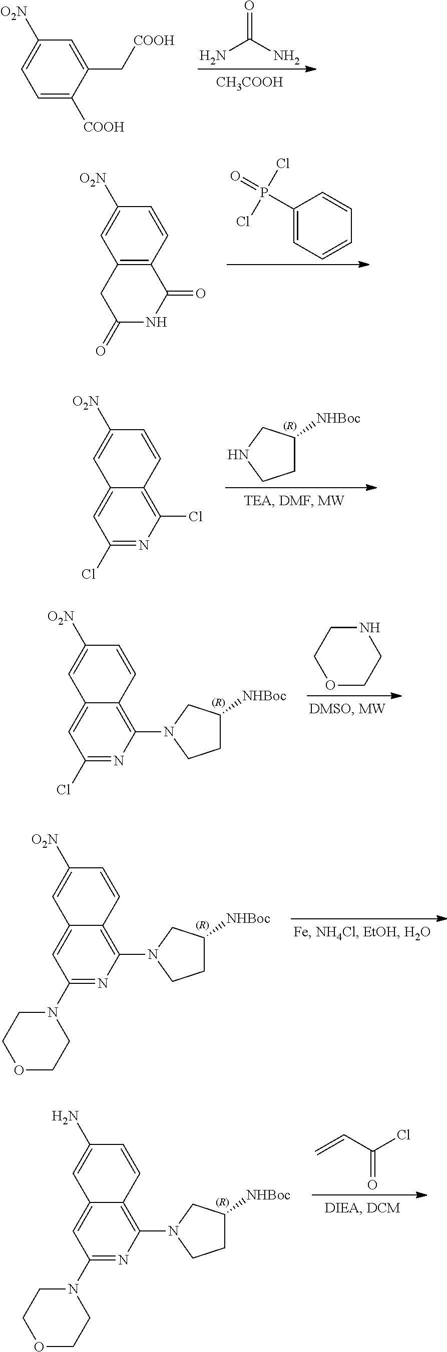

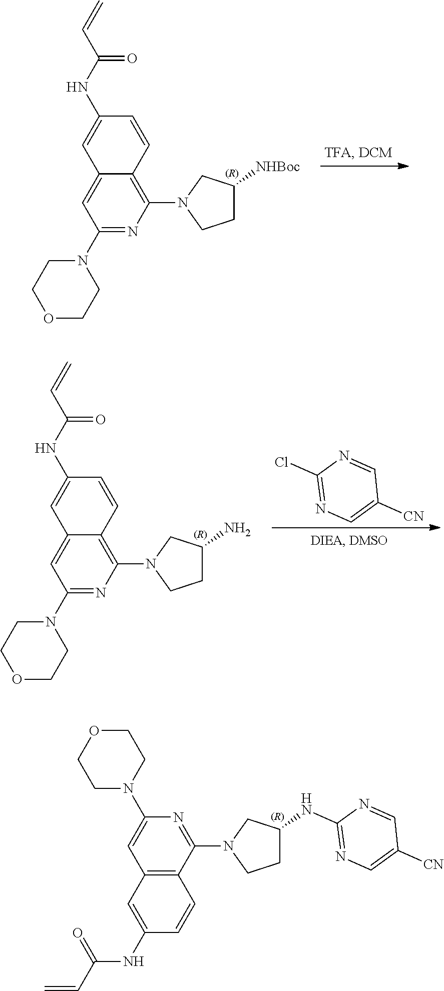

Step 1: 6-nitroisoquinoline-1,3(2114H)-dione

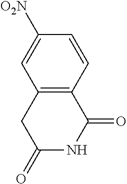 [SMILES: O=C1Cc2cc([N+](=O)[O-])ccc2C(=O)N1]

A mixture of 2-(carboxymethyl)-4-nitrobenzoic acid (45.0 g, 200.0 mmol) and CH₃COOH (500 mL) was stirred at 110° C. for 0.5 h. Then the reaction mixture was cooled to 90° C., urea (71.0 g, 1.42 mol) was added. The reaction mixture was stirred at 110° C. for 4 h. The solution was cooled to RT, H₂O (500 mL) was added. The mixture was stirred at RT for 0.5 h, filtered and concentrated to afford 6-nitroisoquinoline-1,3(2H,4H)-dione (23.0 g, 56%) as a brown solid.

Step 2: 1,3-dichloro-6-nitroisoquinoline

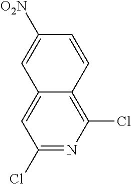 [SMILES: O=[N+]([O-])c1ccc2c(Cl)nc(Cl)cc2c1]

A mixture of 6-nitroisoquinoline-1,3(2H,4H)-dione (14.0 g, 67.9 mmol) and phenylphosphonic dichloride (200 mL) was stirred at 140° C. for 4 h. To the solution was added water (200 mL) and extracted with EA (200 mL*2). The combined organic layers were washed with brine (100 mL), dried over Na₂SO₄, filtered and concentrated in vacuo. The residue was purified by column (PE:EA=10:1) to afford 1,3-dichloro-6-nitroisoquinoline (19.0 g, 70%) as a yellow solid. [M+H] Calc'd for C₉H₄Cl₂N₂O₂, 242.9; Found, 242.9

Step 3: (R)-tert-butyl (1-(3-chloro-6-nitroisoquinolin-1-yl)pyrrolidin-3-yl)carbamate

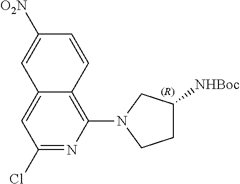

To a solution of 1,3-dichloro-6-nitroisoquinoline (5.0 g, 20.6 mmol) and (R)-tert-butyl pyrrolidin-3-ylcarbamate (3.5 g, 20.6 mmol) in DMF (50 mL) was added TEA (2.1 g, 20.6 mmol) at RT. The mixture was stirred at 140° C. under microwave for 2 h. The residue was added water (100 mL) and extracted with EA (100 mL*2). The combined organic layer was washed with water (100 mL*2) and brine (100 mL*2), dried over Na₂SO₄, filtered and concentrated in-vacuo. The residue was purified by cloumn (PE:EA=5:1) to afford (R)-tert-butyl (1-(3-chloro-6-nitroisoquinolin-1-yl)pyrrolidin-3-yl)carbamate (4.8 g, 60%) as a brown solid. [M+H] Calc'd for C₁₈H₂₁ClN₄O₄, 393.1; Found, 393.1.

Step 4: (R)-tert-butyl (1-(3-morpholino-6-nitroisoquinolin-1-yl)pyrrolidin-3-yl)carbamate

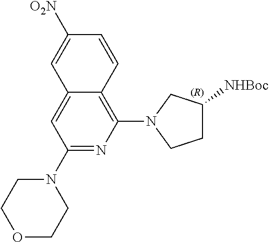

A solution of (R)-tert-butyl (1-(3-chloro-6-nitroisoquinolin-1-yl)pyrrolidin-3-yl)carbamate (1.0 g, 2.5 mmol) and morpholine (7.0 mL) in DMSO (7.0 mL) was stirred at 155° C. under microwave for 3 h. The reaction mixture was diluted with water (10 mL) and extracted with EA (10 mL*2). The combined extracts were washed with water (20 mL*2) and brine (20 mL), dried over anhydrous sodium sulfate, filtered and concentrated in vacuo. The residue was purified by silica cloumn (PE:EA=2:1) to afford (R)-tert-butyl (1-(3-morpholino-6-nitroisoquinolin-1-yl)pyrrolidin-3 -yl)carbamate (1.1 g, 97%) as a brown solid. [M+H] Calc'd for C₂₂H₂₉N₅O₅, 444.2; Found, 444.2

Step 5: (R)-tert-butyl (1-(6-amino-3-morpholinoisoquinolin-1-yl)pyrrolidin-3-yl)carbamate

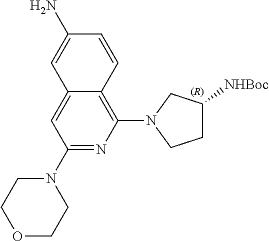

To a solution of (R)-tert-butyl (1-(3-morpholino-6-nitroisoquinolin-1-yl)pyrrolidin-3-yl)carbamate (1.1 g, 2.48 mmol) and NH₄Cl (1.3 g, 2.48 mmol) in EtOH (20 mL) and H₂O (3 mL) was added iron dust (1.4 g, 2.48 mmol) at RT. The reaction mixture was stirred at 80° C. for 4 h. Then the reaction mixture was cooled to RT, filtered and concentrated in vacuo. The residue was purified by cloumn (PE:EA=1:1) to afford (R)-tert-butyl (1-(6-amino-3-morpholinoisoquinolin-1-yl)pyrrolidin-3-yl)carbamate (400 mg, 39%) as a grey solid. [M+H] Calc'd for C₂₂H₃₁N₅O₃, 414.2 Found, 414.2.

Step 6: (R)-tert-butyl (1-(6-acrylamido-3-morpholinoisoquinolin-1-yl)pyrrolidin-3-yl)carbamate

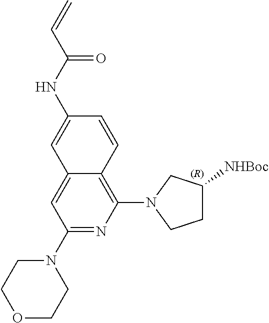

A solution of (R)-tert-butyl (1-(6-amino-3-morpholinoisoquinolin-1-yl)pyrrolidin-3-yl)carbamate (250 mg, 0.6 mmol) and DIEA (234 mg, 1.8 mmol) in DCM (15 mL) was stirred at 0° C. for 10 min. Then acryloyl chloride (55 mg, 0.6 mmol) was added slowly. The mixture was stirred at RT for 0.5 h. The mixture was diluted with water (10 mL) and extracted with EA (10 mL*2). The combined organic layer was washed with brine (10 mL), dried over Na₂SO₄, filtered and concentrated in-vacuo. The residue was purified by cloumn (PE:EA=1:1) to afford (R)-tert-butyl (1-(6-acrylamido-3-morpholinoisoquinolin-1-yl)pyrrolidin-3-yl)carbamate (120 mg, 42%) as a yellow solid. [M+H] Calc'd for C₂₅H₃₃N₅O₄, 468.3; Found, 468.3

Step 7: (R)—N-(1-(3-aminopyrrolidin-1-yl)-3-morpholinoisoquinolin-6-yl)acrylamide TFA salt

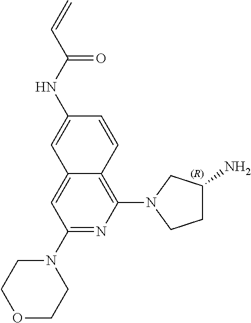

A solution of (R)-tert-butyl (1-(6-acrylamido-3-morpholinoisoquinolin-1-yl)pyrrolidin-3-yl)carbamate (120 mg, 0.26 mmol) and TFA (5 mL) in DCM (5 mL) was stirred at RT for 1 h. The reaction mixture was concentrated in vacuo to afford a TFA salt of (R)—N-(1-(3-aminopyrrolidin-1-yl)-3-morpholinoisoquinolin-6-yl)acrylamide (80 mg, 85%) as a brown oil. [M+H] Calc'd for C₂₀H₂₅N₅O₂, 368.2; Found, 368.2

Step 8: (R)—N-(1-(3-((5-cyanopyrimidin-2-yl)amino)pyrrolidin-1-yl)-3-morpholinoisoquinolin- 6-yl)acrylamide

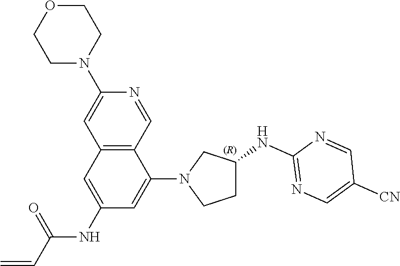

To a solution of (R)—N-(1-(3-aminopyrrolidin-1-yl)-3-morpholinoisoquinolin-6-yl)acrylamide (80 mg, 0.22 mmol) and 2-chloropyrimidine-5-carbonitrile (31 mg, 0.22 mmol) in DMSO (3 mL) was added DIEA (141 mg, 1.09 mmol) at RT. The mixture was stirred at 40° C. for 2 h. The mixture was added water (10 mL) and extracted with EA (10 mL*2). The combined organic layer was washed with brine (10 mL*2), dried over Na₂SO₄, filtered and concentrated. The residue was purified prep-HPLC (0.2% HCOOH) to afford HCOOH salt of (R)—N-(1-(3-((5-cyanopyrimidin-2-yl)amino)pyrrolidin-1-yl)-3-morpholinoisoquinolin-6-yl)acrylamide (14.3 mg, 14%) as a yellow solid. ¹H NMR (400 MHz, DMSO-d₆): d 2.04-2.06 (m, 1H), 2.22-2.24 (m, 1H), 3.48-3.49 (m, 4H), 3.69-3.74 (m, 5H), 3.80-4.05 (m, 3H), 4.51-4.53 (m, 1H), 5.78 (dd, J=2.0, 11.6 Hz, 1H), 6.17 (s, 1H), 6.26-6.30 (m, 1H), 6.44-6.51 (m, 1H), 7.19 (dd, J=9.6, 11.2 Hz, 1H), 7.97-7.99 (m, 2H), 8.64-8.69 (m, 2H), 8.76 (d, J=2.4 Hz, 1H), 10.23 (s, 1H). [M+H] Calc'd for C₂₅H₂₆N₈O₂, 471.3 Found, 471.3

### Example 38: Synthesis of (R,E)—N-(4-(34(5-chloro-4-(1H-indol-3-yl)pyrimidin-2-ynamino)pyrrolidin-1-yl)-2-methylciuinazolin-7-yl)-4-(dimethylamino)but-2-enamide

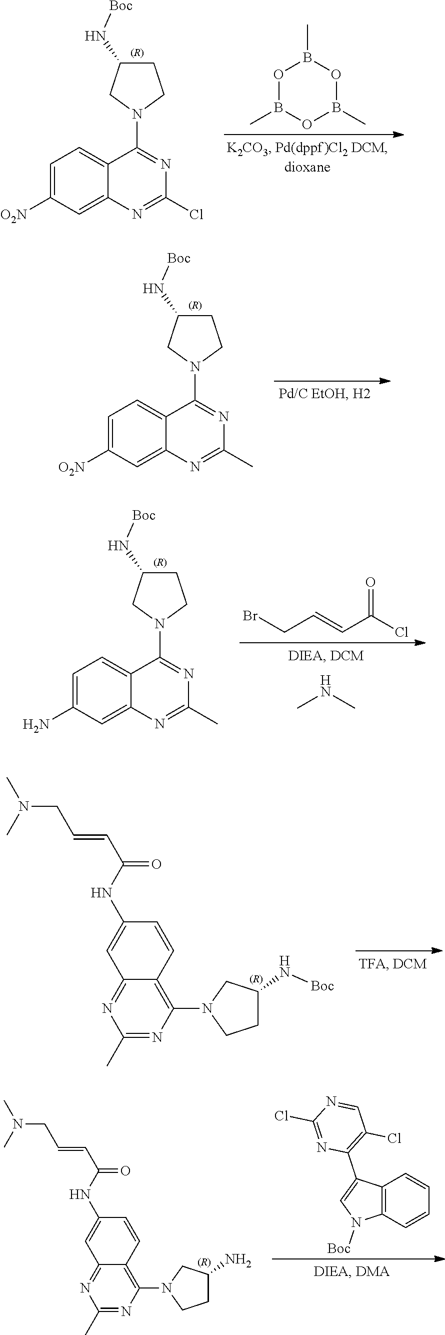

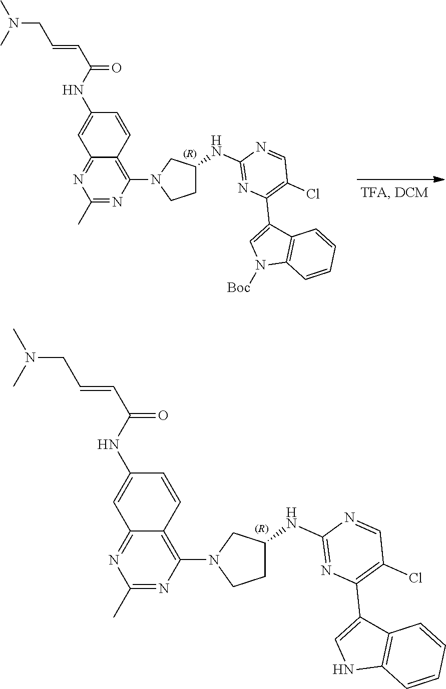

Step 1: (R)-tert-butyl(1-(2-methyl-7-nitroquinazolin-4-yl)pyrrolidin-3-yl)carbamate

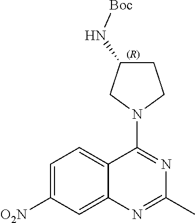

To a solution of (R)-tert-butyl (1-(2-chloro-7-nitroquinazolin-4-yl)pyrrolidin-3-yl)carbamate (12.0 g, 30.5 mmol) and K₂CO₃ (12.7 g, 91.5 mmol) in 1,4-dioxane (100 mL) was added 2,4,6-trimethyl-1,3,5,2,4,6-trioxatriborinane (13.0 g, 45.8 mmol) at 0° C. Then Pd(dppf)Cl₂ DCM (2.5 g, 3.0 mmol) was added under N₂ at RT. The mixture was stirred at 140° C. in sealed tube for 6 h. Then the reaction mixture was cooled to RT, filtered and concentrated. The residue was purified by cloumn (PE/EA=1:1) to afford ((R)-tert-butyl (1-(2-methyl-7-nitroquinazolin-4-yl)pyrrolidin-3-yl)carbamate (5.5 g, 48%) as a yellow solid. [M+H] Calc'd for C₁₈H₂₃N₅O₄, 374.1; Found, 374.1

Step 2: (R)-tert-butyl (1-(7-amino-2-methylquinazolin-4-yl)pyrrolidin-3-yl)carbamate

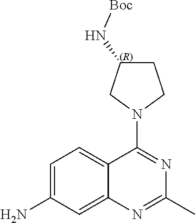

A solution of (R)-tert-butyl (1-(2-methyl-7-nitroquinazolin-4-yl)pyrrolidin-3-yl)carbamate (3.0 g, 8.0 mmol) and Pd/C (1.6 g, 10%) in EtOH (40 ml) was stirred at RT for 3 h under H₂. The mixture was filtered. The organic layer was concentrated in vacuum to give the crude (R)-tert-butyl (1-(7-amino-2-methylquinazolin-4-yl)pyrrolidin-3 -yl)carbamate (2.7 g) as a yellow solid. [M+H] Calc'd for C₁₈H₂₅N₅O₄, 344.2; Found, 344.2

Step 3: (R,E)-tert-butyl (1-(7-(4-(dimethylamino)but-2-enamido)-2-methylquinazolin-4-yl)pyrrolidin-3-yl)carbamate

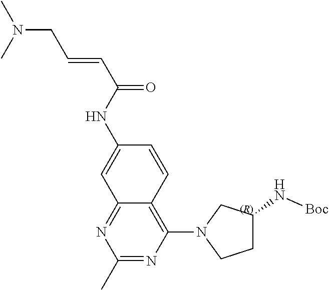

To a solution of DIEA (12.0 mL, 67.6 mmol) and (R)-tert-butyl (1-(7-amino-2-methylquinazolin-4-yl)pyrrolidin-3-yl)carbamate (5.8 g, 16.9 mmol) in DCM (100 mL) was added(E)-4-bromobut-2-enoyl chloride (5.1 g, 31.4 mmol) in an ice-bath. The reaction mixture was stirred at RT for 2 h then dimethylamine (37.8 mL, 2.0 M in THF, 67.6 mmol) was added and the reaction mixture was stirred at RT for 2 h. The mixture was diluted with H₂O (50 mL) and extracted with DCM (50 mL*2). The combined organic layer was washed with brine (50 mL), dried over Na₂SO₄, filtered and concentrated in-vacuo. and purified by column (DCM:MeOH=10:1) to give (R,E)-tert-butyl (1-(7-(4-(dimethylamino)but-2-enamido)-2-methylquinazolin-4-yl)pyrrolidin-3-yl)carbamate (2.5 g, 33%) as a black solid. [M+H] Calc'd for C₄H₃₄N₆O₃, 455.3; Found, 455.3

Step 4: (R,E)—N-(4-(3-aminopyrrolidin-1-yl)-2-methylquinazolin-7-yl)-4-(dimethylamino)but-2-enamide

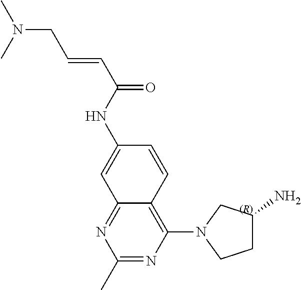

A solution of (R,E)-tert-butyl (1-(7-(4-(dimethylamino)but-2-enamido)-2-methylquinazolin-4-yl)pyrrolidin-3-yl)carbamate (800 mg, 1.76 mmol) and TFA (3 mL) in DCM (6 mL) was stirred at RT for 2 h. The mixture was concentrated to afford TFA salt of (R,E)—N-(4-(3-aminopyrrolidin-1-yl)-2-methylquinazolin-7-yl)-4-(dimethylamino)but-2-enamide (1.0 g, 100%) as a grey solid. [M+H] Calc'd for C₁₉H₂₆N₆O, 355.2 Found, 355.2.

Step 5: (R,E)-tert-butyl 3-(5-chloro-2-((1-(7-(4-(dimethylamino)but-2-enamido)-2-methylquinazolin-4-yl)pyrrolidin-3-yl)amino)pyrimidin-4-yl)-1H-indole-1-carboxylate

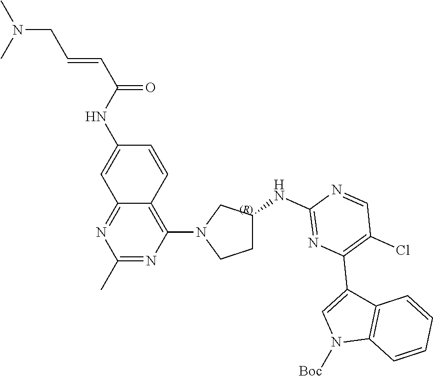

To a solution of (R,E)—N-(4-(3-aminopyrrolidin-1-yl)-2-methylquinazolin-7-yl)-4-(dimethylamino)but-2-enamide (520 mg, 1.46 mmol) and tert-butyl 3-(2,5-dichloropyrimidin-4-yl)-1H-indole-1-carboxylate (531 mg, 1.46 mmol) in DMA (6 mL) was added DIEA (1.56 mL, 8.79 mmol) at RT. The reaction mixture was stirred at 90° C. for 10 h. The reaction mixture was cooled and added water (10 mL), filtered and concentrated to give afford (R,E)-tert-butyl 3-(5-chloro-2-((1-(7-(4-(dimethylamino)but-2-enamido)-2-methylquinazolin-4-yl)pyrrolidin-3-yl)amino)pyrimidin-4-yl)-1H-indole-1-carboxylate (600 mg, 60%) as a yellow solid. [M+H] Calc'd for C₃₆H₄₀ClN₉O₃, 682.3; Found, 682.3

Step 6: (R,E)—N-(4-(3-5-chloro-4-(1H-indol -3-yl)pyrimidin-2-yl)amino)pyrrolidin-1-yl)-2-methylquinazolin-7-yl)-4-(dimethylamino)but-2-enamide HCOOH salt

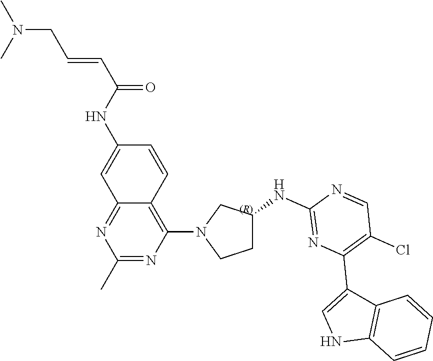

A solution of (R,E)-tert-butyl 3-(5-chloro-2-((1-(7-(4-(dimethylamino)but-2-enamido)-2-methylquinazolin-4-yl)pyrrolidin-3-yl)amino)pyrimidin-4-yl)-1H-indole-1-carboxylate (600 mg, 0.88 mmol) and TFA (1 mL) in DCM (3 mL) was stirred at RT for 1 day. The reaction mixture was concentrated in-vacuo. The residue was dissolved in EA (10 mL) and adjusted pH to 9 with DIEA and concentrated in-vacuo. The residue was purified by prep-HPLC (0.1% HCOOH) to afford HCOOH salt of (R,E)—N-(4-(3-((5-chloro-4-(1H-indol-3-yppyrimidin-2-yl)amino)pyrrolidin-1-yl)-2-methylquinazolin-7-yl)-4-(dimethylamino)but-2-enamide (2.0 mg, 0.4%) as a yellow solid. ¹H NMR (400 MHz, DMSO-d₆): δ 1.94-2.00 (m, 2H), 2.33 (s, 6H), 2.41 (s, 3H), 3.09-3.11 (m, 2H), 3.92-4.29 (m, 4H), 4.55-4.60 (m, 1H), 6.30-6.34 (m, 1H), 6.76-6.83 (m, 1H), 7.10-7.22 (m, 2H), 7.48-7.64 (m, 2H), 7.65-7.67(m, 1H), 8.04 (s, 1H), 8.17-8.19 (m, 3H), 8.31 (m, 1H), 8.47 (s, 1H), 10.34 (m, 1H), 11.83-11.85 (m, 1H). [M+H] Calc'd for C₃₁H₃₂C1N₉O, 582.2 Found, 582.2.

### Example 39: Synthesis of (R)—N-(4-(34(5-cyanopyrimidin-2-yl)amino)pyrrolidin-1-yl)-2-methylquinazolin-7-yl)-N-methylacrylamide

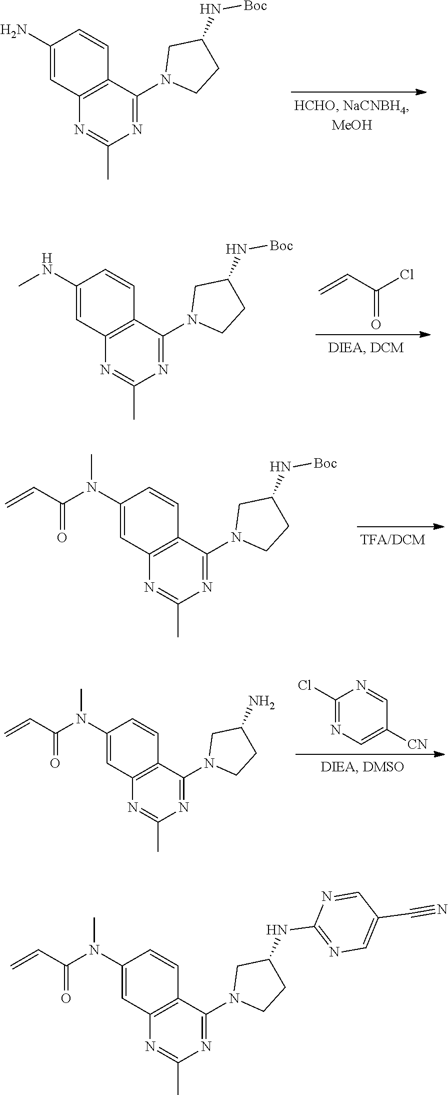

Step 1: (R)-tert-butyl (1-(2-methyl-7-(methylamino)quinazolin-4-yl)pyrrolidin-3-yl)carbamate

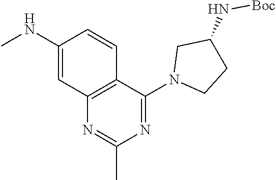 [SMILES: CNc1ccc2c(N3CC[C@@H](NC(=O)OC(C)(C)C)C3)nc(C)nc2c1]

To a mixture of ((R)-tert-butyl (1-(7-amino-2-methylquinazolin-4-yl)pyrrolidin-3-yl)carbamate (656 mg, 2.00 mmol) in MeOH (40 mL) was added HCHO (699 mg, 37% in H₂O, 23.3 mmol) at room temperature. After stirring for 30 min, NaCNBH₄ (2.89 g, 46.6 mmol) was added to the mixture at 0° C. The reaction mixture was stirred at 60° C. for 16 h. The mixture was concentrated. To the residue was added DCM (20 mL), washed with 0.5N HCl (10 mL), dried over Na₂SO₄, filtered and concentrated. The residue was purified by FCC (DCM/MeOH=10:1) to afford (R)-tert-butyl (1-(2-methyl-7-(methylamino)quinazolin-4-yl)pyrrolidin-3-yl)carbamate (500 mg, 25%) as a off white solid. [M+H] Calc'd for C₁₉H₂₇N₅O₂, 358.2; Found, 358.2.

Step 2: (R)-tert-butyl (1-(2-methyl-7-(N-methylacrylamido)quinazolin-4-yl)pyrrolidin-3-yl)carbamate

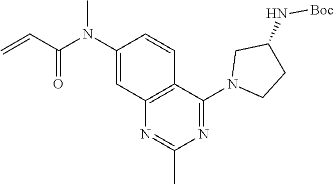 [SMILES: C=CC(=O)N(C)c1ccc2c(N3CC[C@@H](NC(=O)OC(C)(C)C)C3)nc(C)nc2c1]

To a solution of (R)-tert-butyl (1-(2-methyl-7-(methylamino)quinazolin-4-yl)pyrrolidin-3-yl)carbamate (600 mg, 1.685 mmol) and DIEA (0.83 mL, 3.0 mmol) in DCM (50 ml) was added acryloyl chloride (0.14 mg, 1.84 mmol) at 0° C. The reaction mixture was stirred at -50° C. for 30 minutes. The mixture was concentrated and purified by FCC (DCM/MeOH=20:1) to afford (R)-tert-butyl (1-(2-methyl-7-(N-methylacrylamido)quinazolin-4-yl)pyrrolidin-3-yl)carbamate (200 mg, 28.9%) as an off white solid. [M+H] Calc'd for C₂₂H₂₉N₅O₃, 412.2; Found, 412.2.

Step 3: (R)—N-(4-(3-aminopyrrolidin-1-yl)-2-methylquinazolin-7-yl)-N-methylacrylamide TFA salt

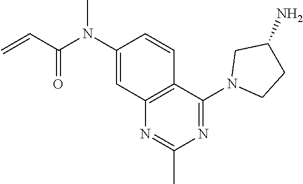 [SMILES: C=CC(=O)N(C)c1ccc2c(N3CC[C@@H](N)C3)nc(C)nc2c1]

A solution of (R)-tert-butyl (1-(2-methyl-7-(N-methylacrylamido)quinazolin-4-yl)pyrrolidin-3-yl)carbamate (200 mg, 0.48 mmol) in TFA/DCM (1 mL/ 10 mL) was stirred at RT for 3 h. The mixture was concentrated to afford TFA salt of (R)—N-(4-(3-aminopyrrolidin-1-yl)-2-methylquinazolin-7-yl)-N-methylacrylamide (151 mg, 100%) as a yellow solid. [M+H] Calc'd for C₁₇H₂₁N₅O, 312.2 Found,312.2.

Step 4: (R)—N-(4-(3-((5-cyanopyrimidin-2-yl)amino)pyrrolidin-1-yl)-2-methylquinazolin-7-yl)-N-methylacrylamide

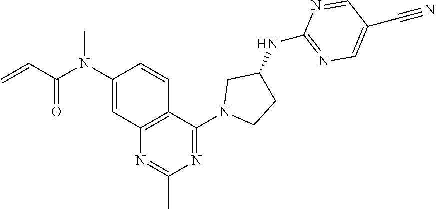 [SMILES: C=CC(=O)N(C)c1ccc2c(N3CC[C@@H](Nc4ncc(C#N)cn4)C3)nc(C)nc2c1]

A solution of TFA salt of (R)—N-(4-(3-aminopyrrolidin-1-yl)-2-methylquinazolin-7-yl)-N-methylacrylamide (140 mg, 0.45 mmol), 2-chloropyrimidine-5-carbonitrile (93 mg, 0.67 mmol) and DIEA (0.37 mL, 2.25 mmol) in DMSO (5 ml) was stirred at 30° C. for 1 h. The mixture was diluted with water (10 mL) and extracted with EA (10 mL*2). The combined extracts were washed with water (10 mL*2) and brine (10 mL), dried over anhydrous sodium sulfate, filtered and concentrated. The residue was purified by prep-HPLC to give the product (R)—N-(4-(3-((5-cyanopyrimidin-2-yl)amino)pyrrolidin-1-yl)-2-methylquinazolin-7-yl)-N-methylacrylamide (85.2 mg, 45.8%) as an off white solid. ¹H NMR (400 MHz, DMSO-d₆): δ 2.12-2.15 (m, 1H), 2.28-2.31 (m, 1H), 2.45 (s, 3H), 3.29-3.34 (m, 3H), 3.86-3.89 (m, 1H), 3.96-3.98 (m, 1H), 4.08-4.11 (m, 1H), 4.19-4.23 (m, 1H), 4.58-4.61 (m, 1H), 5.60-5.63 (m, 1H), 6.18-6.20 (m, 2H), 7.26 (dd, J=2.0, 8.0 Hz, 1H) ,7.46(d, J=2.0 Hz, 1H), 8.25(d, J=9.2 Hz, 1H), 8.67-8.70 (m, 2H), 8.79 (s, 1H). [M+H] Calc'd for C₂₂H₂₂N₈O, 415.1; Found, 415.1.

### Example 40: Synthesis of (R)—N-(4-(3-((5-bromopyrimidin-2-yl)amino)pyrrolidin-1-yl)-2-methylquinazolin-7-yl)-N-methylacrylamide

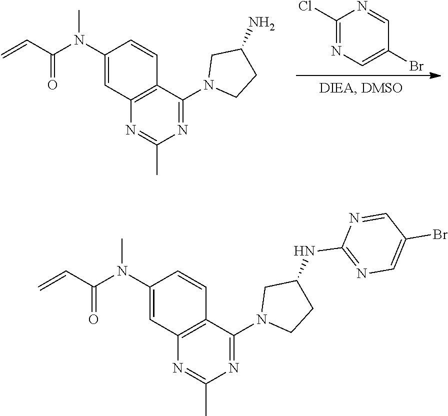 [SMILES: C=CC(=O)N(C)c1ccc2c(N3CC[C@@H](N)C3)nc(C)nc2c1 ; Clc1ncc(Br)cn1 ; C=CC(=O)N(C)c1ccc2c(N3CC[C@@H](Nc4ncc(Br)cn4)C3)nc(C)nc2c1]

A solution of TFA salt of (R)—N-(4-(3-aminopyrrolidin-1-yl)-2-methylquinazolin-7-yl)-N-methylacrylamide (220 mg, 0.70 mmol), 5-bromo-2-chloropyrimidine (218 mg, 1.13 mmol) and DIEA (0.18 mL, 1.10 mmol) in DMSO (5 ml) was stirred at 80° C. in microwave for 2 h. The mixture was diluted with water (20 mL) and extracted with EA (20 mL*2). The combined extracts were washed with water (50 mL*2) and brine (100 mL), dried over anhydrous sodium sulfate, filtered and concentrated. The residue was purified by prep-HPLC to give (R)—N-(4-(3-((5-bromopyrimidin-2-yl)amino)pyrrolidin-1-yl)-2-methylquinazolin-7-yl)-N-methylacrylamide (47.3 mg, 14.2%) as an off white solid. ¹H NMR (400 MHz, DMSO-d₆): δ 2.07-2.09 (m, 1H), 2.10-2.12 (m, 1H), 2.46 (s, 3H), 3.26-3.324 (m, 3H), 3.84-3.88 (m, 1H), 3.96-3.98 (m, 1H), 4.09-4.11 (m, 1H), 4.18-4.21 (m, 1H), 4.45-4.46 (m, 1H), 5.61-5.64 (m, 1H), 6.19-6.21(m, 2H), 7.33 (dd, J=2.0, 8.0 Hz, 1H) ,7.45(d, J=2.4 Hz, 1H) 7.87(d, J=8.0 Hz, 1H), 8.27 (d, J=8.0 Hz, 1H), 8.43 (s, 2H). [M+H] Calc'd for C₂₁H₂₂BrN₇O, 468.1; Found, 468.1

### Example 41: Synthesis of (R)—N-(4-(3-((5-cyanopyrimidin-2-yl)amino)pyrrolidin-1-yl)-2-(4-methylpiperazin-1-yl)quinazolin-7-yl)-N-methylacrylamide

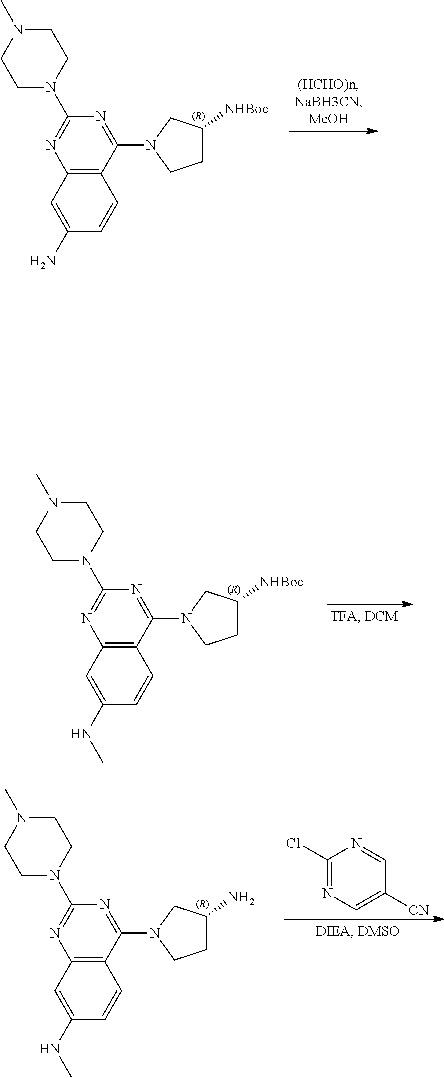

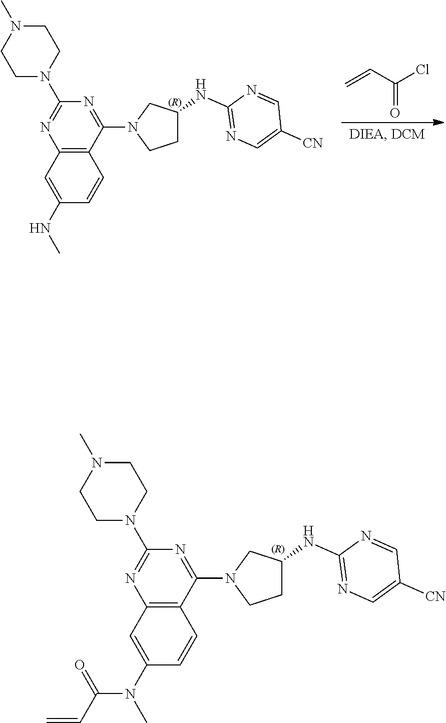

Step 1: (R)-tert-butyl (1-(7-(methylamino)-2-(4-methylpiperazin-1-yl)quinazolin-4-yl)pyrrolidin-3-yl)carbamate

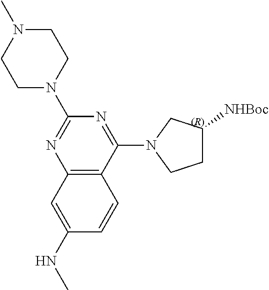

A solution of (R)-tert-butyl (1-(7-amino-2-(4-methylpiperazin-1-yl)quinazolin-4-yl)pyrrolidin-3-yl)carbamate (6.0 g, 14.0 mmol) and (HCHO)n (1.7 g, 21.0 mmol) in MeOH (300 mL) was stirred at RT for 1 h. Then NaBH₃CN (2.7 g, 42.0 mmol) was added. The mixture was stirred at 40° C. overnight. The reaction mixture was removed the solvent. The residue was purified by prep-HPLC to give the (R)-tert-butyl (1-(7-(methylamino)-2-(4-methylpiperazin-1-yl)quinazolin-4-yl)pyrrolidin-3-yl)carbamate (1.3 g, 21%) as a yellow solid. [M+H] Calc'd for C₂₃H₃₅N₇O₂, 442.3; Found, 442.3

Step 2: (R)-4-(3-aminopyrrolidin-1-yl)-N-methyl-2-(4-methylpiperazin-1-yl)quinazolin-7-amine TFA salt

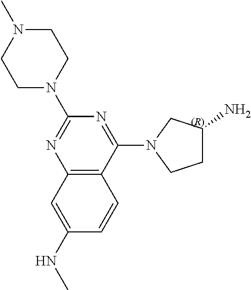

A solution of (R)-tert-butyl (1-(7-(methylamino)-2-(4-methylpiperazin-1-yl)quinazolin-4-yl)pyrrolidin-3-yl)carbamate (100 mg, 0.23 mmol) and TFA (0.5 mL) in DCM (5 mL) was stirred at RT for 2 h. The mixture was concentrated to afford TFA salt of (R)-4-(3-aminopyrrolidin-1-yl)-N-methyl-2-(4-methylpiperazin-1-yl)quinazolin-7-amine (77 mg) as yellow oil. [M+H] Calc'd for C₁₈H₂₇N₇, 342.2 Found,342.2.

Step 3: (R)-2-((1-(7-(methylamino)-2-(4-methylpiperazin-1-yl)quinazolin-4-yl)pyrrolidin-3-yl)amino)pyrimidine-5-carbonitrile

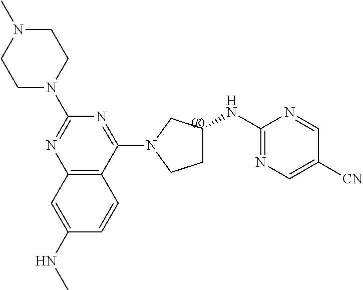

A solution of (R)-4-(3-aminopyrrolidin-1-yl)-N-methyl-2-(4-methylpiperazin-1-yl)quinazolin-7-amine (77 mg, 0.22 mmol) and 2-chloropyrimidine-5-carbonitrile (32 mg, 0.22 mmol), DIEA (117 mg, 0.99 mmol) in DMSO (5 mL) was stirred at 30° C. for 2 h. The mixture was diluted with water (10 mL) and extracted with DCM (10 mL*2). The combined organic layer was washed with H₂O (10 mL*2) and brine, dried over Na₂SO₄, filtered and concentrated. The residue was purified by prep-HPLC to give (R)-2-((1-(7-(methylamino)-2-(4-methylpiperazin-1-yl)quinazolin-4-yl)pyrrolidin-3-yl)amino)pyrimidine-5-carbonitrile (60 mg, 60%) as a yellow solid. [M+H] Calc'd for C₂₃H₈N₁₀, 445.3 Found, 445.3.

Step 4: (R)—N-(4-(3-((5-cyanopyrimidin-2-yl)amino)pyrrolidin-1-yl)-2-(4-methylpiperazin-1-yl)quinazolin-7-yl)-N-methylacrylamide

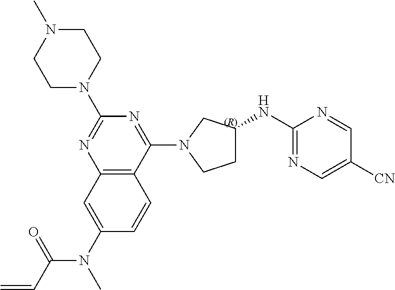

A solution of (R)-2-(1-(7-(methylamino)-2-(4-methylpiperazin-1-yl)quinazolin-4-yl)pyrrolidin-3-yl)amino)pyrimidine-5-carbonitrile (250 mg, 0.56 mmol) and DIEA (218 mg, 1.69 mmol) in DCM (20 mL) was stirred at 0° C. for 10 min. Then acryloyl chloride (77 mg, 0.85 mmol) was added slowly. The mixture was stirred at RT for 1 h. The residue was concentrated and purified by prep-HPLC to afford (R)—N-(4-(3-((5-cyanopyrimidin-2-yl)amino)pyrrolidin-1-yl)-2-(4-methylpiperazin-1-yl)quinazolin-7-yl)-N-methylacrylamide (119 mg, 43%) as a yellow solid. ¹H NMR (400 MHz, DMSO-d₆): δ 2.07-2.11 (m, 1H), 2.20-2.33 (m, 8H), 3.29 (s, 3H), 3.75-4.17 (m, 8H), 4.57-4.59 (m, 1H), 5.59 (t, J=6.4 Hz, 1H), 6.17-6.18 (m, 2H), 6.92 (dd, J=1.6, 8.8 Hz, 1H), 7.11 (d, J=2.0 Hz, 1H), 8.04 (d, J=8.8 Hz, 1H), 8.66-8.70(m, 2H), 8.78 (s, 1H). [M+H] Calc'd for C₂₆H₃₀N₁₀O, 499.2; Found, 499.2

### Example 42: Synthesis of (R)—N-(1-(3-((5-cyanopyrimidin-2-yl)amino)pyrrolidin-1-yl)-3-(1-methyl-1,2,3,6-tetrahydropyridin-4-yl)isoquinolin-6-yl)-N-methylacrylamide

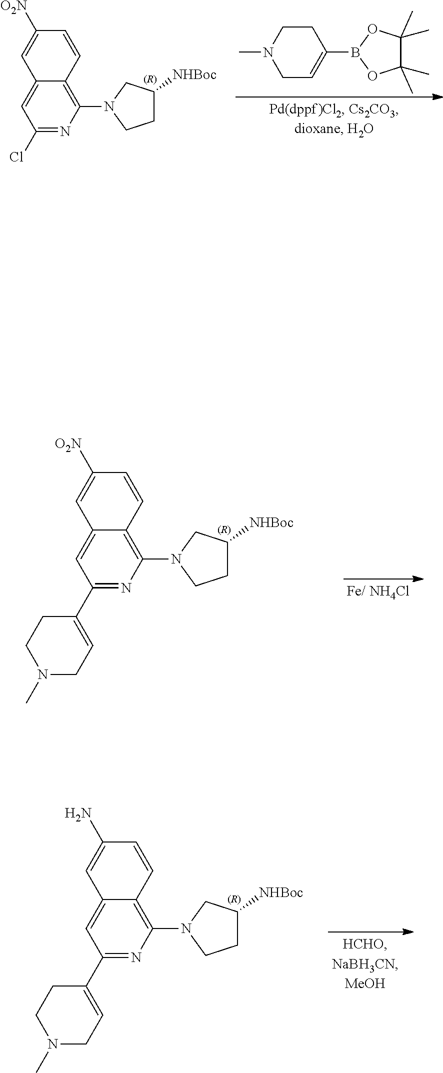

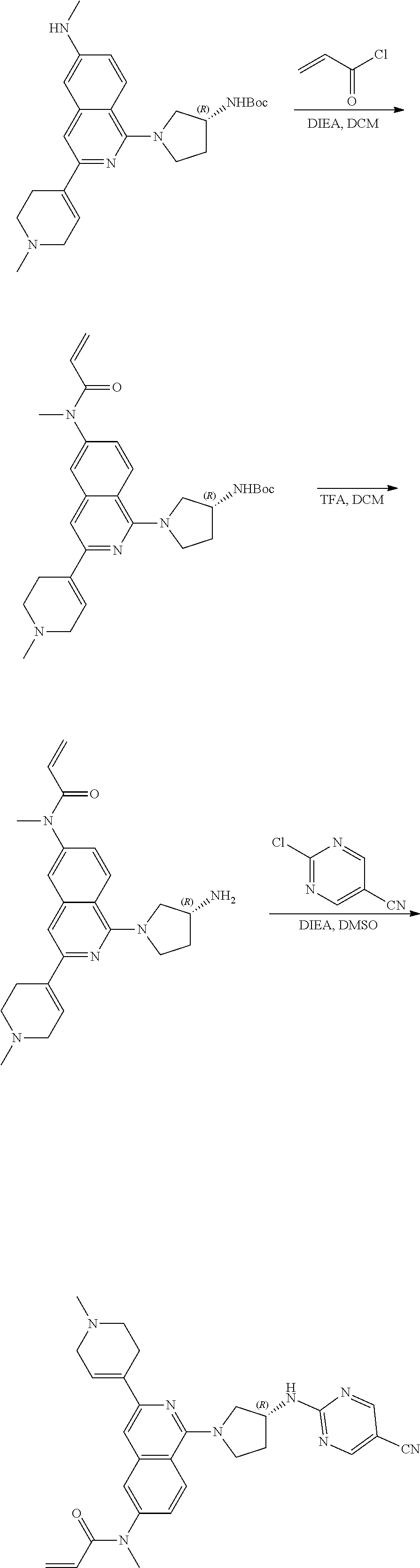

Step 1: (R)-tert-butyl (1-(3-(1-methyl-1,2,3,6-tetrahydropyridin-4-yl)-6-nitroisoquinolin-1-yl)pyrrolidin-3-yl)carbamate

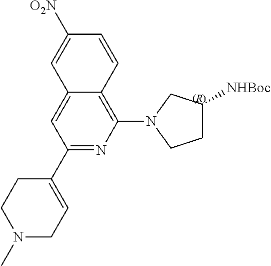

To a solution of (R)-tert-butyl (1-(3-chloro-6-nitroisoquinolin-1-yl)pyrrolidin-3-yl)carbamate (2.5 g, 6.4 mmol) and 1-methyl-4-(4,4,5,5-tetramethyl-1,3,2-dioxaborolan-2-yl)-1,2,3,6-tetrahydropyridine (1.6 g, 7.0 mmol) in dioxane (50 mL) and H₂O (5 mL) was added Pd(dppf)C12 (466 mg, 0.64 mmol) and Cs₂CO₃ (4.15 g, 12.7 mmol) under N₂. The mixture was stirred at 105° C. for 5 h. The residue was filtered and concentrated. The residue was purified by column (PE:EA=10:1) to afford the (R)-tert-butyl (1-(3-(1-methyl-1,2,3,6-tetrahydropyridin-4-yl)-6-nitroisoquinolin-1-yl)pyrrolidin-3-yl)carbamate (1.8 g, 62%) as a brown solid. [M+H] Calc'd for C₂₄H₃₁N₅O₄, 454.2; Found, 454.2

Step 2: (R)-tert-butyl (1-(6-amino-3-(1-methyl-1,2,3,6-tetrahydropyridin-4-yl)isoquinolin-1-yl)pyrrolidin-3-yl)carbamate

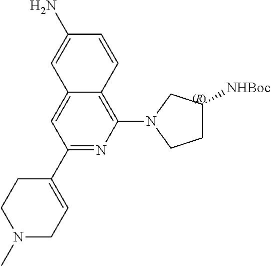

To a solution of (R)-tert-butyl (1-(3-(1-methyl-1,2,3,6-tetrahydropyridin-4-yl)-6-nitroisoquinolin-1-yl)pyrrolidin-3-yl)carbamate (1.8 g, 3.97 mmol) and NH₄C₁ (2.2 g, 3.97 mmol) in EtOH (100 mL) and H₂O (10 mL) was added iron dust (2.2 g, 3.97 mmol) at RT. The mixture was stirred at 80° C. for 4 h. The reaction mixture was filterd and concentrated. The residue was purified by cloumn (DCM:MeOH=10:1) to afford (R)-tert-butyl (1-(6-amino-3-(1-methyl-1,2,3,6-tetrahydropyridin-4-yl)isoquinolin-1-yl)pyrrolidin-3-yl)carbamate (1.6 g, 85%) as a brown solid. [M+H] Calc'd for C₂₄H₃₃N₅O₂, 424.3 Found, 424.3

Step 3: (R)-tert-butyl (1-(3-(1-methyl-1,2,3,6-tetrahydropyridin-4-yl)-6-(methylamino)isoquinolin-1-yl)pyrrolidin-3-yl)carbamate

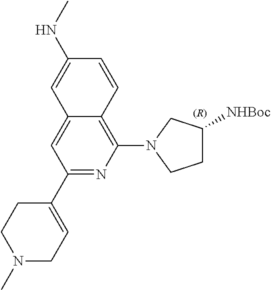

A solution of (R)-tert-butyl (1-(6-amino-3-(1-methyl-1,2,3,6-tetrahydropyridin-4-yl)isoquinolin-1-yl)pyrrolidin-3-yl)carbamate (1.6 g, 3.78 mmol) and HCHO (210 mg, 30% in water, 7.09 mmol) in MeOH (120 mL) was stirred at RT for 1 h. Then NaBH₃CN (894 mg, 14.2 mmol) was added. The mixture was stirred at 40° C. overnight. The reaction mixture was removed the solvent. The residue was purified by silica column (PE:EA 32 10:1) to give (R)-tert-butyl (1-(3-(1-methyl-1,2,3,6-tetrahydropyridin-4-yl)-6-(methylamino)isoquinolin-1-yl)pyrrolidin-3-yl)carbamate (1.0 g, 61%) as a brown solid. [M+H] Calc'd for C₂₅H₃₅N₅O₂, 438.3; Found, 438.3

Step 4: (R)-tert-butyl (1-(3-(1-methyl-1,2,3,6-tetrahydropyridin-4-yl)-6-(N-methylacrylamido)isoquinolin-1-yl)pyrrolidin-3-yl)carbamate

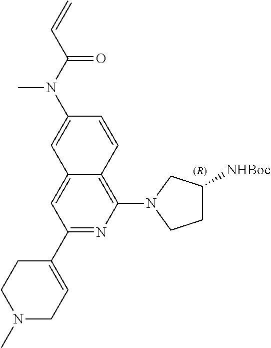

A solution of (R)-tert-butyl (1-(3-(1-methyl-1,2,3,6-tetrahydropyridin-4-yl)-6-(methylamino)isoquinolin-1-yl)pyrrolidin-3-yl)carbamate (900 mg, 2.06 mmol) and DIEA (797 mg, 6.18 mmol) in DCM (50 mL) was stirred at 0° C. for 10 min. Then acryloyl chloride (185 mg, 2.06 mmol) was added slowly at 0° C. The mixture was stirred at RT for 1 h. The residue was concentrated and purified by column (DCM:MeOH=10:1) to afford (R)-tert-butyl (1-(3-(1-methyl-1,2,3,6-tetrahydropyridin-4-yl)-6-(N-methylacrylamido)isoquinolin-1-yl)pyrrolidin-3-yl)carbamate (600 mg, 59%) as a brown solid. [M+H] Calc'd for C₂₈H₃₇N₅O₃, 492.3; Found, 492.3

Step 5: (R)—N-(1-(3-aminopyrrolidin-1-yl)-3-(1-methyl-1,2,3,6-tetrahydropyridin-4-yl)isoquinolin-6-yl)-N-methylacrylamide TFA salt

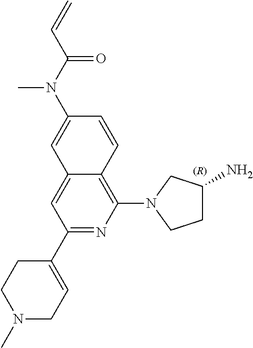

A solution of (R)-tert-butyl (1-(3-(1-methyl-1,2,3,6-tetrahydropyridin-4-yl)-6-(N-methylacrylamido)isoquinolin-1-yl)pyrrolidin-3-yl)carbamate (600 mg, 1.22 mmol) and TFA (100 mL) in DCM (100 mL) was stirred at RT for 1 h. The mixture was concentrated to afford TFA salt of (R)—N-(1-(3-aminopyrrolidin-1-yl)-3-(1-methyl-1,2,3,6-tetrahydropyridin-4-yl)isoquinolin-6-yl)-N-methylacrylamide (450 mg, 94%) as brown oil. [M+H] Calc'd for C₂₃H₂₉N₅O₂, 392.2 Found, 392.2.

Step 6: (R)—N-(1-(3-((5-cyanopyrimidin-2-yl)amino)pyrrolidin-1-yl)-3-(1-methyl-1,2,3,6-tetrahydropyridin-4-ypisoquinolin-6-yl)-N-methylaerylamide HCOOH salt

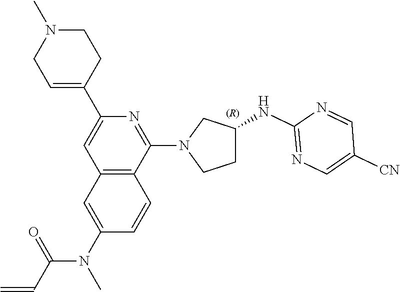

A solution of (R)—N-(1-(3-aminopyrrolidin-1-yl)-3-(1-methyl-1,2,3,6-tetrahydropyridin-4-yl)isoquinolin-6-yl)-N-methylacrylamide (150 mg, 0.38 mmol) and 2-chloropyrimidine-5-carbonitrile (54 mg, 0.38 mmol) and DIEA (247 mg, 1.92 mmol) in DMSO (5 mL) was stirred at 40° C. for 1 h. The mixture was diluted with water (15 mL) and extracted with DCM (15 mL*2). The combined organic layer was washed with H₂O (15 mL*2) and brine, dried over Na₂SO₄, filtered and concentrated. The residue was purified by prep-HPLC (0.2% HCOOH) to give HCOOH salt of (R)—N-(1-(3 -((5-cyanopyrimidin-2-yl)amino)pyrrolidin-1-yl)-3 -(1-methyl-1,2,3,6-tetrahydropyridin-4-yl)isoquinolin-6-yl)-N-methylacrylamide (47.3 mg, 25%) as a brown solid. ¹H NMR (400 MHz, DMSO-d₆): δ 2.08-2.11 (m, 1H), 2.20-2.24 (m, 1H), 2.40 (s, 3H), 2.60-2.73 (m, 4H), 3.22-3.32 (m, 5H), 3.80-3.84 (m, 1H), 3.88-3.93 (m, 1H), 3.99-4.06 (m, 1H), 4.15-4.18 (m, 1H), 4.52-4.60 (m, 1H), 5.60-5.66 (m, 1H), 6.15-6.23 (m, 2H), 6.85 (s, 1H), 7.11 (s, 1H), 7.28-7.33 (m, 1H), 7.64 (s, 1H), 8.15 (s, 1H), 8.27-8.29 (m, 1H), 8.63-8.80 (m, 3H). [M+H] Calc'd for C₂₈H₃₀N₈O, 495.3 Found, 495.3.

### Example 43: Synthesis of (R)—N-(4-(3-((5-bromopyrimidin-2-yl)amino)pyrrolidin-1-yl)-2-(4-methylpiperazin-1-yl)quinazolin-7-yl)-N-methylacrylamide

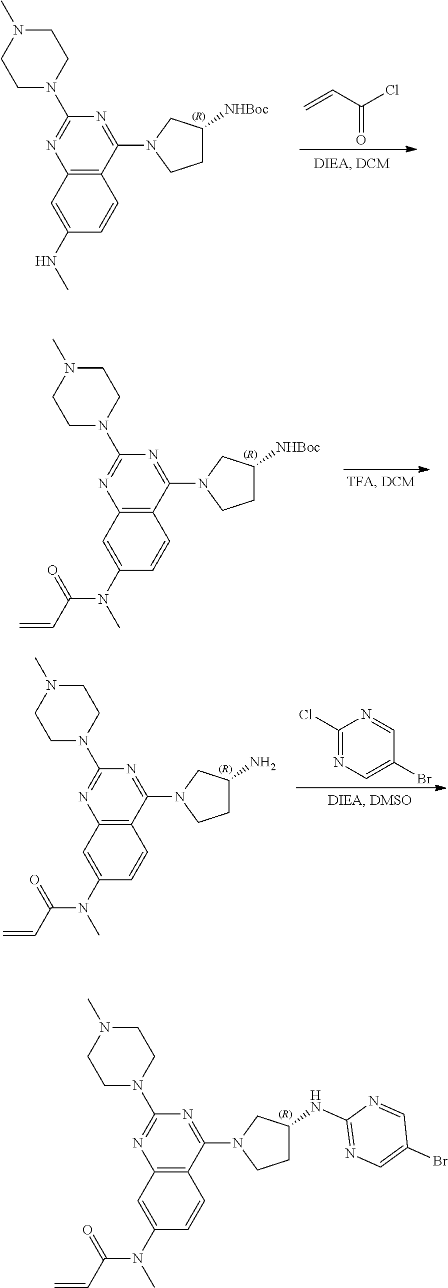

Step 1: (R)-tert-butyl (1-(7-(N-methylacrylamido)-2-(4-methylpiperazin-1-yl)quinazolin-4-yl)pyrrolidin-3-yl)carbamate

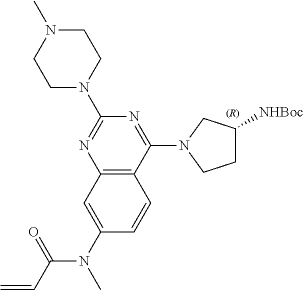

A solution of (R)-tert-butyl (1-(7-(methylamino)-2-(4-methylpiperazin-1-yl)quinazolin-4-yl)pyrrolidin-3-yl)carbamate (400 mg, 0.91 mmol) and DIEA (235 mg, 1.81 mmol) in DCM (15 mL) was stirred at 0° C. for 10 min. Then acryloyl chloride (100 mg, 1.09 mmol) was added slowly. The mixture was stirred at 0-RT overnight. The reaction mixture was added water (20 mL) and extracted with EA (20 mL*2). The combined organic layer was washed with brine (15 mL*2), dried over Na₂SO₄, filtered and concentrated to afford (R)-tert-butyl (1-(7-(N-methylacrylamido)-2-(4-methylpiperazin-1-yl)quinazolin-4-yl)pyrrolidin-3-yl)carbamate (440 mg, 98%) as a yellow solid. [M+H] Calc'd for C₂₆H₃₇N₇O₃, 496.3; Found, 496.3

Step 2: (R)—N-(4-(3-aminopyrrolidin-1-yl)-2-(4-methylpiperazin-1-yl)quinazolin-7-yl)-N-methylacrylamide TFA salt

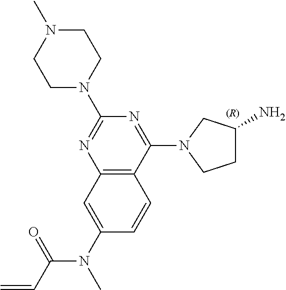

A solution of (R)-tert-butyl (1-(7-(N-methylacrylamido)-2-(4-methylpiperazin-1-yl)quinazolin-4-yl)pyrrolidin-3-yl)carbamate (440 mg, 0.69 mmol) and TFA (2 mL) in DCM (10 mL) was stirred at RT for 2 h. The mixture was concentrated to afford the crude TFA salt of (R)—N-(4-(3-aminopyrrolidin-1-yl)-2-(4-methylpiperazin-1-yl)quinazolin-7-yl)-N-methylacrylamide (440 mg) as yellow oil. [M+H] Calc'd for C₂₁H₂₉N₇O, 395.2 Found,395.2.

Step 3: (R)—N-(4-(3-((5-bromopyrimidin-2-yl)amino)pyrrolidin-1-yl)-2-(4-methylpiperazin-1-yl)quinazolin-7-yl)-N-methylacrylamide

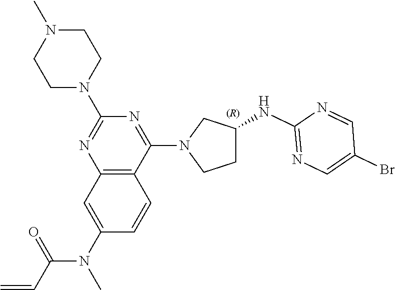

A solution of (R)—N-(4-(3 -aminopyrrolidin-1-yl)-2-(4-methylpiperazin-1-yl)quinazolin-7-yl)-N-methylacrylamide (200 mg, 0.51 mmol) and 5-bromo-2-chloropyrimidine (147 mg, 0.76 mmol), DIEA (130 mg, 1.02 mmol) in DMSO (5 mL) was stirred at 70° C. under microwave for 2 h. The mixture was diluted with water (15 mL) and extracted with EA (15 mL*2). The combined organic layer was washed with brine (15 mL), dried over Na₂SO₄, filtered and concentrated. The residue was purified by prep-HPLC to give (R)—N-(4-(3-((5-bromopyrimidin-2-yl)amino)pyrrolidin-1-yl)-2-(4-methylpiperazin-1-yl)quinazolin-7-yl)-N-methylacrylamide (15.1 mg, 5.4%) as a white solid. ¹HNIVIR (400 MHz, CD₃OD): δ 2.07-2.21 (m, 1H), 2.32-2.40 (m, 4H), 2.51-2.53 (m, 4H), 3.40 (s, 3H), 3.87-3.91 (m, 5H), 3.99-4.05 (m, 1H), 4.10-4.14 (m, 1H), 4.23-4.27 (m, 1H), 4.57-4.59 (m, 1H), 5.62 (dd, J=3.2, 9.6 Hz, 1H), 6.26-6.34 (m, 2H), 6.98 (dd, J=2.0, 8.4 Hz, 1H), 7.24 (d, J=2.4 Hz, 1H), 8.16 (d, J=9.2 Hz, 1H), 8.36 (s, 2H). [M+H] Calc'd for C₂₅H₃₀BrN₉O, 552.2 Found, 552.2.

### Example 44: (R)—N-(1-(3-((5-ethynylpyrimidin-2-yl)amino)pyrrolidin-1-yl)isoquinolin-6-yl)-N-methylacrylamide

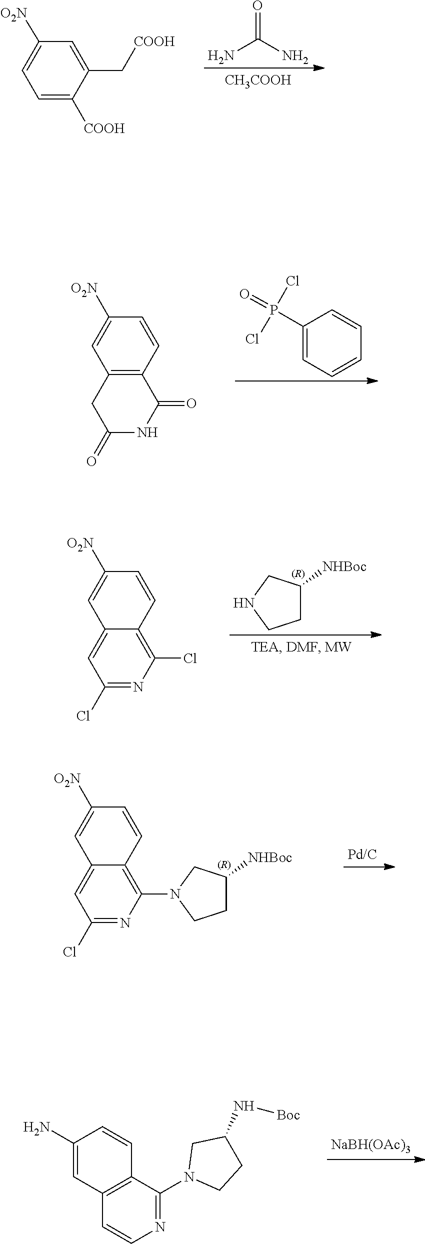

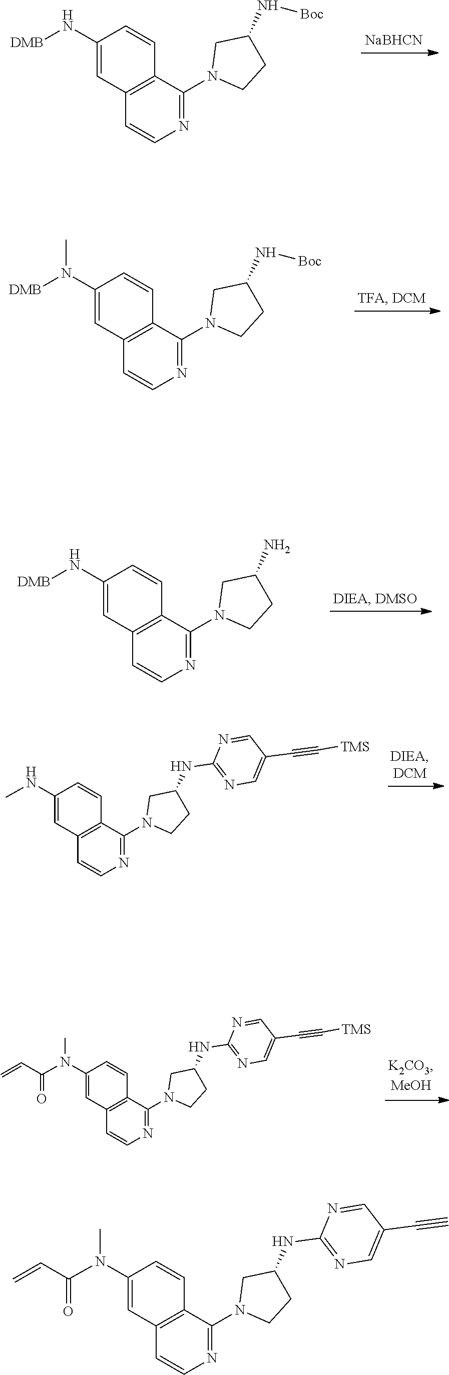

Step 1: 6-nitroisoquinoline-1,3(2H,4H)-dione

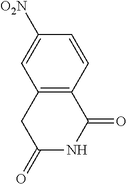 [SMILES: O=C1Cc2cc([N+](=O)[O-])ccc2C(=O)N1]

A mixture of 2-(carboxymethyl)-4-nitrobenzoic acid (45.0 g, 200.0 mmol) and CH₃COOH (500 mL) was stirred at 110° C. for 0.5 h. Then the reaction mixture was cooled to 90° C. and urea (71.0 g, 1.42 mol) was added. The reaction mixture was stirred at 110° C. for 4 h. The solution was cooled to RT and H₂O (500 mL) was added. The mixture was stirred at RT for 0.5 h, filtered and concentrated to afford 6-nitroisoquinoline-1,3(2H,4H)-dione (23.0 g, 56%) as a brown solid.

Step 2: 1,3-dichloro-6-nitroisoquinoline

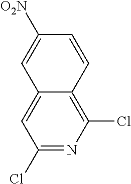 [SMILES: O=[N+]([O-])c1ccc2c(Cl)nc(Cl)cc2c1]

A mixture of 6-nitroisoquinoline-1,3(2H,4H)-dione (14.0 g, 67.9 mmol) and phenylphosphonic dichloride (200 mL) was stirred at 140° C. for 4 h. The reaction mixture was cooled, added water (200 mL) and extracted with EA (200 mL*2). The combined organic layers were washed with brine (100 mL), dried over Na₂SO₄, filtered and concentrated in vacuo. The residue was purified by column (PE:EA=10:1) to afford 1,3-dichloro-6-nitroisoquinoline (19.0 g, 70%) as a yellow solid. [M+H] Calc'd for C₉H₄Cl₂N₂O₂, 242.9; Found, 242.9

Step 3: (R)-tert-butyl (1-(3-chloro-6-nitroisoquinolin-1-yl)pyrrolidin-3-yl)carbamate

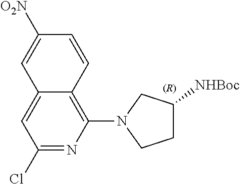

To a solution of 1,3-dichloro-6-nitroisoquinoline (5.0 g, 20.6 mmol) and (R)-tert-butyl pyrrolidin-3-ylcarbamate (3.5 g, 20.6 mmol) in DMF (50 mL) was added TEA (2.1 g, 20.6 mmol) at rt. The reaction mixture was stirred at 140° C. under microwave for 2 h. The reaction mixture was cooled, added water (100 mL) and extracted with EA (100 mL*2). The combined organic layer was washed with water (100 mL*2) and brine (100 mL2), dried over Na₂SO₄, filtered and concentrated in vacuo. The residue was purified by cloumn (PE:EA=5:1) to afford (R)-tert-butyl (1-(3-chloro-6-nitroisoquinolin-1-yl)pyrrolidin-3-yl)carbamate (4.8 g, 60%) as a brown solid. [M+H] Calc'd for C₁₈H₂₁ClN₄O₄, 393.1; Found, 393.1.

Step 4: (R)-tert-butyl (1-(6-aminoisoquinolin-1-yl)pyrrolidin-3-yl)carbamate

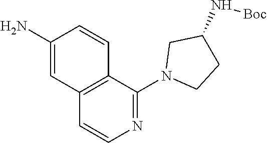 [SMILES: CC(C)(C)OC(=O)N[C@@H]1CCN(c2nccc3cc(N)ccc23)C1]

To a solution of (R)-tert-butyl (1-(3-chloro-6-nitroisoquinolin-1-yl)pyrrolidin-3-yl)carbamate (5.0 g, 12.8 mmol) in EtOH (150 mL) was added Pd/C (500 mg, 5%) at rt. The reaction mixture was stirred at rt under hydrogen atmosphere overnight. The reaction mixture was filtered. The filtrate was concentrated to obtain the crude product (3.8 g, 90%) as a yellow solid. [M+H] MS Calc'd C₁₈H₂₄N₄O₂, 329.4; Found:329.4.

Step 5: (R)-tert-butyl (1-(6-((2,4-dimethoxybenzyl)amino)isoquinolin-1-yl)pyrrolidin-3-yl)carbamate

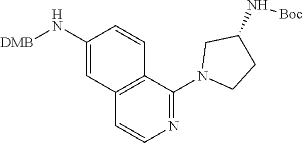

To a solution of (R)-tert-butyl (1-(6-aminoisoquinolin-1-yl)pyrrolidin-3-yl)carbamate (330 mg, 1.0 mmol) in DCE (50 mL) was added 2,4-dimethoxybenzaldehyde (184 mg, 1.1 mmol) and Na BH(OAc)₃ (640 mg, 3.0 mmol) at rt. The reaction mixture was stirred at rt overnight. The residue was quenched with sodium bicarbonate aqueous solution (50 mL) and extracted with EA (20 mL*2). The combined organic layer was washed with water (20 mL*2) and brine (20 mL*2), dried over Na₂SO₄, filtered and concentrated in vacuo. The residue was purified by cloumn (PE:EA=1:1) to afford (R)-tert-butyl (1-(6-((2,4-dimethoxybenzyl)amino)isoquinolin-1-yl)pyrrolidin-3-yl)carbamate (200 mg, 42%) as a yellow solid. [M+H] Calc'd for C₂₇H₃₄N₄O₄, 479.2; Found, 479.2.

Step 6: (R)-tert-butyl (1-(6-(2,4-dimethoxybenzyl)(methyl)amino)isoquinolin-1-yl)pyrrolidin-3-yl)carbamate

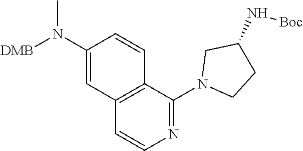

To a solution of (R)-tert-butyl (1-(6-((2,4-dimethoxybenzyl)amino)isoquinolin-1-yl)pyrrolidin-3-yl)carbamate (200 mg, 0.42 mmol) in MeOH (5 mL) was added acetaldehyde (720 mg, 8.3 mmol) and NaBHCN (105 mg, 1.7 mmol) at rt. The reaction mixture was refluxed overnight. The reaction mixture was cooled, quenched with added water (20 mL) and extracted with EA (10 mL*2). The combined organic layer was washed with water (10 mL*2) and brine (10 mL*2), dried over Na₂SO₄, filtered and concentrated in vacuo. The residue was purified by C18 cloumn (ACN/H₂O=40%-90%) to afford (R)-tert-butyl (1-(6-((2,4-dimethoxybenzyl)(methyl)amino)isoquinolin-1-yl)pyrrolidin-3-yl)carbamate (100 mg, 50%) as a yellow solid. [M+H] Calc'd for C₂₈H₃₆N₄O₄, 493.3; Found, 493.3.

Step 7: (R) -1-(6-(methylamino)naphthalen-1-yl)pyrrolidin-3-amine

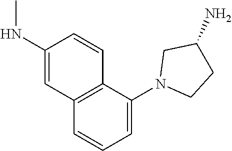 [SMILES: CNc1ccc2c(N3CC[C@@H](N)C3)cccc2c1]

To a solution of (R)-tert-butyl (1-(6-((2,4-dimethoxybenzyl)(methyl)amino)isoquinolin-1-yl)pyrrolidin-3-yl)carbamate (200 mg, 0.41 mmol) in DCM (5 mL) was added TFA (1 mL), the mixture was stirred at rt for 2 h. Then the reaction mixture was removed the solvent to give the crude (R)-1-(6-(methylamino)naphthalen-1-yl)pyrrolidin-3-amine (200 mg) as yellow oil.

Step 8: (R)—N-methyl-1-(3-((5-((trimethylsilyl)ethynyl)pyrimidin-2-yl)amino)pyrrolidin-1-yl)isoquinolin-6-amine

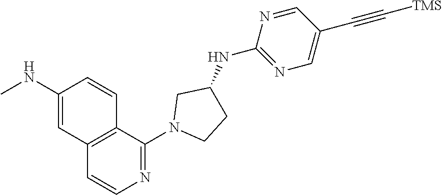 [SMILES: CNc1ccc2c(N3CC[C@@H](Nc4ncc(C#C[Si](C)(C)C)cn4)C3)nccc2c1]

The crude (R)-1-(6-(methylamino)naphthalen-1-yl)pyrrolidin-3-amine (200 mg) was dissolved in DMSO (5 mL) at rt. And DIEA (210 mg, 1.6 mmol) was added. The mixture was stirred at rt for 10 min. Then 2-chloro-5-((trimethylsilyl)ethynyl)pyrimidine (85 mg, 0.41 mmol) was added. The mixture was stirred at 40° C. overnight. The reaction mixture was added water (20 mL) and extracted with EA (10 mL*2). The combined organic layer was washed with water (10 mL*2) and brine (10 mL*2), dried over Na₂SO₄, filtered and concentrated in vacuo. The residue was purified by cloumn (DCM:MeOH=20:1) to afford (R)—N-methyl-1-(3-((5-((trimethylsilyl)ethynyl)pyrimidin-2-yl)amino)pyrrolidin-1-yl)isoquinolin-6-amine (140 mg, 82%) as yellow oil. [M+H] Calc'd for C₂₃H₂₈N₆Si, 417.2; Found, 417.2.

Step 9: (R)—N-methyl-N-(1-(3-((5-((trimethylsilyl)ethynyl)pyrimidin-2-yl)amino)pyrrolidin-1-yl)isoquinolin-6-yl)acrylamide

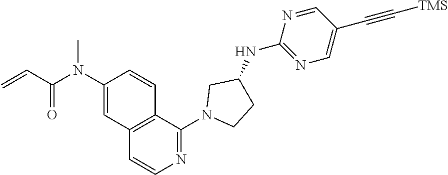 [SMILES: C=CC(=O)N(C)c1ccc2c(N3CC[C@@H](Nc4ncc(C#C[Si](C)(C)C)cn4)C3)nccc2c1]

To a solution of (R)—N-methyl-1-(3-((5-((trimethylsilyl)ethynyl)pyrimidin-2-yl)amino)pyrrolidin-1-yl)isoquinolin-6-amine (140 mg, 0.34 mmol) in DCM (5 mL) was added DIEA (90 mg, 0.68 mmol) at rt. Then acryloyl chloride (30 mg, 0.34 mmol) was added dropwised at ice-bath. The reaction mixture was stirred at rt for 2 h. The mixture was quenched with ammonium chloride aqueous solution (20 mL) and extracted with EA (10 mL*2). The combined organic layer was washed with water (10 mL*2) and brine (10 mL*2), dried over Na₂SO₄, filtered and concentrated in vacuo to afford the crude (R)—N-methyl-N-(1-(3-((5-((trimethylsilyl)ethynyl)pyrimidin-2-yl)amino)pyrrolidin-1-yl)isoquinolin-6-yl)acrylamide (100 mg, 63%) as a yellow solid. [M+H] Calc'd for C₂₆H₃₀N₆OSi, 471.2; Found, 471.2.

Step 10: (R)—N-(1-(3-((5-ethynylpyrimidin-2-yl)amino)pyrrolidin-1-yl)isoquinolin-6-yl)-N-methylacrylamide

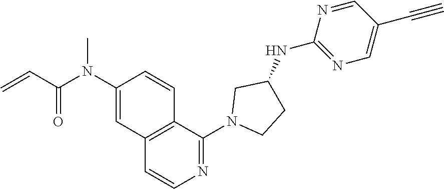 [SMILES: C#Cc1cnc(N[C@@H]2CCN(c3nccc4cc(N(C)C(=O)C=C)ccc34)C2)nc1]

To a solution of (R)—N-methyl-N-(1-(3-((5-((trimethylsilyl)ethynyl)pyrimidin-2-yl)amino)pyrrolidin-1-yl)isoquinolin-6-yl)acrylamide (100 mg, 0.21 mmol) in MeOH (5 mL) was added K₂CO₃ (30 mg, 0.21 mmol) at ice-bath. The reaction mixture was stirred at rt for 10 min. The reaction mixuture was added H₂O (20 mL) and extracted with EA (10 mL*2). The combined organic layer was washed with water (10 mL*2) and brine (10 mL*2), dried over Na₂SO₄, filtered and concentrated in vacuo to give a residue which was purified by C18 column (ACN/H₂O=10%-80%) to afford (R)—N-(1-(3-((5-ethynylpyrimidin-2-yl)amino)pyrrolidin-1-yl)isoquinolin-6-yl)-N-methylacrylamide (20.2 mg, 23%) as a white solid. ¹H NMR (400 MHz, CD₃OD): δ 2.18-2.21 (m, 1H), 2.37-2.40 (m, 1H), 3.45 (s, 3H), 3.64 (s, 1H), 3.80-3.84 (m, 1H), 3.94-3.97 (m, 1H), 4.05-4.08 (m, 1H), 4.19-4.24 (m, 1H), 4.62-4.66 (m, 1H), 6.63 (dd, J=2.0, 10.4 Hz, 1H), 6.31-6.36 (m, 2H), 7.08 (d, J=6.0 Hz, 1H), 7.38 (dd, J=2.4, 8.8 Hz, 1H), 7.63 (d, J=2.4 Hz, 1H) , 7.95 (d, J=5.6 Hz, 1H), 8.40-8.43 (m, 3H). [M+H] MS Calc'd C₂₃H₂₂N₆O, 399.1; Found: 399.1

### Example 45: (R)—N-(4-(azetidin-1-yl)-1-(3-((5-cyanopyrimidin-2-yl)amino)pyrrolidin-1-yl)phthalazin-6-yl)acrylamide formate

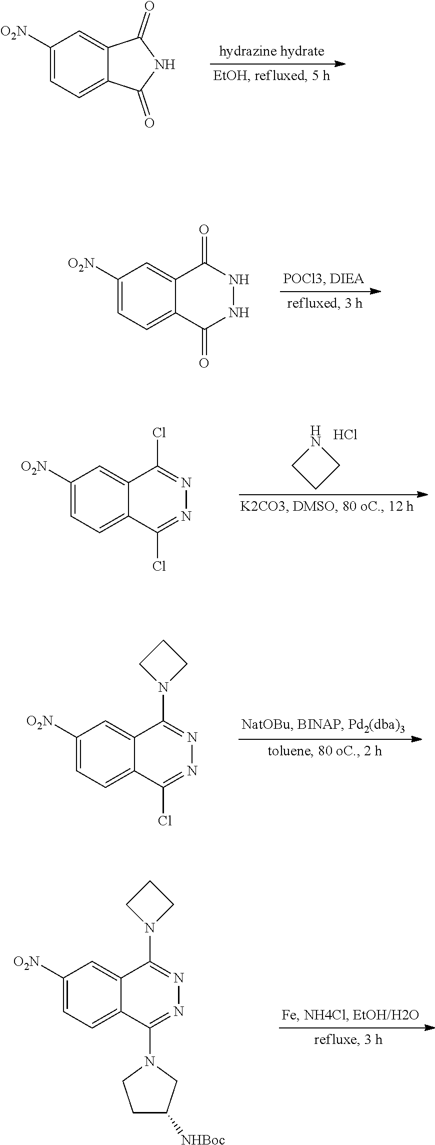

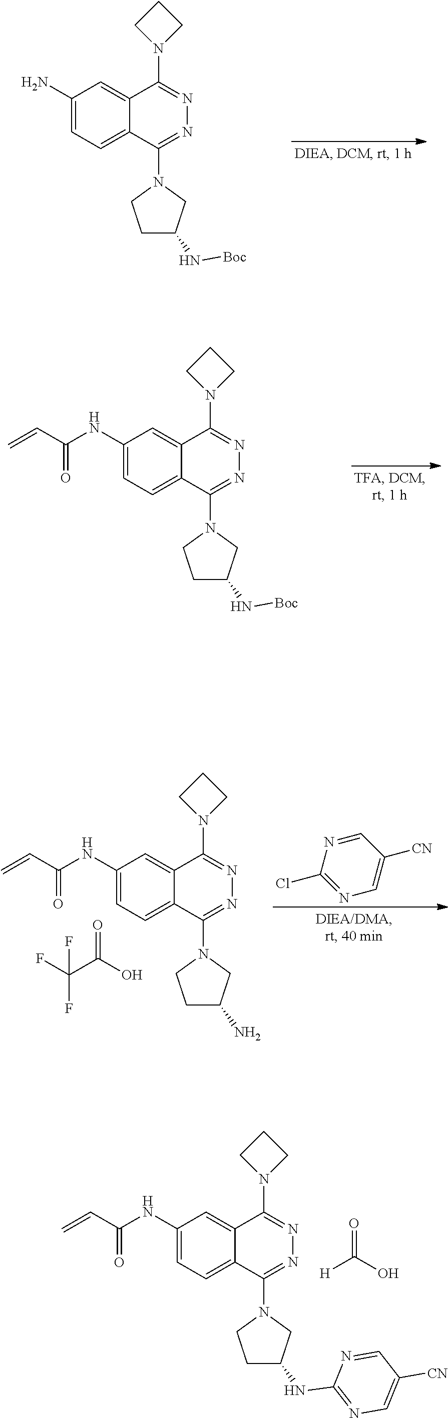

Step 1: 6-nitro-2,3-dihydrophthalazine-1,4-dione

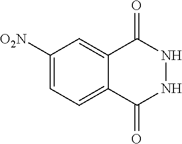 [SMILES: O=c1[nH][nH]c(=O)c2cc([N+](=O)[O-])ccc12]

To a solution of 5-nitroisoindoline-1,3-dione (50.0 g, 0.26 mol) in EtOH (500 mL) was added hydrazine hydrate (52.0 g, 1.04 mol) at rt. Then the reaction mixture was refluxed for 5 h. After cooled to rt, the reaction mixture was poured into water (300 mL) and the solid was filtered and washed with water (50 mL*3). The filtered cake was dried to afford 6-nitro-2,3-dihydrophthalazine-1,4-dione (43.0 g, 80%) as a yellow solid. [M+H] MS Calc'd C₈H₅N₃O₄, 208.0; Found: 208.0.

Step 2: 1,4-dichloro-6-nitrophthalazine

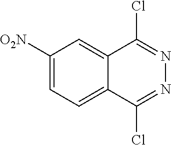 [SMILES: O=[N+]([O-])c1ccc2c(Cl)nnc(Cl)c2c1]

To a solution of 6-nitro-2,3-dihydrophthalazine-1,4-dione (5.0 g, 24.1 mmol) in POCl₃ (40 mL) was added DIEA (6.23 g, 48.3 mmol) at rt. The reaction mixture was refluxed for 3 h. The reaction mixture was cooled to rt and concentrated. The residue was dissolved in DCM (100 mL) and washed with sat. NaHCO₃ (50 mL*2) and brine (50 mL), dried over anhydrous sodium sulfate, filtered and concentrated to afford 1,4-dichloro-6-nitrophthalazine (5.0 g, 85%) as an orange solid. [M+H] MS Calc'd C₈H₃Cl₂N₃O₂, 243.9; Found: 243.9.

Step 3: 4-(azetidin-1-yl)-1-chloro-6-nitrophthalazine

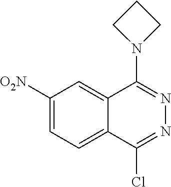 [SMILES: O=[N+]([O-])c1ccc2c(Cl)nnc(N3CCC3)c2c1]

To a solution of 1,4-dichloro-6-nitrophthalazine (5.0 g, 0.024 mol) in DMSO (50 mL) was added azetidine hydrochloride (2.2 g, 0.024 mol) and K₂CO₃ (10.0 g, 0.072 mol) at rt. Then the reaction mixture was stirred at 80° C. for 12 h. The reaction mixture was cooled to rt, diluted with water (150 mL) and extracted with EA (80 mL*3). The combined organic layers were washed with brine (100 mL), dried over anhydrous sodium sulfate, filtered and concentrated. The residue purified by silica gel column chromatography (PE:EA=2:1) to afford 4-(azetidin-1-yl)-1-chloro-6-nitrophthalazine (0.9 g, 16.6%) as a red solid. [M+H] MS Calc'd C₁₁H₉ClN₄O₂, 265.1; Found: 265.1.

Step 4: (R)-tert-butyl(1-(4-(azetidin-1-yl)-6-nitrophthalazin-1-yl)pyrrolidin-3-yl)carbamate

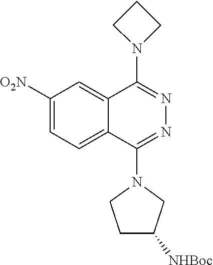 [SMILES: CC(C)(C)OC(=O)N[C@@H]1CCN(c2nnc(N3CCC3)c3cc([N+](=O)[O-])ccc23)C1]

To a solution of 4-(azetidin-1-yl)-1-chloro-6-nitrophthalazine (1.3 g, 4.9 mmol) and (R)-tert-butyl pyrrolidin-3-ylcarbamate (3.67 g, 19.7 mmol) in toluene (50 mL) was added BINAP (123 mg, 0.2 mmol), Pd₂(dba)₃ (57 mg, 0.06 mmol) and t-BuONa (950 mg, 9.8 mmol) at room temperature under N₂ atmosphere. Then the reaction mixture was stirred at 80° C. for 2 h. The reaction mixture was cooled to room temperature and filtered. The filtrate was concentrated and purified by silica gel column chromatography (PE:EA=1:1) to give (R)-tert-butyl(1-(4-(azetidin-1-yl)-6-nitrophthalazin-1-yl)pyrrolidin-3-yl)carbamate (400 mg, 20%) as a yellow solid. [M+H] MS Calc'd C₂₀H₂₆N₆O₄, 415.2; Found: 415.2.

Step 5: (R)—N-(4-(azetidin-1-yl)-1-(3-((5-bromopyrimidin-2-yl)amino)pyrrolidin-1-yl)phthalazin-6-yl)acrylamide

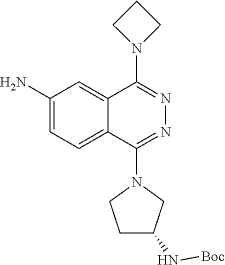 [SMILES: CC(C)(C)OC(=O)N[C@@H]1CCN(c2nnc(N3CCC3)c3cc(N)ccc23)C1]

To a solution of (R)-tert-butyl (1-(4-(azetidin-1-yl)-6-nitrophthalazin-1-yl)pyrrolidin-3-yl)carbamate (400 mg, 0.97 mmol) in EtOH (30 mL) and H₂O (10 mL) was added Fe (540 mg, 9.70 mmol) and NH₄Cl (525 mg, 9.70 mmol) at rt. Then the reaction mixture was refluxed for 3 h. After cooled to rt, the reaction mixture was filtered and the filtrate was concentrated. The residue was adjusted pH 8 with sat.NaHCO₃ and extracted with EA (20 mL*3). The combined organic layers were washed with brine (20 mL), dried over anhydrous sodium sulfate, filtered and concentrated to afford (R)—N-(4-(azetidin-1-yl)-1-(3-((5-bromopyrimidin-2-yl)amino)pyrrolidin-1-yl)phthalazin-6-yl)acrylamide (300 mg, 80%) as a yellow solid. [M+H] MS Calc'd C₂₀H₂₈N₆O₂, 385.2; Found: 385.2.

Step 6: (R)-tert-butyl (1-(6-acrylamido-4-(azetidin-1-yl)phthalazin-1-yl)pyrrolidin-3-yl)carbamate

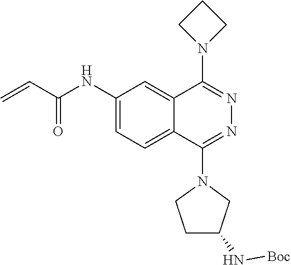 [SMILES: C=CC(=O)Nc1ccc2c(N3CC[C@@H](NC(=O)OC(C)(C)C)C3)nnc(N3CCC3)c2c1]

To a solution of (R)—N-(4-(azetidin-1-yl)-1-(3-((5-bromopyrimidin-2-yl)amino)pyrrolidin-1-yl)phthalazin-6-yl)acrylamide (300 mg, 0.78 mmol) in DCM (15 mL) was added acryloyl chloride (105 mg, 1.17 mmol) and DIEA (305 mg, 2.34 mmol) at 0° C. Then the mixture was warmed to rt and stirred for 1 h. The reaction mixture was quenched with sat. NH₄Cl (50 mL). The separate organic layer was concentrated and purified by C18 column (ACN:H₂O=5% to 40%) to afford (R)-tert-butyl (1-(6-acrylamido-4-(azetidin-1-yl)phthalazin-1-yl)pyrrolidin-3-yl)carbamate (80 mg, 24%) as yellow oil. [M+H] MS Calc'd C₂₃H₃₀N₆O₃, 439.2; Found: 439.2.

Step 7: (R)—N-(1-(3-aminopyrrolidin-1-yl)-4-(azetidin-1-yl)phthalazin-6-yl)acrylamide 2,2,2-trifluoroacetate

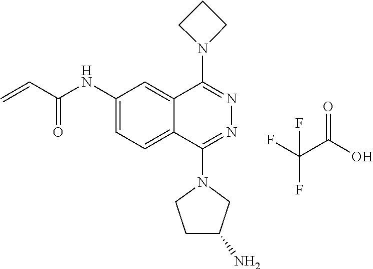

To a solution of (R)-tert-butyl (1-(6-acrylamido-4-(azetidin-1-yl)phthalazin-1-yl)pyrrolidin-3-yl)carbamate (80 mg, 0.18 mmol) in DCM (5 mL) was added TFA (1 mL) at rt. The reaction mixture was stirred at room temperature for 1 h. The reaction mixture was concentrated to give (R)—N-(1-(3-aminopyrrolidin-1-yl)-4-(azetidin-1-yl)phthalazin-6-yl)acrylamide 2,2,2-trifluoroacetate (80 mg, 100%) as yellow oil. [M+H] Calc'd for C₁₈H₂₂N₆O, 339.2; Found, 339.2.

Step 8: (R)—N-(4-(azetidin-1-yl)-1-(3-((5-cyanopyrimidin-2-yl)amino)pyrrolidin-1-yl)phthalazin-6-yl)acrylamide formate

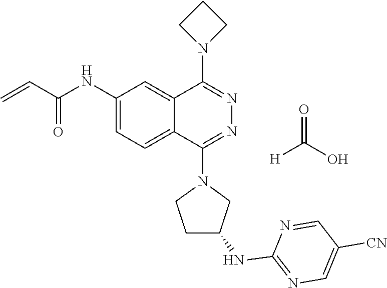 [SMILES: C=CC(=O)Nc1ccc2c(N3CC[C@@H](Nc4ncc(C#N)cn4)C3)nnc(N3CCC3)c2c1 ; O=CO]

To a solution of (R)—N-(1-(3-aminopyrrolidin-1-yl)-4-(azetidin-1-yl)phthalazin-6-yl)acrylamide 2,2,2-trifluoroacetate (75 mg, 0.22 mmol) in DMA (3 mL) was added 2-chloropyrimidine-5-carbonitrile (15 mg, 0.11 mmol) and DIEA (85 mg, 0.66 mmol) at rt. Then the mixture was stirred at rt for 40 min. The reaction mixture was quenched with water (10 mL) and extracted with EA (10 mL*3). The combined organic layers were washed with brine (10 mL), dried over anhydrous sodium sulfate, filtered and concentrated. The residue was purified by prep-HPLC to afford (R)—N-(4-(azetidin-1-yl)-1-(3 -((5-cyanopyrimidin-2-yl)amino)pyrrolidin-1-yl)phthalazin-6-yl)acrylamide formate (19 mg, 19%) as a yellow solid. ¹H NMR (400 MHz, DMSO-d6): δ 2.04-2.10 (m, 3H), 2.22-2.32 (m, 1H), 3.49-3.89 (m, 6H), 4.08-4.10 (m, 1H), 4.50-4.54 (m, 1H), 5.85-5.88 (m, 1H), 6.36 (dd, J=2.0, 16.8 Hz, 1H), 6.50 (dd, J=10.0, 16.8 Hz, 1H), 8.12-8.18 (m, 2H), 8.37 (s, 1H), 8.64-8.76 (m, 3H), 8.92 (s, 1H), 11.06 (s, 1H). [M+H] MS Calc'd C₂₃H₂₃N₉O, 442.2; Found: 442.2

### Example 46: Synthesis of (R)—N-(1-(3-((5-bromopyrimidin-2-yl)amino)pyrrolidin-1-yl)-3-(1-methylpiperidin-4-yl)isoquinolin-6-yl)-N-methylacrylamide

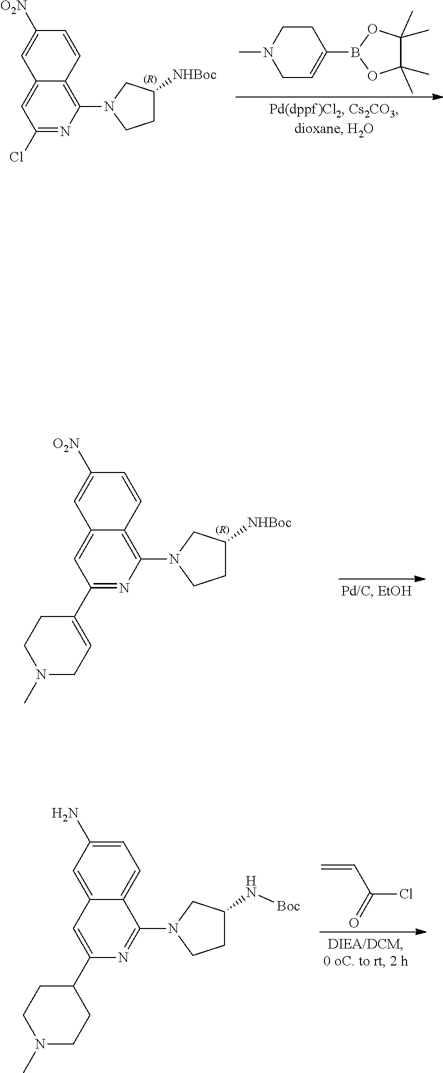

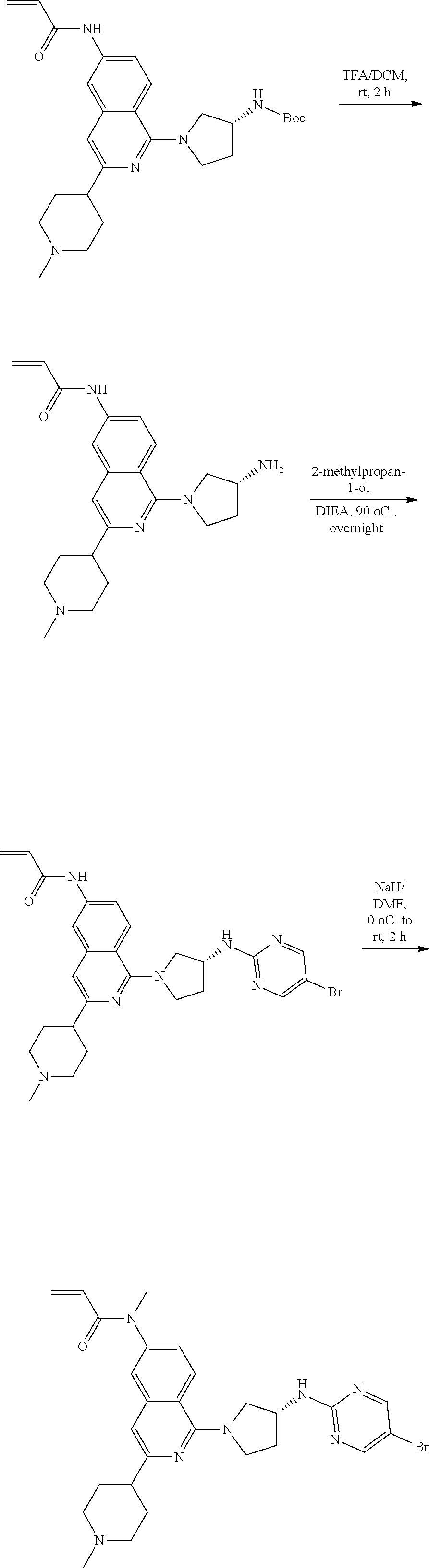 [SMILES: C=CC(=O)Nc1ccc2c(N3CC[C@@H](NC(=O)OC(C)(C)C)C3)nc(C3CCN(C)CC3)cc2c1 ; C=CC(=O)Nc1ccc2c(N3CC[C@@H](N)C3)nc(C3CCN(C)CC3)cc2c1 ; C=CC(=O)Nc1ccc2c(N3CC[C@@H](Nc4ncc(Br)cn4)C3)nc(C3CCN(C)CC3)cc2c1 ; C=CC(=O)N(C)c1ccc2c(N3CC[C@@H](Nc4ncc(Br)cn4)C3)nc(C3CCN(C)CC3)cc2c1]

Step 1: (R)-tert-butyl (1-(3-(1-methyl-1,2,3,6-tetrahydropyridin-4-yl)-6-nitroisoquinolin-1-yl)pyrrolidin-3-yl)carbamate

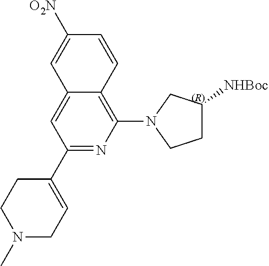

To a solution of (R)-tert-butyl (1-(3-chloro-6-nitroisoquinolin-1-yl)pyrrolidin-3-yl)carbamate (2.5 g, 6.4 mmol) and 1-methyl-4-(4,4,5,5-tetramethyl-1,3,2-dioxaborolan-2-yl)-1,2,3,6-tetrahydropyridine (1.6 g, 7.0 mmol) in dioxane (50 mL) and H₂O (5 mL) was added Pd(dppf)Cl₂ (466 mg, 0.64 mmol) and Cs₂CO₃ (4.15 g, 12.7 mmol) under N₂. The reaction mixture was stirred at 105° C. for 5 h. The reaction mixture was cooled, filtered and concentrated. The residue was purified by column (PE:EA=10:1) to afford the (R)-tert-butyl (1-(3-(1-methyl-1,2,3,6-tetrahydropyridin-4-yl)-6-nitroisoquinolin-1-yl)pyrrolidin-3-yl)carbamate (1.8 g, 62%) as a brown solid. [M+H] Calc'd for C₂₄H₃₁N₅O₄, 454.2; Found, 454.2

Step 2: (R)-tert-butyl (1-(6-amino-3-(1-methylpiperidin-4-yl)isoquinolin-1-yl)pyrrolidin-3-yl)carbamate

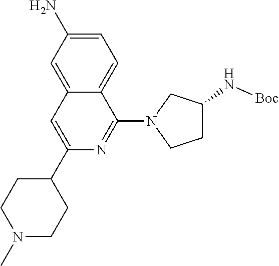 [SMILES: CN1CCC(c2cc3cc(N)ccc3c(N3CC[C@@H](NC(=O)OC(C)(C)C)C3)n2)CC1]

A solution of (R)-tert-butyl (1-(3-(1-methyl-1,2,3,6-tetrahydropyridin-4-yl)-6-nitroisoquinolin-1-yl)pyrrolidin-3-yl)carbamate (1.8 g, 3.97 mmol) and Pd/C (200 mg, 5%) in EtOH (100 mL) was stirred at rt for 4 h under H₂ (1 atm). The reaction mixture was filterd and concentrated. The residue was purified by cloumn (DCM:MeOH=10:1) to afford (R)-tert-butyl (1-(6-amino-3-(1-methylpiperidin-4-yl)isoquinolin-1-yl)pyrrolidin-3-yl)carbamate (1.6 g, 85%) as a brown solid. [M+H] Calc'd for C₂₄H₃₅N₅O₂, 426.3 Found, 426.3

Step 3: (R)-tert-butyl (1-(6-acrylamido-3-(1-methylpiperidin-4-yl)isoquinolin-1-yl)pyrrolidin-3-yl)carbamate

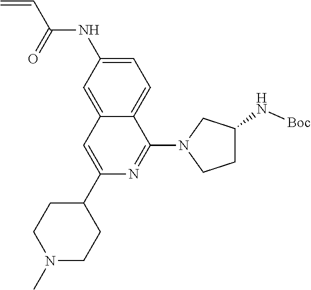 [SMILES: C=CC(=O)Nc1ccc2c(N3CC[C@@H](NC(=O)OC(C)(C)C)C3)nc(C3CCN(C)CC3)cc2c1]

To a solution of (R)-tert-butyl (1-(6-amino-3-(1-methylpiperidin-4-yl)isoquinolin-1-yl)pyrrolidin-3-yl)carbamate (4.0 g, 9.4 mmol) and DIEA (3.0 g, 23.5 mmol) in DCM (100 mL) was added acryloyl chloride (1.0 g, 11.3 mmol) at 0° C. Then the reaction mixture was warmed to rt and stirred for 2 h. The mixture was quench with sat.NH₄Cl (30 mL). The separate organic layer was dried over anhydrous sodium sulfate, filtered and concentrated under reduced pressure. The residue was purified by silica gel column chromatography (DCM/MeOH=10/1) to give (R)-tert-butyl (1-(6-acrylamido-3-(1-methylpiperidin-4-yl)isoquinolin-1-yl)pyrrolidin-3-yl)carbamate (1.5 g, 32%) as a white solid. [M+H] MS Calc'd C₂₇H₃₇N₅O₃, 480.3; Found: 480.3.

Step 4: (R)—N-(1-(3-aminopyrrolidin-1-yl)-3-(1-methylpiperidin-4-yl)isoquinolin-6-yl)acrylamide

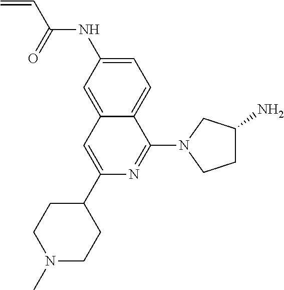 [SMILES: C=CC(=O)Nc1ccc2c(N3CC[C@@H](N)C3)nc(C3CCN(C)CC3)cc2c1]

To a solution of (R)-tert-butyl (1-(6-acrylamido-3-(1-methylpiperidin-4-yl)isoquinolin-1-yl)pyrrolidin-3-yl)carbamate (1.40 g, 2.92 mmol) in DCM (15 mL) was added TFA (5 mL) at rt. The reaction mixture was stirred at room temperature for 2 h. The reaction mixture was concentrated and the residue was adjusted pH 9 with sat. NaHCO₃. Then the mixture was filtered and the filtrate was concentrated to give a residue which was purified by C18 column (ACN:H₂O=5% to 40%) to give (R)—N-(1-(3-aminopyrrolidin-1-yl)-3-(1-methylpiperidin-4-yl)isoquinolin-6-yl)acrylamide (600 mg, 55%) as a white solid. [M+H] Calc'd for C₂₂H₂₉N₅O, 380.2; Found, 380.2.

Step 5: (R)—N-(1-(3-((5-bromopyrimidin-2-yDamino)pyrrolidin-1-yl)-3-(1-methylpiperidin-4-yl)isoquinolin-6-yl)acrylamide

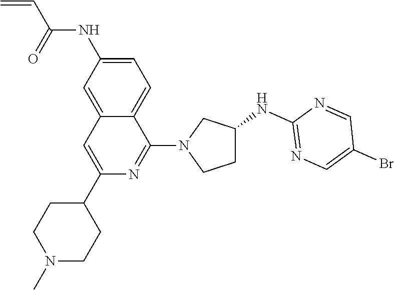 [SMILES: C=CC(=O)Nc1ccc2c(N3CC[C@@H](Nc4ncc(Br)cn4)C3)nc(C3CCN(C)CC3)cc2c1]

To a solution of (R)—N-(1-(3-aminopyrrolidin-1-yl)-3-(1-methylpiperidin-4-yl)isoquinolin-6-yl)acrylamide (370 mg, 0.98 mmol) in 2-methylpropan-1-ol (10 mL) was added 5-bromo-2-chloropyrimidine (284 mg, 1.46 mmol) and DIEA (380 mg, 2.94 mmol) at rt. Then the mixture was stirred at 90° C. overnight. The reaction mixture was cooled to rt and concentrated. The residue was purified by prep-HPLC to afford (R)—N-(1-(3-((5-bromopyrimidin-2-yl)amino)pyrrolidin-1-yl)-3-(1-methylpiperidin-4-yl)isoquinolin-6-yl)acrylamide (50 mg, 10%) as a yellow solid. [M+H] MS Calc'd C₂₆H₃₀BrN₇O, 536.2; Found: 536.2

Step 6: (R)—N-(1-(3-((5-bromopyrimidin-2-yl)amino)pyrrolidin-1-yl)-3-(1-methylpiperidin-4-yl)isoquinolin-6-yl)-N-methylacrylamide

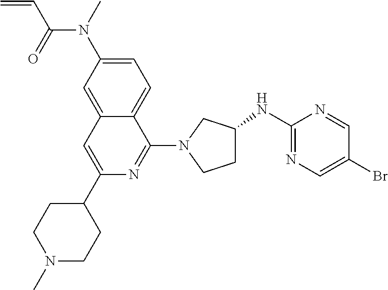 [SMILES: C=CC(=O)N(C)c1ccc2c(N3CC[C@@H](Nc4ncc(Br)cn4)C3)nc(C3CCN(C)CC3)cc2c1]

To a stirred solution of (R)—N-(1-(3-((5-bromopyrimidin-2-yl)amino)pyrrolidin-1-yl)-3-(1-methylpiperidin-4-yl)isoquinolin-6-yl)acrylamide (40 mg, 0.074 mmol) in anhydrous N,N-dimethylformamide (3 mL) was added 60% wt. sodium hydride in mineral oil (36 mg, 0.089 mmol) in portions at 0° C. under nitrogen atmosphere. The reaction mixture was allowed to warm to rt and stirred at rt for 10 min. Then CH₃I (13 mg, 0.089 mmol) was added and stirred at rt for 2 h. After the reaction mixture was quenched with water and extracted with DCM (10 mL*3). The combined organic layers were washed with brine (10 mL), dried over anhydrous sodium sulfate, filtered and concentrated. The residue was purified by prep-HPLC to afford (R)—N-(1-(3-((5-bromopyrimidin-2-yl)amino)pyrrolidin-1-yl)-3-(1-methylpiperidin-4-yl)isoquinolin-6-yl)-N-methylacrylamide (3.2 mg, 7.8%) as a white solid. ¹H NMR (400 MHz, CD₃OD): δ 1.99-2.07 (m, 5H), 2.22-2.25 (m, 1H), 2.58 (s, 3H), 2.68-2.75 (m, 3H), 3.21-3.31 (m, 5H), 3.70-3.74 (m, 1H), 3.86 -3.95 (m, 2H), 4.05-4.08 (m, 1H), 4.42-4.45 (m, 1H), 5.49 (dd, J=2.0, 11.6 Hz, 1H), 6.17-6.22 (m, 2H), 6.83 (s, 1H), 7.18 (dd, J=2.0, 8.8 Hz, 1H), 7.44 (d, J=2.0 Hz, 1H), 8.21-8.23 (m, 3H). [M+H] MS Calc'd C₂₇H₃₂BrN₇O, 550.2; Found: 550.2

### Example 47: (R)—N-(1-(3-((6-ethynyl-1,2,4-triazin-3-yl)amino)pyrrolidin-1-yl)isoquinolin-6-yl)acrylamide

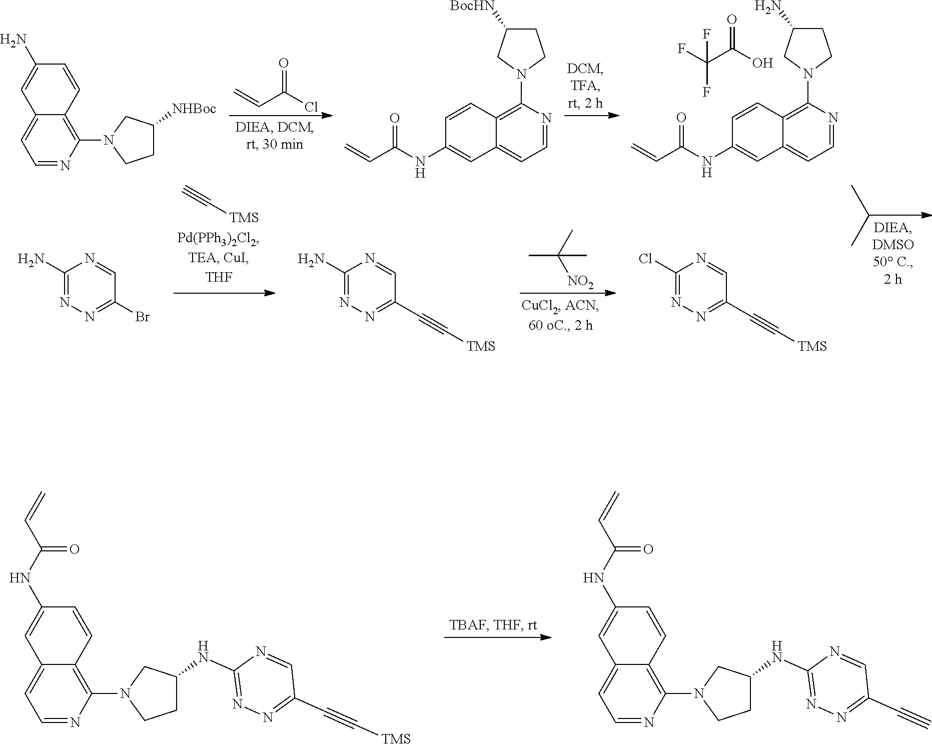

Step 1: (R)-tert-butyl (1-(6-acrylamidoisoquinolin-1-yl)pyrrolidin-3-yl)carbamate

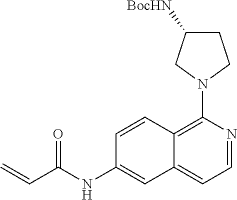 [SMILES: C=CC(=O)Nc1ccc2c(N3CC[C@@H](NC(=O)OC(C)(C)C)C3)nccc2c1]

To a solution of (R)-tert-butyl (1-(6-aminoisoquinolin-1-yl)pyrrolidin-3-yl)carbamate (2.0 g, 6.1 mmol) and DIEA (2.0 g, 15.2 mmol) in DCM (50 mL) was added acryloyl chloride (607 mg, 6.7 mmol) at 0° C. Then the reaction mixture was warmed to rt and stirred for 0.5 h. The mixture was quenched sat. NH₄Cl (30 mL). The separate organic layer was dried over anhydrous sodium sulfate, filtered and concentrated under reduced pressure. The residue was purified by silica gel column chromatography (PE/EA=1/1) to give (R)-tert-butyl (1-(6-acrylamidoisoquinolin-1-yl)pyrrolidin-3-yl)carbamate (1.8 g, 77%) as a white solid. [M+H] MS Calc'd C₂₁H₂₆N₄O₃, 383.2; Found: 383.2.

Step 2: (R)—N-(1-(3-aminopyrrolidin-1-yl)isoquinolin-6-yl)acrylamide 2,2,2-trifluoroacetate

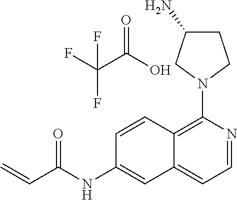

To a solution of (R)-tert-butyl (1-(6-acrylamidoisoquinolin-1-yl)pyrrolidin-3-yl)carbamate (1.8 g, 4.7 mmol) in DCM (60 mL) was added TFA (4 mL). The reaction mixture was stirred at room temperature for 2 h. The reaction mixture was concentrated to give (R)—N-(1-(3-aminopyrrolidin-1-yl)isoquinolin-6-yl)acrylamide 2,2,2-trifluoroacetate (1.7 g, 100%) as brown oil. [M+H] Calc'd for C₁₆H₁₈N₄O, 283.2; Found, 283.2.

Step 3: 6-((trimethylsilyl)ethynyl)-1,2,4-triazin-3-amine

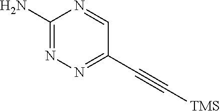 [SMILES: C[Si](C)(C)C#Cc1cnc(N)nn1]

To a mixture of 6-bromo-1,2,4-triazin-3-amine (2.00 g, 11.49 mmol) and CuI (654 mg, 3.45 mmol) and Pd(PPh₃)₂Cl₂ (805 mg, 1.14 mmol) in THF (20 mL) and TEA (50 mL) was added ethynyltrimethylsilane (4.50 g, 45.91 mmol) at rt. The reaction mixture was stirred at 90° C. for 16 h under nitrogen atmosphere. After cooling down to room temperature, the solvent was removed to give a residue which was purified by flash (PE/EA=1/3) to afford 6-((trimethylsilyl)ethynyl)-1,2,4-triazin-3-amine (1.60 g, 72%) as an off-white solid. [M+H] MS Calc'd C₈H₁₂N₄Si, 193.1; Found: 193.1

Step 4: 3-chloro-6-((trimethylsilyl)ethynyl)-1,2,4-triazine

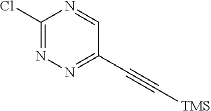 [SMILES: C[Si](C)(C)C#Cc1cnc(Cl)nn1]

To a solution of 6-((trimethylsilyl)ethynyl)-1,2,4-triazin-3-amine (1.6 g, 8.33 mmol) and 2-methyl-2-nitropropane (1.5 g, 12.49 mmol) in ACN (50 mL) was added CuCl₂(1.3 g, 9.9 mmol) at rt. The reaction mixture was stirred at 60° C. for 2 h. After cooling down to room temperature, the mixture was filtered and the filtrate was concentrated to give a residue. The residue was dissolved in EA (50 mL) and washed with H₂O (15 mL) and brine (15 mL). The organic layer was dried over anhydrous sodium sulfate, filtered and concentrated under reduced pressure. The residue was purified by silica gel column chromatography (PE/EA=1/1) to afford 3-chloro-6-((trimethylsilyl)ethynyl)-1,2,4-triazine (0.75 g, 42%) as an off-white solid. [M+H] MS Calc'd C₈H₁₀ClN₃Si, 212.0; Found: 212.0.

Step 5: (R)—N-(1-(3-((6-((trimethylsilyl)ethynyl)-1,2,4-triazin-3-yl)amino)pyrrolidin-1-yl)isoquinolin-6-yl)acrylamide

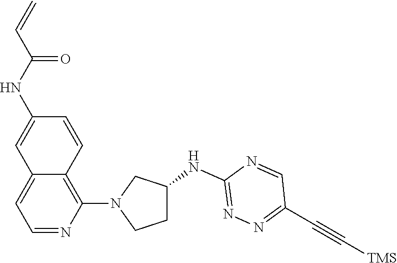 [SMILES: C=CC(=O)Nc1ccc2c(N3CC[C@@H](Nc4ncc(C#C[Si](C)(C)C)nn4)C3)nccc2c1]

To a solution of (R)—N-(1-(3-aminopyrrolidin-1-yl)isoquinolin-6-yl)acrylamide 2,2,2-trifluoroacetate (1.0 g, 3.54 mmol) in DMSO (15 mL) was added 3-chloro-6-((trimethylsilyl)ethynyl)-1,2,4-triazine (650 mg, 2.19 mmol) and DIEA (1.8 g, 14.2 mmol) at rt. Then the mixture was stirred at 50° C. for 2 h. The reaction mixture was cooled, quenched with water (15 mL) and extracted with EA (10 mL*3). The combined organic layers were washed with brine (100 mL), dried over anhydrous sodium sulfate, filtered and concentrated. The residue was purified by silica gel column chromatography (100% EA) to afford (R)—N-(1-(3-((6-((trimethylsilyl)ethynyl)-1,2,4-triazin-3-yl)amino)pyrrolidin-1-yl)isoquinolin-6-yl)acrylamide (1.0 g, 62%) as a yellow solid. [M+H] MS Calc'd C₂₄H₂₇N₇OSi, 458.2; Found: 458.2.

Step 6: (R)—N-(1-(3-((6-ethynyl-1,2,4-triazin-3-yl)amino)pyrrolidin-1-yl)isoquinolin-6-yl)acrylamide

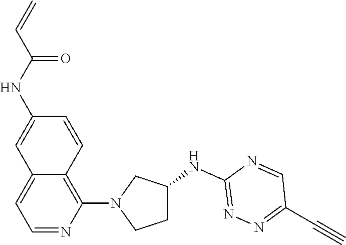 [SMILES: C#Cc1cnc(N[C@@H]2CCN(c3nccc4cc(NC(=O)C=C)ccc34)C2)nn1]

To a solution of (R)—N-(1-(3-((6-((trimethylsilyl)ethynyl)-1,2,4-triazin-3-yl)amino)pyrrolidin-1-yl)isoquinolin-6-yl)acrylamide (500 mg, 1.1 mmol) in THF (20 mL) was added a solution of TBAF in THF (1.6 mL, 1.0 M, 1.6 mol). Then the reaction mixture was stirred at rt for 0.5 h. The reaction mixture was quenched with water (30 mL) and extracted with EA (10 mL*3). The combined organic layers were washed with brine (20 mL), dried over anhydrous sodium sulfate, filtered and concentrated. The residue purified by silica gel column chromatography (PE/EA=1/1) to afford (R)—N-(1-(3-((6-ethynyl-1,2,4-triazin-3-yl)amino)pyrrolidin-1-yl)isoquinolin-6-yl)acrylamide (222.9 mg, 52.9%) as a yellow solid. ¹H NMR (400 MHz, DMSO-d6): δ 2.08-2.12 (m, 1H), 2.27-2.30 (m, 1H), 3.32-4.10 (m, 4H), 4.64 (s, 2H), 5.81 (dd, J=2.0, 10.0 Hz, 1H), 6.32 (dd, J=2.0, 16.8 Hz, 1H), 6.50 (dd, J=10.0, 16.8 Hz, 1H), 6.97 (d, J=5.6 Hz, 1H), 7.58 (dd, J=2.0, 9.2 Hz, 1H), 7.88 (d, J=5.6 Hz, 1H), 8.20-8.21 (m, 2H), 8.42 (br s, 2H), 10.42 (s, 1H). [M+H] MS Calc'd C₂₁H₁₉N₇O, 386.2; Found: 386.2

### Example 48: (R)—N-(1-(3-((5-ethynylpyrimidin-2-yl)amino)pyrrolidin-1-yl)isoquinolin-6-yl)acrylamide

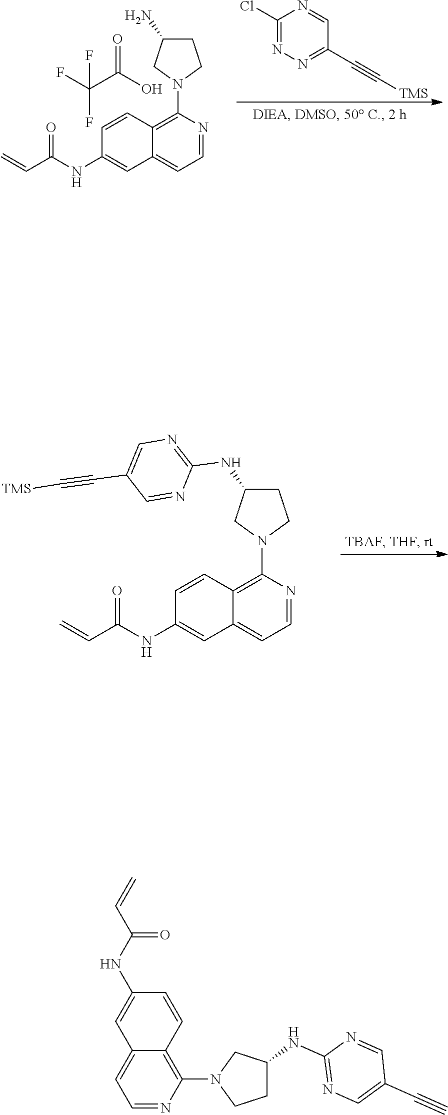

Step 1: (R)—N-(1-(3-((5-((trimethylsilyl)ethynyl)pyrimidin-2-yl)amino)pyrrolidin-1-yl)isoquinolin-6-yl)acrylamide

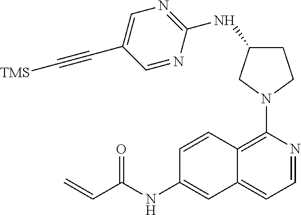 [SMILES: C=CC(=O)Nc1ccc2c(N3CC[C@@H](Nc4ncc(C#C[Si](C)(C)C)cn4)C3)nccc2c1]

To a solution of (R)—N-(1-(3-aminopyrrolidin-1-yl)isoquinolin-6-yl)acrylamide 2,2,2-trifluoroacetate (500 mg, 1.77 mmol) in DMSO (15 mL) was added 2-chloro-5-((trimethylsilyl)ethynyl)pyrimidine (483 mg, 2.30 mmol) and DIEA (915 mg, 7.09 mmol) at rt. Then the mixture was stirred at 50° C. for 2 h. The reaction mixture was cooled, quenched with water (15 mL) and extracted with EA (10 mL*3). The combined organic layers were washed with brine (100 mL), dried over anhydrous sodium sulfate, filtered and concentrated. The residue was purified by silica gel column chromatography (PE/EA=1/1) to afford (R)—N-(1-(3-((5-((trimethylsilyl)ethynyl)pyrimidin-2-yl)amino)pyrrolidin-1-yl)isoquinolin-6-yl)acrylamide (380 mg, 39%) as a yellow solid. [M+H] MS Calc'd C₂₅H₂₈N₆OSi, 457.2; Found: 457.2.

Step 2: (R)—N-(1-(3-((5-ethynylpyrimidin-2-yl)amino)pyrrolidin-1-yl)isoquinolin-6-yl)acrylamide

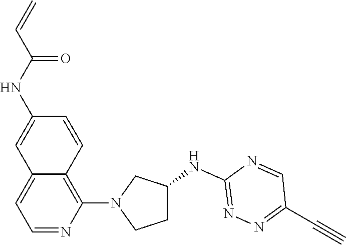 [SMILES: C#Cc1cnc(N[C@@H]2CCN(c3nccc4cc(NC(=O)C=C)ccc34)C2)nn1]

To a solution of (R)—N-(1-(3-((5-((trimethylsilypethynyl)pyrimidin-2-yl)amino)pyrrolidin-1-yl)isoquinolin-6-yl)acrylamide (180 mg, 0.40 mmol) in THF (10 mL) was added a solution of TBAF in THF (0.50 mL, 0.50 mmol). Then the mixture was stirred for 2 h at rt. The reaction mixture was quenched with water (30 mL) and extracted with EA (10 mL*3). The combined organic layers were washed with brine (20 mL), dried over anhydrous sodium sulfate, filtered and concentrated. The residue purified by prep-HPLC to afford (R)—N-(1-(3-((5-ethynylpyrimidin-2-yl)amino)pyrrolidin-1-yl)isoquinolin-6-yl)acrylamide (165.5 mg, 57.7%) as a white solid. ¹H NMR (400 MHz, DMSO-d₆): δ 2.02-2.07 (m, 1H), 2.22-2.26 (m, 1H), 3.70-3.74 (m, 1H), 3.82-3.86 (m, 1H), 3.91-3.95 (m, 1H), 4.04-4.08 (m, 1H), 4.25 (s, 1H), 4.45-4.50 (m, 1H), 5.81 (dd, J=2.0, 10.0 Hz, 1H), 6.31 (dd, J=2.0, 17.2 Hz, 1H), 6.48 (dd, J=10.0, 16.8 Hz, 1H), 6.96 (d, J=5.6 Hz, 1H), 7.57 (dd, J=2.0, 9.2 Hz, 1H), 7.87 (d, J=5.6 Hz, 1H), 8.01 (d, J=6.4 Hz, 1H), 8.20-8.21 (m, 2H), 8.43 (br s, 2H), 10.41 (s, 1H). [M+H] MS Calc'd C₂₂H₂₀N₆O, 385.2; Found: 385.2

### Example 49: (R)—N-(1-(34(5-cyanopyrimidin-2-yl)amino)pyrrolidin-1-yl)phthalazin-6-yl)acrylamide

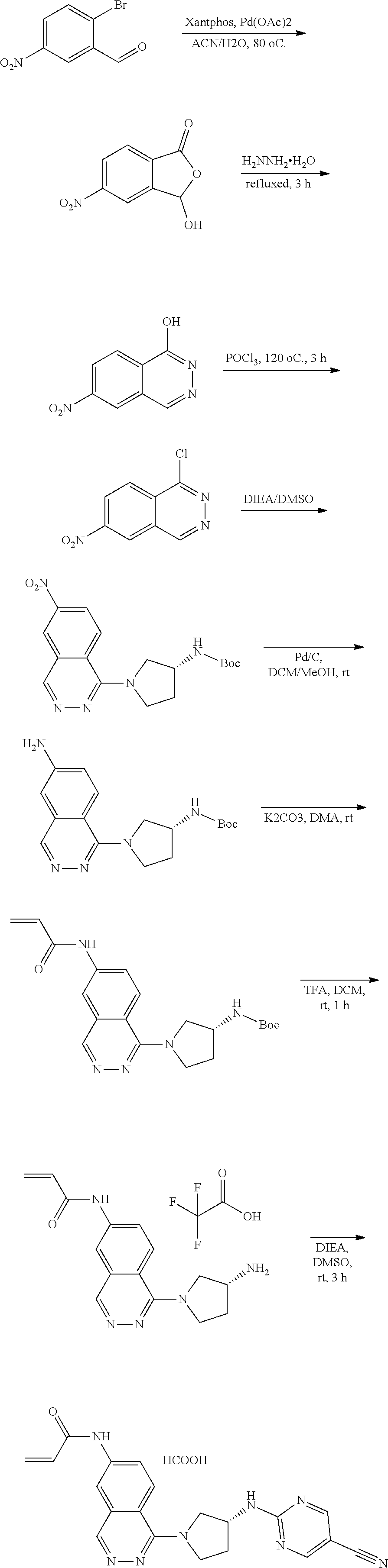

Step 1: 3-hydroxy-5-nitroisobenzofuran-1(3H)-one

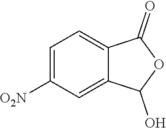 [SMILES: O=C1OC(O)c2cc([N+](=O)[O-])ccc21]

To a solution of 2-bromo-5-nitrobenzaldehyde (10.0 g, 43.5 mmol) in ACN (300 mL) was added xantphos (2.5 g, 4.35 mmol), Pd (OAc)2 (500 mg, 2.18 mmol), TEA (30 mL) and H₂O (40 mL) at rt. Then the reaction mixture was stirred at 80° C. overnight under CO atmosphere. The reaction mixture was cooled to room temperature and filtered. The filtrate was concentrated and purified by C18 column (ACN:H₂O=5% to 40%) to give 3-hydroxy-5-nitroisobenzofuran-1(3H)-one (7.5 g, 88%) as a yellow solid. [M+H] MS Calc'd C₈H₅NO₅, 196.0; Found: 196.1.

Step 2: 6-nitrophthalazin-1-ol

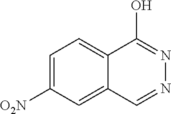 [SMILES: O=[N+]([O-])c1ccc2c(O)nncc2c1]

To a solution of 3-hydroxy-5-nitroisobenzofuran-1(3H)-one (7.0 g, 35.9 mmol) in EtOH (100 mL) was added hydrazine hydrate (3.6 g, 71.8 mmol) at rt. Then the mixture was refluxed for 3 h. After cooled to rt, the reaction mixture was concentrated and the residue was poured into water (100 mL), and the solid was filtered and washed with water (10 mL*3). The filtered cake was dried to afford 6-nitrophthalazin-1-ol (4.0 g, 58%) as a yellow solid. [M+H] MS Calc'd C₈H₅N₃O₃, 192.0; Found: 192.0

Step 3: 1-chloro-6-nitrophthalazine

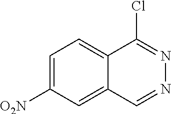 [SMILES: O=[N+]([O-])c1ccc2c(Cl)nncc2c1]

A solution of 6-nitrophthalazin-1-ol (3.0 g, 15.7 mmol) in POCl₃ (30 mL) was stirred for 3 h at 120° C. The mixture was cooled to rt and concentrated. The residue was quenched with ice water (30 mL) and extracted with EA (10 mL*3). The combined organic layers were dried over anhydrous sodium sulfate, filtered and concentrated to afford 1-chloro-6-nitrophthalazine (2.8 g, 85%) as an orange solid. [M+H] MS Calc'd C8H4ClN3O2, 210.0; Found: 210.0.

Step 4: (R)-tert-butyl (1-(6-nitrophthalazin-1-yl)pyrrolidin-3-yl)carbamate

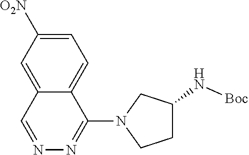 [SMILES: CC(C)(C)OC(=O)N[C@@H]1CCN(c2nncc3cc([N+](=O)[O-])ccc23)C1]

To a solution of 1-chloro-6-nitrophthalazine (2.8 g, 13.3 mmol) in DMSO (20 mL) was added (R)-tert-butyl pyrrolidin-3-ylcarbamate (2.5 g, 13.3 mmol) and DIEA (3.4 g, 26.6 mmol) at rt. Then the mixture was stirred at 60° C. overnight. The reaction mixture was cooled to rt, diluted with water (60 mL) and extracted with EA (30 mL*3). The combined organic layers were washed with brine (100 mL), dried over anhydrous sodium sulfate, filtered and concentrated. The residue was purified by silica gel column chromatography (PE:EA=1:1 to DCM:MeOH=10:1) to afford (R)-tert-butyl (1-(6-nitrophthalazin-1-yl)pyrrolidin-3-yl)carbamate (3.0 g, 62%) as a yellow solid. [M+H] MS Calc'd C₁₇H₂₁N₅O₄, 360.2; Found: 360.2.

Step 5: (R)-tert-butyl (1-(6-aminophthalazin-1-yl)pyrrolidin-3-yl)carbamate

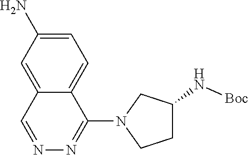 [SMILES: CC(C)(C)OC(=O)N[C@@H]1CCN(c2nncc3cc(N)ccc23)C1]

To a solution of (R)-tert-butyl (1-(6-aminophthalazin-1-yl)pyrrolidin-3-yl)carbamate (2.8 g, 7.8 mmol) in DCM (100 mL) and MeOH (100 mL) was added 10% palladium on charcoal wt (280 mg) at rt. The mixture was stirred overnight at room temperature under hydrogen atmosphere. The reaction mixture was filtered. The filtrate was concentrated. The residue was purified by silica gel column chromatography (DCM:MeOH=10:1) to afford (R)-tert-butyl (1-(6-aminophthalazin-1-yl)pyrrolidin-3-yl)carbamate (1.6 g, 64%) as a yellow solid. [M+H] MS Calc'd C₁₇H₂₃N₅O₂, 330.2; Found: 330.2.

Step 6: (R)-tert-butyl (1-(6-acrylamidophthalazin-1-yl)pyrrolidin-3-yl)carbamate

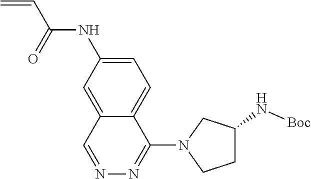 [SMILES: C=CC(=O)Nc1ccc2c(N3CC[C@@H](NC(=O)OC(C)(C)C)C3)nncc2c1]

To a solution of (R)-tert-butyl (1-(6-aminophthalazin-1-yl)pyrrolidin-3-yl)carbamate (600 mg, 1.82 mmol) in DMA (10 mL) was added acryloyl chloride (250 mg, 2.73 mmol) and K₂CO₃ (503 mg, 3.35 mmol) at 0° C. Then the mixture was warmed to rt and stirred at rt overnight. The reaction mixture was quenched with water (30 mL) and extracted with EA (10 mL*3). The combined organic layers were washed with brine (20 mL), dried over anhydrous sodium sulfate, filtered and concentrated. The residue purified by prep-HPLC to afford to (R)-tert-butyl (1-(6-acrylamidophthalazin-1-yl)pyrrolidin-3-yl)carbamate (100 mg, 14%) as a yellow solid. [M+H] MS Calc'd C₂₀H₂₅N₅O₃, 384.2; Found: 384.2.

Step 7: (R)—N-(1-(3-aminopyrrolidin-1-yl)phthalazin-6-yl)acrylamide 2,2,2-trifluoroacetate

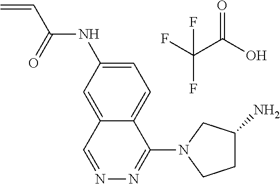

To a solution of (R)-tert-butyl (1-(6-acrylamidophthalazin-1-yl)pyrrolidin-3-yl)carbamate (40 mg, 0.10 mmol) in DCM (5 mL) was added TFA (2 mL). The reaction mixture was stirred at room temperature for 1 h. The reaction mixture was concentrated to give (R)—N-(1-(3-aminopyrrolidin-1-yl)phthalazin-6-yl)acrylamide 2,2,2-trifluoroacetate (40 mg, 100%) as yellow oil. [M+H] Calc'd for C₁₅H₁₇N₅O, 284.2; Found, 284.2.

Step 8: (R)—N-(1-(3-((5-cyanopyrimidin-2-yl)amino)pyrrolidin-1-yl)phthalazin-6-yl)acrylamide formate

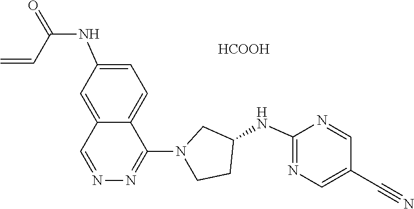 [SMILES: C=CC(=O)Nc1ccc2c(N3CC[C@@H](Nc4ncc(C#N)cn4)C3)nncc2c1 ; "HCOOH"]

To a solution of (R)—N-(1-(3-aminopyrrolidin-1-yl)phthalazin-6-yl)acrylamide 2,2,2-trifluoroacetate (100 mg, 0.20 mmol) in DMSO (5 mL) was added 2-chloropyrimidine-5-carbonitrile (28 mg, 0.20 mmol) and DIEA (80 mg, 0.60 mmol) at rt. Then the mixture was stirred at rt for 3 h. The reaction mixture was quenched with water (15 mL) and extracted with EA (10 mL*3). The combined organic layers were washed with brine (100 mL), dried over anhydrous sodium sulfate, filtered and concentrated. The residue was purified by prep-HPLC to afford (R)—N-(1-(3-((5-cyanopyrimidin-2-yl)amino)pyrrolidin-1-yl)phthalazin-6-yl)acrylamide formate (8.8 mg, 9%) as a yellow solid. ¹H NMR (400 MHz, DMSO-d6): δ 2.11-2.14 (m, 1H), 2.28-2.32 (m, 1H), 3.81-3.83 (m, 1H), 3.87-3.93 (m, 1H), 4.00-4.05 (m, 1H), 4.13-4.18 (m, 1H), 4.57-4.61 (m, 1H), 5.85 (dd, J=1.6, 10.0 Hz, 1H), 6.35 (dd, J=2.0, 16.8 Hz, 1H), 6.50 (dd, J=10.0, 16.8 Hz, 1H), 7.89 (dd, J=2.4, 9.2 Hz, 1H), 8.16 (s, 1H), 8.26 (d, J=9.2 Hz, 1H), 8.38 (d, J=2.0 Hz, 1H), 8.68-8.78 (m, 3H), 8.92 (s, 1H), 10.65 (s, 1H). [M+H] MS Calc'd C₂₀H₁₈N₈O, 387.2; Found: 387.2.

### Example 50: (R)—N-(1-(3-((5-bromopyrimidin-2-yl)amino)pyrrolidin-1-yl)phthalazin-6-yl)acrylamide

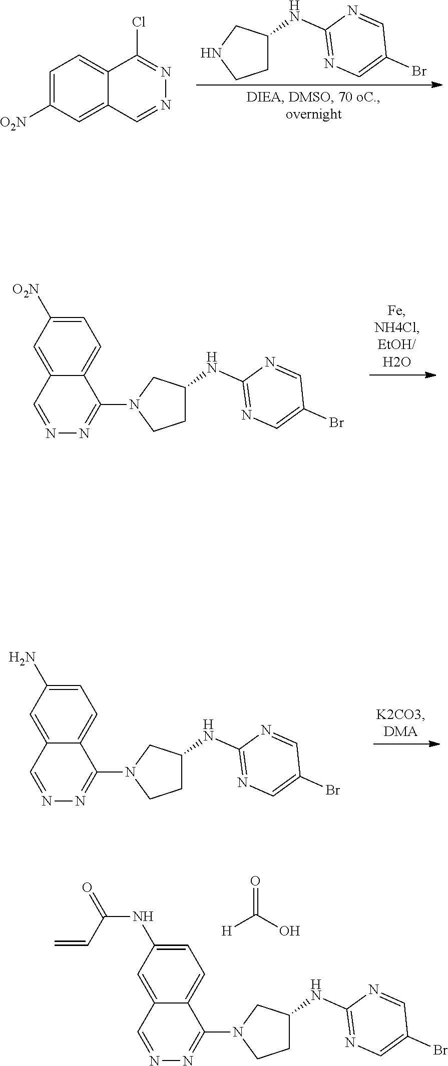

Step 1: (R)-5-bromo-N-(1-(6-nitrophthalazin-1-yl)pyrrolidin-3-yl)pyrimidin-2-amine

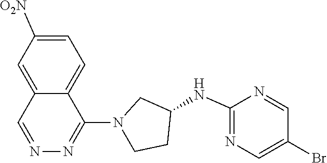 [SMILES: O=[N+]([O-])c1ccc2c(N3CC[C@@H](Nc4ncc(Br)cn4)C3)nncc2c1]

To a solution of 1-chloro-6-nitrophthalazine (210 mg, 1.0 mmol) in DMSO (5 mL) was added (R)-5-bromo-N-(pyrrolidin-3-yl)pyrimidin-2-amine (242 mg, 1.0 mmol) and DIEA (390 mg, 3.0 mmol) at rt. Then the mixture was stirred at 70° C. overnight. The reaction mixture was cooled to rt, diluted with water (15 mL) and extracted with EA (10 mL*3). The combined organic layers were washed with brine (10 mL), dried over anhydrous sodium sulfate, filtered and concentrated to afford ((R)-5-bromo-N-(1-(6-nitrophthalazin-1-yl)pyrrolidin-3-yl)pyrimidin-2-amine (350 mg, 84%) as a yellow solid. [M+H] MS Calc'd C₁₆H₁₄BrN₇O₂, 416.2; Found: 416.2.

Step 2: (R)-1-(3-((5-bromopyrimidin-2-l)amino)pyrrolidin-1-yl)phthalazin-6-amine

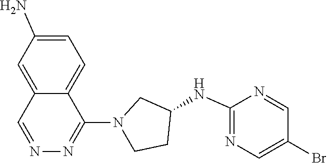 [SMILES: Nc1ccc2c(N3CC[C@@H](Nc4ncc(Br)cn4)C3)nncc2c1]

To a solution of ((R)-5-bromo-N-(1-(6-nitrophthalazin-1-yl)pyrrolidin-3-yl)pyrimidin-2-amine (200 mg, 0.48 mmol) in EtOH (15 mL) and H₂O (5 mL) was added Fe (270 mg, 4.80 mmol) and NH₄Cl (270 mg, 5.00 mmol) at rt. Then the reaction mixture was refluxed for 3 h. After cooled to rt, the reaction mixture was filtered and the filtrate was concentrated. The residue was adjusted to pH 8 with sat. NaHCO₃ and extracted with EA (20 mL*3). The combined organic layers were washed with brine (20 mL), dried over anhydrous sodium sulfate, filtered and concentrated. The residue was purified by C18 column (ACN:H₂O=5% to 40%) to give (R)-1-(3-((5-bromopyrimidin-2-yl)amino)pyrrolidin-1-yl)phthalazin-6-amine (70 mg, 38%) as a yellow solid. [M+H] MS Calc'd C₁₆H₁₆BrN₇, 386.2; Found: 386.2.

Step 3: (R)—N-(1-(3-((5-bromopyrimidin-2-yl)amino)pyrrolidin-1-yl)phthalazin-6-yl)acrylamide formate

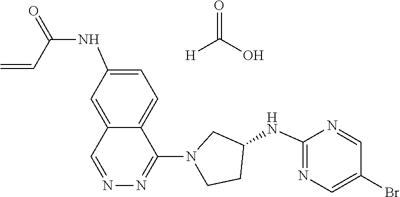 [SMILES: C=CC(=O)Nc1ccc2c(N3CC[C@@H](Nc4ncc(Br)cn4)C3)nncc2c1 ; O=CO]

To a solution of (R)-1-(3-((5-bromopyrimidin-2-yl)amino)pyrrolidin-1-yl)phthalazin-6-amine (300 mg, 0.78 mmol) in DMA (10 mL) was added acryloyl chloride (141 mg, 1.55 mmol) and K₂CO₃ (225 mg, 1.55 mmol) at 0° C. Then the mixture was warmed to rt and stirred overnight. The reaction mixture was quenched with water (30 mL) and extracted with EA (10 mL*3). The combined organic layers were washed with brine (20 mL), dried over anhydrous sodium sulfate, filtered and concentrated. The residue purified by prep-HPLC to afford (R)—N-(1-(3-((5-bromopyrimidin-2-yl)amino)pyrrolidin-1-yl)phthalazin-6-yl)acrylamide formate (37.6 mg, 11%) as a white solid. ¹H NMR (400 MHz, DMSO-d₆): δ 2.06-2.11 (m, 1H), 2.24-2.29 (m, 1H), 3.78-3.80 (m, 1H), 3.86-3.92 (m, 1H), 3.99-4.04 (m, 1H), 4.11-4.16 (m, 1H), 4.43-4.48 (m, 1H), 5.85 (dd, J=1.6, 10.0 Hz, 1H), 6.35 (dd, J=1.6, 12.8 Hz, 1H), 6.50 (dd, J=10.4, 17.2 Hz, 1H), 7.85-7.90 (m, 2H), 8.18 (s, 1H), 8.26 (d, J=9.2 Hz, 1H), 8.36-8.41 (m, 3H), 8.91 (s, 1H), 10.66 (s, 1H). [M+H] MS Calc'd C₁₉H₁₈BrN₇O, 440.1; Found: 440.1

### Example 51: (R)—N-(1-(34(5-ethynylpyrimidin-2-yl)amino)pyrrolidin-1-yl)phthalazin-6-yl)acrylamide

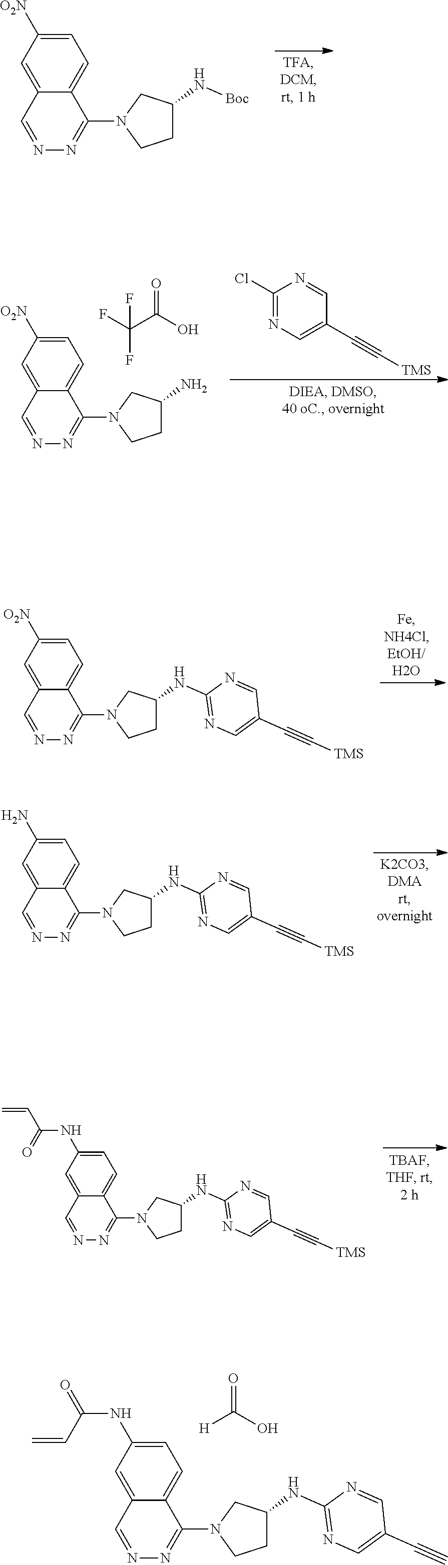

Step 1: (R)-1-(6-nitrophthalazin-1-yl)pyrrolidin-3-amine 2,2,2-trifluoroacetate

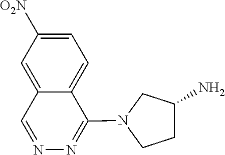 [SMILES: N[C@@H]1CCN(c2nncc3cc([N+](=O)[O-])ccc23)C1]

To a solution of (R)-tert-butyl (1-(6-nitrophthalazin-1-yl)pyrrolidin-3-yl)carbamate (100 mg, 0.28 mmol) in DCM (5 mL) was added TFA (1 mL). The reaction mixture was stirred at room temperature for 1 h. The reaction mixture was concentrated to give (R)-1-(6-nitrophthalazin-1-yl)pyrrolidin-3-amine 2,2,2-trifluoroacetate (100 mg, 100%) as yellow oil. [M+H] Calc'd for C₁₂H₁₃N₅O₂, 260.1; Found, 260.2.

Step 2: (R)—N-(1-(6-nitrophthalazin-1-yl)pyrrolidin-3-yl)-5-((trimethylsilyl)ethynyl)pyrimidin-2-amine

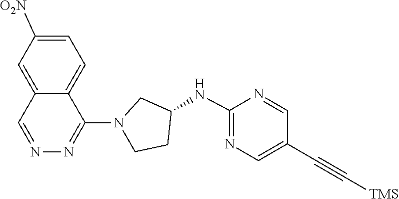 [SMILES: C[Si](C)(C)C#Cc1cnc(N[C@@H]2CCN(c3nncc4cc([N+](=O)[O-])ccc34)C2)nc1]

To a solution of (R)-1-(6-nitrophthalazin-1-yl)pyrrolidin-3-amine 2,2,2-trifluoroacetate (100 mg, 0.27 mmol) in DMSO (5 mL) was added 2-chloro-5-((trimethylsilyl)ethynyl)pyrimidine (65 mg, 0.30 mmol) and DIEA (145 mg, 1.1 mmol) at rt. Then the mixture was stirred at 40° C. overnight. The reaction mixture was quenched with water (15 mL) and extracted with EA (10 mL*3). The combined organic layers were washed with brine (100 mL), dried over anhydrous sodium sulfate, filtered and concentrated. The residue was purified by silica gel column chromatography (PE:EA=1:1) to afford (R)—N-(1-(6-nitrophthalazin-1-yl)pyrrolidin-3-yl)-5-((trimethylsilyl)ethynyl)pyrimidin-2-amine (50 mg, 42%) as a yellow solid. [M+H] MS Calc'd C₂₁H₂₃N₇O₂Si, 434.2; Found: 434.2

Step 3: (R)-1-(3-((5-((trimethylsilyl)ethynyl)pyrimidin-2-yl)amino)pyrrolidin-1-yl)phthalazin-6-amine

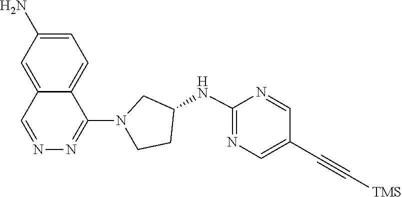 [SMILES: C[Si](C)(C)C#Cc1cnc(N[C@@H]2CCN(c3nncc4cc(N)ccc34)C2)nc1]

To a solution of (R)—N-(1-(6-nitrophthalazin-1-yl)pyrrolidin-3-yl)-5-((trimethylsilyl)ethynyl)pyrimidin-2-amine (270 mg, 0.62 mmol) in EtOH (30 mL) and H₂O (10 mL) was added Fe (350 mg, 6.2 mmol) and NH₄Cl (350 mg, 6.5 mmol) at rt. Then the mixture was refluxed for 3 h. After cooled to rt, the reaction mixture was filtered and the filtrate was concentrated. The residue was adjusted pH 8 with sat. NaHCO₃ and extracted with EA (20 mL*3). The combined organic layers were washed with brine (20 mL), dried over anhydrous sodium sulfate, filtered and concentrated to give (R)-1-(3-((5-((trimethylsilyl)ethynyl)pyrimidin-2-yl)amino)pyrrolidin-1-yl)phthalazin-6-amine (240 mg, 96%) as a yellow solid. [M+H] MS Calc'd C₂₁H₂₅N₇Si, 404.2; Found: 404.2

Step 4: (R)—N-(1-(3-((5-((trimethylsilyl)ethynyl)pyrimidin-2-yl)amino)pyrrolidin-1- yl)phthalazin-6-yl)acrylamide

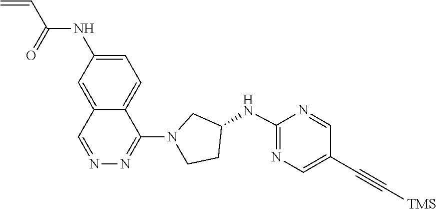 [SMILES: C=CC(=O)Nc1ccc2c(N3CC[C@@H](Nc4ncc(C#C[Si](C)(C)C)cn4)C3)nncc2c1]

To a solution of (R)-1-(3-((5-bromopyrimidin-2-yl)amino)pyrrolidin-1-yl)phthalazin-6-amine (240 mg, 0.59 mmol) in DMA (10 mL) was added acryloyl chloride (108 mg, 1.20 mmol) and K₂CO₃ (165 mg, 1.20 mmol) at 0° C. Then the mixture was warmed to rt and stirred overnight. The reaction mixture was quenched with water (30 mL) and extracted with EA (10 mL*3). The combined organic layers were washed with brine (20 mL), dried over anhydrous sodium sulfate, filtered and concentrated. The residue purified by prep-HPLC to afford (R)—N-(1-(3-((5-((trimethylsilyl)ethynyl)pyrimidin-2-yl)amino)pyrrolidin-1-yl)phthalazin-6-yl)acrylamide (20 mg, 7.5%) as a yellow solid. [M+H] MS Calc'd C₂₄H₂₇N₇OSi, 458.2; Found: 458.2.

Step 5: (R)—N-(1-(3-((5-ethynylpyrimidin-2-yl)amino)pyrrolidin-1-yl)phthalazin-6-yl)acrylamide formate

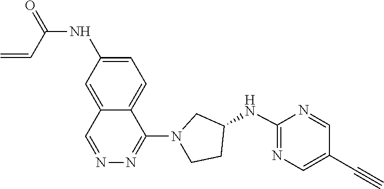 [SMILES: C#Cc1cnc(N[C@@H]2CCN(c3nncc4cc(NC(=O)C=C)ccc34)C2)nc1]

To a solution of (R)—N-(1-(3-((5-((trimethylsilypethynyl)pyrimidin-2-yl)amino)pyrrolidin-1-yl)phthalazin-6-yl)acrylamide (30 mg, 0.066 mmol) in THF (10 mL) was added a solution of TBAF in THF (0.07 mL, 0.07 mmol). Then the mixture was stirred at rt for 2 h. The reaction mixture was quenched with water (30 mL) and extracted with EA (10 mL*3). The combined organic layers were washed with brine (20 mL), dried over anhydrous sodium sulfate, filtered and concentrated. The residue purified by prep-HPLC to afford (R)—N-(1-(3-((5-ethynylpyrimidin-2-yl)amino)pyrrolidin-1-yl)phthalazin-6-yl)acrylamide formate (12.3 mg, 50%) as a yellow solid. ¹H NMR (400 MHz, DMSO-d₆): δ 2.07-2.12 (m, 1H), 2.25-2.30 (m, 1H), 3.78-3.82 (m, 1H), 3.87-3.93 (m, 1H), 4.00-4.04 (m, 1H), 4.12-4.16 (m, 1H), 4.51-4.55 (m, 1H), 5.85 (dd, J=2.0, 10.0 Hz, 1H), 6.35 (dd, J=2.0, 17.2 Hz, 1H), 6.50 (dd, J=10.0, 16.8 Hz, 1H), 7.89 (dd, J=2.0, 9.2 Hz, 1H), 8.05 (d, J=6.4Hz, 1H), 8.15 (s, 1H), 8.26 (d, J=9.2 Hz, 1H), 8.37-8.44 (m, 3H), 8.91 (s, 1H), 10.65 (s, 1H). [M+H] MS Calc'd C₂₁H₁₉N₇O, 386.2; Found: 386.2.

### Example 52: Synthesis of (R)—N-(4-(azetidin-1-yl)-1-(3-((5-bromopyrimidin-2-yl)amino)pyrrolidin-1-yl)phthalazin-6-yl)acrylamide

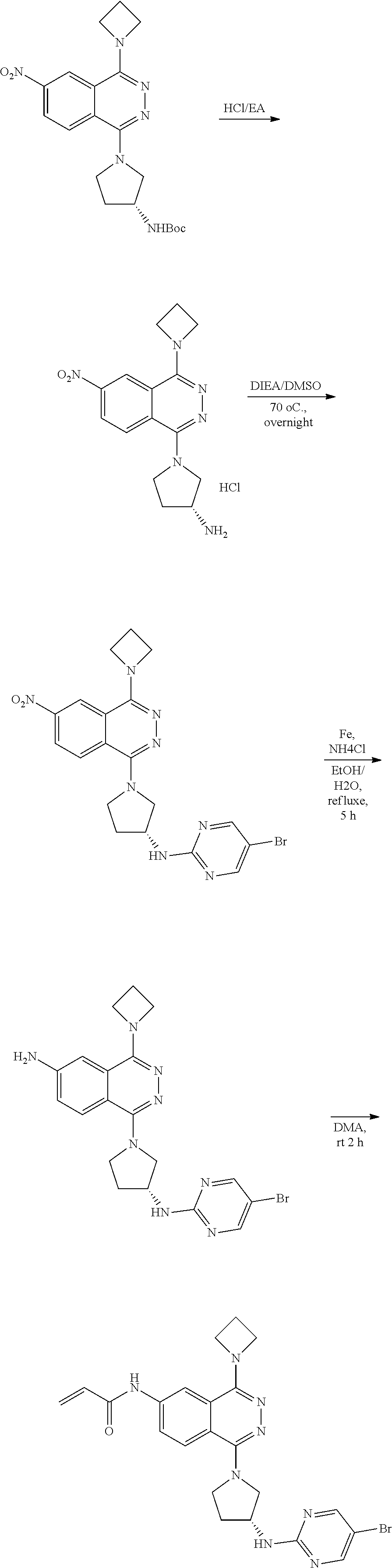 [SMILES: CC(C)(C)OC(=O)N[C@@H]1CCN(c2nnc(N3CCC3)c3cc([N+](=O)[O-])ccc23)C1 ; N[C@@H]1CCN(c2nnc(N3CCC3)c3cc([N+](=O)[O-])ccc23)C1 ; Cl ; O=[N+]([O-])c1ccc2c(N3CC[C@@H](Nc4ncc(Br)cn4)C3)nnc(N3CCC3)c2c1 ; Nc1ccc2c(N3CC[C@@H](Nc4ncc(Br)cn4)C3)nnc(N3CCC3)c2c1 ; C=CC(=O)Nc1ccc2c(N3CC[C@@H](Nc4ncc(Br)cn4)C3)nnc(N3CCC3)c2c1]

Step 1: (R)-1-(4-(azetidin-1-yl)-6-nitrophthalazin-1-yl)pyrrolidin-3-amine hydrochloride

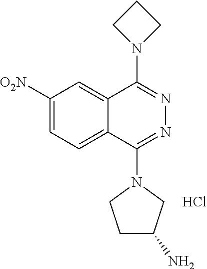 [SMILES: N[C@@H]1CCN(c2nnc(N3CCC3)c3cc([N+](=O)[O-])ccc23)C1 ; Cl]

To a solution of (R)-tert-butyl(1-(4-(azetidin-1-yl)-6-nitrophthalazin-1-yl)pyrrolidin-3-yl)carbamate (400 mg, 0.96 mmol) in EA (5 mL) was added 4 M HCl/EA (15 mL) at room temperature. The reaction mixture was stirred at room temperature for 2 h. The reaction mixture was concentrated under vacuum to give the crude (R)-1-(4-(azetidin-1-yl)-6-nitrophthalazin-1-yl)pyrrolidin-3-amine hydrochloride (340 mg, 100%) as a yellow solid. [M+H] MS Calc'd C₁₅H₁₈N₆O₂, 315.2; Found: 315.2.

Step 2: (R)—N-(1-(4-(azetidin-1-yl)-6-nitrophthalazin-1-yl)pyrrolidin-3-yl)-5-bromopyrimidin-2-amine

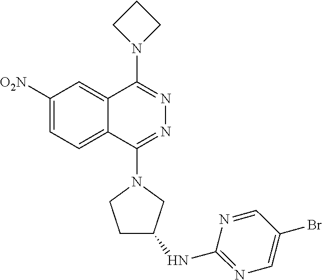 [SMILES: O=[N+]([O-])c1ccc2c(N3CC[C@@H](Nc4ncc(Br)cn4)C3)nnc(N3CCC3)c2c1]

To a solution of (R)-1-(4-(azetidin-1-yl)-6-nitrophthalazin-1-yl)pyrrolidin-3-amine hydrochloride (340 mg, 0.97 mmol) in DMSO (10 mL) was added 5-bromo-2-chloropyrimidine (190 mg, 0.97 mmol) and DIEA (380 mg, 2.91 mmol) at rt. Then the mixture was stirred at 70° C. overnight. The reaction mixture was cooled to rt, diluted with water (30 mL) and extracted with EA (20 mL*3). The combined organic layers were washed with brine (20 mL), dried over anhydrous sodium sulfate, filtered and concentrated. The residue purified by silica gel column chromatography (DCM:MeOH=20:1) to afford (R)—N-(1-(4-(azetidin-1-yl)-6-nitrophthalazin-1-yl)pyrrolidin-3-yl)-5-bromopyrimidin-2-amine (240 mg, 53%) as a yellow solid. [M+H]MS Calc'd C₁₉H₁₉BrN₈O₂, 471.1; Found: 471.1.

Step 3: (R)-4-(azetidin-1-yl)-1-(3-((5-bromopyrimidin-2-yl)amino)pyrrolidin-1-yl)phthalazin-6-amine

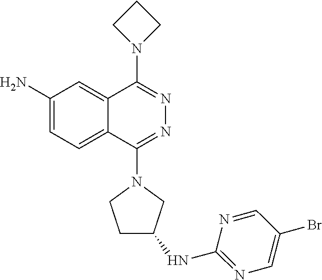 [SMILES: Nc1ccc2c(N3CC[C@@H](Nc4ncc(Br)cn4)C3)nnc(N3CCC3)c2c1]

To a solution of (R)—N-(1-(4-(azetidin-1-yl)-6-nitrophthalazin-1-yl)pyrrolidin-3-yl)-5-bromopyrimidin-2-amine (240 mg, 0.51 mmol) in EtOH (30 mL) and H₂O (10 mL) was added Fe (285 mg, 5.1 mmol) and NH₄C1 (275 mg, 5.1 mmol) at rt. Then the mixture was refluxed for 5 h. After cooled to rt, the reaction mixture was filtered and the filtrate was concentrated. The residue was adjusted pH 8 with sat. NaHCO₃ and extracted with EA (20 mL*3). The combined organic layers were washed with brine (20 mL), dried over anhydrous sodium sulfate, filtered and concentrated to afford (R)-4-(azetidin-1-yl)-1-(3 -((5-bromopyrimidin-2-yl)amino)pyrrolidin-1-yl)phthalazin-6-amine (200 mg, 89%) as a yellow solid. [M+H] MS Calc'd C₁₉H₂₁BrN₈, 441.1; Found: 441.1.

Step 4: (R)—N-(4-(azetidin-1-yl)-1-(3-((5-bromopyrimidin-2-yl)amino)pyrrolidin-1-yl)phthalazin-6-yl)acrylamide formate

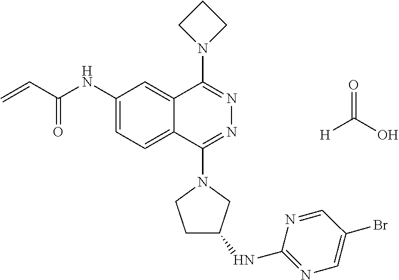 [SMILES: C=CC(=O)Nc1ccc2c(N3CC[C@@H](Nc4ncc(Br)cn4)C3)nnc(N3CCC3)c2c1 ; O=CO]

To a solution of (R)-4-(azetidin-1-yl)-1-(3-((5-bromopyrimidin-2-yl)amino)pyrrolidin-1-yl)phthalazin-6-amine (200 mg, 0.45 mmol) in DMA (5 mL) was added acryloyl chloride (83 mg, 0.91 mmol) and K₂CO₃ (126 mg, 0.91 mmol) at 0° C. Then the mixture was warmed to rt and stirred for 2 h. The reaction mixture was filtered and the filtrate was purified by prep-HPLC to afford to (R)—N-(4-(azetidin-1-yl)-1-(3 -((5-bromopyrimidin-2-yl)amino)pyrrolidin-1-yl)phthalazin-6-yl)acryl amide formate (88.5 mg, 39%) as a yellow solid. ¹H NMR (400 MHz, CD₃OD): δ 2.14-2.19 (m, 1H), 2.31-2.40 (m, 3H), 3.67-3.72 (m, 3H), 3.80-3.84 (m, 1H), 3.93-3.97 (m, 1H), 4.06-4.10 (m, 1H), 4.35 (t, J=5.6 Hz, 2H), 4.56 (t, J=4.2 Hz, 1H), 5.93 (t, J=5.6 Hz, 1H), 6.53 (d, J=6.0 Hz, 2H), 8.01 (dd, J=2.0, 9.2 Hz, 1H), 8.36-8.40 (m, 3H), 8.51 (s, 1H), 8.90 (d, J=2.4 Hz, 1H). [M+H] MS Calc'd C₂₂H₂₃BrN₈O, 495.1; Found: 495.1.

### Example 53: (R)—N-(4-(azetidin-1-yl)-1-(34(6-ethynyl-1,2,4-triazin-3-yl)amino)pyrrolidin-1-yl)phthalazin-6-yl)acrylamide

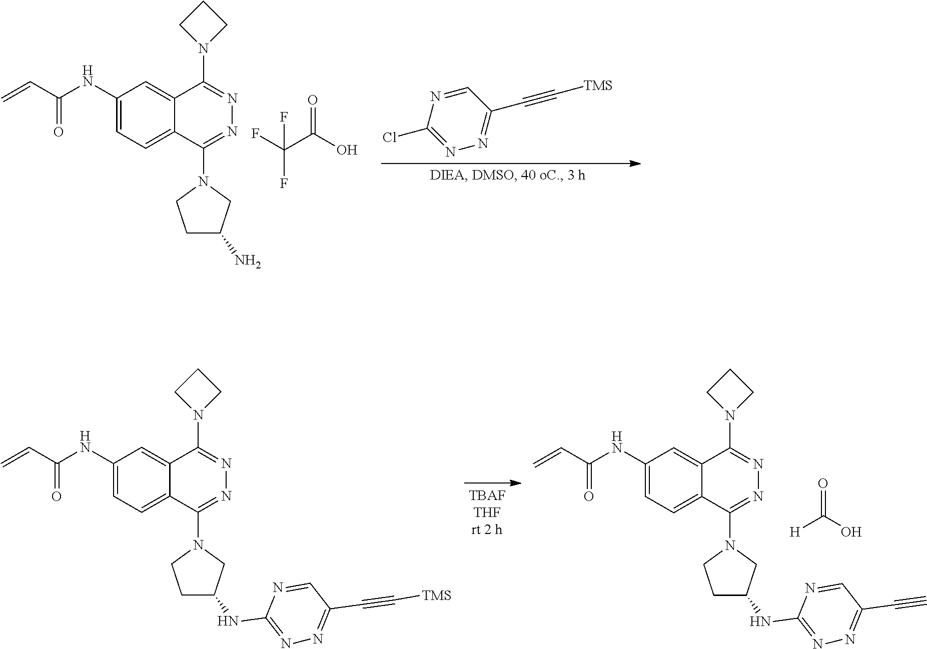

Step 1: (R)—N-(4-(azetidin-1-yl)-1-(3-((6-((trimethylsilyl)ethynyl)-1,2,4-triazin-3-yl)amino)pyrrolidin-1-yl)phthalazin-6-yl)acrylamide

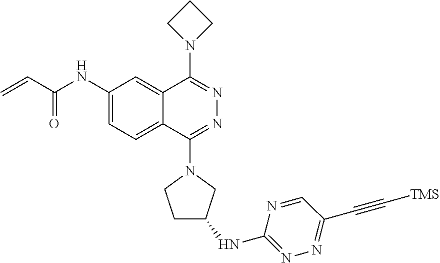 [SMILES: C=CC(=O)Nc1ccc2c(N3CC[C@@H](Nc4ncc(C#C[Si](C)(C)C)nn4)C3)nnc(N3CCC3)c2c1]

To a solution of (R)—N-(1-(3-aminopyrrolidin-1-yl)-4-(azetidin-1-yl)phthalazin-6-yl)acrylamide 2,2,2-trifluoroacetate (80 mg, 0.18 mmol) in DMSO (5 mL) was added 3-chloro-6-((trimethylsilyl)ethynyl)-1,2,4-triazine (37 mg, 0.18 mmol) and DIEA (70 mg, 0.54 mmol) at rt. Then the mixture was stirred at 40° C. for 3 h. The reaction mixture was quenched with water (15 mL) and extracted with EA (10 mL*3). The combined organic layers were washed with brine (100 mL), dried over anhydrous sodium sulfate, filtered and concentrated. The residue was purified by prep-HPLC to afford (R)—N-(4-(azetidin-1-yl)-1-(3-((6-((trimethylsilyl)ethynyl)-1,2,4-triazin-3-yl)amino)pyrrolidin-1-yl)phthalazin-6-yl)acrylamide (17 mg, 18%) as a yellow solid. [M+H] MS Calc'd C₂₆H₃₁N₉OSi, 514.2; Found: 514.2.

Step 2: (R)—N-(4-(azetidin-1-yl)-1-(3-((6-ethynyl-1,2,4-triazin-3-yl)amino)pyrrolidin-1-yl)phthalazin-6-vl)acrylamide formate

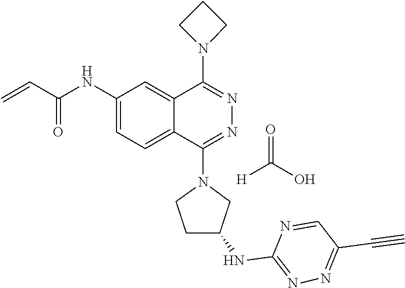

To a solution of (R)—N-(4-(azetidin-1-yl)-1-(3-((6-((trimethylsilyl)ethynyl)-1,2,4-triazin-3-yl)amino)pyrrolidin-1-yl)phthalazin-6-yl)acrylamide (17 mg, 0.033 mmol) in THF (5 mL) was added a solution of TBAF in THF (0.05 mL, 0.05 mmol). Then the mixture was stirred at rt for 2 h. The reaction mixture was quenched with water (15 mL) and extracted with EA (5 mL*3). The combined organic layers were washed with brine (10 mL), dried over anhydrous sodium sulfate, filtered and concentrated. The residue purified by prep-HPLC to afford (R)—N-(4-(azetidin-1-yl)-1-(3-((6-ethynyl-1,2,4-triazin-3-yl)amino)pyrrolidin-1-yl)phthalazin-6-yl)acrylamide formate (9.5 mg, 65%) as a yellow solid. ¹H NMR (400 MHz, CD₃OD): δ 2.25-2.28 (m, 1H), 2.44-2.49 (m, 1H), 2.55-2.63 (m, 2H), 3.77-3.89 (m, 2H), 3.87-4.01 (m, 1H), 4.03 (s, 1H), 4.06-4.18 (m, 1H), 4.57 (t, J=8.0 Hz, 4H), 4.74 (br s, 1H), 5.90 (dd, J=4.8, 7.2 Hz, 1H), 6.49-6.51 (m, 2H), 7.96 (dd, J=2.4, 8.8 Hz, 1H), 8.38-8.48 (m, 2H), 8.92 (s, 1H), 8.93 (s, 1H). [M+H] MS Calc'd C₂₃H₂₃N₉O, 442.2; Found: 442.2.

### Example 54: (R)—N-(4-(3-cyanoazetidin-1-yl)-1-(3-((5-cyanopyrimidin-2-yl)amino)pyrrolidin-1-yl)phthalazin-6-yl)acrylamide

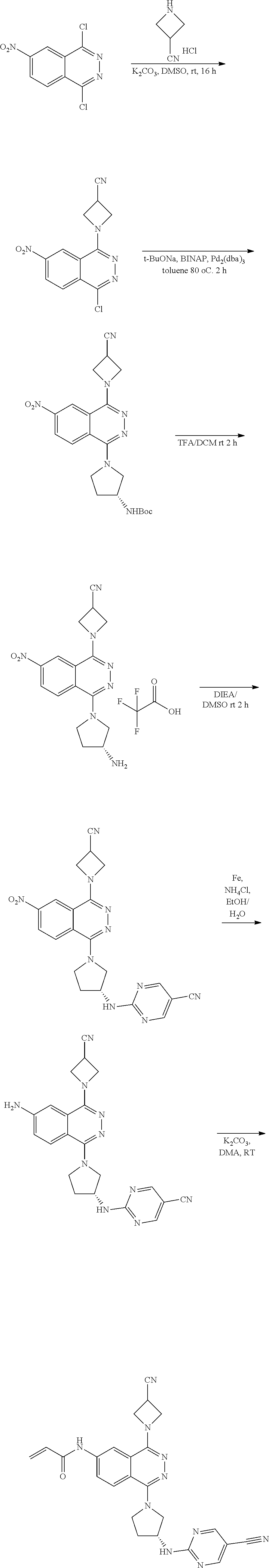

Step 1: 1-(4-chloro-7-nitrophthalazin-1-yl)azetidine-3-carbonitrile

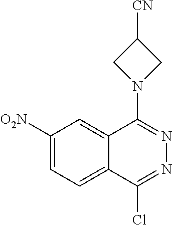 [SMILES: N#CC1CN(c2nnc(Cl)c3ccc([N+](=O)[O-])cc23)C1]

To a solution of 1,4-dichloro-6-nitrophthalazine (6.0 g, 24.5 mmol) in DMSO (40 mL) was added azetidine-3-carbonitrile hydrochloride (2.9 g, 24.5 mmol) and K₂CO₃ (10.1 g, 73.7 mmol) at rt. Then the mixture was stirred at rt for 16 h. The reaction mixture was cooled to rt, diluted with water (100 mL) and extracted with EA (50 mL*3). The combined organic layers were washed with brine (50 mL), dried over anhydrous sodium sulfate, filtered and concentrated. The residue purified by silica gel column chromatography (PE:EA=1:1) to afford 1-(4-chloro-7-nitrophthalazin-1-yl)azetidine-3-carbonitrile (2.9 g, 26%) as a brown solid. [M+H] MS Calc'd C₁₂H₈ClN₅O₂, 290.0; Found: 290.0.

Step 2: (R)-tert-butyl (1-(4-(3-eyanoazetidin-1-yl)-6-nitrophthalazin-1-yl)pyrrolidin-3-yl)carbamate

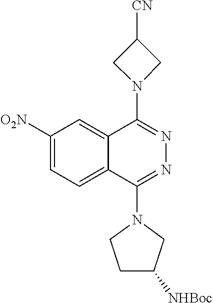 [SMILES: CC(C)(C)OC(=O)N[C@@H]1CCN(c2nnc(N3CC(C#N)C3)c3cc([N+](=O)[O-])ccc23)C1]

To a solution of 1-(4-chloro-7-nitrophthalazin-1-yl)azetidine-3-carbonitrile (2.7 g, 9.3 mmol) and (R)-tert-butyl pyrrolidin-3-ylcarbamate (6.9 g, 37.2 mmol) in toluene (100 mL) was added BINAP (463 mg, 0.74 mmol), Pd₂(dba)₃ (214 mg, 0.37 mmol) and t-BuONa (1.79 mg, 18.62 mmol) at room temperature under N₂ atmosphere. Then the mixture was stirred at 80° C. for 12 h. The reaction mixture was cooled to room temperature and filtered. The filtrate was concentrated and purified by silica gel column chromatography (PE:EA=1:1) to give (R)-tert-butyl (1-(4-(3-cyanoazetidin-1-yl)-6-nitrophthalazin-1-yl)pyrrolidin-3-yl)carbamate (0.6 g, 14%) as a brown solid. [M+H] MS Calc'd C₂₁H₂₅N₇O₄, 440.2; Found: 440.2.

Step 3: (R)-1-(4-(3-aminopyrrolidin-1-yl)-7-nitrophthalazin-1-yl)azetidine-3-carbonitrile 2,2,2-trifluoroacetate

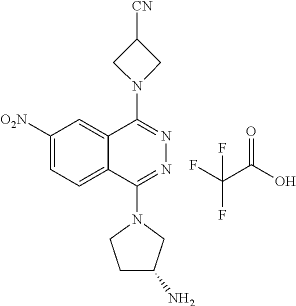

To a solution of (R)-tert-butyl (1-(4-(3-cyanoazetidin-1-yl)-6-nitrophthalazin-1-yl)pyrrolidin-3-yl)carbamate (500 mg, 1.14 mmol) in DCM (5 mL) was added TFA (2.5 mL). The reaction mixture was stirred at room temperature for 2 h. The reaction mixture was concentrated to give (R)-1-(4-(3-aminopyrrolidin-1-yl)-7-nitrophthalazin-1-yl)azetidine-3-carbonitrile 2,2,2-trifluoroacetate (386 mg, crude) as brown oil. [M+H] Calc'd for C₁₆H₁₇N₇O₂, 340.1; Found, 340.1.

Step 4: (R)-2-((1-(4-(3-cyanoazetidin-1-yl)-6-nitrophthalazin-1-yl)pyrrolidin-3-yl)amino)pyrimidine-5-carbonitrile

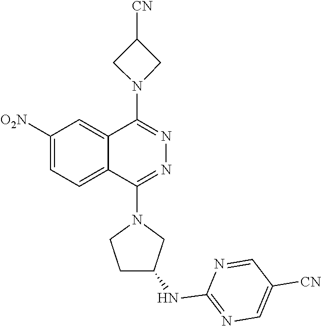 [SMILES: N#Cc1cnc(N[C@@H]2CCN(c3nnc(N4CC(C#N)C4)c4cc([N+](=O)[O-])ccc34)C2)nc1]

To a solution of (R)-1-(4-(3-aminopyrrolidin-1-yl)-7-nitrophthalazin-1-yl)azetidine-3-carbonitrile 2,2,2-trifluoroacetate (386 mg, 1.14 mmol) in DMSO (10 mL) was added 2-chloropyrimidine-5-carbonitrile (174 mg, 1.25 mmol) and DIEA (734 g, 5.69 mmol) at rt. Then the mixture was stirred at rt for 2 h. The reaction mixture was quenched with water (15 mL) and extracted with EA (10 mL*3). The combined organic layers were washed with brine (10 mL), dried over anhydrous sodium sulfate, filtered and concentrated. The residue was purified by silica gel column chromatography (PE:EA=1:1) to afford (R)-2-((1-(4-(3-cyanoazetidin-1-yl)-6-nitrophthalazin-1-yl)pyrrolidin-3-yl)amino)pyrimidine-5-carbonitrile (251 mg, 50%) as a brown solid. [M+H] MS Calc'd C₂₁H₁₈N₁₀O₂, 443.2; Found: 443.2.

Step 5: (R)-2-((1-(6-amino-4-(3-cyanoazetidin-1-yl)phthalazin-1-yl)pyrrolidin-3-yl)amino)pyrimidine-5-carbonitrile

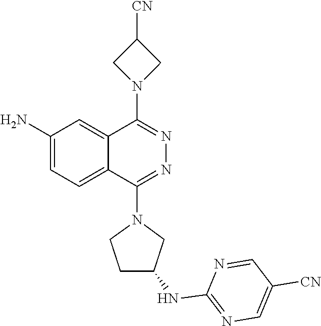 [SMILES: N#Cc1cnc(N[C@@H]2CCN(c3nnc(N4CC(C#N)C4)c4cc(N)ccc34)C2)nc1]

To a solution of (R)-2-((1-(4-(3-cyanoazetidin-1-yl)-6-nitrophthalazin-1-yl)pyrrolidin-3-yl)amino)pyrimidine-5-carbonitrile (250 mg, 0.57 mmol) in EtOH (15 mL) and H₂O (15 mL) was added Fe (216 mg, 5.66 mmol) and NH₄Cl (202 mg, 5.66 mmol) at rt. Then the mixture was stirred at 60° C. for 2 h. After cooled to rt, the reaction mixture was filtered and the filtrate was concentrated. The residue was adjusted to pH 8 with sat. NaHCOaq and extracted with EA (20 mL*3). The combined organic layers were washed with brine (10 mL), dried over anhydrous sodium sulfate, filtered and concentrated. The residue was purified by silica gel column chromatography (DCM:MeOH=20:1) to give (R)-2-((1-(6-amino-4-(3-cyanoazetidin-1-yl)phthalazin-1-yl)pyrrolidin-3-yl)amino)pyrimidine-5-carbonitrile (200 mg, 61%) as a white solid. [M+H] MS Calc'd C₂₁H₂₀N₁₀, 413.2; Found: 413.2.

Step 6: (R)—N-(4-(3-cyanoazetidin-1-yl)-1-(3-((5-cyanopyrimidin-2-yl)amino)pyrrolidin-1-yl)phthalazin-6-yl)acrylamide

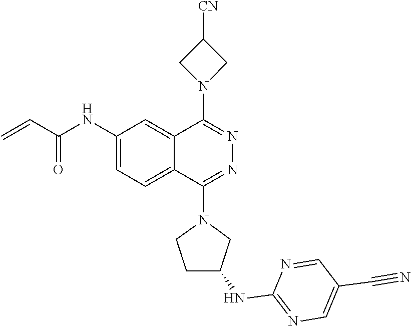 [SMILES: C=CC(=O)Nc1ccc2c(N3CC[C@@H](Nc4ncc(C#N)cn4)C3)nnc(N3CC(C#N)C3)c2c1]

To a solution of (R)-2-((1-(6-amino-4-(3-cyanoazetidin-1-yl)phthalazin-1-yl)pyrrolidin-3-yl)amino)pyrimidine-5-carbonitrile (150 mg, 0.36 mmol) in DMA (5 mL) was added acryloyl chloride (65 mg, 0.72 mmol) and K₂CO₃ (100 mg, 0.72 mmol) at 0° C. Then the mixture was warmed to rt and stirred for 30 min. The reaction mixture was quenched with water (30 mL) and extracted with DCM (10 mL*3). The combined organic layers were washed with brine (20 mL), dried over anhydrous sodium sulfate, filtered and concentrated. The residue purified by prep-HPLC to afford (R)—N-(4-(3-cyanoazetidin-1-yl)-1-(3-((5-cyanopyrimidin-2-yl)amino)pyrrolidin-1-yl)phthalazin-6-yl)acrylamide (79.2 mg, 46%) as an off-white solid. ¹H NMR (400 MHz, DMSO-d₆): δ 2.04-2.09 (m, 1H), 2.25-2.30 (m, 1H), 3.62-4.01 (m, 5H), 4.29-4.34 (m, 2H), 4.44-4.57 (m, 3H), 5.87 (dd, J=2.0, 10.0 Hz, 1H), 6.37 (dd, J=1.6, 16.8 Hz, 1H), 6.49 (dd, J=10.0, 16.8 Hz, 1H), 7.96 (dd, J=2.0, 9.2 Hz, 1H), 8.20 (d, J=9.2 Hz, 1H), 8.43 (d, J=2.0 Hz, 1H), 8.66-8.76 (m, 3H), 10.69 (s, 1H). [M+H] MS Calc'd C₂₄H₂₂N₁₀O, 467.2; Found: 467.2.

### II. Biological Evaluation

### Example 1—Assay condition 2 (thiol containing conditions)

Objective: The IC₅₀ profile of test compounds was determined using protein kinases. IC₅₀ values were measured by testing 10 concentrations (1×10⁻⁰⁴M, 3×10⁻⁰⁵M, 1×10⁻⁰⁵M, 3×10⁻⁰⁶M, 1×10⁻⁰⁶M, 3×10⁻⁰⁷M, 1×10⁻⁰⁷M, 3×10⁻⁰⁸M, 1×10⁻⁰⁸M, and 3×10⁻⁰⁹M) of each compound in singlicate.

Test compounds: The compounds were provided as pre-weighed powders in vials. The compounds were dissolved to 1×10⁻⁰²M by adding DMSO. 100 μl of each of the resulting stock solutions were transferred into column 2 of four 96 well “master plates”

Prior to testing, the 1×10⁻⁰²M stock solutions in column 2 of the master plates were subjected to a serial, semi-logarithmic dilution using 100% DMSO as a solvent. This resulted in 10 distinct concentrations, with a dilution endpoint of 3×10⁻⁰⁷M/100% DMSO in column 12. Column 1 and 7 were filled with 100% DMSO as controls. Subsequently, 2×10 μl from each well of the serial diluted copy plates were aliquoted with a 96 channel pipettor into two identical sets of “compound dilution plates”.

In the process, 90 μl H₂O were added to each well of a set of compound dilution plates. To minimize potential precipitation, the H₂O was added to each plate only a few minutes before the transfer of the compound solutions into the assay plates. Each plate was shaken thoroughly, resulting in a “compound dilution plate/ 10% DMSO”.

For the assays, 5 μl solution from each well of the compound dilution plates/10% DMSO were transferred into the assay plates. The final volume of the assay was 50 μl. All compounds were tested at 10 final assay concentrations in the range from 1×10⁻⁰⁴M to 3×10⁻⁰⁹M, in singlicate. The final DMSO concentration in the reaction cocktails was 1% in all cases.

Recombinant protein kinases: All protein kinases were expressed in Sf9 insect cells or in E.coli as recombinant GST-fusion proteins or His-tagged proteins, either as full-length or enzymatically active fragments. All kinases were produced from human cDNAs and purified by either GSH-affinity chromatography or immobilized metal. Affinity tags were removed from a number of kinases during purification. The purity of the protein kinases was examined by SDS-PAGE/Coomassie staining, the identity was checked by mass spectroscopy.

Protein kinase assay: A radiometric protein kinase assay (³³PanQinase® Activity Assay) was used for measuring the kinase activity. All kinase assays were performed in 96-well FlashPlates™ from PerkinElmer (Boston, MA, USA) in a 50 μl reaction volume. The reaction cocktail was pipetted in four steps in the following order:
20 μl of assay buffer (standard buffer)
5μl of ATP solution (in H₂O)
5μl of test compound (in 10% DMSO)
20 μl enzyme/substrate mix

The assay for all protein kinases contained 70 mM HEPES-NaOH pH 7.5, 3 mM MgCl₂, 3 mM MnCl2, 3 μM Na-orthovanadate, 1.2 mM DTT, 50 μg/ml PEG20000, ATP (variable concentrations, corresponding to the apparent ATP-K. of the respective kinase), [γ-³³P]-ATP (approx. 9×10⁰⁵cpm per well), protein kinase, and substrate.

The following amounts of enzyme and substrate were used per well:

| | Kinase | Kinase | ATP | | | |
|---|---|---|---|---|---|---|
| Kinase | Conc. | Conc. | Conc. | Substrate | Substrate | Substrate |
| Name | ng/50 μl | nM * | μM | Name | Lot | μg/50 μl |
| CDK12 | 100 | 14.7 | 0.3 | RBER- | 036 | 2 |
| wt/CycK | | | | IRStide | | |

* Maximal molar enzyme assay concentrations, implying enzyme preparations exclusively containing 100% active enzyme

The reaction cocktails were incubated at 30° C. for 60 minutes. The reaction was stopped with 50 μl of 2% (v/v) H₃PO₄, plates were aspirated and washed two times with 200 μl 0.9% (w/v) NaCl. Incorporation of ³³Pi was determined with a microplate scintillation counter (Microbeta, Wallac). All assays were performed with a BeckmanCoulter/SAGIANTM Core System.

Evaluation of raw data: The median value of the counts in column 1 (n=8) of each assay plate was defined as “low control”. This value reflects unspecific binding of radioactivity to the plate in the absence of a protein kinase but in the presence of the substrate. The median value of the counts in column 7 of each assay plate (n=8) was taken as the “high control”, i.e. full activity in the absence of any inhibitor. The difference between high and low control was taken as 100% activity.

As part of the data evaluation the low control value from a particular plate was subtracted from the high control value as well as from all 80 “compound values” of the corresponding plate. The residual activity (in %) for each well of a particular plate was calculated by using the following formula:

Res. Activity (%)=100×[(cpm of compound−low control)/(high control−low control)]

The residual activities for each concentration and the compound IC₅₀ values were calculated using Quattro Workflow V3.1.1 (Quattro Research GmbH, Munich, Germany). The fitting model for the IC₅₀ determinations was “Sigmoidal response (variable slope)” with parameters “top” fixed at 100% and “bottom” at 0%. The fitting method used was a least-squares fit.

Results: The IC₅₀ values for all compounds are compiled in Table 1. This table shows all IC₅₀ values calculated, as well as the Hill slopes of the corresponding curves. All IC₅₀ values that were out of range of the tested concentrations (<3×10⁻⁰⁹M; >1×10⁻⁰⁴M) are marked grey. A Hill slope higher than −0.4 is indicative that the curve is not sigmoidal, very flat or not descending.

### Example 2 —Assay condition 1 (thiol-free conditions)

The IC₅₀ profile of compounds was determined using protein kinase in a customized, thiol free assay. IC₅₀ values were measured by testing 10 concentrations (1×10⁻⁰⁵ M to 3×10⁻¹⁰ M) of each test compound in singlicate against each kinase of interest. Prior to testing, the 1×10⁻⁰³M stock solutions in column 2 of the master plates were subjected to a serial, semi-logarithmic dilution using 100% DMSO as a solvent. This resulted in 10 distinct concentrations, with a dilution endpoint of 3×10⁻⁰⁸ M/100% DMSO in column 12. Column 1 and 7 were filled with 100% DMSO as controls. Subsequently, 2×10 microliter from each well of the serial diluted copy plates were aliquoted with a 96 channel pipettor into two identical sets of “compound dilution plates”. All plates were barcoded for automated identification and tracking purposes. IC50 values were measured by testing 10 concentrations (1×10⁻⁰⁵ M to 3×10⁻¹⁰ M) of each compound in singlicate. All compounds were stored as powder until being solubilized in DMSO. Solubilized compounds were stored as 1×10⁻⁰² M/100% DMSO stock solutions. Prior to the assay process, 90 microliters of H₂O were added to each well of a set of compound dilution plates. To minimize potential precipitation, the H₂O was added to each plate only a few minutes before the transfer of the compound solutions into the assay plates. Each plate was shaken thoroughly, resulting in compound dilution plates with a final of 10% DMSO. For each assay, 5 microliters of solution from each well of the compound dilution plates/10% DMSO were transferred into the assay plate. The final volume of the assay was 50 μl. All compounds were tested at 10 final assay concentrations in the range from 1×10⁻⁰⁵ M to 3×10⁻¹⁰ M, in singlicate. The final DMSO concentration in the reaction cocktails was 1% in all cases. A radiometric protein kinase assay (33PanQinase® Activity Assay) was used for measuring the kinase activity of the protein kinase. All kinase assays were performed in 96-well FlashPlates™ from PerkinElmer (Boston, Mass., USA) in a 50 microliter reaction volume. The reaction cocktail was pipetted in four steps in the following order: 20 microliter of assay buffer (standard buffer) 5 microliter of ATP solution (in H₂O) 5 microliter of test compound (in 10% DMSO) 20 microliter enzyme/subtrate mix. Each assay for the protein kinase contained 70 mM HEPES-NaOH pH 7.5, 3 mM MgCl₂, 3 mM MnCl₂, 3 microM Na-orthovanadate, 1 mM TCEP, 50 μg/ml PEG20000, ATP (corresponding to the apparent ATP-Km of the kinase, see Table A), [gamma-33P]-ATP (approx. 6×10×E5 cpm per well), with the protein kinase and relevant substrate being used in pre-determined amounts, depending on the kinase in question. For all experiments labeled as “Thiol-free”, all glutathione was exchanged from protein preparations so as to be removed from the assay and final buffer conditions contained no thiol- containing reagents. This was done so there would be no interference with the key cysteines in the proteins of interest.

For data analysis, the median value of the counts in column 1 (n=8) of each assay plate was defined as “low control”. This value reflects unspecific binding of radioactivity to the plate in the absence of a protein kinase but in the presence of the substrate. The median value of the counts in column 7 of each assay plate (n=8) was taken as the “high control”, i.e. full activity in the absence of any inhibitor. The difference between high and low control was taken as 100% activity. As part of the data evaluation the low control value from a particular plate was subtracted from the high control value as well as from all 80 “compound values” of the corresponding plate. The residual activity (in %) for each well of a particular plate was calculated by using the following formula:

Res. Activity (%)=100×[(cpm of compound−low control)/(high control−low control)]

The residual activities for each concentration and the compound IC₅₀ values were calculated using Quattro Workflow V3.1.1 (Quattro Research GmbH, Munich, Germany; www.quattroresearch.com). The fitting model for the IC₅₀ determinations was “Sigmoidal response (variable slope)” with parameters “top” fixed at 100% and “bottom” at 0%. The fitting method used was a least-squares fit. As a parameter for assay quality, the Z′-factor (Zhang et al., J. Biomol. Screen. 2: 67-73, 1999) for the low and high controls of each assay plate (n=8) was used. ProQinase's criterion for repetition of an assay plate is a Z′-factor below 0.4 (Iversen et al., J. Biomol. Screen. 3: 247-252, 2006).

Representative data for exemplary compounds disclosed in Table 1 is presented in the following

Table 4.

TABLE 4

| Synthetic | | |
|---|---|---|
| Chemistry | Condition 1 | Condition 2 |
| Example | CDK12 IC₅₀ | CDK12 IC₅₀ |
| 1 | B | ND |
| 2 | A | ND |
| 3 | A | ND |
| 4 | A | ND |
| 5 | A | ND |
| 6 | A | ND |
| 7 | B | ND |
| 8 | A | ND |
| 9 | A | ND |
| 10 | A | ND |
| 11 | A | ND |
| 12 | A | ND |
| 13 | A | ND |
| 14 | A | ND |
| 15 | A | ND |
| 16 | A | ND |
| 17 | ND | ND |
| 18 | A | ND |
| 19 | A | ND |
| 20 | A | ND |
| 21 | A | ND |
| 22 | B | ND |
| 23 | ND | ND |
| 24 | A | ND |
| 25 | A | ND |
| 26 | A | ND |
| 27 | A | ND |
| 28 | A | ND |
| 29 | B | ND |
| 30 | A | ND |
| 31 | A | ND |
| 32 | ND | B |
| 33 | ND | A |
| 34 | A | ND |
| 35 | ND | A |
| 36 | ND | A |
| 37 | ND | A |
| 38 | ND | ND |
| 39 | ND | A |
| 40 | ND | A |
| 41 | ND | A |
| 42 | ND | A |
| 43 | A | ND |
| 44 | A | ND |
| 45 | A | ND |
| 46 | ND | ND |
| 47 | A | ND |
| 48 | A | ND |
| 49 | A | ND |
| 50 | A | ND |
| 51 | A | ND |
| 52 | A | ND |
| 53 | A | ND |
| 54 | A | ND |

Note:
Biochemical assay IC₅₀ data are designated within the following ranges:
A: ≤0.10 μM
B: >0.10 μM to ≤1.0 μM
C: >1.0 μMto ≤10 μM
D: >10 μM to ≤30 μM
E: >30 μM to ≤100 μM

### III. Preparation of Pharmaceutical Dosage Forms

### Example 1: Oral capsule

The active ingredient is a compound of Table 1, or a pharmaceutically acceptable salt thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for injection

The active ingredient is a compound of Table 1, or a pharmaceutically acceptable salt thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1. A compound, or pharmaceutically acceptable salt or solvate thereof, having the structure of Formula (I): 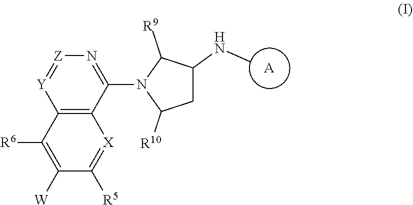
wherein,
Ring A is an optionally substituted heteroaryl selected from pyridine, pyrazine, pyrimidine, quinoline, isoquinoline, quinazoline, pyrazolopyridine, pyrazolopyrimidine, thienopyrimidine, thienopyridine, pyridopyridine, pyridopyrimidine, or triazene;
W is selected from a group having the structure: 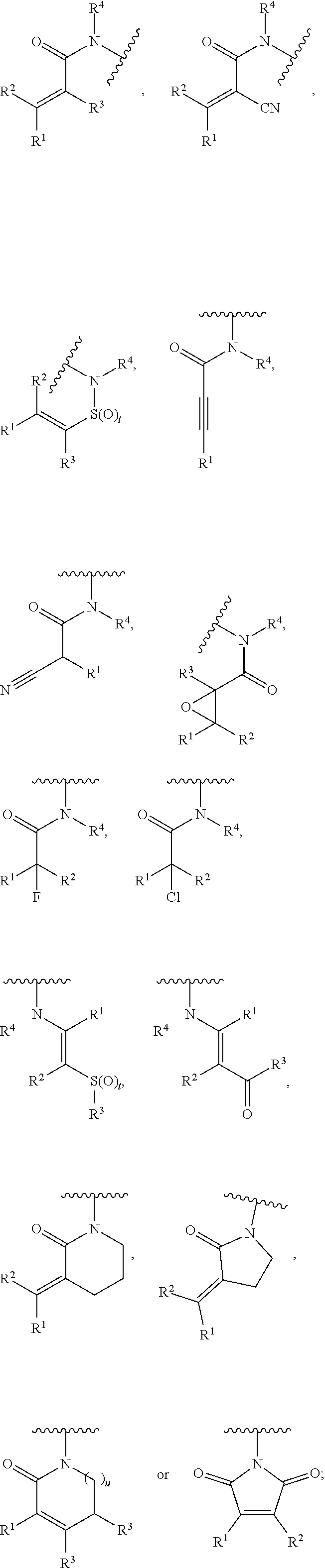
t is 1, or 2; u is 0, 1, or 2;
R¹ R², and R³ are each independently selected from hydrogen, optionally substituted C1-C4 alkyl, or optional substituted heterocyclyl(alkyl);
R⁴ is hydrogen, or optionally substituted C1-C4 alkyl, or optionally, if R³ is optionally substituted C1-C4 alkyl and R⁴ is optionally substituted C1-C4 alkyl, then R³ and R⁴ together join to form a ring;
R⁵ is selected from hydrogen, —CN, —NH₂, halogen, optionally substituted C1-C4 alkyl, optionally substituted C1-C4 alkoxy, or optionally substituted C1-C4 aminoalkyl;
R⁶ is selected from hydrogen, —CN, —NH₂, halogen, optionally substituted C1-C4 alkyl, optionally substituted C1-C4 alkoxy, or optionally substituted C1-C4 aminoalkyl;
X is N or C—H;
Y is N, or C-L1-R¹¹;
Z is N, or C-L2-R⁷;
L1 and L2 are each independently a bond, —O—, or —N(R⁸)—;
R⁷ is selected from hydrogen, —CN, halogen, optionally substituted C1-C4 alkyl, optionally substituted C3-C7 carbocyclyl, optionally substituted carbocyclyl(alkyl), optionally substituted heterocyclyl, optionally substituted heterocyclyl(alkyl);
R⁸ is hydrogen, or optionally substituted C1-C4 alkyl;
R⁹ is selected from hydrogen, or optionally substituted C1-C4 alkyl;
R¹⁰ is selected from hydrogen, or optionally substituted C1-C4 alkyl; and
R¹¹ is selected from hydrogen, —CN, halogen, —NH₂, optionally substituted C1-C4 alkyl, optionally substituted C3-C7 carbocyclyl, optionally substituted carbocyclyl(alkyl), optionally substituted heterocyclyl, or optionally substituted heterocyclyl(alkyl).

2. (canceled)

3. (canceled)

4. (canceled)

5. (canceled)

6. The compound, or pharmaceutically acceptable salt or solvate thereof, of claim 1, wherein W is

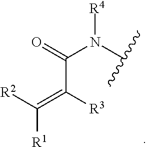

7-21. (canceled) 22. (canceled) 23. (canceled)

24. The compound of claim 1, or pharmaceutically acceptable salt or solvate thereof, wherein R⁵ is hydrogen.

25. The compound of claim 1, or pharmaceutically acceptable salt or solvate thereof, wherein R⁶ is hydrogen.

26. (canceled)

27. (canceled)

28. The compound of claim 1, or pharmaceutically acceptable salt or solvate thereof, wherein Y is N or C—H.

29. (canceled)

30. (canceled)

31. (canceled)

32. (canceled)

33. (canceled)

34. (canceled)

35. (canceled)

36. (canceled)

37. (canceled)

38. The compound of claim 1, or pharmaceutically acceptable salt or solvate thereof, wherein Z is C-L2-R⁷.

39. (canceled)

40. (canceled)

41. (canceled)

42. (canceled)

43. The compound of claim 38, or pharmaceutically acceptable salt or solvate thereof, wherein R⁷ is hydrogen, optionally substituted C1-C4 alkyl, or optionally substituted heterocyclyl.

44. (canceled)

45. (canceled)

46. (canceled)

47. (canceled)

48. The compound of claim 1, or pharmaceutically acceptable salt or solvate thereof, wherein Ring A is an optionally substituted pyrimidine.

49-62. (canceled)

63. The compound of claim 48, or pharmaceutically acceptable salt or solvate thereof, wherein Ring A is optionally substituted with a substituent selected from hydrogen, −CN, halogen, optionally substituted C1-C4 alkyl, optionally substituted C1-C6 alkenyl, optionally substituted C1-C6 alkynyl, optionally substituted C3-C7 carbocyclyl, optionally substituted carbocyclyl(alkyl), optionally substituted heterocyclyl, optionally substituted heterocyclyl(alkyl), optionally substituted C1-C4 alkoxy, optionally substituted C6 aryloxy, −NH₂, —OH, or optionally substituted C1-C4 aminoalkyl.

64. The compound of claim 1, or pharmaceutically acceptable salt or solvate thereof, wherein Ring A is selected from: 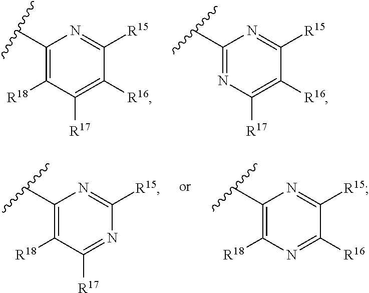
wherein,
R¹⁵ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R²²)₂, —N(R²²)CO₂R²¹, —N(R²²)CON(R²²)₂, —N(R²²)COR²¹, —OC(O)N(R²²)₂, —OSO₂N(R²²)₂, or —N(R²²)SO₃R²¹;
R¹⁶ is selected from hydrogen, halogen, —CN, optionally substituted alkyl, optionally substituted alkenyl, optionally substituted alkynyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R²²)₂, —N(R²²)CO₂R²¹, —N(R²²)CON(R²²)₂, —N(R²²)COR²¹, —OC(O)N(R²²)₂, —OSO₂N(R²²)₂, or —N(R²²)SO₃R²¹;
R¹⁷ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R²²)₂, —N(R²²)CO₂R²¹, —N(R²²)CON(R²²)₂, —N(R²²)COR²¹, —OC(O)N(R²²)₂, —OSO₂N(R²²)₂, or —N(R²²)SO₃R²¹;
R¹ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, —N(R²²)SO₂N(R²²)₂, —CON(R²²)₂, —N(R²²)CO₂R²¹, —N(R²²)CON(R²²)₂, —N(R²²)COR²¹, —OC(O)N(R²²)₂, —OSO₂N(R²²)₂, or —N(R²²)SO₃R²¹;
each R²¹ is independently selected from alkyl, cycloalkyl, cycloalkylalkyl, aryl, aralkyl, heterocyclyl, heterocyclylalkyl, heteroaryl, or heteroarylalkyl; and
each R²² is independently selected from hydrogen, alkyl, cycloalkyl, cycloalkylalkyl, aryl, aralkyl, heterocyclyl, heterocyclylalkyl, heteroaryl, or heteroarylalkyl.

65. (canceled)

66. (canceled)

67. The compound of claim 64, or pharmaceutically acceptable salt or solvate thereof, wherein R¹⁵ is selected from hydrogen, halogen, —CN, and optionally substituted alkyl.

68. The compound of claim 64, or pharmaceutically acceptable salt or solvate thereof, wherein, R¹⁵ is hydrogen.

69. The compound of claim 64, or pharmaceutically acceptable salt or solvate thereof, wherein R¹⁶ is selected from hydrogen, halogen, —CN, optionally substituted alkyl, optionally substituted fluoroalkyl, optionally substituted alkoxy, optionally substituted cycloalkyl, optionally substituted heterocyclyl, optionally substituted cycloalkylalkyl, optionally substituted cycloalkyl-O—, optionally substituted aryl, optionally substituted aralkyl, optionally substituted heteroaryl, optionally substituted heteroaralkyl, optionally substituted aryloxy, optionally substituted aralkyloxy, optionally substituted heteroaryloxy, optionally substituted heteroaralkyloxy, —OR²², —N(R²²)₂, —SO₂R²¹, —N(R²²)SO₂R²¹, —SO₂N(R²²)₂, SO₂N(R²²)₂, N(R²²)SO₂N(R²²)₂, —CON(R²²)₂, —N(R²²)CO₂R²¹, N(R²²)CON(R²²)₂, —N(R²²)COR²¹, —C(O)N(R²²)₂, —OSO₂N(R²²)₂, or —N(R²²)SO₃R²¹.

70. The compound of claim 64, or pharmaceutically acceptable salt or solvate thereof, wherein R¹⁶ is selected from hydrogen, halogen, —CN, and optionally substituted alkyl.

71. The compound of claim 64, or pharmaceutically acceptable salt or solvate thereof, wherein R¹⁶ is hydrogen.

72. The compound of claim 64, or pharmaceutically acceptable salt or solvate thereof, wherein R¹⁶ is selected from optionally substituted alkyl, optionally substituted alkenyl, or optionally substituted alkynyl.

73. The compound of claim 64, or pharmaceutically acceptable salt or solvate thereof, wherein R¹⁶ is selected from optionally substituted alkynyl.

74. The compound of claim 64, or pharmaceutically acceptable salt or solvate thereof, wherein Ring A is: 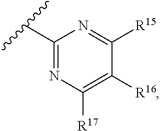
and R¹⁶ is not hydrogen.

75. The compound of claim 64, or pharmaceutically acceptable salt or solvate thereof, wherein Ring A is: 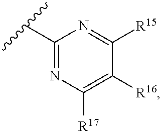
and R¹⁶ is halogen.

76. The compound of claim 64, or pharmaceutically acceptable salt or solvate thereof, wherein Ring A is: 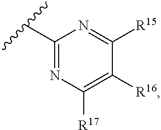
and R¹⁶ is selected from optionally substituted alkyl, optionally substituted alkenyl, or optionally substituted alkynyl.

77. The compound of claim 64, or pharmaceutically acceptable salt or solvate thereof, wherein Ring A is: 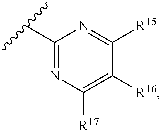
R¹⁵ is hydrogen, R¹⁶ is selected from optionally substituted alkynyl, and R¹⁷ is hydrogen or optionally substituted alkoxy.

78. The compound of claim 64, or pharmaceutically acceptable salt or solvate thereof, wherein Ring A is: 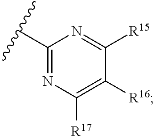
R¹⁵ is hydrogen, R¹⁶ is selected from halogen, —CN, optionally substituted alkyl, optionally substituted fluoroalkyl, optionally substituted alkenyl, optionally substituted alkynyl, and R¹⁷ is hydrogen or optionally substituted alkoxy.

79. The compound of claim 64, or pharmaceutically acceptable salt or solvate thereof, wherein R¹⁷ is hydrogen.

80. The compound of claim 64, or pharmaceutically acceptable salt or solvate thereof, wherein R¹ is selected from hydrogen, halogen, —CN, and optionally substituted alkyl.

81. The compound of claim 64, or pharmaceutically acceptable salt or solvate thereof, wherein R¹ is hydrogen.

82. The compound of claim 64, or pharmaceutically acceptable salt or solvate thereof, wherein R¹⁵ and R¹⁶ are hydrogen.

83. The compound of claim 64, or pharmaceutically acceptable salt or solvate thereof, wherein R¹⁷ and R¹⁸ are hydrogen.

84. The compound of claim 64, or pharmaceutically acceptable salt or solvate thereof, wherein R¹⁵ and R¹⁷ are hydrogen.

85. The compound of claim 1, or pharmaceutically acceptable salt or solvate thereof, having the structure of a compound provided in Table 1.

86. A pharmaceutical composition comprising a compound of claim 1, or pharmaceutically acceptable salt or solvate thereof, and a pharmaceutically acceptable excipient.

87. (canceled)

88. (canceled)

89. (canceled)

90. (canceled)

91. (canceled)

92. A method of treating cancer in a patient in need thereof comprising administering to the patient a compound as described in claim 1, or pharmaceutically acceptable salt or solvate thereof.

93. (canceled)

94. (canceled)


SEQUENCE LISTING (16 sequences)

SEQ ID NO: 1 - protein, 1490 aa, Homo sapiens
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVILPSAEQTTLEASSTPADMQNILAVLLSQLMKTQEPAGSLEENNSDKNSGPQGPRRTPTMPQEEAAACPPHILPPEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 2 - protein, 1481 aa, Homo sapiens
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVILPSAEQTTLEASSTPADMQNILAVLLSQLMKTQEPAGSLEENNSDKNSGPQGPRRTPTMPQEEAAEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 3 - protein, 1201 aa, Homo sapiens
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVILPSAEQTTLEASST

SEQ ID NO: 4 - protein, 159 aa, Homo sapiens
  MOD_RES (1)..(1): Any amino acid
XADITQQLNQSELAVLLNLLQSQTDLSIPQMAQLLNIHSNPEMQQQLEALNQSISALTEATSQQQDSETMAPEESLKEAPSAPVILPSAEQTTLEASSTPADMQNILAVLLSQLMKTQEPAGSLEENNSDKNSGPQGPRRTPTMPQEEAAGRSNGGNAL

SEQ ID NO: 5 - protein, 1490 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSVPQMAQLLNIHSNPEMQQQLEALNQSISALTEATSQQQDSETMAPEESLKEAPSAPVILPSAEQTTLEASSTPADMQNILAVLLSQLMKTQEPAGSLEENNSDKNSGPQGPRRTPTMPQEEAAACPPHILPPEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 6 - protein, 1490 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVIQPSAEQTTLEASSTPADMQNILAVLLSQLMKTQEPAGSLEENNSDKNSGPQGPRRTPTMPQEEAAACPPHILPPEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 7 - protein, 1490 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVILPSAEQMTLEASSTPADMQNILAVLLSQLMKTQEPAGSLEENNSDKNSGPQGPRRTPTMPQEEAAACPPHILPPEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 8 - protein, 1481 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSVPQMAQLLNIHSNPEMQQQLEALNQSISALTEATSQQQDSETMAPEESLKEAPSAPVILPSAEQTTLEASSTPADMQNILAVLLSQLMKTQEPAGSLEENNSDKNSGPQGPRRTPTMPQEEAAEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 9 - protein, 1481 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVIQPSAEQTTLEASSTPADMQNILAVLLSQLMKTQEPAGSLEENNSDKNSGPQGPRRTPTMPQEEAAEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 10 - protein, 1481 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPSRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVILPSAEQMTLEASSTPADMQNILAVLLSQLMKTQEPAGSLEENNSDKNSGPQGPRRTPTMPQEEAAEKRPPEPPGPPPPPPPPPLVEGDLSSAPQELNPAVTAALLQLLSQPEAEPPGHLPHEHQALRPMEYSTRPRPNRTYGNTDGPETGFSAIDTDERNSGPALTESLVQTLVKNRTFSGSLSHLGESSSYQGTGSVQFPGDQDLRFARVPLALHPVVGQPFLKAEGSSNSVVHAETKLQNYGELGPGTTGASSSGAGLHWGGPTQSSAYGKLYRGPTRVPPRGGRGRGVPY

SEQ ID NO: 11 - protein, 1201 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSVPQMAQLLNIHSNPEMQQQLEALNQSISALTEATSQQQDSETMAPEESLKEAPSAPVILPSAEQTTLEASST

SEQ ID NO: 12 - protein, 1201 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVIQPSAEQTTLEASST

SEQ ID NO: 13 - protein, 1201 aa, Artificial Sequence
  Description of Artificial Sequence Synthetic polypeptide
MPNSERHGGKKDGSGGASGTLQPSSGGGSSNSRERHRLVSKHKRHKSKHSKDMGLVTPEAASLGTVIKPLVEYDDISSDSDTFSDDMAFKLDRRENDERRGSDRSDRLHKHRHHQHRRSRDLLKAKQTEKEKSQEVSSKSGSMKDRISGSSKRSNEETDDYGKAQVAKSSSKESRSSKLHKEKTRKERELKSGHKDRSKSHRKRETPKSYKTVDSPKRRSRSPHRKWSDSSKQDDSPSGASYGQDYDLSPSRSHTSSNYDSYKKSPGSTSRRQSVSPPYKEPSAYQSSTRSPSPYSRRQRSVSPYSRRRSSSYERSGSYSGRSPSPYGRRRSSSPFLSKRSLSRSPLPRKSMKSRSRSPAYSRHSSSHSKKKRSSSRSRHSSISPVRLPLNSSLGAELSRKKKERAAAAAAAKMDGKESKGSPVFLPRKENSSVEAKDSGLESKKLPRSVKLEKSAPDTELVNVTHLNTEVKNSSDTGKVKLDENSEKHLVKDLKAQGTRDSKPIALKEEIVTPKETETSEKETPPPLPTIASPPPPLPTTTPPPQTPPLPPLPPIPALPQQPPLPPSQPAFSQVPASSTSTLPPSTHSKTSAVSSQANSQPPVQVSVKTQVSVTAAIPHLKTSTLPPLPLPPLLPGDDDMDSPKETLPSKPVKKEKEQRTRHLLTDLPLPPELPGGDLSPPDSPEPKAITPPQQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIPQMAQLLNIHSNPEMQQQLEALNQSISALTEATSQQQDSETMAPEESLKEAPSAPVILPSAEQMTLEASST

SEQ ID NO: 14 - protein, 159 aa, Artificial Sequence
  Description of Artificial Sequence Synthetic polypeptide
  MOD_RES (1)..(1): Any amino acid
XADITQQLNQSELAVLLNLLQSQTDLSVPQMAQLLNIHSNPEMQQQLEALNQSISALTEATSQQQDSETMAPEESLKEAPSAPVILPSAEQTTLEASSTPADMQNILAVLLSQLMKTQEPAGSLEENNSDKNSGPQGPRRTPTMPQEEAAGRSNGGNAL

SEQ ID NO: 15 - protein, 159 aa, Artificial Sequence
  Description of Artificial Sequence Synthetic polypeptide
  MOD_RES (1)..(1): Any amino acid
XADITQQLNQSELAVLLNLLQSQTDLSIPQMAQLLNIHSNPEMQQQLEALNQSISALTEATSQQQDSETMAPEESLKEAPSAPVIQPSAEQTTLEASSTPADMQNILAVLLSQLMKTQEPAGSLEENNSDKNSGPQGPRRTPTMPQEEAAGRSNGGNAL

SEQ ID NO: 16 - protein, 159 aa, Artificial Sequence
  Description of Artificial Sequence Synthetic polypeptide
  MOD_RES (1)..(1): Any amino acid
XADITQQLNQSELAVLLNLLQSQTDLSIPQMAQLLNIHSNPEMQQQLEALNQSISALTEATSQQQDSETMAPEESLKEAPSAPVILPSAEQMTLEASSTPADMQNILAVLLSQLMKTQEPAGSLEENNSDKNSGPQGPRRTPTMPQEEAAGRSNGGNAL